(19) United States
(12) Patent Application Publication
(10) Pub. No.: US 2026/0341475 A1
(43) Pub. Date: Oct. 29, 2026

(54) Automated Routing of Cytological Smears for Selective Human Review Based on Automatic Cell Classification

(71) Applicant: Scopio Labs Ltd., Tel-Aviv (IL)

(72) Inventors: Erez Naaman, Tel Aviv (IL); Itai Hayut, Tel Aviv (IL); Amit Meitus, Tel Aviv (IL); Ittai Madar, Tel Aviv (IL); Ofek Aizenman, Holon (IL); Orna Bregman Amitai, Tel-Aviv (IL); Anat Shkolyar, Modi'in-Maccabim-Re'ut (IL); Amir Shaham, Givat Shmuel (IL); Roni Hilla Agami Leibowitz, Nir Galim (IL); Bar Zelingher, Shefayim (IL)

(73) Assignee: Scopio Labs Ltd., Tel-Aviv (IL)

(21) Appl. No.: 19/657,525

(22) Filed: Apr. 24, 2026

Related U.S. Application Data
(60) Provisional application No. 63/795,230, filed on Apr. 25, 2025.
(60) Provisional application No. 63/795,532, filed on Apr. 27, 2025.
(60) Provisional application No. 63/889,964, filed on Sep. 29, 2025.

(51) Int. Cl. G16H 30/40; G16H 10/60; G16H 30/20; G16H 50/30

(52) U.S. Cl. CPC G16H 30/40; G16H 10/60; G16H 30/20; G16H 50/30

(57) ABSTRACT

Clinical laboratories face a growing mismatch between rising cytological smear volumes and limited specialist capacity. Methods and systems are disclosed for managing the human review workload associated with cytological smear analysis. An automatic cell classifier determines, from images of cytological smears obtained from multiple patients, hematological cell or cell type quantification metrics (HCQMs) for multiple abnormal cell types. The metrics are compared against respective predetermined thresholds, and samples are selectively presented or not presented as part of a default human review workflow based on the resulting pattern of threshold crossings, so that samples sharing the same count of crossings can be routed differently according to which abnormal cell types are involved. Performance of the automatic cell classifier may be monitored over time by comparison with human review results, and one or more thresholds are increased when performance improves beyond a predetermined level, reducing review volume while maintaining analysis quality.

### CROSS-REFERENCE TO RELATED APPLICATIONS

This application claims priority to: U.S. Provisional Patent Application No. 63/795,230, filed 25 Apr. 2025; U.S. Provisional Patent Application No. 63/795,532, filed 27 Apr. 2025; and U.S. Provisional Patent Application No. 63/889,964, filed 29 Sep. 2025.

### BACKGROUND

Cytological smears, including blood smears, bone marrow smears, and smears prepared from body fluids, play a central role in clinical diagnostics. Microscopic examination of cellular morphology, cell type distributions, and the presence or absence of atypical cellular elements provides information relevant to the diagnosis and monitoring of a wide range of conditions, including hematological malignancies such as leukemia and lymphoma, anemias of varied etiology, infections, bleeding and coagulation disorders, and bone marrow dysfunction. Findings from a cytological smear can substantially influence clinical decision-making, from the identification of conditions requiring urgent intervention, such as acute leukemia presenting with circulating blast cells, to more nuanced determinations regarding disease progression, treatment response, and differential diagnosis. The clinical utility of cytological smear examination thus extends from urgent, life-critical diagnostic scenarios to routine monitoring of chronic conditions.

The worldwide volume of cytological smears prepared and examined each year is substantial and continues to rise. Population growth, aging demographics in developed economies, expansion of cancer and hematology screening programs, rising prevalence of chronic diseases with hematological implications, broader access to laboratory services in emerging economies, and the routine incorporation of blood-based diagnostics into primary and specialty care all contribute to steadily increasing demand. Commercial reference laboratories, hospital laboratories, physician office laboratories, and point-of-care testing facilities each face sustained growth in the number of cytological smears requiring skilled examination, with large reference laboratories in particular processing high daily caseloads.

Manual microscopic examination of cytological smears is a skilled, time-intensive activity performed by professionals such as medical laboratory scientists, cytotechnologists, hematologists, and hematopathologists. Developing reliable morphological judgment across the full range of cell types, including challenging abnormal cell types and rare findings, typically requires years of formal education combined with extensive practical experience. The supply of qualified specialists has not matched the growth in sample volumes. Laboratory workforce studies in multiple regions report persistent vacancy rates for cytology and hematology positions, rising average age of practicing specialists, and attrition from the workforce that outpaces entry. Geographic disparities in specialist distribution further strain review capacity, with many rural and underserved areas lacking convenient access to experienced reviewers. These workforce dynamics are structural rather than transient, and projections for many healthcare systems indicate that they will persist or intensify over the coming decades.

The resulting imbalance between the volume of cytological smears to be examined and the capacity of trained specialists available to examine them carries significant technical and commercial implications. Skilled microscopy is among the costlier components of laboratory operations, and specialist time is an economically constrained resource that laboratories must allocate across a growing and diverse caseload. Laboratories aiming to scale their diagnostic services face a bottleneck that is not readily resolved by expanding headcount, because training pipelines for qualified reviewers extend over years. Reimbursement structures in many healthcare systems do not readily expand with rising per-sample review costs, placing operational and commercial strain on laboratories across public and private sectors. Technically, each cytological smear can contain large numbers of cells across many cell types, with the clinically relevant information often concentrated in a small subset of cells, or in the relative proportions and distributions of different cell populations.

### SUMMARY

Identifying which smears warrant the attention of a specialist, and allocating specialist effort accordingly, is a challenge of both technical and commercial importance to the healthcare industry. In certain implementations, systems and methods are provided for managing the human review workload associated with cytological smear analysis, addressing throughput pressures through selective routing of samples to human review based on quantitative metrics determined from images of cytological smears.

In certain implementations, a computer-implemented method for cytological smear human review workload management is provided. The method comprises receiving a first image of a first cytological smear associated with a first sample obtained from a first patient and receiving a second image of a second cytological smear associated with a second sample obtained from a second patient. The method further comprises determining, by an automatic cell classifier from the first image, first and second hematological cell or cell type quantification metrics (HCQMs) for the first patient, the first and second HCQMs being for first and second abnormal cell types, respectively, the second abnormal cell type being different from the first abnormal cell type, and determining, by the automatic cell classifier from the second image, the first and second HCQMs for the second patient. The method further comprises receiving first and second predetermined thresholds associated with the first and second HCQMs, respectively, comparing the first and second HCQMs for the first patient with the first and second predetermined thresholds, respectively, and identifying that the first HCQM is above the first predetermined threshold and the second HCQM is below the second predetermined threshold, and comparing the first and second HCQMs for the second patient with the first and second predetermined thresholds, respectively, and identifying that the first HCQM is below the first predetermined threshold and the second HCQM is above the second predetermined threshold. The method further comprises presenting, as part of a default human review workflow, cells associated with the first sample, and not presenting, as part of the default human review workflow, cells associated with the second sample.

In certain implementations, a system for cytological smear human review workload management is provided. The system comprises a computer configured to receive a first image of a first cytological smear associated with a first sample obtained from a first patient and to receive a second image of a second cytological smear associated with a second sample obtained from a second patient. The computer is further configured to determine, by an automatic cell classifier from the first image, first and second hematological cell or cell type quantification metrics (HCQMs) for the first patient, the first and second HCQMs being for first and second abnormal cell types, respectively, the second abnormal cell type being different from the first abnormal cell type, and to determine, by the automatic cell classifier from the second image, the first and second HCQMs for the second patient. The computer is further configured to receive first and second predetermined thresholds associated with the first and second HCQMs, respectively, to compare the first and second HCQMs for the first patient with the first and second predetermined thresholds, respectively, and identify that the first HCQM is above the first predetermined threshold and the second HCQM is below the second predetermined threshold, and to compare the first and second HCQMs for the second patient with the first and second predetermined thresholds, respectively, and identify that the first HCQM is below the first predetermined threshold and the second HCQM is above the second predetermined threshold. The computer is further configured to cause cells associated with the first sample to be presented as part of a default human review workflow and to cause cells associated with the second sample to be excluded from presentation as part of the default human review workflow.

In certain implementations, a computer-implemented method for adaptive management of cytological smear human review workload is provided. The method comprises receiving results from an automatic cell classifier for cytological smears of multiple patients over time and monitoring performance of the automatic cell classifier by comparing the results with human review results. The method further comprises adjusting one or more predetermined thresholds for presentation of cells for human review based on the monitored performance, the one or more predetermined thresholds being associated with hematological cell or cell type quantification metrics (HCQMs) for abnormal cell types, and increasing at least one of the one or more predetermined thresholds when the performance of the automatic cell classifier improves beyond a predetermined performance level, thereby reducing a number of cytological smears presented for human review while maintaining analysis quality.

### BRIEF DESCRIPTION OF THE DRAWINGS

The embodiments are herein described by way of example only, with reference to the accompanying drawings.

FIG. 1 illustrates an embodiment of a block diagram of an exemplary system architecture for cytological smear analysis in accordance with various embodiments of the present disclosure;

FIG. 2A illustrates an embodiment of a block diagram of a system configured to control preparation of an additional cytological smear;

FIG. 2B illustrates an embodiment of a computer-implemented method for automatically preparing an additional cytological smear based on quantitative classification confidence analysis;

FIG. 3 illustrates an embodiment of a computer-implemented method for ordering another cytological smear;

FIG. 4 illustrates an embodiment of a computer-implemented method for ordering another cytological smear to improve statistical sampling significance;

FIG. 5 illustrates an embodiment of a computer-implemented method for preparing additional slides based on inadequate smear length;

FIG. 6 illustrates an embodiment of a computer-implemented method for preparing additional slides based on staining inadequacy;

FIG. 7A illustrates an embodiment of a computer-implemented method for preparing additional slides;

FIG. 7B illustrates an embodiment of a block diagram of a system for preparing additional slides;

FIG. 8A illustrates an embodiment of a computer-implemented method for preparing images for human review;

FIG. 8B illustrates an embodiment of a block diagram of a system for preparing images for human review;

FIG. 9 illustrates an embodiment of a computer-implemented method for differential human review of cytological smear analysis;

FIG. 10 illustrates an embodiment of a computer-implemented method for presenting images of cytological smears to human reviewers;

FIG. 11 illustrates an embodiment of a computer-implemented method for cytological smear human review workload management;

FIG. 12A illustrates an embodiment of a computer-implemented method for adaptive management of cytological smear human review workload;

FIG. 12B illustrates an embodiment of a computer-implemented method for preparing images for human review utilizing a decision support system (DSS);

FIG. 12C illustrates an embodiment of a block diagram of a system for preparing images for human review utilizing a DSS;

FIG. 13 illustrates an embodiment of a computer-implemented method for threshold adjustment according to contextual information;

FIG. 14 illustrates an embodiment of a computer-implemented method for analyzing combinations of parameters in cytological smears;

FIG. 15 illustrates an embodiment of a computer-implemented method for adjusting thresholds based on statistical significance of cell populations;

FIG. 16 illustrates an embodiment of a computer-implemented method for integrating medical history in cytological smear analysis;

FIG. 17A illustrates an embodiment of a computer-implemented method for medication-aware automatic cytological smear analysis;

FIG. 17B illustrates an embodiment of a block diagram of a system for medication-aware automatic cytological smear analysis;

FIG. 18A illustrates an embodiment of a computer-implemented method for comparative analysis of cytological smears;

FIG. 18B illustrates an embodiment of a block diagram of a system for comparative analysis of cytological smears;

FIG. 19 illustrates an embodiment of a computer-implemented method for medication-aware presentation of cytological analysis for human review;

FIG. 20 illustrates an embodiment of a computer-implemented method for implementing expertise-based review hierarchies in cytological smear analysis;

FIG. 21A illustrates an embodiment of a computer-implemented method for progressive cytological reporting with human verification;

FIG. 21B illustrates an embodiment of a block diagram of a system for progressive cytological reporting with human verification;

FIG. 22A illustrates an embodiment of a computer-implemented method for combining results from cytological smears;

FIG. 22B illustrates an embodiment of a block diagram of a system for combining results from cytological smears; and

FIG. 23A and FIG. 23B are schematic illustrations of possible embodiments of computers able to realize one or more of the embodiments discussed herein that include a computer.

### DETAILED DESCRIPTION

The term “cytological smear” refers to a sample comprising cells derived from fluid or aspirated biological sources, specifically blood, bone marrow, or body fluids, distributed across any surface or medium suitable for examination. This includes glass slides, plastic surfaces, microfluidic devices, digital imaging surfaces, or any other current or future medium that could be used for cytological examination. The sample may undergo any form of processing, preparation, and/or staining designed to facilitate visualization and analysis of cellular components. Additionally, a cytological smear “associated with a patient” refers to a cytological smear prepared from biological material obtained from the patient.

In the context of cytological smear, the term “image” refers to any representation of a cytological smear or a subset thereof, regardless of acquisition method, visualization technique, or data format. This includes but is not limited to: microscope images, digital scans, spectral data representations, multidimensional data sets, single continuous images, multiple images at varying resolutions, digitally reconstructed or stitched images, composite images constructed from multiple smaller images or snapshots, discontinuous images or collections of snapshots that capture relevant cells or areas, images processed using any enhancement techniques (such as for clarity, contrast adjustment, magnification variation, or color modification), computer-processed representations, or any combination thereof. The representation may characterize the entire smear, specific areas of interest, specific cell types, selected cell or cells, and/or any other features relevant to the analysis being performed, whether rendered visually or existing as analyzable data structures.

The term “cell type” refers to various classifications applicable to cells within cytological smears, which may enable automatic analysis systems to apply different thresholds and/or conditions, including for rare or unique cell types or phenomena. In this context, the term cell type may also be referred to as cell classification, cytological entity, cellular phenotype, morphological class, cell category, cell population, or similar terminology in the industry. Cell types include, but are not limited to: traditional hematological classifications (such as RBCs, lymphocytes, monocytes, neutrophils, eosinophils, basophils, platelets, or their subtypes); phenomenon types (such as rouleaux formations, cellular aggregates, smudge cells, phagocytosis events, or other observable events or patterns with diagnostic significance); developmental or maturation stages observable in smears; reactive or pathological states identifiable through morphological features; morphological classifications (including size and shape variations like anisocytosis or poikilocytosis); cells exhibiting specific inclusions or structural characteristics; cells grouped by mutual characteristics such as similar classification confidence levels (CCLs), spatial location on the slide, or visual similarity without assignment to conventional cell types; computationally derived or AI-identified cell categories that may not correspond to traditional classifications but are distinguishable through image analysis algorithms; statistically identified cell clusters or populations within parameter spaces; quantitatively defined cellular phenotypes based on measurable features; pattern-based classifications that emerge from machine learning analysis of cytological images; unclassified cells for which the automatic analysis system cannot determine a specific cell type with sufficient confidence, whether due to ambiguous morphological features, novel presentations, or insufficient training data. Such unclassified cells may be: (i) categorized as “unclassified proximal to” or “unclassified consistent with” a specific cell type when the cell's features most closely resemble that cell type but fail to meet the requisite classification confidence threshold (such as “unclassified proximal to neutrophil” for cells demonstrating neutrophilic characteristics but lacking definitive neutrophil markers, or “unclassified consistent with monocyte” for cells exhibiting some monocytic features with atypical morphology); or (ii) designated as “generally unclassified” when features do not sufficiently correspond to any recognized cell type and/or when there is no need for extra classification for the unclassified cells; and any cell presentations that may be relevant to automated diagnostic analysis of cytological smears, regardless of the imaging technology, resolution, acquisition method, or analytical approach used for their detection and classification. This includes but is not limited to cells identified through any form of microscopy, super-resolution imaging, multi-camera systems, image stitching or reconstruction technologies, computational enhancement techniques, or any other current or future technologies for visualizing and analyzing cytological specimens.

The term “Classification Confidence Level” (CCL) refers to a measure which may be indicative of the degree of certainty associated with the classification of cells and/or cell types in a cytological smear or any value used as a measure of how likely a cytological smear classification model is correct. CCL may be expressed as a percentage, probability, score, rank, relative comparison, statistical interval, distribution, or fuzzy set membership. The CCL may be associated with individual cells, groups of cells, regions within a cytological smear, or the entire cytological smear. CCL may be derived through any algorithmic or computational approach, including machine learning, deep learning, ensemble methods, Bayesian inference, or knowledge-based systems. In machine learning contexts, CCL may be computed based on scores calculated by models trained to detect, classify, or segment cells based on extracted features from cytological smear images. CCL interpretation may follow standard or inverse scaling; in standard scaling, higher CCL values indicate higher classification confidence; in inverse scaling, lower CCL values indicate higher classification confidence; the system may employ either approach provided the interpretation remains consistent throughout the implementation. Additionally, CCL may be calibrated using validation methods such as confusion matrices, statistical metrics, algorithm performance data, or reference standards. A CCL threshold may serve as a decision criterion that triggers a task, such as presenting for human review or ordering another slide.

The term “rare cell” refers to any morphological phenomenon or cell type whose normal range in a healthy individual is less than 5% (and optionally less than 2%) relative to the sample type and cell line to which it belongs, including cell lines such as leukocytes, erythrocytes, and thrombocytes. Examples of rare cells include: (i) normal rare cells that occur in healthy individuals (such as basophils among leukocytes); (ii) pathological rare cells that appear in disease states (such as blast cells or abnormal lymphocytes); (iii) immature or precursor forms that are uncommon in peripheral blood (such as metamyelocytes or nucleated erythrocytes); (iv) cells exhibiting unusual morphological features or inclusions (such as hypersegmented neutrophils or erythrocytes containing Howell-Jolly bodies); or (v) cells that may transiently appear in response to specific physiological or pathological conditions (such as reactive lymphocytes during viral infections or band neutrophils during acute bacterial infections).

The term “abnormal cell” refers to any morphological phenomenon or cell type not expected to appear in a cytological smear of a healthy individual relative to the sample type, including cell lines such as leukocytes, erythrocytes, and thrombocytes. Examples of abnormal cells include: (i) pathological rare cells that appear in disease states (such as blast cells or abnormal lymphocytes); (ii) immature or precursor forms that are uncommon in peripheral blood (such as metamyelocytes or nucleated erythrocytes); (iii) cells exhibiting unusual morphological features or inclusions (such as hypersegmented neutrophils or erythrocytes containing Howell-Jolly bodies); or (iv) cells that may transiently appear in response to specific physiological or pathological conditions (such as reactive lymphocytes during viral infections or band neutrophils during acute bacterial infections). Certain cells may be classified as abnormal in one context but normal in another, depending on patient demographics, clinical circumstances, and established reference ranges for the specific patient population.

The term “abundant cell” refers to a cell type whose upper normal range in a healthy individual is greater than 5% relative to the sample type and cell line to which it belongs, including cell lines such as leukocytes, erythrocytes, and thrombocytes. Examples of abundant cells include: (i) neutrophils, which typically comprise 50-70% of total leukocytes; (ii) lymphocytes, which generally represent 20-40% of leukocytes; (iii) erythrocytes, which constitute the predominant cell type in peripheral blood; and (iv) specific cell subtypes that maintain substantial presence within their respective lineages under normal physiological conditions.

In the context of cytological smear analysis, the terms “classifier” or “automatic cell classifier” refer herein to any algorithmic, software, computational, firmware, or hardware implementation, alone or in any combination, that receive data representing cells in an image of a cytological smear and produces information indicative of cell type or related cellular characteristics. A classifier may employ any analytical or computational approach, including, but not limited to, rule based reasoning, statistical inference, machine learning techniques, deep learning networks, neural architectures, ensemble methods, or any other methodology suitable for classification. The classifier may operate on any data format or feature set derived from the image, including raw pixel intensities, pre processed image regions, or numerically extracted descriptors. Processing may occur in one or more sequential, parallel, or iterative stages and may incorporate detection and localisation sub functions where relevant. Outputs of the classifier may include: cell type assignments for individual cells, groups of cells, or regions within the smear; Classification Confidence Levels (CCLs)-probability values, scores, ranks, distributions, or other quantitative indicators of confidence associated with classifier assignments; derived metrics such as cell count statistics or cell population proportions; and/or auxiliary data useful for downstream decision logic, such as flags for abnormal morphology or indicators for human review routing. The classifier may execute as software on a general-purpose processor, as firmware on embedded controller, as dedicated logic on ASIC or FPGA, or in any heterogeneous combination thereof. It may reside within an analysis server, an imaging system, or a distributed computing environment. It may interface with an automated slide maker stainer and/or other laboratory equipment, and it may be configured to adapt one or more of its parameters based on medical history, treatment status, and/or protocol specific inputs when such adjustments are deemed beneficial by the system logic.

In the context of cytological smear analysis, the term “hematological cell or cell type quantification metric” (HCQM) refers to one or more values derived from analyzing a cytological smear image (or a subset thereof) to represent the presence, abundance, concentration, density, or relative proportion of a specific blood cell type. An HCQM may be based on one or more of: (i) absolute numerical count (also referred to as a total count) representing the total number of cells of the specific type identified; (ii) a normalized count representing the number of cells per unit area (e.g., cells per square millimeter) or per unit volume (e.g., cells per microliter); (iii) a relative count (which may be expressed as a percentage representation or proportion) representing the proportion of cells of the specific type relative to a reference cell population (e.g., percentage of lymphocytes among total white blood cells); (iv) an estimated count derived from statistical sampling of representative areas of the smear and extrapolated to the entire sample; (v) a density-weighted count that accounts for variations in cell distribution across different regions of the smear; (vi) an adjusted count that incorporates correction factors for sample preparation variables such as smear thickness or monolayer length; or (vii) a calculated value derived from algorithmic transformations of observed data. Different methods for determining the HCQM may be appropriate in different analytical contexts. The system may select a single method or combine multiple methods based on factors including, but not limited to, the specific cell type being analyzed, the diagnostic procedure being performed, or a combination thereof. The determination of the HCQM may involve analyzing the entire image or one or more specific subsets thereof.

The term “Effective Numerical Aperture” (ENA) refers to a measure of resolving power of a microscope or imaging system that provides resolution capabilities equivalent to an objective lens having that numerical aperture (NA). In this context, resolving power refers to the ability to distinguish between adjacent details in an image and/or to distinguish a detail from its background. A microscope or imaging system may have an ENA that is higher than the NA of its objective lens. For example, an imaging apparatus may include a computational microscope configured to reconstruct an image from a plurality of images captured under different illumination conditions, wherein the reconstructed image corresponds to an ENA that is higher than the NA of the objective lens utilized in the imaging apparatus. In another example, an imaging system may operate without a physical lens by computationally reconstructing an image from diffraction patterns, wherein the resulting reconstructed image exhibits a measurable ENA. In some embodiments, the NA of a microscope objective lens may correspond to the ENA of images captured by the microscope. The objective lens may include any suitable type of lens including, but not limited to, an immersion lens designed to operate with an immersion medium having a refractive index greater than one, or a dry lens designed to operate in air.

The term “Electronic Medical Information System” refers to any computerized system, software, database, network, or digital platform designed to collect, store, manage, transmit, process, and/or provide access to patient-related medical, clinical, and/or health information. Electronic Medical Information Systems include, but are not limited to, Electronic Health Records (EHR), Electronic Medical Records (EMR), Laboratory Information Systems (LIS), Hospital Information Systems (HIS), Practice Management Systems (PMS), Clinical Decision Support Systems (CDSS), Patient Portals, and/or any other digital system that maintains patient medical history data and/or clinical information that could be relevant to cytological analysis and/or patient diagnosis. Information stored in Electronic Medical Information Systems may include current and/or past analysis results, metadata and/or images of cytological smears, wherein said information may be stored in an analyzing system performing cytological analysis, in a system communicatively coupled to the analyzing system, or distributed across multiple interconnected systems that collectively manage patient information.

Sentences in the form of “presenting, as part of default human review workflow” refer to any method of making cytological smear images, cells within those images, and/or analysis results of those images available to human reviewers (such as hematologists, pathologists, or laboratory technicians) through standard laboratory review processes. This presentation includes making the information viewable to human reviewers on displays and may include, but is not limited to: digital work queues or lists displaying pending cytological smears requiring review; case prioritization systems showing urgency levels of samples; digital slide viewing interfaces for examining and navigating cytological smear images; result dashboards consolidating automated analysis findings awaiting verification; notification systems alerting reviewers to samples requiring attention through emails, messages, or notifications on computers, phones, tablets, or any medium with a display capable of presenting such indications; laboratory information system (LIS) interfaces displaying assigned work; review tracking systems monitoring completion status; electronic worklists organizing daily or shift review responsibilities; case routing interfaces directing specific samples to appropriate reviewers based on expertise; and/or result verification interfaces for confirming or modifying automated classifications. The presented information may include not only the images that were analyzed by an automated cell classifier, but also may include another image of the same cytological smear or another image of a different cytological smear made from a sample obtained from the same patient. Additionally, the presented information may include CCL values, thresholds used in the analysis, aggregated information from automated analysis, information about the patient, their medical history, information from previous reviews, and any other information which may assist in the review process. The review may be conducted for various purposes, such as providing a final report, verifying automated findings, making diagnostic decisions, or fulfilling quality assurance requirements.

Sentences in the form of “presenting, as part of default human review workflow” explicitly exclude self-initiated reviews, wherein a human reviewer independently chooses to examine cytological smears without system assignment or directive. Examples of such self-initiated reviews include: when a reviewer decides to examine a sample outside their assigned workload; when a reviewer selects additional cases for personal educational purposes; or when a reviewer performs discretionary checks beyond system-designated requirements. Such sentences also explicitly exclude reviewing samples that have been cleared through automation, wherein the system has determined that human review is unnecessary based on predetermined criteria. Examples of such excluded reviews include: samples that the automated system has classified with confidence levels exceeding human review thresholds; cases that meet established parameters for automated verification according to laboratory protocols; samples determined to be within normal ranges that do not trigger human review flags; or specimens processed through automated verification pathways designed to reduce reviewer workload for routine cases. Additionally excluded from default human review workflow are bypass reviews conducted via manual intervention to circumvent standard workflow.

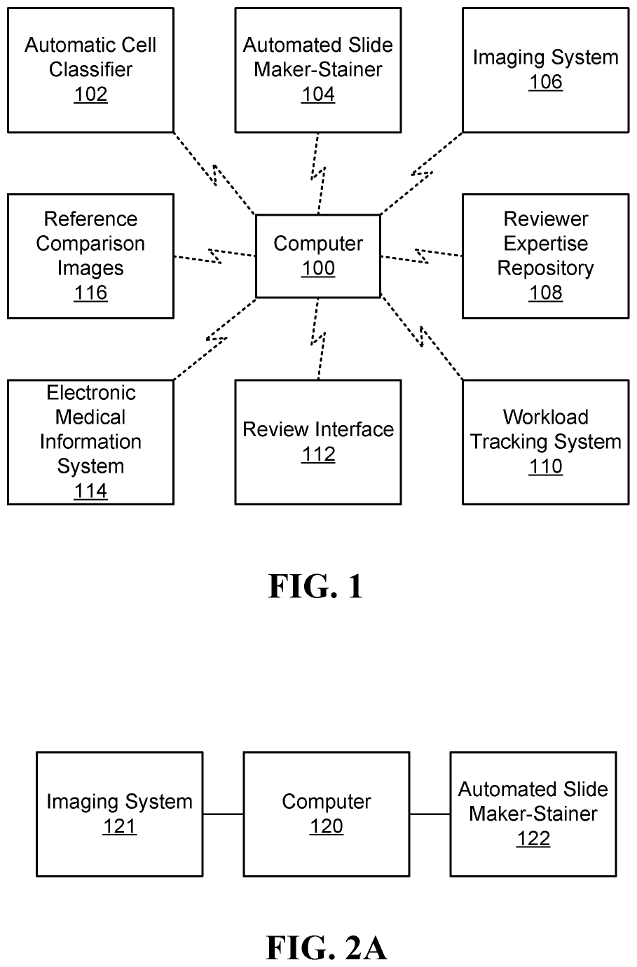

FIG. 1 illustrates an embodiment of a block diagram of an exemplary system architecture for cytological smear analysis in accordance with various embodiments of the present disclosure. The system may include one or more of the illustrated components, configured to interact with one another to perform various functions related to cytological smear processing, analysis, and/or reporting. At the center of the system is computer 100, which may include one or more processors configured to execute instructions and perform various operations described herein. The computer 100 may further include memory for storing data, instructions, parameters, thresholds, and other information utilized during operation. The memory may include volatile memory, non-volatile memory, or combinations thereof. Computer 100 also includes one or more communication interfaces enabling data exchange with other system components through wired connections, wireless connections, network connections, or any combination thereof. Additionally, computer 100 may include or be communicatively coupled to one or more non-transitory computer-readable media storing instructions that, when executed by the one or more processors, cause the computer to perform operations described throughout this disclosure. The computer 100 is communicatively coupled to an automatic cell classifier 102, which may be implemented as hardware, software, firmware, or combinations thereof. The automatic cell classifier 102 is configured to receive data representing cells in images of cytological smears and produce information indicative of cell types or related cellular characteristics. The automatic cell classifier 102 may determine classification confidence levels (CCLs) associated with classified cells. An automated slide maker-stainer 104 is communicatively coupled to the computer 100 and configured to prepare cytological smears according to specified protocols. The automated slide maker-stainer 104 may prepare additional cytological smears based on instructions received from computer 100, potentially using modified protocols determined by the computer 100 based on analysis results. An imaging system 106 is communicatively coupled to the computer 100 and configured to capture images of cytological smears. In some embodiments, the imaging system 106 may include multiple imaging devices with different effective numerical apertures (ENAs), enabling capture of both overview images and high-resolution images suitable for detailed cell-level analysis.

The system may further include an optional reviewer expertise repository 108 communicatively coupled to the computer 100. This repository stores information about human reviewers' areas of expertise, specializations, experience levels, and other relevant characteristics that enable appropriate routing of cases requiring human review based on case-specific requirements. An optional workload tracking system 110 monitors and tracks the current workload of human reviewers, allowing the computer 100 to implement load-balanced distribution of cases requiring human review. The workload tracking system 110 may track review completion times, quality metrics, review queues, and other workload-related parameters. An optional review interface 112 is communicatively coupled to the computer 100 and provides a means for human reviewers to access and analyze cytological smear images, view classification results, and provide verification or corrections. The review interface 112 may present cytological smear images alongside contextual information, reference images, or patient-specific data to facilitate informed review. The computer 100 may also interface with an optional electronic medical information system 114, which provides patient-specific information such as medical history, medication information, treatment status, and previous test results, which enables context-aware analysis and reporting of cytological smear results. Additionally, an optional reference comparison images repository 116 may be communicatively coupled to the computer 100 and store standard images of various cell types and conditions. These reference images may be used during both automated analysis and human review to facilitate accurate classification and verification.

The components illustrated in FIG. 1 may be implemented using various hardware and software configurations and are not limited to any particular implementation. The connections between components may be implemented through any suitable communication channels, protocols, or mechanisms. Furthermore, additional components not shown in FIG. 1 may be included in various embodiments, and some components shown may be combined or further subdivided in alternative embodiments. Different embodiments may use subsets of the components illustrated in FIG. 1, implementing the components utilized for specific functionality while omitting others.

The following implementation addresses the challenge of achieving sufficient statistical reliability in analysis results, or when there is uncertainty about the accuracy of a classifier's output. When a sample approaches but does not reach certain CCL thresholds, the system can request additional slides as needed, which may reduce the total cost of operation by requesting new slides for borderline cases where another sample is expected to improve confidence in results. The embodiment supports analysis of rare cell types and conditions where higher confidence levels may be beneficial.

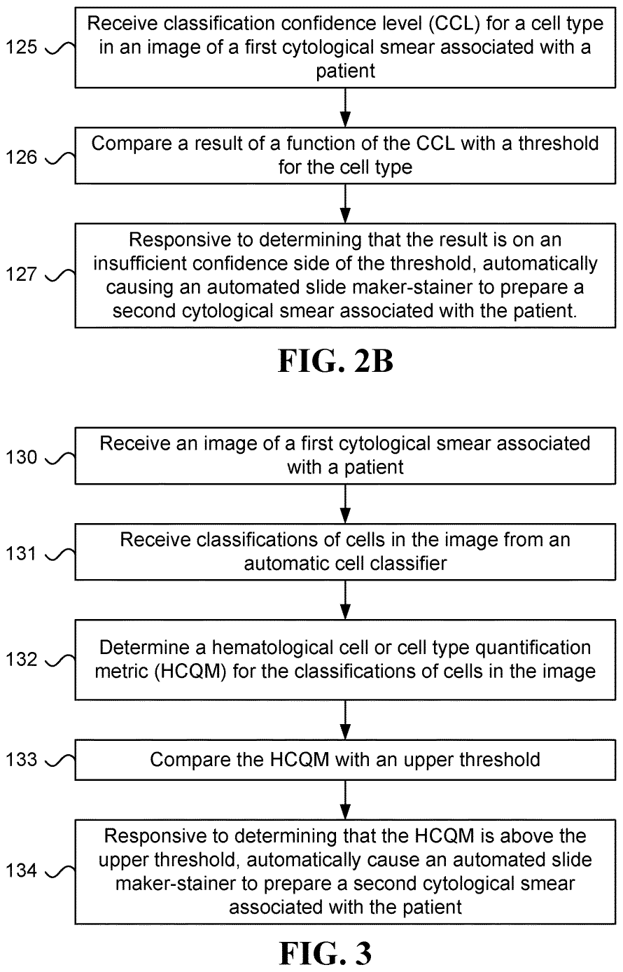

In various implementations, a computer-implemented method for automatically preparing an additional cytological smear based on quantitative classification confidence analysis, comprising: receiving, from an automatic cell classifier, a classification confidence level (CCL) for a cell type in an image of a first cytological smear associated with a patient; comparing a result of a function of the CCL with a threshold for the cell type; and responsive to determining that the result is on an insufficient confidence side of the threshold, automatically causing an automated slide maker-stainer to prepare a second cytological smear associated with the patient. FIG. 2B illustrates an embodiment of a computer-implemented method for automatically preparing an additional cytological smear based on quantitative classification confidence analysis. The method includes the following steps. In step 125, receive classification confidence level (CCL) for a cell type in an image of a first cytological smear associated with a patient. In step 126, compare a result of a function of the CCL with a threshold for the cell type. Optionally the comparing of the result is performed by a processor. And in step 127, responsive to determining that the result is on an insufficient confidence side of the threshold, automatically causing an automated slide maker-stainer to prepare a second cytological smear associated with the patient.

Expressions in the form of “result of a function of a Variable”, such as “result of a function of the CCL”, refer herein to any mathematically or algorithmically derived quantity obtained, at least in part, from the Variable, including a function that returns the Variable itself. Taking CCL as an example, such a function may be calculated by applying one or more arithmetic, logical, statistical, probabilistic or computational operations to the CCL, optionally together with one or more additional parameters. For example, such operations may include scaling, weighting or normalizing the CCL—such as multiplying the CCL by a coefficient, dividing by a reference value or converting the CCL to a percentage; adding, subtracting or otherwise combining the CCL with an offset or bias term; transforming the CCL through linear, non-linear, monotonic or non-monotonic mappings—such as logarithmic, exponential, sigmoid, logit, square-root or power-law transformations; computing a statistical measure or confidence index that incorporates the CCL—such as a moving average, a rolling median, a variance-adjusted score, a Bayesian posterior probability or a z-score; generating an aggregate or composite metric that merges the CCL with one or more other features, such as image-quality indicators, cell-population statistics or patient-specific parameters; or evaluating the CCL within a machine-learning, rule-based, look-up table, or fuzzy-logic model to produce a derived value indicative of overall classification reliability. Unless expressly stated otherwise, no limitation is placed on the nature, complexity or number of operations used by the function of the Variable, nor on the hardware, firmware or software in which those operations may execute. Accordingly, any comparison that employs the Variable itself or any value derived therefrom by one or more of the foregoing operations falls within the scope of “result of a function of the Variable”.

As used herein, the phrase “the result is on an insufficient confidence side of the threshold” covers both possible orientations of the result scale. When the result is defined such that a higher numerical value corresponds to higher confidence, the insufficient confidence side corresponds to values that fall below the threshold. Conversely, when the result is defined such that a lower numerical value corresponds to higher confidence, the insufficient confidence side corresponds to values that exceed the threshold.

As used herein, “insufficient confidence” may refer to results from the classifier which raise a concern about the accuracy of the classification, such as: scores for a subset of cells that fall close to a decision threshold, a subset of cells receiving similar scores for cell types with known potential confusion between them, and/or any other measure indicating that the results of a classifier are less than optimal. Additionally, “insufficient confidence” may refer to a low level of confidence resulting from the statistics or quantification metrics related to the cell type, such as: having too many unclassified cells, having ratios between cell populations of cell types with known potential confusion between them, which reflect a potential issue with the classification, or having a quantification metric for a cell type, which approaches a threshold value.

This embodiment may be integrated with human confirmation or oversight processes without departing from the automated or semiautomated decision-making framework. For example, the system may automatically cause preparation of the second cytological smear while simultaneously generating alerts or notifications for laboratory personnel to review the CCL results and preparation decision. In clinical environments requiring human oversight, the system may automatically initiate the slide preparation process while providing real-time status updates to technologists or pathologists, allowing for intervention if warranted. The automatic preparation may proceed concurrently with human review processes, such as quality assurance protocols where laboratory supervisors verify threshold settings for the cell type or confirm that patient-specific factors have been appropriately considered in the threshold determination. Additionally, the system may integrate with laboratory information management systems to automatically log preparation decisions while flagging cases for human attention when the result of a function of the CCL falls within predetermined ranges requiring clinical correlation. The system may provide a user interface that enables laboratory personnel to monitor and control the comparing of the result of a function of the CCL with the threshold for the cell type, allowing users to adjust threshold parameters, review the result of a function of the CCL in real-time, override automatic preparation decisions when clinically warranted, and establish custom threshold settings for specific cell types or patient populations. Such user interface controls may operate in conjunction with the automated comparing process, providing laboratory staff with visibility into the decision-making logic while maintaining the efficiency of automated preparation workflows for additional cytological smears.

Optionally, the first cytological smear may have been prepared by various entities and at various times or locations relative to the analysis. For example, the smear may be prepared by automated machinery, by laboratory personnel such as a technician, or through a combination of automated and manual processes. The preparation may occur at the same facility where the analysis is performed, such as in an integrated laboratory. Alternatively, the preparation may take place at a separate location, for instance, a point-of-care clinic or a different laboratory, with the prepared smear subsequently being transported to the analysis facility. The timing of preparation relative to analysis may also vary; in some cases, a smear may be analyzed shortly after it is made, while in other cases a delay may exist.

In some implementations, the method further comprises determining at least two different thresholds based on clinical criteria and/or classifier performance related to at least two cell types, respectively; and setting the different thresholds for the at least two cell types, respectively. In some implementations, the method further comprises receiving a second image of the second cytological smear associated with the patient; analyzing the second image to calculate a second CCL (CCL2) for the cell type in the second image; and applying a modified threshold for causing preparation of an additional cytological smear associated with the patient based on a result of a function of the CCL2, thereby reducing unnecessary preparation of additional cytological smears associated with the patient. Optionally, the modified threshold applied to the second cytological smear is less restrictive than the threshold applied to the first cytological smear to reduce the likelihood of entering unnecessary loops of slide preparation when the result of a function of the CCL falls within a certain range. By establishing this threshold relationship, the system creates an adaptive decision boundary that becomes more tolerant after the first analysis, taking into account that multiple samples with similar results may represent an inherent characteristic of the patient's sample or reduced odds of the classifier performing with higher confidence on additional slides, rather than a preparation inadequacy. This may reduce unnecessary resource utilization while maintaining appropriate diagnostic caution for genuinely ambiguous cases.

In some implementations, the method further comprises adjusting the modified threshold based on at least one of: patient medical history, patient demographic information, previous cytological smear results of the patient, or treatment status of the patient, for: triggering more cytological smears for borderline cases for patients with known disorders that manifest in cells, and triggering fewer cytological smears for patients undergoing repeated monitoring with established baselines to account for individual variations established in previous analyses. Optionally, the modified threshold may be adjusted upward or downward according to various patient-specific factors. For patients with known disorders that manifest in cells, thresholds may be increased to trigger additional cytological smears for borderline cases, as diagnostic certainty may be particularly valuable in these clinical contexts. Conversely, for patients undergoing repeated monitoring with established baselines, thresholds may be decreased to account for individual variations established in previous analyses, recognizing that some patients consistently present with certain cellular characteristics that may appear as borderline under standard thresholds. The adjustment may involve applying specific percentage modifications to standard thresholds based on categorization of the patient's clinical profile, which may allow for more effective resource allocation by focusing additional testing on cases where patient history suggests that greater analytical certainty may provide meaningful clinical value. In some implementations, the method further comprises adjusting the modified threshold applied to the second cytological smear based on historical CCL patterns observed in previous analyses associated with the patient; and utilizing the modified threshold when historical CCL patterns associated with the patient indicate similar characteristics across previous analyses.

In some implementations, the method further comprises adjusting the modified threshold applied to the second cytological smear based on a comparison between the result of the function of the CCL for the first cytological smear and historical CCL patterns from previous analyses associated with the patient; and utilizing the modified threshold when the result of the function of the CCL for the first cytological smear is consistent with historical CCL patterns associated with the patient. Optionally, when analyzing the second cytological smear, the system may reference historical CCL patterns from the patient's previous analyses or medical records to inform the selection of appropriate thresholds. When the result of the function of the CCL for the first cytological smear falls within patterns consistently observed in the patient's prior testing, this may suggest that the result reflects the patient's typical cellular presentation rather than an analytical limitation. In such cases, the modified threshold reduces the amount of unnecessary additional cytological smear preparation, considering that the consistent pattern likely represents a stable characteristic of the patient's cells. This modified threshold makes it more likely that the result of the function of the CCL2 is not on the insufficient confidence side of the threshold, thus leading to refraining from causing preparation of additional cytological smears. This historical pattern integration may be particularly valuable for patients with chronic conditions or those undergoing regular monitoring, where establishing personalized baseline expectations may improve analytical efficiency while maintaining appropriate clinical vigilance.

In some implementations, the method further comprises comparing cell and/or cell type properties and/or distributions in the first and second cytological smears to determine consistency between the cytological smears; and applying the modified threshold when the comparison indicates high consistency between the first and second cytological smears, thereby reducing the likelihood of causing preparation of additional cytological smears when results show high consistency between multiple samples. Optionally, the system evaluates consistency between multiple cytological smears to make more informed decisions about additional smear requirements. When two consecutive cytological smears show highly consistent results, this may suggest that the observed cellular patterns represent a physiological state rather than a preparation or classification inadequacy. The system may implement various comparison methods to assess consistency between the first and second cytological smears. For example, the system may calculate a correlation coefficient between corresponding cell population distributions across the first and second cytological smears. When this correlation coefficient exceeds a predetermined value (for example, 0.9 or greater), the system may automatically adjust the threshold to cause preparation of additional cytological smears, recognizing that further sampling is less likely to yield significantly different results. Consistent findings across multiple samples typically increase confidence in the reliability of the results, even when individual results of functions of the CCL are on the insufficient confidence side of standard thresholds. The specific threshold adjustment may be a function of the consistency strength, which may be valuable for monitoring stable chronic conditions where minor variations are expected but significant deviations would require clinical attention.

In some implementations, the method further comprises receiving a second image of the second cytological smear associated with the patient; analyzing the second image; and causing preparation of a third cytological smear associated with the patient based on at least one of: detection of inadequate cell quantity, identification of preparation artifacts affecting classification accuracy, or detection of rare cell types requiring statistical confirmation. Optionally, the system may implement multiple criteria beyond analysis of the result of a function of the CCL to determine when additional cytological smears are warranted. These criteria may include quantitative assessments of cell populations, quality evaluations of the preparation, or detection of specific cell types requiring further investigation. For example, when inadequate quantities of certain cell types are detected, the system may cause preparation of an additional cytological smear to achieve statistically significant sampling, even when classification confidence levels are acceptable. Similarly, when preparation artifacts such as staining inconsistencies or smearing irregularities affect a significant subset of the smear, additional preparation may be warranted regardless of the result values. For rare cell types that have potential diagnostic significance, such as blast cells or abnormal lymphocytes, the system may require additional sampling for confirmation when these cells are detected at borderline levels. Optionally, the system may typically limit the total number of additional preparations to between one and three slides regardless of analytical outcomes, possibly because persistent analytical challenges after multiple preparations may require alternative diagnostic approaches.

In some implementations, the method further comprises analyzing CCLs from multiple patients; identifying patterns indicating systematic classification challenges; determining causes for the identified patterns; and adjusting the threshold based on the determined causes to optimize additional cytological smear preparation rates. Optionally, the system may implement a population-based learning mechanism to refine threshold settings based on aggregate analysis across multiple patients. By collecting and analyzing CCL patterns from diverse patient populations, the system may identify systematic classification challenges that affect certain cell types or preparation conditions. For example, if analysis reveals that specific staining protocols typically result in results of functions of the CCL on the insufficient confidence side of the threshold for particular cell types across multiple patients, the system may determine that the issue stems from preparation variables rather than genuine classification uncertainty. In such cases, the system may adjust thresholds accordingly or modify preparation protocols to address the root causes. This population-level analysis may help distinguish between issues requiring additional sampling and those better addressed through systemic improvements to classification algorithms or preparation methods. The threshold adjustments may be implemented gradually through rolling averages or more immediately through rule-based interventions when clear patterns emerge, creating a system that improves preparation efficiency while maintaining diagnostic reliability.

In some implementations, the method further comprises collecting data from multiple patients regarding effectiveness of additional cytological smear preparation decisions; determining whether the additional cytological smears provided diagnostic value beyond the first cytological smears; and dynamically adjusting the threshold, based on the determination, such as to cause preparation of fewer future additional cytological smears when the additional cytological smears frequently did not provide additional diagnostic value, and to cause preparation of more future additional cytological smears when the additional cytological smears frequently provided significant diagnostic value. Optionally, the system may implement an adaptive learning mechanism that refines threshold settings based on the observed utility of additional cytological smears. By analyzing outcomes across multiple patients, the system evaluates whether causing preparation of additional cytological smears provided meaningful diagnostic benefits in specific classification scenarios. When analysis indicates that additional cytological smears frequently failed to yield significant new diagnostic information despite results being on the insufficient confidence side of the threshold for certain cell types or conditions, the system may adjust the threshold to cause preparation of fewer future additional cytological smears for these scenarios, reducing unnecessary additional preparations. Conversely, when analysis reveals that additional cytological smears frequently provided valuable diagnostic clarification, especially for specific cell types or clinical contexts, the system may adjust the threshold to cause preparation of more future additional cytological smears in similar future cases. This feedback loop creates a system that refines thresholds based on empirical evidence of diagnostic utility, potentially improving resource allocation while maintaining diagnostic quality. The adaptive adjustments may be implemented through various mechanisms including statistical weighting algorithms, rule-based modifications, or machine learning approaches that identify complex patterns in preparation effectiveness.

In some implementations, the method further comprises collecting data from multiple patients regarding effectiveness of additional cytological smear preparation decisions; determining whether the additional cytological smears provided diagnostic value beyond the first cytological smears; and dynamically adjusting the threshold, based on the determination, such as to cause preparation of fewer future additional cytological smears when the additional cytological smears frequently did not provide additional diagnostic value, thereby reducing unnecessary preparation of future additional cytological smears.

In some implementations, the method further comprises receiving multiple cell types from the automatic cell classifier, associating a first threshold with a relatively rare cell type selected from the multiple cell types, wherein the second threshold is configured such that a greater proportion of results of functions of CCLs fall on the sufficient confidence side of the second threshold compared to the first threshold, thereby causing preparation of fewer additional cytological smears for the relatively abundant cell type compared to the relatively rare cell type. The threshold may be established with consideration for the relative rarity of different cell types. Since rare cell types typically appear in smaller quantities within samples, achieving the same CCL as for abundant cell types may be more challenging from a statistical or classifier perspective. For at least a subset of the cell types, the system may implement a relationship between cell rarity and thresholds, where rarer cells are associated with thresholds that cause preparation of more additional cytological smears compared to more common cell types. This adjustment accounts for the statistical improbability of achieving high CCLs for rare cell populations even in well-prepared samples, and may prevent excessive causation of additional cytological smear preparation while maintaining appropriate assessment standards for diagnostically significant rare cell populations. The specific threshold adjustment may vary based on factors such as the clinical significance of the cell type, the degree of rarity, and statistical confidence requirements for the particular analysis.

The relationship between thresholds for different cell types may be established to reflect different likelihoods of causing preparation of additional cytological smears. When a threshold is described as having a lower probability to cause preparation of additional cytological smears compared to another threshold, this may be implemented through various mechanisms. In one implementation, the threshold values may be positioned such that, based on typical distributions of CCL values for each cell type, a smaller percentage of analyzed samples will have results of functions of CCLs that fall on the insufficient confidence side of the threshold. In another implementation, the thresholds may be defined through empirical analysis of historical data, where the system determines threshold values that historically resulted in different frequencies of additional smear preparation across patient populations. The system may also implement dynamic threshold relationships where the relative positioning of thresholds is maintained to achieve predetermined ratios of additional smear preparation between cell types, such as configuring thresholds such that rare cell types trigger additional smears at a predetermined rate higher than abundant cell types under typical operating conditions. These probability relationships may be expressed through various metrics including percentage of cases triggering additional preparation, statistical likelihood based on CCL distributions, empirical frequencies from clinical data, or mathematical functions relating threshold positions to preparation outcomes.

In some implementations, the method further comprises receiving a second image of the second cytological smear associated with the patient; identifying rare cell types present in both the first and second cytological smears; combining cell classification data from both cytological smears for the identified rare cell types; and calculating a combined hematological cell or cell type quantification metric across both cytological smears to increase accuracy of classification for the rare cell types. When analyzing rare cell types, the system may integrate multiple samples to leverage data from both the first and second cytological smears. This process may begin with identifying which specific rare cell types are present in both samples, and for these identified rare populations, cell classification data from both cytological smears may be combined, such as through a multi-step process. First, the system may aggregate the hematological cell or cell type quantification metric, such as raw cell counts, from both samples. Second, it may apply normalization techniques to account for any preparation differences between the samples. Finally, it may calculate classification statistics based on this combined dataset. This process effectively increases the sample size available for analysis of rare cell populations, potentially providing more reliable classification results than would be possible with either sample alone.

In some implementations of the method, the causing of the automated slide maker-stainer to prepare the second cytological smear comprises: identifying specific classification confidence deficiencies in the image; selecting at least one preparation parameter from a group comprising: sample dilution ratio, staining duration, stain concentration, smearing speed, smearing angle, and smearing pressure; and modifying the selected preparation parameter to address the identified classification confidence deficiencies. Optionally, specific classification confidence deficiencies in the first sample are identified, and targeted protocol modifications to address these issues are applied. Rather than causing preparation of another sample with identical preparation parameters, the system may analyze the nature of the limitations in the results of functions of the CCL in the first sample. For instance, if the result is on the insufficient confidence side of the threshold due to inadequate staining contrast, the system may modify staining parameters. Similarly, if cell distribution issues affect the CCL, smearing parameters may be adjusted. The system may select one or more specific preparation parameters such as sample dilution ratio, staining duration, stain concentration, smearing speed, smearing angle, or smearing pressure, and modify them based on the identified classification confidence deficiencies. This process may enhance the likelihood that the second sample will resolve the specific limitations identified in the first sample, potentially reducing the likelihood of requiring additional samples beyond the second preparation.

In some implementations, the method further comprises determining the threshold based on a statistical confidence interval required for clinical diagnosis of conditions associated with the cell type; receiving a second image of the second cytological smear associated with the patient; analyzing the second image; and calculating an aggregate result based on cell classification data from both the first and second cytological smears. The threshold may be established based on statistical requirements associated with clinical applications. Different medical conditions may require different levels of statistical certainty for diagnostic purposes based on their clinical significance, treatment implications, and standard of care considerations. The system may establish thresholds that reflect these varying statistical requirements, with potentially serious conditions associated with more stringent confidence intervals than conditions with less critical immediate consequences. When analyzing the second cytological smear, the system may integrate classification data from both the first and second cytological smears, effectively increasing the statistical sample size and potentially achieving higher aggregate confidence than would be possible with either cytological smear alone. For example, if the classifier produces uncertain results for some of the cells in the first smear, using a second smear may improve the ability to provide proper metrics for cell types. This process may align laboratory practices with clinical needs by facilitating achievement of the statistical confidence appropriate for specific medical decisions, creating a framework for determining when additional samples may be necessary while improving the utility of samples already collected.

In some implementations, the method further comprises preparing the first cytological smear utilizing the automated slide maker-stainer or another automated slide maker-stainer. In various embodiments, the automated slide maker-stainer may include one or more machines to prepare cytological smears for analysis. In a first example, the automated slide maker-stainer is implemented as an integrated single machine that performs both slide-making functions (which may include sample handling, smearing, and spreading) and staining functions (which may include application of one or more staining reagents and subsequent processing) within a unified hardware platform. In a second example, the automated slide maker-stainer includes a first machine for slide-making operations and a second machine for staining operations, with the slides being transferred between the first and second machines. Additional configurations may include modular systems where multiple slide-making and/or staining modules operate in conjunction with control systems; distributed systems where slide preparation and staining functions occur at different physical locations; or automated systems incorporating possible manual intervention points. In some implementations of the method, a non-transitory computer-readable medium comprising instructions that, when executed by a processor, cause the processor to perform the computer-implemented method.

In various implementations, a system configured to control preparation of an additional cytological smear, comprising: a computer configured to: receive an image of a first cytological smear associated with a patient; execute an automatic cell classifier configured to calculate a classification confidence level (CCL) for a cell type in the image; compare a result of a function of the CCL with a threshold for the cell type; and responsive to the comparison indicating that the result is on an insufficient confidence side of the threshold, automatically cause an automated slide maker-stainer to prepare a second cytological smear associated with the patient. FIG. 2A illustrates an embodiment of a block diagram of a system configured to control preparation of an additional cytological smear. The system includes a computer 120 that receives images from imagine system 121. The computer is further coupled to an automated slide maker-stainer 122. In some implementations of the system, the computer is further configured to: determine at least two different thresholds based on clinical criteria and/or classifier performance related to at least two cell types, respectively; and set the different thresholds for the at least two cell types, respectively.

In some implementations of the system, the computer is further configured to: receive a second image of the second cytological smear associated with the patient; analyze the second image to calculate a second CCL (CCL2) for the cell type in the second image; and apply a modified threshold to cause preparation of an additional cytological smear associated with the patient based on a result of a function of the CCL2. In some implementations of the system, the computer is further configured to adjust the modified threshold based on at least one of: patient medical history, patient demographic information, previous cytological smear results of the patient, or treatment status of the patient, to: trigger more cytological smears for borderline cases for patients with known disorders that manifest in cells, and trigger fewer cytological smears for patients undergoing repeated monitoring with established baselines to account for individual variations established in previous analyses. In some implementations of the system, the computer is further configured to: adjust the modified threshold applied to the second cytological smear based on historical CCL patterns observed in previous analyses associated with the patient; and utilize the modified threshold when historical CCL patterns associated with the patient indicate similar characteristics across previous analyses. In some implementations of the system, the computer is further configured to: adjust the modified threshold applied to the second cytological smear based on a comparison between the result of the function of the CCL for the first cytological smear and historical CCL patterns from previous analyses associated with the patient; and utilize the modified threshold when the result of the function of the CCL for the first cytological smear is consistent with historical CCL patterns associated with the patient. In some implementations of the system, the computer is further configured to: compare cell and/or cell type properties and/or distributions in the first and second cytological smears to determine consistency between the cytological smears; and apply the modified threshold when the comparison indicates high consistency between the first and second cytological smears.

In some implementations of the system, the computer is further configured to: receive a second image of the second cytological smear associated with the patient; analyze the second image; and cause preparation of a third cytological smear associated with the patient based on at least one of: detection of inadequate cell quantity, identification of preparation artifacts affecting classification accuracy, or detection of rare cell types requiring statistical confirmation. In some implementations of the system, the computer is further configured to: analyze CCLs from multiple patients; identify patterns indicating systematic classification challenges; determine causes for the identified patterns; and adjust the threshold based on the determined causes to optimize additional cytological smear preparation rates.

In some implementations of the system, the computer is further configured to: collect data from multiple patients regarding effectiveness of additional cytological smear preparation decisions; determine whether the additional cytological smears provided diagnostic value beyond the first cytological smears; and dynamically adjust the threshold, based on the determination, such as to cause preparation of fewer future additional cytological smears when the additional cytological smears frequently did not provide additional diagnostic value, and to cause preparation of more future additional cytological smears when the additional cytological smears frequently provided significant diagnostic value.

In some implementations of the system, the computer is further configured to: collect data from multiple patients regarding effectiveness of additional cytological smear preparation decisions; determine whether the additional cytological smears provided diagnostic value beyond the first cytological smears; and dynamically adjust the threshold, based on the determination, such as to cause preparation of fewer future additional cytological smears when the additional cytological smears frequently did not provide additional diagnostic value, thereby reducing unnecessary preparation of future additional cytological smears. In some implementations of the system, the computer is further configured to: receive multiple cell types from the automatic cell classifier, associate a first threshold with a relatively rare cell type selected from the multiple cell types, wherein the second threshold is configured such that a greater proportion of results of functions of CCLs fall on the sufficient confidence side of the second threshold compared to the first threshold, thereby causing preparation of fewer additional cytological smears for the relatively abundant cell type compared to the relatively rare cell type. In some implementations of the system, the computer is further configured to: receive a second image of the second cytological smear associated with the patient; identify rare cell types present in both the first and second cytological smears; combine cell classification data from both cytological smears for the identified rare cell types; and calculate a combined hematological cell or cell type quantification metric across both cytological smears to increase accuracy of classification for the rare cell types.

In some implementations of the system, the computer is further configured to: identify specific classification confidence deficiencies in the image; select at least one preparation parameter from a group comprising: sample dilution ratio, staining duration, stain concentration, smearing speed, smearing angle, and smearing pressure; and modify the selected preparation parameter to address the identified classification confidence deficiencies. In some implementations of the system, the computer is further configured to: determine the threshold based on a statistical confidence interval required for clinical diagnosis of conditions associated with the cell type; receive a second image of the second cytological smear associated with the patient; analyze the second image; and calculate an aggregate result based on cell classification data from both the first and second cytological smears. In some implementations, the system further comprises the automated slide maker-stainer, and wherein the automated slide maker-stainer is configured to prepare the first cytological smear.

The following implementation improves the reliability of analysis by promoting appropriate representation of cell types when hematological cell or cell type quantification metric (HCQM) is outside a predetermined acceptable range (e.g., either too few or too many, or a percentage which is too low or too high) which may affect analytical reliability. By ordering another slide when certain cell types appear inadequately represented or overrepresented, the system can enhance reliability, offer standardized assessment of cell quantity appropriateness, and support consistent analysis across samples in laboratory settings, which helps laboratories manage variability in sample quality without requiring manual intervention.

In various implementations, a computer-implemented method for ordering another cytological smear, comprising: receiving an image of a first cytological smear associated with a patient; receiving from an automatic cell classifier, classifications of cells in the image; determining a hematological cell or cell type quantification metric (HCQM) for the classifications of cells in the image; comparing the HCQM with an upper threshold; and responsive to determining that the HCQM is above the upper threshold, automatically causing an automated slide maker-stainer to prepare a second cytological smear associated with the patient. FIG. 3 illustrates an embodiment of a computer-implemented method for ordering another cytological smear. The method includes the following steps. In step 130, receive an image of a first cytological smear associated with a patient. In step 131, receive classifications of cells in the image from an automatic cell classifier. In step 132, determine a hematological cell or cell type quantification metric (HCQM) for the classifications of cells in the image. In step 133, compare the HCQM with an upper threshold. And in step 134, responsive to determining that the HCQM is above the upper threshold, automatically cause an automated slide maker-stainer to prepare a second cytological smear associated with the patient.

The comparison of the HCQM with an upper threshold (which may be an upper limit of a clinical normal range) enables detection of potentially anomalous cell quantities that may indicate either a patient condition or a classification error. This threshold comparison may identify situations where the classifier has produced a result with high confidence, but the result itself is clinically suspicious due to unexpectedly high cell counts. By automatically causing preparation of a second cytological smear when the HCQM exceeds the upper threshold, the system facilitates verification of potentially abnormal findings, which addresses cases where the classifier confidently reports results that fall outside expected physiological ranges, prompting additional sampling to distinguish between genuine patient conditions and potential classification artifacts.

In some implementations, the method further comprises determining the upper threshold based on statistical analysis of cell type distributions in a reference population, wherein the automatic causing of the automated slide maker-stainer to prepare the second cytological smear enables verification of whether the HCQM above the upper threshold results from a patient condition or a classification error. The upper threshold may be determined through statistical analysis of cell type distributions in reference populations, which can establish quantitative boundaries between expected and potentially anomalous findings. This approach may incorporate data from diverse reference groups including healthy individuals, patients with known conditions, or specific demographic cohorts to create statistically valid thresholds. The verification process enabled by preparing a second cytological smear may help distinguish between actual patient conditions and potential classification errors when the HCQM exceeds the upper threshold. For example, consistently elevated cell counts across multiple smears might suggest a genuine physiological condition, while significant discrepancies between smears might indicate classification artifacts in the original analysis. The reference population data may be periodically updated to reflect evolving understanding of cell distribution patterns across different patient populations.

In some implementations of the method, the first cytological smear is a blood smear, the HCQM is for cells, which are generally similar to white blood cells, classified as unclassified cells, and setting the upper threshold between 3% and 10% of the total white blood cells classified in the image. These unclassified cells represent WBCs whose morphological features prevent confident assignment to standard cell type categories by the automatic cell classifier. An elevated percentage of unclassified cells can indicate either technical issues with the smear preparation or clinically significant abnormal cells that fall outside typical classification parameters. The system may implement specific thresholds for unclassified cells, typically set between 3% and 10% of total WBCs, reflecting levels where either technical adequacy or diagnostic clarity may be compromised. When unclassified cells exceed this threshold, preparing another smear may resolve the classification uncertainty by providing additional cellular representations. In cases where the high unclassified cell percentage stems from technical factors such as suboptimal staining or smearing artifacts, the second preparation may yield improved classification results. Alternatively, when the unclassified cells represent abnormal cells with unusual morphological features, the additional smear provides more cellular material for detailed examination, potentially revealing consistent abnormal features that may have diagnostic significance.

In some implementations of the method, the HCQM is for white blood cells (WBCs), and further comprising setting the upper threshold to at least 1000 WBCs. Optionally, the upper threshold may correspond to clinically significant leukocytosis that may warrant verification through additional sampling. Elevated WBC counts above this threshold may be associated with various conditions including infections, inflammatory processes, hematological malignancies, or physiological stress responses. The preparation of a second smear when this threshold is exceeded may help confirm whether the elevation represents a genuine finding or potentially a classification anomaly. The specific threshold value may be adjusted based on factors such as patient demographics, clinical context, or laboratory-specific reference ranges. Optionally, the system might employ graduated thresholds that trigger different verification protocols based on the magnitude of WBC elevation, with moderate elevations receiving standard verification and extreme elevations receiving expedited or enhanced verification protocols.

In some implementations, the method further comprises determining the upper threshold based on clinical guidelines associated with the HCQM. In some implementations, the method further comprises adjusting the clinical guidelines based on a diagnostic procedure being performed, wherein at least some of the diagnostic procedures have different threshold requirements according to their respective statistical confidence requirements. The HCQMs may be selected based on clinical guidelines for cell populations and/or percentages. This is often desirable for the system to reflect real-world needs and incorporate research findings and field experience, thereby making the results significant. The clinical guidelines may be selected based on the particular diagnostic procedure being performed, recognizing that different procedures may require different levels of statistical confidence. For example, screening procedures may employ more sensitive thresholds that potentially identify more cases requiring verification, while confirmatory procedures may utilize more specific thresholds to reduce unnecessary additional testing. The implementation may incorporate guideline-recommended statistical confidence requirements, which can vary based on the clinical significance of the parameter being measured or the consequences of potential misclassification.

In some implementations, the method further comprises analyzing the at least a subset of the image to determine respective hematological cell types quantification metrics (HCQMs) for multiple cell types associated with clinical significance weights; comparing the respective HCQMs against corresponding upper thresholds; calculating a weighted score based on the clinical significance weights and an extent to which at least some of the HCQMs exceed their corresponding upper thresholds; and causing the automated slide maker-stainer to prepare the second cytological smear when the weighted score exceeds a predetermined value. This multi-parameter analysis approach may recognize that different cell types carry varying diagnostic importance, with some abnormalities having more significant clinical implications than others. By assigning clinical significance weights to different cell types, the system can prioritize findings based on their potential impact on patient care. The weighted score calculation may incorporate both the magnitude of threshold exceedance and the clinical importance of the parameters involved. For example, a moderate elevation in a highly significant cell type may generate a higher weighted score than a substantial elevation in a less clinically significant parameter. This nuanced approach may help optimize additional smear preparation by triggering verification primarily when clinically meaningful abnormalities are detected, potentially reducing unnecessary testing while maintaining appropriate vigilance for significant findings.

In some implementations, the method further comprises receiving a second image of the second cytological smear associated with the patient; analyzing at least a subset of the second image to determine a second HCQM; combining the HCQM and the second HCQM to a combined HCQM; and responsive to determining that the combined HCQM is below the upper threshold, refraining from causing the automated slide maker-stainer to prepare a third cytological smear associated with the patient. This combined HCQM approach may leverage the additional data points provided by the second smear to potentially increase statistical confidence in the overall assessment. If this combined HCQM falls below the established upper threshold, the system may refrain from preparing a third cytological smear, recognizing that sufficient data has been gathered to support classification confidence. This may help prevent unnecessary additional smear preparation when the expanded dataset already provides adequate statistical validity. The combination method may implement various statistical techniques including averaging, weighted combinations based on sample quality, or more complex statistical modeling that accounts for inter-sample variability. This process may optimize resource utilization by limiting additional sample preparation to situations where meaningful clinical benefit may be derived from the additional data, while still maintaining appropriate analytical thoroughness for potentially significant findings.

In some implementations, the method further comprises receiving a second image of the second cytological smear associated with the patient; analyzing at least a subset of the second image to determine a second HCQM; comparing the second HCQM with the upper threshold; and responsive to determining that the second HCQM is below the upper threshold, refraining from causing the automated slide maker-stainer to prepare a third cytological smear associated with the patient. The system may separately analyze the second cytological smear to determine whether the initial threshold exceedance was consistent across multiple samples. When the second HCQM falls below the upper threshold, this may suggest that the initial exceedance could have been due to sample-specific factors rather than a genuine patient condition, potentially eliminating the need for further sampling. By refraining from preparing a third smear in such cases, the system may reduce unnecessary testing while maintaining appropriate verification protocols. This comparison between first and second smear results may identify cases where technical variability, sampling inconsistency, or transient physiological fluctuations may have influenced the initial findings. The assessment of the second smear may incorporate quality indicators such as staining adequacy, cell distribution, or morphological clarity to evaluate the reliability of the second HCQM determination.

In some implementations, the method further comprises receiving a second image of the second cytological smear associated with the patient; analyzing at least a subset of the second image to determine a second HCQM; comparing the second HCQM with a modified upper threshold that is different from the upper threshold used for the first cytological smear; and responsive to determining that the second HCQM is above the modified upper threshold, causing the automated slide maker-stainer to prepare a third cytological smear associated with the patient. The modified threshold approach may recognize that the context for interpreting the second smear differs from that of the initial analysis. For example, the confirmation of an elevated HCQM in a second sample may carry different statistical or clinical implications than the initial finding. The modified threshold may be adjusted based on factors such as the magnitude of the initial exceedance, the clinical significance of the parameter being measured, or specific verification requirements associated with the particular cell type being analyzed. If the second HCQM exceeds this modified threshold, the system may proceed with preparing a third cytological smear to further investigate the persistent finding. This progressive threshold adjustment may reflect a tiered verification strategy that applies increasingly stringent criteria as multiple samples continue to show threshold exceedances, potentially identifying cases requiring more thorough investigation while limiting additional sampling for transient or artifactual elevations.

In some implementations, the method further comprises determining the modified upper threshold based on at least one of: results from analysis of the first cytological smear, a medical condition of the patient, prior medical history of the patient, or analysis requirements associated with the patient. These factors may include the specific results from the first cytological smear analysis, such as the magnitude of threshold exceedance or the presence of additional abnormal parameters. The patient's known medical conditions may influence threshold adjustment, potentially modifying expectations for certain cell populations based on documented diagnoses. Prior medical history, including baseline values from previous analyses, may inform personalized thresholds that account for the patient's established patterns. Analysis requirements associated with the patient, such as monitoring protocols for specific conditions or heightened vigilance during critical treatment phases, may also factor into threshold modifications. This patient-specific context may allow for more relevant threshold determination that balances the need for appropriate verification against the goal of minimizing unnecessary testing. The threshold adjustment process may incorporate algorithms that weight these various factors according to their relevance for the specific parameters being evaluated.

In some implementations, the method further comprises determining the upper threshold based on statistical confidence required for meaningful analysis associated with the HCQM. Parameters with high clinical significance, where misclassification may significantly impact patient care, may be associated with thresholds that reflect higher statistical confidence requirements. Conversely, parameters with more ambiguous clinical implications may employ thresholds that balance verification needs with resource considerations. The threshold determination process may incorporate statistical methods such as reference range derivation, outlier detection algorithms, or distribution modeling techniques appropriate for the specific parameter being evaluated. This statistically informed approach may optimize the balance between verification thoroughness and testing efficiency by targeting additional sampling toward cases where statistical uncertainty may have meaningful clinical consequences. The specific statistical methodologies may be selected based on the known distribution characteristics of the cell parameters and the clinical context in which they are being evaluated.

In some implementations, the method further comprises transmitting data related to selection of preparation parameters to the automated slide maker-stainer for preparing the second cytological smear; and selecting preparation parameters based on characteristics of the HCQM that exceeds the upper threshold. The transmitting of data related to selection of preparation parameters may encompass multiple implementation approaches. In a first implementation, the system may transmit the specific preparation parameters themselves, providing explicit instructions to the automated slide maker-stainer regarding exactly how to prepare the second slide. In a second implementation, the system may transmit information about the specific anomalies detected (such as “excessive cell count” or “abnormal cell density”), allowing the automated slide maker-stainer to determine appropriate preparation parameters, optionally based on its internal algorithms. In a third implementation, the system may transmit partial parameter information along with problem indicators, creating a collaborative decision process where some parameters are explicitly specified while others are determined by the automated slide maker-stainer based on the nature of the identified issues. The specific implementation may vary based on the capabilities of the automated slide maker-stainer, the complexity of the preparation adjustments required, and the system architecture coupling the analysis engine to the preparation hardware.

In some implementations of the method, the preparation parameters comprise at least one of: sample dilution ratio, sample concentration technique, smearing speed, smearing angle, smearing pressure, staining duration, or stain concentration. In some implementations of the method, the HCQM is for a rare cell type, and further comprising setting the upper threshold at or above a clinical normal range upper threshold defined for the rare cell type. For rare cell types with significant clinical implications, the system may set upper thresholds with consideration for established clinical reference ranges. When the HCQM exceeds these clinically significant thresholds, it often warrants preparation of another cytological smear to confirm the finding, as elevated levels of rare cells frequently correlate with pathological conditions. The upper threshold may be set equal to or slightly above the upper limit of the clinical normal range to trigger additional verification when potentially abnormal levels are detected, which may help validate findings that may influence diagnostic decisions while avoiding unnecessary additional smear preparations for results that fall within expected normal variations. For example, in cases where the presence of immature cell forms exceeds typical reference ranges, the system triggers additional smear preparation to provide sufficient cellular evidence for confident diagnosis, particularly important for cell types associated with conditions such as leukemia or serious infection.

In some implementations of the method, the first cytological smear is a blood smear, the HCQM is for blast cells, and setting the upper threshold between 1% and 5% of the white blood cells classified in the image. Blast cells represent immature WBC precursors that, when present in peripheral blood above certain levels, often indicate serious hematological conditions including leukemia. The system may implement specific thresholds for blast cells, typically set between 1% and 5% of total white blood cells, reflecting clinically significant ranges where additional confirmation is needed for accurate diagnosis. When the HCQM for blast cells exceeds this threshold, the system may automatically trigger preparation of another blood smear to verify this finding. The additional smear may help distinguish true blast cells from morphologically similar cells that might be misclassified, such as reactive lymphocytes or monocytes with atypical features, thereby increasing diagnostic confidence for this clinically significant parameter.

In some implementations of the method, the first cytological smear is a blood smear, the HCQM is for aberrant lymphocytes, and setting the upper threshold between 1% and 5% of the white blood cells classified in the image. Aberrant lymphocytes, which display morphological or immunophenotypic deviations from normal lymphocyte characteristics, may indicate various pathological conditions including viral infections, lymphoproliferative disorders, or certain autoimmune diseases. The system may implement specific upper thresholds for aberrant lymphocytes, typically set between 1% and 5% of total WBCs, reflective of levels where clinical significance increases substantially. When the HCQM for aberrant lymphocytes exceeds this threshold, automatically triggering another blood smear preparation may help confirm these findings before potentially initiating further diagnostic workup. The confirmation provided by the second smear can enhance diagnostic reliability, such as when distinguishing between transient reactive processes and more serious hematological malignancies. In some implementations of the method, a non-transitory computer-readable medium comprising instructions that, when executed by a processor, cause the processor to perform the computer-implemented method.

In various implementations, a system for ordering another cytological smear, comprising: a computer configured to: receive an image of a first cytological smear associated with a patient; automatically classify cells in the image; determine a hematological cell or cell type quantification metric (HCQM) for the classifications of cells in the image; compare the HCQM with an upper threshold; and responsive to the comparison indicating that the HCQM is above the upper threshold, automatically cause an automated slide maker-stainer to prepare a second cytological smear associated with the patient.

In some implementations of the system, the computer is further configured to determine the upper threshold based on statistical analysis of cell type distributions in a reference population, wherein the automated slide maker-stainer preparing the second cytological smear enables verification of whether the HCQM above the upper threshold results from a patient condition or a classification error. In some implementations of the system, the first cytological smear is a blood smear, the HCQM is for cells, which are generally similar to white blood cells, classified as unclassified cells, and wherein the upper threshold is set between 3% and 10% of the total white blood cells classified in the image. In some implementations of the system, the HCQM is for white blood cells (WBCs), and wherein the upper threshold is set to at least 1000 WBCs.

In some implementations of the system, the computer is further configured to determine the upper threshold based on clinical guidelines associated with the HCQM. In some implementations of the system, the computer is further configured to adjust the clinical guidelines based on a diagnostic procedure being performed, wherein at least some of the diagnostic procedures have different threshold requirements according to their respective statistical confidence requirements.

In some implementations of the system, the computer is further configured to: analyze at least a subset of the image to determine respective hematological cell types quantification metrics (HCQMs) for multiple cell types associated with clinical significance weights; compare the respective HCQMs against corresponding upper thresholds; calculate a weighted score based on the clinical significance weights and an extent to which at least some of the HCQMs exceed their corresponding upper thresholds; and responsive to determining that the weighted score exceeds a predetermined value, cause the automated slide maker-stainer to prepare the second cytological smear.

In some implementations of the system, the computer is further configured to: receive a second image of the second cytological smear associated with the patient; analyze at least a subset of the second image to determine a second HCQM; combine the HCQM and the second HCQM to a combined HCQM; and responsive to determining that the combined HCQM is below the upper threshold, refrain from causing the automated slide maker-stainer to prepare a third cytological smear associated with the patient.

In some implementations of the system, the computer is further configured to: receive a second image of the second cytological smear associated with the patient; analyze at least a subset of the second image to determine a second HCQM; compare the second HCQM with the upper threshold; and responsive to determining that the second HCQM is below the upper threshold, refrain from causing the automated slide maker-stainer to prepare a third cytological smear associated with the patient.

In some implementations of the system, the computer is further configured to: receive a second image of the second cytological smear associated with the patient; analyze at least a subset of the second image to determine a second HCQM; compare the second HCQM with a modified upper threshold that is different from the upper threshold used for the first cytological smear; and responsive to determining that the second HCQM is above the modified upper threshold, cause the automated slide maker-stainer to prepare a third cytological smear associated with the patient. In some implementations of the system, the computer is further configured to determine the modified upper threshold based on at least one of: results from analysis of the first cytological smear, a medical condition of the patient, prior medical history of the patient, or analysis requirements associated with the patient.

In some implementations of the system, the computer is further configured to determine the upper threshold based on statistical confidence required for meaningful analysis associated with the HCQM. In some implementations of the system, the computer is further configured to: transmit data related to selection of preparation parameters to the automated slide maker-stainer for preparing the second cytological smear; and select preparation parameters based on characteristics of the HCQM that exceeds the upper threshold. In some implementations of the system, the preparation parameters comprise at least one of: sample dilution ratio, sample concentration technique, smearing speed, smearing angle, smearing pressure, staining duration, or stain concentration.

In some implementations of the system, the HCQM is for a rare cell type, and wherein the upper threshold is set at or above a clinical normal range upper threshold defined for the rare cell type. In some implementations of the system, the first cytological smear is a blood smear, the HCQM is for blast cells, and wherein the upper threshold is set between 1% and 5% of the white blood cells classified in the image. In some implementations of the system, the first cytological smear is a blood smear, the HCQM is for aberrant lymphocytes, and wherein the upper threshold is set between 1% and 5% of the white blood cells classified in the image.

The following implementation improves the reliability of abnormal cell analysis by promoting adequate statistical sampling when HCQM for diagnostically significant abnormal cell type is present in the image but falls below a threshold, such as a limit of clinical normal range, which may affect analytical reliability and diagnostic confidence. By automatically causing preparation of an additional cytological smear when abnormal cell types appear inadequately represented for statistical significance, the system can enhance diagnostic reliability and support consistent quantitative analysis of abnormal cell populations across samples in laboratory settings. This approach can help laboratories manage the inherent statistical challenges associated with abnormal cell detection and quantification without requiring manual intervention, particularly in contexts where the presence and accurate enumeration of small numbers of specific abnormal cell types may have clinical implications. The system's ability to distinguish between classifier confidence issues and statistical sampling adequacy provides a more comprehensive analytical quality framework, addressing the unique challenges posed by diagnostically significant abnormal cell populations.

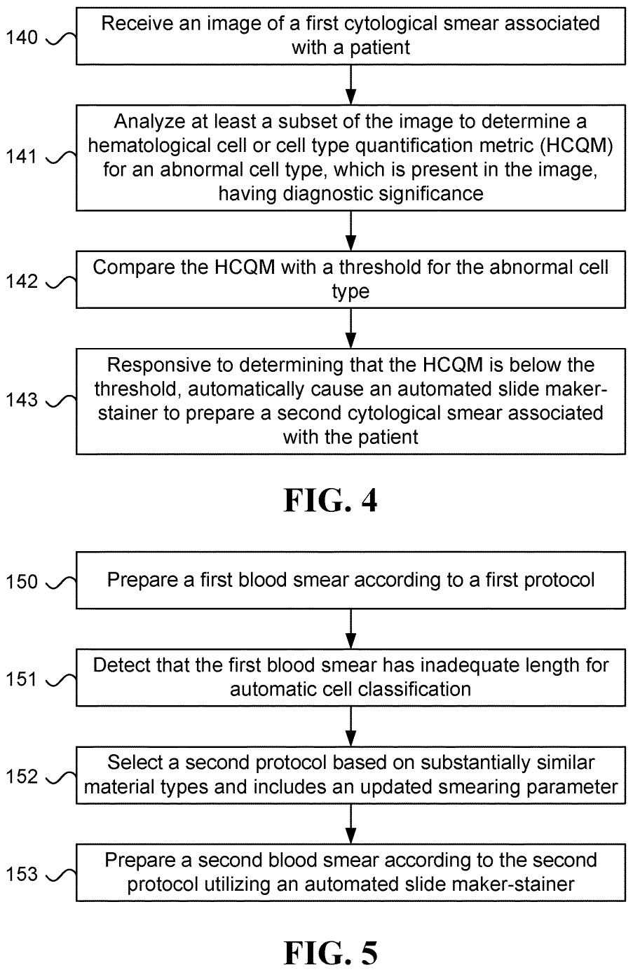

In various implementations, a computer-implemented method for ordering another cytological smear to improve statistical sampling significance, comprising: receiving an image of a first cytological smear associated with a patient; analyzing at least a subset of the image to determine a hematological cell or cell type quantification metric (HCQM) for an abnormal cell type, which is present in the image, having diagnostic significance; comparing the HCQM with a threshold for the abnormal cell type; and responsive to determining that the HCQM is below the threshold, automatically causing an automated slide maker-stainer to prepare a second cytological smear associated with the patient. FIG. 4 illustrates an embodiment of a computer-implemented method for ordering another cytological smear to improve statistical sampling significance. The method includes the following steps. In step 140, receive an image of a first cytological smear associated with a patient. In step 141, analyze at least a subset of the image to determine a hematological cell or cell type quantification metric (HCQM) for an abnormal cell type, which is present in the image, having diagnostic significance. In step 142, compare the HCQM with a threshold for the abnormal cell type. And in step 143, responsive to determining that the HCQM is below the threshold, automatically cause an automated slide maker-stainer to prepare a second cytological smear associated with the patient.

For abnormal cell types, such as blast cells or abnormal lymphocytes, adequate statistical sampling is helpful for diagnostic accuracy even when classifiers operate with high confidence on individual cells. The system may recognize that certain abnormal cell types have diagnostic significance, and their accurate quantification may require larger sample sizes than was measured in the first cytological smear. By comparing the HCQM for these abnormal cell types against thresholds, the system identifies situations where statistical uncertainty due to small sample size may affect diagnostic reliability, optionally regardless of classifier performance on individual cells, to improve statistical confidence in quantitative assessments at the population level.

In some implementations, the method further comprises receiving a second image of the second cytological smear associated with the patient; analyzing the second image to determine a second HCQM for the abnormal cell type; comparing the second HCQM with the threshold; comparing a difference between the HCQM and the second HCQM with a predetermined difference threshold; and responsive to determining that the second HCQM remains below the threshold and that the difference between the HCQM and the second HCQM exceeds the predetermined difference threshold, automatically causing the automated slide maker-stainer to prepare a third cytological smear associated with the patient; whereby the preparation of the third cytological smear is caused even when a classification confidence level for the abnormal cell type is not on an insufficient confidence side of a classification confidence threshold. The system may recognize that for abnormal cell types with critical diagnostic implications, statistical sampling considerations may necessitate additional cytological smears even when the classifier demonstrates high confidence in identifying individual cells. This provision addresses scenarios where the challenge may not be classification accuracy but rather statistical power derived from adequate sample size. Additionally, the system may consider the variance between measurements from different samples, as significant differences between the first and second HCQMs may indicate sample heterogeneity or preparation parameter variability that warrants further investigation. When such significant differences are detected, a third sample may provide clarification about which measurement more accurately represents the patient's condition. For example, in cases where the presence of even small numbers of certain abnormal cell types (such as blast cells in leukemia monitoring) has significant clinical implications, the system may require confirmation through multiple independent samples to achieve the statistical confidence necessary for diagnostic or treatment decisions, particularly when measurements from initial samples show meaningful discrepancies.

In some implementations, the method further comprises receiving a second image of the second cytological smear associated with the patient; analyzing the second image to determine a second HCQM for the abnormal cell type; comparing the second HCQM with the threshold; comparing a difference between the HCQM and the second HCQM with a predetermined difference threshold; and responsive to determining that the second HCQM remains below the threshold and that the difference between the HCQM and the second HCQM does not exceed the predetermined difference threshold, refraining from causing the automated slide maker-stainer to prepare a third cytological smear associated with the patient; whereby the consistency between the HCQM and the second HCQM indicates that further sampling may not yield significantly different results. When analyzing multiple cytological smears for abnormal cell types, the system may evaluate consistency between measurements to estimate whether additional sampling would provide meaningful diagnostic value. When the first and second HCQMs show minimal variance (not exceeding a predetermined difference threshold), this consistency may indicate that the measured quantities likely represent the actual biological state rather than sampling or preparation anomalies. In such scenarios, where consistent results below the threshold are observed across multiple samples, the system may refrain from causing preparation of another cytological smear, recognizing that additional sampling may not yield significantly different information. This approach may help prevent unnecessary resource utilization while maintaining confidence in diagnostic assessments based on the consistency of existing measurements. The predetermined difference threshold may be established based on factors such as known biological variation, system measurement precision, and/or clinical significance of potential quantification differences.

In some implementations, the method further comprises detecting the abnormal cell type at a borderline level in the first cytological smear, wherein borderline level is defined as a quantity of the abnormal cell type that is proximate to but does not reach a clinical diagnostic threshold; and automatically causing the automated slide maker-stainer to prepare the second cytological smear for confirmation of the borderline level of the abnormal cell type. The system may implement specialized handling for scenarios where abnormal cell types are detected at borderline levels—defined as quantities that approach but do not reach established clinical diagnostic thresholds. These borderline levels represent a distinct analytical challenge from both clear positive and clear negative findings, as they occupy a decision boundary where slight variations in sampling or classification could potentially alter clinical interpretation. For diagnostically significant abnormal cell types, such as blast cells or abnormal lymphocytes, these borderline levels may warrant particular attention. The borderline levels may be defined through various mechanisms, including: absolute cell counts within a specified range of the clinical threshold, detection patterns that exhibit statistical characteristics associated with sampling limitations rather than definitive presence or absence, and/or detection of morphological variants that share some but not all definitive characteristics of the diagnostically significant cell type. By automatically causing preparation of additional cytological smears in these borderline scenarios, the system may help resolve diagnostic uncertainty through increased sampling, potentially preventing both false positives and false negatives in diagnostic determinations.

In some implementations, the method further comprises analyzing the at least a subset of the image to determine respective hematological cell types quantification metrics (HCQMs) for multiple abnormal cell types associated with clinical significance weights; comparing the respective HCQMs against corresponding thresholds; calculating a weighted score based on the clinical significance weights and an extent to which at least some of the HCQMs fall below their corresponding thresholds; and automatically causing the automated slide maker-stainer to prepare the second cytological smear when the weighted score exceeds a predetermined value. The system may implement a multi-parameter analysis approach that recognizes different abnormal cell types may carry varying diagnostic importance, with some abnormalities having more significant clinical implications than others. By assigning clinical significance weights to different abnormal cell types, the system may prioritize findings based on their potential impact on patient diagnosis or care. The weighted score calculation may incorporate both the magnitude of threshold deviation (how far below the threshold the HCQM falls) and the clinical importance of the parameters involved. For example, a moderate insufficiency in a highly significant cell type may generate a higher weighted score than a substantial insufficiency in a less clinically significant parameter. The clinical significance weights may be determined based on various factors including: the pathological implications of the cell type, the specificity of the cell type for particular conditions, the rarity of the cell type in normal samples, and/or the prognostic value associated with the cell type's presence or quantity. This weighted approach may help optimize additional smear preparation by triggering verification primarily when clinically meaningful abnormalities are detected at insufficient levels for statistical confidence, potentially reducing unnecessary testing while maintaining appropriate vigilance for significant findings that require adequate statistical power for accurate quantification.

In some implementations, the method further comprises determining the threshold based on clinical guidelines associated with the abnormal cell type having diagnostic significance. The thresholds for abnormal cell types may be selected based on established clinical guidelines, which may reflect research findings, consensus recommendations, regulatory guidelines and field experience, thereby making the results clinically significant and aligned with professional standards. These clinical guidelines may be sourced from various authoritative bodies including: professional medical societies, regulatory agencies, academic institutions, laboratory accreditation organizations, and/or published literature reflecting best practices in hematology and laboratory medicine. The system may incorporate guideline-recommended statistical thresholds that identify minimum sample sizes needed for reliable detection or quantification of diagnostically significant abnormal cell types. The implementation may account for guideline variations across different regions, laboratory settings, or clinical contexts, potentially allowing for configuration based on the specific guidelines applicable to a particular implementation environment. Additionally, the system may include mechanisms for threshold updates as clinical guidelines evolve over time, ensuring alignment with current medical knowledge and practices. This approach may help standardize additional sampling decisions across laboratory operations while maintaining compliance with established medical standards for diagnostic reliability.

In some implementations, the method further comprises adjusting the clinical guidelines based on a diagnostic procedure being performed, and wherein at least some of the diagnostic procedures have different threshold requirements according to their respective statistical confidence requirements for the abnormal cell type. The system may recognize that different diagnostic procedures may require different levels of statistical confidence, and may adjust thresholds accordingly. For example, screening procedures may employ more sensitive thresholds that identify more cases requiring additional sampling to achieve sufficient statistical power, while confirmatory procedures may utilize more specific thresholds to reduce unnecessary additional testing. These adjustments may reflect the different consequences of potential statistical uncertainty in various clinical contexts. In a diagnostic setting for suspected hematological malignancies, the system may implement stricter thresholds requiring higher statistical confidence compared to routine monitoring scenarios where established baselines already exist. Similarly, in post-treatment monitoring procedures, the system may implement heightened sensitivity for minimal residual disease detection, requiring additional sampling at lower HCQM levels compared to initial diagnostic procedures. The threshold adjustments may be applied through various mechanisms, including pre-configured procedure-specific profiles, algorithmic adjustments based on the indicated procedure type, or reference to procedure-specific guidelines maintained within the system. This context-sensitive threshold management may help optimize the balance between diagnostic thoroughness and laboratory efficiency across diverse clinical applications.

In some implementations, the method further comprises receiving a second image of the second cytological smear associated with the patient; analyzing at least a subset of the second image to determine a second HCQM for the abnormal cell type; combining the HCQM and the second HCQM to a combined HCQM; and responsive to determining that the combined HCQM exceeds the threshold, refraining from causing the automated slide maker-stainer to prepare a third cytological smear associated with the patient. The combined HCQM approach may leverage the additional data points provided by the second cytological smear to potentially increase statistical confidence in the overall assessment. This approach recognizes that while individual cytological smears may contain insufficient quantities of abnormal cells for statistical reliability, the combined data from multiple smears may collectively achieve the required statistical power. The combination method may implement various statistical techniques including: simple aggregation of cell counts across samples, weighted combinations based on sample quality indicators, statistical pooling methods that account for inter-sample variability, and/or more complex statistical modeling approaches. If this combined HCQM exceeds the established threshold, indicating sufficient statistical sampling across the combined cytological smears, the system may refrain from preparing a third cytological smear, recognizing that adequate data has been gathered to support classification confidence. This process may optimize resource utilization by limiting additional sample preparation to situations where meaningful clinical benefit may be derived from the additional data, while still maintaining appropriate analytical thoroughness for potential diagnostic findings. The combined assessment approach may be particularly valuable for rare abnormal cell types, where adequate sampling from a single preparation may be challenging due to the inherent rarity of the cell population.

In some implementations, the method further comprises receiving a second image of the second cytological smear associated with the patient; analyzing at least a subset of the second image to determine a second HCQM for the abnormal cell type; comparing the second HCQM with a modified threshold that is different from the threshold used for the first cytological smear; and responsive to determining that the second HCQM is below the modified threshold, automatically causing the automated slide maker-stainer to prepare a third cytological smear associated with the patient. The modified threshold approach may recognize that the context for interpreting the second cytological smear differs from that of the initial analysis. This contextual difference may arise from various factors including: the fact that a second sample was needed suggests potential variability in cell distribution, the presence of two data points provides additional statistical information, and/or laboratory efficiency considerations may warrant adjusting thresholds for subsequent analyses. The modified threshold may be adjusted upward or downward depending on the specific analytical scenario and findings from the first cytological smear. For example, if the initial analysis suggested borderline presence of an abnormal cell type, the modified threshold may be adjusted to require stronger evidence before triggering a third cytological smear. Conversely, if the second cytological smear shows meaningful divergence from the first, suggesting potential sampling variability, the modified threshold may be adjusted to more readily trigger an additional sample to resolve the inconsistency. This adaptive threshold approach may help create a progressive sampling strategy that balances the need for statistical confidence against resource considerations, potentially refining the sampling decision process based on accumulated evidence from multiple cytological smears.

In some implementations, the method further comprises determining the modified threshold based on at least one of: results from analysis of the first cytological smear, a medical condition of the patient, prior medical history of the patient, or analysis requirements associated with the patient. The system may implement context-aware threshold adjustments that incorporate patient-specific and analysis-specific factors when determining the modified threshold for the second cytological smear. These factors may include the specific results from the first cytological smear analysis, such as the magnitude of threshold deviation, distribution patterns of the abnormal cells, or quality indicators from the first cytological smear. The patient's known medical conditions may influence threshold adjustment, potentially modifying expectations for certain abnormal cell populations based on documented diagnoses or current treatments. Prior medical history, including baseline values from previous analyses, may inform personalized thresholds that account for the patient's established cellular presentation patterns. Analysis requirements associated with the patient, such as monitoring protocols for specific conditions, suspected diagnoses requiring confirmation, or heightened vigilance during critical treatment phases, may also factor into threshold modifications. The threshold adjustment process may incorporate algorithms that weight these various factors according to their relevance for the specific parameters being evaluated, potentially allowing for more nuanced decision-making regarding the need for additional sampling. This patient-specific context may allow for more relevant threshold determination that balances the need for appropriate statistical confidence against the goal of minimizing unnecessary testing.

In some implementations, the method further comprises determining the threshold based on statistical confidence required for meaningful analysis associated with the abnormal cell type having diagnostic significance. The threshold determination process may incorporate consideration of the statistical confidence requirements associated with different abnormal cell types and their clinical applications. Parameters with high clinical significance, where statistical uncertainty may significantly impact patient care, may be associated with thresholds that reflect higher statistical confidence requirements. Conversely, parameters with more ambiguous clinical implications may employ thresholds that balance verification needs with resource considerations. The threshold determination process may incorporate statistical methods such as: minimum sample size calculations, confidence interval derivation, statistical power analysis for detection of clinically meaningful changes, and/or distribution modeling techniques appropriate for the specific abnormal cell type being evaluated. Different abnormal cell types may require different minimum sample sizes based on their expected frequency distribution, morphological distinctiveness, clinical normal range, and diagnostic importance. For example, a threshold for blast cells may require a larger statistical sample than for less critical cell types due to the significant treatment implications of blast cell quantification. This statistically informed approach may optimize the balance between sampling thoroughness and testing efficiency by targeting additional sampling toward cases where statistical uncertainty may have meaningful clinical consequences.

In some implementations, the method further comprises transmitting data related to selection of preparation parameters to the automated slide maker-stainer for preparing the second cytological smear; and selecting the preparation parameters based on characteristics of the HCQM that falls below the first threshold. The transmitting of data related to the selection of preparation parameters may encompass multiple implementation approaches. In a first implementation, the system may transmit the specific preparation parameters themselves, providing explicit instructions to the automated slide maker-stainer regarding exactly how to prepare the second slide. In a second implementation, the system may transmit information about the specific inadequacies detected (such as “HCQM for the abnormal cell type that falls below the threshold”), allowing the automated slide maker-stainer to determine appropriate preparation parameters, optionally based on its internal algorithms. In a third implementation, the system may transmit partial parameter information along with problem indicators, creating a collaborative decision process where some parameters are explicitly specified while others are determined by the automated slide maker-stainer based on the nature of the identified issues. The specific implementation may vary based on the capabilities of the automated slide maker-stainer, the complexity of the preparation adjustments required, and the system architecture coupling the analysis engine to the preparation hardware.

In some implementations of the method, the cytological smear is a blood smear and the preparation parameters comprise at least one of: sample dilution ratio, sample concentration technique, smearing speed, smearing angle, smearing pressure, staining duration, or stain concentration. The preparation parameters for blood smears may be selectively adjusted to address specific inadequacies identified in the first cytological smear. Sample dilution ratio adjustments may alter the density of cells to achieve optimal spacing for analysis, with higher dilution potentially used when cell crowding affects visualization and lower dilution potentially used when sparse cell distribution limits statistical sampling. Sample concentration techniques may include methods such as buffy coat preparation or centrifugation parameters that may enhance the representation of specific cell populations, potentially increasing the likelihood of capturing sufficient quantities of rare abnormal cell types. Smearing speed, angle, and pressure parameters may affect the distribution pattern of cells across the slide and the mechanical forces applied to cells during preparation, potentially influencing cell morphology preservation and distribution uniformity. Staining duration and concentration parameters may affect the contrast and visibility of cellular components, potentially enhancing the classifier's ability to identify specific abnormal cell types based on their staining characteristics. The specific parameter adjustments may be determined based on analysis of the inadequacies in the first cytological smear, potentially implementing complementary preparation approaches to address identified limitations. For example, if the first cytological smear showed inadequate representation of abnormal cell types due to suspected dilution issues, the system may adjust parameters to optimize cell concentration in the second preparation. In some implementations of the method, a non-transitory computer-readable medium comprising instructions that, when executed by a processor, cause the processor to perform the computer-implemented method.

In various implementations, a system for ordering another cytological smear to improve statistical sampling significance, comprising: a computer configured to: receive an image of a first cytological smear associated with a patient; analyze at least a subset of the image to determine a hematological cell or cell type quantification metric (HCQM) for an abnormal cell type, which is present in the image, having diagnostic significance; compare the HCQM with a threshold for the abnormal cell type; and responsive to determining that the HCQM is below the threshold, automatically cause an automated slide maker-stainer to prepare a second cytological smear associated with the patient. In some implementations of the system, the computer is further configured to: receive a second image of the second cytological smear associated with the patient; analyze the second image to determine a second HCQM for the abnormal cell type; compare the second HCQM with the threshold; compare a difference between the HCQM and the second HCQM with a predetermined difference threshold; and responsive to determining that the second HCQM remains below the threshold and that the difference between the HCQM and the second HCQM exceeds the predetermined difference threshold, automatically cause the automated slide maker-stainer to prepare a third cytological smear associated with the patient; whereby the preparation of the third cytological smear is caused even when a classification confidence level for the abnormal cell type is not on an insufficient confidence side of a classification confidence threshold.

In some implementations of the system, the computer is further configured to: receive a second image of the second cytological smear associated with the patient; analyze the second image to determine a second HCQM for the abnormal cell type; compare the second HCQM with the threshold; compare a difference between the HCQM and the second HCQM with a predetermined difference threshold; and responsive to determining that the second HCQM remains below the threshold and that the difference between the HCQM and the second HCQM does not exceed the predetermined difference threshold, refrain from causing the automated slide maker-stainer to prepare a third cytological smear associated with the patient; whereby the consistency between the HCQM and the second HCQM indicates that further sampling may not yield significantly different results.

In some implementations of the system, the computer is further configured to: detect the abnormal cell type at a borderline level in the first cytological smear, wherein borderline level is defined as a quantity of the abnormal cell type that is proximate to but does not reach a clinical diagnostic threshold; and automatically cause the automated slide maker-stainer to prepare the second cytological smear for confirmation of the borderline level of the abnormal cell type.

In some implementations of the system, the computer is further configured to: analyze the at least a subset of the image to determine respective hematological cell types quantification metrics (HCQMs) for multiple abnormal cell types associated with clinical significance weights; compare the respective HCQMs against corresponding thresholds; calculate a weighted score based on the clinical significance weights and an extent to which at least some of the HCQMs fall below their corresponding thresholds; and automatically cause the automated slide maker-stainer to prepare the second cytological smear when the weighted score exceeds a predetermined value.

In some implementations of the system, the computer is further configured to determine the threshold based on clinical guidelines associated with the abnormal cell type having diagnostic significance. In some implementations of the system, the computer is further configured to adjust the clinical guidelines based on a diagnostic procedure being performed, and wherein at least some of the diagnostic procedures have different threshold requirements according to their respective statistical confidence requirements for the abnormal cell type. In some implementations of the system, the computer is further configured to: receive a second image of the second cytological smear associated with the patient; analyze at least a subset of the second image to determine a second HCQM for the abnormal cell type; combine the HCQM and the second HCQM to a combined HCQM; and responsive to determining that the combined HCQM exceeds the threshold, refrain from causing the automated slide maker-stainer to prepare a third cytological smear associated with the patient.

In some implementations of the system, the computer is further configured to: receive a second image of the second cytological smear associated with the patient; analyze at least a subset of the second image to determine a second HCQM for the abnormal cell type; compare the second HCQM with a modified threshold that is different from the threshold used for the first cytological smear; and responsive to determining that the second HCQM is below the modified threshold, automatically cause the automated slide maker-stainer to prepare a third cytological smear associated with the patient. In some implementations of the system, the computer is further configured to determine the modified threshold based on at least one of: results from analysis of the first cytological smear, a medical condition of the patient, prior medical history of the patient, or analysis requirements associated with the patient.

In some implementations of the system, the computer is further configured to determine the threshold based on statistical confidence required for meaningful analysis associated with the abnormal cell type having diagnostic significance. In some implementations of the system, the computer is further configured to: transmit data related to selection of preparation parameters to the automated slide maker-stainer for preparing the second cytological smear; and select the preparation parameters based on characteristics of the HCQM that falls below the threshold. In some implementations of the system, the cytological smear is a blood smear and the preparation parameters comprise at least one of: sample dilution ratio, sample concentration technique, smearing speed, smearing angle, smearing pressure, staining duration, or stain concentration.

In various implementations, a computer-implemented method for ordering another cytological smear to improve statistical sampling significance, comprising: receiving an image of a first cytological smear associated with a patient; analyzing at least a subset of the image to determine a hematological cell or cell type quantification metric (HCQM) for an abundant cell type having diagnostic significance; comparing the HCQM with a clinical normal range for abundant cell type; and responsive to determining that the HCQM is at least a predetermined amount below lower limit of the clinical normal range, automatically causing an automated slide maker-stainer to prepare a second cytological smear associated with the patient. For abundant cell types, such as neutrophils, lymphocytes, or erythrocytes, abnormally low quantities may indicate various pathological conditions, and the accuracy of these deficiency assessments may benefit from statistical confirmation. The system may recognize when the HCQM for these abundant cell types falls significantly below established clinical normal ranges, potentially triggering additional sampling to verify these findings. The predetermined amount below the lower limit may be defined in various ways, including: as an absolute value below the lower limit, as a percentage deviation from the lower limit, as a statistical measure such as a number of standard deviations below the lower limit, and/or through more complex algorithms incorporating multiple reference parameters. This approach may address scenarios where deficiencies in normally abundant cell populations require verification due to their diagnostic significance, such as neutropenia potentially indicating bone marrow disorders, immune dysfunction, or medication effects. By automatically causing preparation of another cytological smear in these cases, the system may help confirm whether the deficiency represents a genuine patient condition or potentially resulted from preparation variations or sampling limitations in the first cytological smear, which may be particularly valuable when treatment decisions depend on accurate quantification of these cellular deficiencies. Additionally, automatically preparing another slide and testing it may reduce the amount of human review required.

In some implementations, the method further comprises receiving a second image of the second cytological smear associated with the patient; analyzing the second image to determine a second HCQM for the abundant cell type; comparing the second HCQM with the clinical normal range; comparing a difference between the HCQM and the second HCQM with a predetermined difference threshold; and responsive to determining that the second HCQM remains at least the predetermined amount below the lower limit of the clinical normal range and that the difference between the HCQM and the second HCQM exceeds the predetermined difference threshold, automatically causing the automated slide maker-stainer to prepare a third cytological smear associated with the patient; whereby the preparation of the third cytological smear is caused even when a classification confidence level for the abundant cell type is not on an insufficient confidence side of a classification confidence threshold. The system may implement a multi-sample analysis approach for abundant cell types that considers both the persistent deficiency across samples and the variance between measurements. When the second HCQM also indicates a significant deficiency (remaining at least the predetermined amount below the lower limit of the clinical normal range) but differs substantially from the first measurement (exceeding the predetermined difference threshold), this discrepancy may suggest sample heterogeneity, preparation variability, or other factors affecting measurement reliability. The predetermined difference threshold may be established through various mechanisms, including: statistical derivation from known biological variation patterns, laboratory-specific performance metrics, and/or clinical significance considerations for the specific cell type. Importantly, this determination may operate independently from classification confidence considerations, recognizing that the challenge may not be in the classifier's ability to identify the cell type but rather in ensuring adequate statistical sampling and measurement consistency. For example, in cases of suspected neutropenia, where neutrophil counts fall significantly below normal ranges but show substantial variation between the first and second samples, a third sample may help establish whether the lower or higher count more accurately represents the patient's condition, potentially affecting critical treatment decisions such as chemotherapy timing or infection risk assessment, even when the classifier demonstrates high confidence in identifying neutrophils in both samples.

In some implementations, the method further comprises receiving a second image of the second cytological smear associated with the patient; analyzing the second image to determine a second HCQM for the abundant cell type; comparing the second HCQM with the clinical normal range; comparing a difference between the HCQM and the second HCQM with a predetermined difference threshold; and responsive to determining that the second HCQM remains at least the predetermined amount below the lower limit of the clinical normal range and that the difference between the HCQM and the second HCQM does not exceed the predetermined difference threshold, refraining from causing the automated slide maker-stainer to prepare a third cytological smear associated with the patient; whereby the consistency between the HCQM and the second HCQM indicates that further sampling may not yield significantly different results. When analyzing multiple cytological smears for deficiencies in abundant cell types, the system may evaluate consistency between measurements to determine whether additional sampling would provide meaningful diagnostic value. When the first and second HCQMs both indicate significant deficiencies (both remaining at least the predetermined amount below the lower limit of the clinical normal range) and show minimal variance between them (not exceeding the predetermined difference threshold), this consistency may suggest that the measured deficiency likely represents the actual biological state rather than sampling or preparation anomalies. This consistency evaluation may incorporate various statistical approaches, including: percent difference calculations, absolute difference measurements, statistical significance testing between measurements, and/or more complex algorithms that account for expected biological and technical variation. In such scenarios, where consistent deficiencies are observed across multiple samples, the system may refrain from causing preparation of a third cytological smear, recognizing that additional sampling may not yield significantly different information while potentially delaying clinical decision-making. This approach may help prevent unnecessary resource utilization while maintaining confidence in diagnostic assessments based on the consistency of existing measurements, which may be particularly valuable in time-sensitive clinical scenarios where treatment decisions for conditions associated with deficiencies in abundant cell types may need to proceed based on confirmed findings without unnecessary delay.

In some implementations, the method further comprises analyzing the at least a subset of the image to determine respective hematological cell types quantification metrics (HCQMs) for multiple abundant cell types associated with clinical significance weights; comparing the respective HCQMs against corresponding clinical normal ranges; calculating a weighted score based on the clinical significance weights and an extent to which at least some of the HCQMs fall below the lower limits of their corresponding clinical normal ranges; and automatically causing the automated slide maker-stainer to prepare the second cytological smear when the weighted score exceeds a predetermined value. The system may implement a multi-parameter analysis approach that recognizes different abundant cell types may carry varying diagnostic importance when deficient. By assigning clinical significance weights to different abundant cell types, the system may prioritize findings based on their potential impact on patient care. These clinical significance weights may be determined through various means, including: established clinical knowledge about the diagnostic significance of deficiencies in specific cell types, input from medical experts, and/or reference to published literature on the implications of various cytopenias. The weighted score calculation may incorporate both the magnitude of deficiency (how far below the lower limit the HCQM falls) and the clinical importance of the parameters involved. For example, a moderate deficiency in a highly significant cell type (such as neutrophils in an immunocompromised patient) may generate a higher weighted score than a substantial deficiency in a less clinically significant parameter. This weighted approach may help optimize additional smear preparation by triggering verification primarily when clinically meaningful deficiencies are detected, potentially reducing unnecessary testing while maintaining appropriate vigilance for significant findings. The predetermined value at which the weighted score triggers additional sampling may be established through various mechanisms, including statistical modeling, clinical validation studies, and/or laboratory-specific optimization processes that balance sensitivity and specificity considerations.

In some implementations, the method further comprises determining the clinical normal range based on clinical guidelines associated with the abundant cell type having diagnostic significance. The clinical normal ranges for abundant cell types may be selected based on established clinical guidelines, which may reflect research findings, regulatory guidelines, consensus recommendations, and field experience. These clinical guidelines may be sourced from various authoritative bodies including: professional medical societies, regulatory agencies, academic institutions, laboratory accreditation organizations, and/or published literature reflecting best practices in hematology and laboratory medicine. The system may incorporate different reference ranges for different patient populations, recognizing that normal ranges may vary based on factors such as age, sex, ethnicity, geographical location, and/or altitude. The implementation may account for guideline variations across different regions, laboratory settings, or clinical contexts, potentially allowing for configuration based on the specific guidelines applicable to a particular implementation environment. Additionally, the system may include mechanisms for range updates as clinical guidelines evolve over time, ensuring alignment with current medical knowledge and practices. The clinical normal ranges may be stored in reference databases within the system, potentially with metadata indicating their source, applicable population characteristics, and/or revision history to maintain transparency and traceability in the assessment process.

In some implementations, the method further comprises receiving a second image of the second cytological smear associated with the patient; analyzing at least a subset of the second image to determine a second HCQM for the abundant cell type; combining the HCQM and the second HCQM to a combined HCQM; and responsive to determining that the combined HCQM is not at least the predetermined amount below the lower limit of the clinical normal range, refraining from causing the automated slide maker-stainer to prepare a third cytological smear associated with the patient. The combined HCQM approach may leverage the additional data points provided by the second cytological smear to potentially increase statistical confidence in the overall assessment for abundant cell types. The combination method may implement various statistical techniques including: averaging of values from both samples, weighted combinations based on sample quality indicators, statistical approaches that account for known sources of variation between samples, and/or more complex algorithmic approaches that optimize the integration of multiple measurements. If this combined HCQM no longer indicates a significant deficiency (not at least the predetermined amount below the lower limit of the clinical normal range), the system may refrain from preparing a third cytological smear, recognizing that the combined data suggests the deficiency may not be as severe as initially indicated by the first sample alone. This approach may address scenarios where measurement variability in individual samples might create false impressions of clinically significant deficiencies, while the combined assessment provides a more statistically robust evaluation. The process may optimize resource utilization by limiting additional sample preparation to situations where meaningful clinical benefit may be derived from the additional data, while still maintaining appropriate analytical thoroughness for potential diagnostic findings. This combined assessment may be particularly valuable in clinical scenarios where treatment decisions depend on accurate quantification of abundant cell populations, such as monitoring for bone marrow suppression during chemotherapy or assessing recovery from medication-induced cytopenias.

In some implementations of the method, the cytological smear is a blood smear, and wherein automatically causing the automated slide maker-stainer to prepare the second cytological smear comprises: selecting one or more preparation parameters for the second cytological smear, wherein the selection is based at least in part on the determination that the HCQM is at least the predetermined amount below the lower limit of the clinical normal range; and transmitting data indicative of the selected one or more preparation parameters to the automated slide maker-stainer for use in preparing the second cytological smear; wherein the one or more preparation parameters comprise at least one of: sample dilution ratio, sample concentration technique, smearing speed, smearing angle, smearing pressure, staining duration, or stain concentration. When deficiencies in abundant cell types are detected, the system may implement targeted protocol modifications for the second cytological smear to specifically address these findings. These modifications may seek to optimize visualization and quantification of the deficient cell populations through various parameter adjustments. The sample dilution ratio may be modified to create optimal cell density on the slide, with potential decreases in dilution when deficiencies are detected to concentrate the available cells and increase the likelihood of capturing sufficient quantities for reliable assessment. Sample concentration techniques may be employed or adjusted, potentially implementing methods such as buffy coat preparation with optimized centrifugation parameters specifically selected to enhance recovery and visualization of deficient cell types. Smearing parameters including speed, angle, and pressure may be modified to create optimal cell distribution patterns that improve the visibility and analyzable area for the deficient cell populations, potentially employing gentler mechanical forces when cellular fragility is a concern. Staining parameters including duration and concentration may be adjusted to enhance the visualization of deficient cell types, potentially employing modified protocols that optimize contrast and differentiation for the specific cell populations of interest. The selection of which parameters to modify may be determined through analysis of the specific deficiency patterns observed in the first cytological smear, potentially implementing complementary preparation approaches targeted at resolving the particular limitations identified. The transmitted data may include explicit parameter values, preparation method identifiers, or protocol selection indicators depending on the capabilities and interface requirements of the automated slide maker-stainer.

In various implementations, a system for ordering another cytological smear to improve statistical sampling significance, comprising: a computer configured to: receive an image of a first cytological smear associated with a patient; analyze at least a subset of the image to determine a hematological cell or cell type quantification metric (HCQM) for an abundant cell type having diagnostic significance; compare the HCQM with a clinical normal range for the abundant cell type; and responsive to determining that the HCQM is at least a predetermined amount below a lower limit of the clinical normal range, automatically cause an automated slide maker-stainer to prepare a second cytological smear associated with the patient.

In some implementations of the system, the computer is further configured to: receive a second image of the second cytological smear associated with the patient; analyze the second image to determine a second HCQM for the abundant cell type; compare the second HCQM with the clinical normal range; compare a difference between the HCQM and the second HCQM with a predetermined difference threshold; and responsive to determining that the second HCQM remains at least the predetermined amount below the lower limit of the clinical normal range and that the difference between the HCQM and the second HCQM exceeds the predetermined difference threshold, automatically cause the automated slide maker-stainer to prepare a third cytological smear associated with the patient; whereby the preparation of the third cytological smear is caused even when a classification confidence level for the abundant cell type is not on an insufficient confidence side of a classification confidence threshold.

In some implementations of the system, the computer is further configured to: receive a second image of the second cytological smear associated with the patient; analyze the second image to determine a second HCQM for the abundant cell type; compare the second HCQM with the clinical normal range; compare a difference between the HCQM and the second HCQM with a predetermined difference threshold; and responsive to determining that the second HCQM remains at least the predetermined amount below the lower limit of the clinical normal range and that the difference between the HCQM and the second HCQM does not exceed the predetermined difference threshold, refrain from causing the automated slide maker-stainer to prepare a third cytological smear associated with the patient; whereby the consistency between the HCQM and the second HCQM indicates that further sampling may not yield significantly different results.

In some implementations of the system, the computer is further configured to: analyze the at least a subset of the image to determine respective hematological cell types quantification metrics (HCQMs) for multiple abundant cell types associated with clinical significance weights; compare the respective HCQMs against corresponding clinical normal ranges; calculate a weighted score based on the clinical significance weights and an extent to which at least some of the HCQMs fall below the lower limits of their corresponding clinical normal ranges; and automatically cause the automated slide maker-stainer to prepare the second cytological smear when the weighted score exceeds a predetermined value. In some implementations of the system, the computer is further configured to determine the clinical normal range based on clinical guidelines associated with the abundant cell type having diagnostic significance.

In some implementations of the system, the computer is further configured to: receive a second image of the second cytological smear associated with the patient; analyze at least a subset of the second image to determine a second HCQM for the abundant cell type; combine the HCQM and the second HCQM to a combined HCQM; and responsive to determining that the combined HCQM is not at least the predetermined amount below the lower limit of the clinical normal range, refrain from causing the automated slide maker-stainer to prepare a third cytological smear associated with the patient.

In some implementations of the system, the cytological smear is a blood smear, and wherein the computer, when configured to automatically cause the automated slide maker-stainer to prepare the second cytological smear, is further configured to: select one or more preparation parameters for the second cytological smear, wherein the selection is based at least in part on the determination that the HCQM is at least the predetermined amount below the lower limit of the clinical normal range; and transmit data indicative of the selected one or more preparation parameters to the automated slide maker-stainer for use in preparing the second cytological smear; wherein the one or more preparation parameters comprise at least one of: sample dilution ratio, sample concentration technique, smearing speed, smearing angle, smearing pressure, staining duration, or stain concentration.

In various embodiments, the method for adjusting an automated slide maker based on spatial distribution of classified cells may encompass multiple approaches for identifying and addressing inadequacies in cytological smears. The determination that a cytological smear is inadequate due to spatial distribution issues may involve analysis of numerous factors, including but not limited to: the length and shape of the smear, the distribution pattern of classified cells, the relative locations of different cell types, the proportion of cells in the monolayer versus the feathered edge, and/or any other spatial characteristics that may affect classification accuracy. This analysis may be performed after cell classification, which may allow the system to evaluate the internal distribution of cells to determine whether the smearing was adequate for cell classification purposes. The cell classification process may involve analyzing large numbers of cells, potentially exceeding 10,000 or 100,000 cells in some implementations, which may provide statistical robustness to the spatial distribution assessment.

The inadequate spatial distribution may manifest in various forms that may raise flags for further investigation, such as smears that are too short or too long, improper ratios of cells between different regions of the smear, cell clumping (including but not limited to rouleau formation and/or platelet clumps), and/or distributions where heavier cells appear predominantly in the monolayer while lighter cells concentrate in the feathered edge. In some cases, a smear may be relatively short but still contain enough properly distributed cells for adequate classification, potentially eliminating the need to produce a new slide even if the smear is shorter than usual. Conversely, a smear may appear sufficiently long but after classification reveal illogical cell distribution or insufficient quantities of certain cell types, which may trigger the preparation of another cytological smear to improve classification accuracy.

The selection of a second protocol with adjusted smearing parameters may involve modifications to one or more operational aspects of the slide maker, potentially including but not limited to: adjustments to the speed of the smearing blade, alterations to the angle of the smearing blade, replacement of the blade, and/or any other parameter adjustments that may improve smearing results. For example, the system may identify a deterioration process in the length of the cytological smear, sometimes due to blade wear, and then recommend changes to one or more smearing parameters with the aim of extending the smear. These protocol adjustments may be determined through various analytical approaches, such as trend analysis from previous smears, assessment of the specific inadequacies identified in the current smear, and/or reference to predefined parameter adjustment guidelines.

The method may optionally include providing recommendations or instructions through various channels, such as automated interfaces that directly adjust slide maker settings, user interfaces that guide technicians through manual adjustments, and/or documentation of recommended protocol changes for future reference. Examples of instructions to laboratory technicians may be modified based on the deviations in spatial distribution of cells, potentially including requests to prepare one or more additional slides, change the smearing blade, run the slide again with a larger scan area, or analyze a larger area of the existing scan. The normal ranges for certain cell types may potentially vary based on smear parameters, as some cells may migrate to different regions of the smear (such as from the monolayer to the feathered edge) based on physical characteristics, which may necessitate adjustments to scan areas to account for different types of cytopenia, short smears, and/or other variations.

The method may generate various types of outputs from the analysis, potentially including quantification of cells and anomalies, identification of rare cell types, and/or comprehensive mapping of the spatial distribution of cells across the slide. From the spatial distribution parameters, it may be possible to deduce that a problem is related to smearing and provide specific instructions on needed corrections, such as whether the smearing needs to be slower or faster, or whether the blade should be replaced. In cases where a cytological smear is identified as being too short yet adequate for cell classification, instead of preparing an additional cytological smear for the same patient, the system may provide an indication of parameter changes needed for subsequent cytological smears (typically for other patients) to extend the smear length. The process of determining inadequacy and selecting adjusted protocols may occur through automated analysis, human review, or combinations thereof, potentially providing flexibility in implementation across various laboratory environments and workflows.

In various embodiments, the computer-implemented method for determining whether to prepare another cytological smear based on artifacts may address quality issues in slide preparation that may compromise automated analysis. The method may involve receiving an image of a cytological smear associated with a patient and data indicative of automatically classified cells or automatically detected artifacts in that image. This data may be generated through various image analysis techniques, pattern recognition algorithms, and/or machine learning models trained to identify cellular structures or preparation artifacts within cytological images. By quantifying the impact of these artifacts on classification accuracy through the calculation of an artifact impact measure, the system may make evidence-based decisions about whether a new slide is needed. This process may reduce manual review time by having the automated system assess slide quality and may allow for a self-monitoring analytical pipeline that can identify quality issues and initiate corrective actions without human intervention.

The artifacts that may be detected and evaluated by the system may include, but are not limited to: rouleaux formations (abnormal stacking of red blood cells), spherocytes (abnormally spherical erythrocytes), smudge cells (damaged cells with distorted morphology), bubbles (air pockets in mounting media), and/or scratches (physical damage to the slide surface). The predetermined threshold for determining when to prepare another cytological smear may be adjusted based on the presence and distribution of these specific artifact types, recognizing that different artifacts may have varying impacts on classification accuracy. The threshold adjustment may incorporate weighted scoring systems that assign different significance levels to different artifact types and/or may implement dynamic threshold modifications based on the spatial relationship between artifacts and classified cells.

When the system determines that the artifact impact reaches the predetermined threshold, it may not only command the preparation of another cytological smear but may also identify the specific parameter that contributed to the artifacts. This parameter identification may involve analyzing the morphological characteristics of the detected artifacts, their distribution patterns, and/or their relationship to specific aspects of the preparation process. Once identified, the system may command the automated slide maker-stainer to prepare the another cytological smear according to a modified protocol that adjusts the specific parameter identified as contributing to the artifacts. These protocol modifications may be derived from predefined correction matrices that map artifact types to relevant preparation parameters, from statistical models that correlate artifact characteristics with optimal preparation adjustments, and/or from adaptive learning systems that refine protocol modifications based on outcomes from previous corrections.

The calculation of artifact impact may be configured to be more sensitive to artifacts affecting regions containing rare cell types compared to artifacts affecting regions containing common cell types. This differential sensitivity recognizes that rare cell types often carry heightened diagnostic significance and that statistical reliability for rare populations requires greater quality control due to their limited representation in the sample. The system may implement this increased sensitivity through various mechanisms, such as applying lower artifact impact thresholds for regions containing rare cells, weighting the artifact impact calculation to give greater importance to artifacts near rare cells, and/or expanding the spatial influence radius of artifacts when they occur near rare cell populations. This targeted sensitivity may help optimize the balance between quality control and unnecessary repeat testing by focusing stringent quality requirements on the cellular populations where they may have high diagnostic value.

The system may adjust artifact impact thresholds based on specific staining quality parameters while maintaining diagnostic reliability. When analyzing staining intensity (which may be measured, for example through color histogram analysis of cellular components), the system may detect suboptimal staining that falls below target reference values but still allows for adequate morphological assessment. In such cases, rather than automatically triggering another smear preparation due to staining inadequacy, the system may increase the predetermined threshold for artifact impact, when the artifact can relate to staining, by a certain percent (e.g., 5-10%) to accommodate the suboptimal staining while avoiding unnecessary repeat procedures. This adjustment is based on the assumption that suboptimal staining may still provide sufficient information for accurate classification when cell morphology remains distinguishable. The determination of whether morphology remains distinguishable may involve analyzing contrast ratios between nuclear and cytoplasmic components, edge detection clarity of cellular boundaries, and/or visibility of specific intracellular structures relevant to classification. This logic may reduce unnecessary smear preparations due to minor staining variations that don't significantly impact diagnostic accuracy.

Cell distribution patterns across a cytological smear can significantly affect the reliability of automated analysis, particularly when uneven distribution creates regions with substantially different cell density. Certain artifacts, such as smearing artifacts that cause cells to cluster abnormally or create cell-free zones, directly affect distribution patterns. When the system detects uneven cell distribution (e.g., measured by variance in cell density across different regions of the smear), it may increase or decrease the predetermined threshold for artifact impact by an appropriate value to reflect the higher statistical requirements needed for the analysis to adequately represent the true cell population despite distribution challenges. In one example, the system may implement this adjustment by mapping cell density across a predetermined area of the smear, calculating variance coefficients, and applying threshold adjustments proportional to the detected distribution irregularity. For instance, minor distribution irregularities may warrant modest threshold adjustment, while severe irregularities may require larger adjustments. This process helps flagging for repeat preparation samples with distribution challenges that may impact statistical reliability.

The system may calculate the artifact impact, which quantifies how various artifacts affect classification accuracy, by evaluating multiple artifact characteristics such as type (with different weights assigned to different artifact categories based on their typical impact on classification), severity (measured by parameters specific to artifact types), and/or distribution (considering what percentage of the analysis area is affected). The resulting artifact impact provides a measure of how significantly the artifacts interfere with reliable classification. The system then may adjust the threshold for ordering another smear as a function of this impact score. This approach may allow for nuanced evaluation of artifact influence that considers not just the presence of artifacts but their specific characteristics and potential to compromise analytical accuracy.

Different categories of artifacts originate from different aspects of the preparation process and may require distinct corrections. The system may implement category-specific protocol adjustments that target the main causes of different artifact types. For staining artifacts (such as stain precipitates, uneven staining, or stain intensity issues), the protocol adjustments may focus on chemical aspects of preparation-such as modifying staining solution quality through additional washing steps, implementing filtration procedures for solutions, adjusting staining duration, or modifying stain concentration. For distribution artifacts (such as insufficient monolayer formation, cell clustering, or inadequate smear length), the protocol adjustments may focus on mechanical aspects of preparation-such as modifying smearing speed, adjusting smearing angle, altering smearing pressure, or changing blood volume used in preparation. This category-specific adjustment recognizes that artifacts and distribution issues may stem from different preparation components and may require different corrective strategies. For scenarios where both staining and distribution artifacts are detected in the same sample, the system may implement a decision tree that prioritizes corrections based on which category of artifact most significantly impacts classification accuracy, and/or may implement a dual-correction that simultaneously addresses both issue categories with complementary protocol adjustments.

When multiple artifact categories are detected in a single cytological smear, the system may prioritize corrective actions on the most impactful issues first, which optionally begins with calculating classification impact scores for at least some of the artifact categories, which quantify how significantly the artifact categories interfere with accurate cell classification. These scores may consider factors such as the percentage of the analysis area affected, the magnitude of CCL reduction associated with the artifacts, and/or the clinical significance of the affected cell types. After determining impact scores, the system prioritizes protocol adjustments addressing the highest-impact artifact category. For example, if staining artifacts are associated with a higher impact score than distribution artifacts in a particular sample, the protocol adjustments would primarily target staining parameters. The system may also implement secondary protocol adjustments for lower-impact artifact categories as long as these adjustments do not undermine the primary corrections. This prioritization may help improve the likelihood that subsequent preparations will successfully address the most significant quality limitations of the original sample.

The following implementation creates a feedback loop between analysis and preparation, enabling the system to adapt from preparation inadequacies. Rather than simply ordering another slide according to the same protocol, the system may identify specific preparation issues and modify protocols accordingly, which may reduce repeated errors and increase efficiency by targeting corrections to the specific issues identified. The embodiment supports continuous improvement of smear preparation protocol and may enhance overall quality while reducing waste from inadequate smear preparations.

In various implementations, a computer-implemented method for preparing additional slides based on inadequate smear length, comprising: preparing a first blood smear associated with a patient according to a first protocol; detecting that the first blood smear has inadequate length for automatic cell classification; selecting a second protocol, wherein the second protocol is based on substantially similar material types as the first protocol, and wherein the second protocol comprises an updated smearing parameter intended to produce a blood smear having adequate length for the automatic cell classification; and preparing, utilizing an automated slide maker-stainer, a second blood smear associated with the patient according to the second protocol. FIG. 5 illustrates an embodiment of a computer-implemented method for preparing additional slides based on inadequate smear length. The method includes the following steps. In step 150, prepare a first blood smear associated with a patient according to a first protocol. In step 151, detect that the first blood smear has inadequate length for automatic cell classification. In step 152, select a second protocol, wherein the second protocol is based on substantially similar material types as the first protocol, and wherein the second protocol comprises an updated smearing parameter intended to produce a blood smear having adequate length for the automatic cell classification. And in step 153, prepare, utilizing an automated slide maker-stainer, a second blood smear associated with the patient according to the second protocol.

This embodiment provides an automated approach for addressing blood smears with inadequate length, which can impact the quality and reliability of automatic cell classification. When a blood smear exhibits length inadequacies-either being too short or too long-certain cell populations may not be properly represented or classified. By detecting length inadequacies and implementing appropriate protocol modifications, the system can produce a subsequent blood smear with improved length characteristics. The first and second blood smears may be prepared using the same automated slide maker-stainer or using different automated slide maker-stainers. Stain type refers to the materials used to stain blood cells which are smeared. Some of the common types of stains include Wright-Giemsa, May-Grunwald, Giemsa, Wright and other possible combinations. These are often accompanied by fixation solutions and buffers. Stain types often include a solution intended to stain acidic elements and a solution intended to stain base elements.

Optionally, the first blood smear may be prepared in a variety of contexts prior to the detection of its inadequate length. For example, its preparation may be performed by personnel within the same organization that conducts the analysis, or it could be performed by a separate entity, such as a different laboratory or clinic. The physical location of the sample preparation may also vary. A smear may be prepared at the same site where the analysis is conducted or at a remote location, possibly requiring transport to the analysis facility. In some embodiments, the preparation and analysis may occur in close temporal proximity, while in other embodiments, a delay may exist between the preparation of the first smear and its subsequent analysis.

In some implementations of the method, the substantially similar material types comprise at least one of: staining reagents from the same chemical family, buffer solutions with comparable pH ranges, fixatives with similar cell preservation mechanisms, slide substrates with comparable surface characteristics, or anticoagulants with similar effects on blood viscosity; and wherein the updated smearing parameter comprises at least one of: smearing speed, smearing angle, smearing pressure, or blood volume. In various embodiments, “substantially similar material types” refers to preparation materials that maintain comparable chemical, physical, and/or functional characteristics when used in cytological smear preparation protocols. In the context of the first and second protocols, substantially similar material types, while potentially not identical, are sufficiently alike in composition and function such that their use in the second protocol does not fundamentally alter the expected outcome of the blood smear preparation beyond the changes intentionally introduced by the updated smearing parameter. For blood smear preparation, substantially similar material types may include: staining reagents from the same chemical family that produce comparable differentiation of cellular components, buffer solutions with comparable pH ranges and chemical compositions, fixatives that preserve cellular structures through similar chemical mechanisms, glass or plastic slide substrates with comparable surface characteristics, and/or diluents or anticoagulants with similar effects on blood viscosity and cellular preservation.

The smearing parameters influence the physical distribution and arrangement of blood cells across the slide. Smearing speed affects how quickly the blood is spread, with faster speeds typically producing longer smears. Smearing angle influences the thickness gradient and formation of the feathered edge. Smearing pressure affects how forcefully the blood is distributed, with higher pressure typically creating longer smears. Blood volume determines the amount of sample available for distribution. By adjusting these parameters, the system can address the specific aspects of the smearing process that contributed to the inadequate length in the first blood smear. Possible methods for creating a smear include, but are not limited to, wedge method, centrifuge, cell printing and sample pressing.

In some implementations of the method, the detecting that the first blood smear has inadequate length comprises determining that the first blood smear is too short, resulting in insufficient cell distribution for reliable automatic cell classification. When a blood smear is too short, the proper formation of the monolayer region may be compromised, which can result in insufficient representation of different cell populations, particularly for rare cell types that require examination of larger sample areas for reliable detection. Additionally, a short smear may contain too few cells for statistically valid automatic classification, especially for differential counts that require minimum cell numbers for diagnostic accuracy. Additionally, a short smear can affect the bulk area which may affect the ability to detect parasites such as Malaria.

In some implementations of the method, the detecting that the first blood smear has inadequate length comprises determining that the first blood smear is too long, resulting in at least one of: absence of a feathered edge due to cells being spread to the edge of a slide, which reduces the ability to identify certain conditions related to white blood cells and platelets, or (ii) excessive spacing between cells, which prevents accurate identification of a monolayer region. When a blood smear is too long, distinct analytical problems can occur as the cells are spread all the way to the edge of the slide without forming a proper feathered edge, which disrupts the normal cellular distribution pattern expected in standard analysis. The feathered edge region is typically used for examining WBC morphology often related to larger cells, and often helpful also in analyzing platelet clumps. Additionally, excessive smear length can create regions where cells are so widely spaced that identifying a proper monolayer region becomes more difficult.

In some implementations of the method, the detecting that the first blood smear has inadequate length comprises utilizing a deep learning model trained on examples of adequate blood smear lengths and inadequate blood smear lengths. Deep learning models provide an effective approach for smear length adequacy assessment by learning from large datasets of previously prepared slides. These models can be trained on diverse examples representing both adequate and inadequate smear lengths, enabling them to recognize subtle patterns that may not be captured by rule-based systems. The deep learning approach may integrate multiple features including overall smear length, cell distribution patterns, monolayer region characteristics, and feathered edge formation that collectively determine whether a smear has appropriate length for reliable automatic cell classification.

In some implementations of the method, the detecting that the first blood smear has inadequate length comprises determining that detection performance of an automatic cell detector applied to an image of the first blood smear is below a predetermined value. The performance of the automatic cell detector can provide a functional assessment of smear length adequacy. When the detector's performance falls below a predetermined value, it may indicate that the physical characteristics of the smear are inhibiting proper cell detection and classification. Performance metrics may include cell locations or distribution, cell detection rates, CCLs, and morphological assessment quality. When these metrics fall below predetermined thresholds, it may suggest that the smear length inadequacy affects analytical quality, providing a practical criterion for triggering the preparation of another blood smear.

In some implementations of the method, the detecting that the first blood smear has inadequate length comprises detecting excessive cell overlap or cell density, and the updated smearing parameter comprises decreasing blood volume or increasing smearing speed. Cell overlap or excessive cell density can interfere with accurate cell identification and classification by creating clusters where individual cells cannot be properly imaged, segmented or analyzed. To address this issue, the system may modify parameters that influence cell distribution density, such as decrease the blood volume used in preparing the second blood smear by a predetermined percentage to reduce overall cell density, and/or increase the smearing speed, which tends to spread cells over a greater area and reduce clustering. These adjustments help create a more appropriate cell density in the second blood smear, enabling better cell separation for improved automatic classification.

In some implementations of the method, the detecting that the first blood smear has inadequate length comprises detecting inadequate feathered edge formation, the updated smearing parameter comprises adjusting smearing angle or decreasing smearing pressure. The feathered edge of a blood smear provides a region where cells are distributed in a monolayer formation, which may be needed for accurate WBC analysis. When this edge is inadequately formed, the system may modify specific smearing parameters to improve feathered edge formation in subsequent preparations. For example, the system may adjust the smearing angle to create a more gradual transition at the feathered edge. Additionally or alternatively, the system may decrease smearing pressure by a specific amount, which can help prevent cells from being pushed too forcefully toward the edge of the slide.

In some implementations, the method further comprises analyzing results of preparing the second blood smear according to the second protocol to determine an effectiveness of the second protocol; and determining, based on analysis of patient-specific factors and protocol performance metrics, whether the inadequate length was caused by unique characteristics of the patient's blood or by limitations in the first protocol that would affect multiple patients. In some implementations, the method further comprises storing the second protocol with an association specifically to the patient when the determining indicates that the inadequate length was caused by unique characteristics of the patient's blood.

In some implementations, the method further comprises updating a default protocol of the automated slide maker-stainer to incorporate at least some aspects of the second protocol when the determining indicates that the inadequate length was caused by limitations in the first protocol that would affect multiple patients. Optionally, the system may implement a comprehensive evaluation framework to distinguish between protocol modifications that should be patient-specific versus those that should be applied more broadly across multiple patients. When evaluating protocol modifications, the system may analyze various data sources such as historical smear length performance across multiple patients, comparative analysis of the patient's current and previous samples, improvement rate between the two smears prepared from the same sample, measured parameters, and medical conditions that could affect blood properties like viscosity or cellular composition. These medical conditions may include, but are not limited to, hyperviscosity syndromes, hyperproteinemia, severe leukocytosis, and/or certain anemias that may alter blood smearing characteristics. The analysis may incorporate multiple factors such as the magnitude of length deviation from acceptable parameters, consistency of length inadequacies across repeated attempts, and/or correlations between specific blood properties and smear length outcomes. If the second protocol improves smear analysis results for a specific patient but similar adjustments haven't benefited other patients with comparable smear inadequacies, the system may classify this as a patient-specific requirement and optionally store the protocol with a direct association to the patient's identifier in a patient protocol database. This patient-specific protocol may be automatically retrieved and applied when future blood smears are prepared for the same patient, which may be helpful for example in cases of chronic diseases where patients undergo frequent tests with abnormal values. Conversely, if similar inadequacies in smear length have occurred across multiple patients with diverse blood characteristics, and the second protocol has demonstrated consistent improvement, the system may update the default protocol parameters for broader application after confirming the second protocol resolves the length inadequacy without introducing other quality issues. The determination process may utilize statistical analysis methods, machine learning algorithms, and/or rule-based systems to identify patterns that indicate whether the inadequacy stems from patient-specific factors or from suboptimal default protocol parameters.

In some implementations, the method further comprises determining whether the second protocol is to be applied only to blood smears associated with the patient, or whether the second protocol is to be applied also to future blood smears associated with other patients as well. In some implementations, the method further comprises storing the second protocol with an association to the patient when the second protocol is to be applied only to blood smears associated with the patient.

In some implementations, the method further comprises updating a default protocol of the automated slide maker-stainer to incorporate at least some aspects of the second protocol when the second protocol is to be applied to future blood smears for other patients. The system may implement a decision-making process to determine the appropriate scope of application for successful protocol adjustments related to smear length, which may include analyzing the uniqueness of the blood properties and assessing the likelihood that similar issues would affect other patients. To facilitate this determination, the system may analyze patterns in smear length inadequacies across patient populations, identify correlations with specific blood properties that affect blood viscosity or cellular distribution, and evaluate the magnitude and type of protocol adjustments required to achieve adequate smear length. The analysis may incorporate multiple data points including, but not limited to: historical protocol performance across diverse patient populations; laboratory-specific variables such as environmental conditions and equipment characteristics; temporal patterns in smear inadequacies; and/or patient demographic or diagnostic information that may correlate with specific blood properties. When the analysis indicates that the length inadequacy is specific to the patient's blood properties (such as unusual viscosity or cellular composition), the system may store the protocol with a specific association to that patient in a dedicated database that maintains patient-protocol associations. This association may include timestamps, version control information, and/or performance metrics to track protocol effectiveness over time. When the analysis indicates that the length inadequacy results from suboptimal default parameters that would likely affect multiple patients with diverse blood characteristics, the system may update a default protocol to incorporate the successful adjustments, potentially through a phased implementation that monitors outcomes across an expanding patient population. The system may optionally maintain multiple default protocols optimized for different patient subgroups or clinical contexts, applying the most appropriate default protocol based on available patient information. This approach may optimize smear quality across the entire patient population while maintaining specialized protocols for patients with unique requirements, potentially enhancing both efficiency and diagnostic reliability.

In some implementations of the method, the detecting that the first blood smear has inadequate length comprises utilizing at least one of the following methods: analyzing cell distributions using a cell detector or classifier on an image, analyzing a smear shape using an image acquired utilizing a relatively high effective numerical aperture capturing device, or analyzing a smear shape using an image acquired utilizing a relatively low effective numerical aperture capturing device. In one example, the relatively low effective numerical aperture capturing device has ENA below 0.4 and a relatively high ENA is above 0.75 and potentially above 1.

This embodiment may be integrated with human confirmation or oversight processes without departing from the automated decision-making framework for addressing inadequate smear length. For example, the system may automatically detect that the first blood smear has inadequate length for automatic cell classification while simultaneously generating alerts for laboratory personnel to review the length assessment and protocol selection decision. The system may provide a user interface that enables laboratory staff to monitor and control the selecting of the second protocol, allowing users to review the updated smearing parameter recommendations, approve or modify protocol adjustments for specific patients, and establish custom protocol libraries for recurring inadequate length scenarios. In clinical environments requiring human oversight, the system may automatically initiate the preparation of the second blood smear according to the second protocol while providing real-time status updates to technologists, allowing for intervention during the automated slide maker-stainer operation if warranted based on clinical judgment. The user interface may display comparative analysis of the first protocol and second protocol parameters, enable manual adjustment of updated smearing parameter values such as smearing speed, smearing angle, smearing pressure, or blood volume, and provide feedback mechanisms for laboratory staff to evaluate the effectiveness of protocol modifications. Additionally, the system may integrate quality assurance workflows where supervisors can review cases where inadequate length was detected, verify that the second protocol comprises substantially similar material types as required, and approve protocol updates for broader application across multiple patients. In some implementations of the method, a non-transitory computer-readable medium comprising instructions that, when executed by a processor, cause the processor to perform the computer-implemented method.

In various implementations, a system for preparing additional slides based on inadequate smear length, comprising: an automated slide maker-stainer; and a computer configured to: control the automated slide maker-stainer to prepare a first blood smear associated with a patient according to a first protocol; detect that the first blood smear has inadequate length for automatic cell classification; select a second protocol, wherein the second protocol is based on substantially similar material types as the first protocol, and wherein the second protocol comprises an updated smearing parameter intended to produce a blood smear having adequate length for the automatic cell classification; and control the automated slide maker-stainer to prepare a second blood smear associated with the patient according to the second protocol.

In some implementations of the system, the substantially similar material types comprise at least one of: staining reagents from the same chemical family, buffer solutions with comparable pH ranges, fixatives with similar cell preservation mechanisms, slide substrates with comparable surface characteristics, or anticoagulants with similar effects on blood viscosity; and wherein the updated smearing parameter comprises at least one of: smearing speed, smearing angle, smearing pressure, or blood volume. In some implementations of the system, the computer is configured to detect that the first blood smear has inadequate length by determining that the first blood smear is too short, resulting in insufficient cell distribution for reliable automatic cell classification.

In some implementations of the system, the computer is configured to detect that the first blood smear has inadequate length by determining that the first blood smear is too long, resulting in at least one of: absence of a feathered edge due to cells being spread to the edge of a slide, which reduces the ability to identify certain conditions related to white blood cells and platelets, or (ii) excessive spacing between cells, which prevents accurate identification of a monolayer region. In some implementations of the system, the computer is configured to detect that the first blood smear has inadequate length by utilizing a deep learning model trained on examples of adequate blood smear lengths and inadequate blood smear lengths. In some implementations of the system, the computer is configured to detect that the first blood smear has inadequate length by determining that detection performance of an automatic cell detector applied to an image of the first blood smear is below a predetermined value. In some implementations of the system, the computer is configured to detect that the first blood smear has inadequate length by detecting excessive cell overlap or cell density, and the updated smearing parameter comprises decreasing blood volume or increasing smearing speed. In some implementations of the system, the computer is configured to detect that the first blood smear has inadequate length by detecting inadequate feathered edge formation, and the updated smearing parameter comprises adjusting smearing angle or decreasing smearing pressure.

In some implementations of the system, the computer is further configured to: analyze results of preparing the second blood smear according to the second protocol to determine an effectiveness of the second protocol; and determine, based on analysis of patient-specific factors and protocol performance metrics, whether the inadequate length was caused by unique characteristics of the patient's blood or by limitations in the first protocol that would affect multiple patients. In some implementations of the system, the computer is further configured to store the second protocol with an association specifically to the patient when the determination indicates that the inadequate length was caused by unique characteristics of the patient's blood. In some implementations of the system, the computer is further configured to update a default protocol of the automated slide maker-stainer to incorporate at least some aspects of the second protocol when the determination indicates that the inadequate length was caused by limitations in the first protocol that would affect multiple patients.

In some implementations of the system, the computer is further configured to determine whether the second protocol is to be applied only to blood smears associated with the patient, or whether the second protocol is to be applied also to future blood smears associated with other patients as well. In some implementations of the system, the computer is further configured to store the second protocol with an association to the patient when the second protocol is to be applied only to blood smears associated with the patient. In some implementations of the system, the computer is further configured to update a default protocol of the automated slide maker-stainer to incorporate at least some aspects of the second protocol when the second protocol is to be applied to future blood smears for other patients. In some implementations of the system, the computer is configured to detect that the first blood smear has inadequate length by utilizing at least one of the following methods: analyzing cell distributions using a cell detector or classifier on an image, analyzing a smear shape using an image acquired utilizing a relatively high effective numerical aperture capturing device, or analyzing a smear shape using an image acquired utilizing a relatively low effective numerical aperture capturing device.

When a cytological smear exhibits staining inadequacies-either being over-stained or under-stained-cellular structures may not be properly visualized for reliable analysis. By detecting staining inadequacies and implementing appropriate protocol modifications, the system may produce a subsequent cytological smear with improved staining characteristics. The embodiment supports assessment of staining quality among samples and facilitates protocol adjustments to improve staining outcomes.

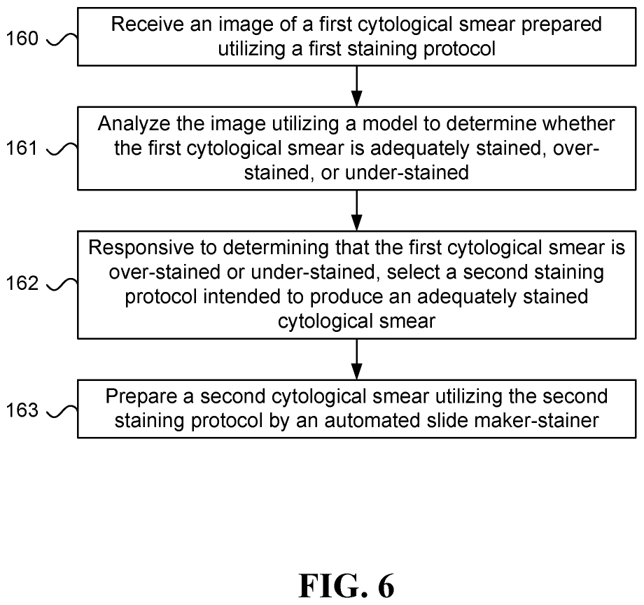

In various implementations, a computer-implemented method for preparing additional slides based on staining inadequacy, comprising: receiving an image of a first cytological smear associated with a patient, wherein the first cytological smear was prepared utilizing a first staining protocol; analyzing the image utilizing a model to determine whether the first cytological smear is adequately stained, over-stained, or under-stained for the purpose of automatic cell classification in the image; responsive to determining that the first cytological smear is over-stained or under-stained, selecting a second staining protocol intended to produce an adequately stained cytological smear, wherein the second staining protocol utilizes the same stain type as the first staining protocol and comprises an updated staining parameter; and preparing, by an automated slide maker-stainer, a second cytological smear associated with the patient, utilizing the second staining protocol. FIG. 6 illustrates an embodiment of a computer-implemented method for preparing additional slides based on staining inadequacy. The method includes the following steps. In step 160, receive an image of a first cytological smear prepared utilizing a first staining protocol. In step 161, analyze the image utilizing a model to determine whether the first cytological smear is adequately stained, over-stained, or under-stained. In step 162, responsive to determining that the first cytological smear is over-stained or under-stained, select a second staining protocol intended to produce an adequately stained cytological smear. And in step 163, prepare a second cytological smear utilizing the second staining protocol by an automated slide maker-stainer. Staining affects the visibility of cellular structures and the colors in the image, which are often important in the differentiation between cell types. The image used for analysis may be a relatively low ENA image of the slide, a high ENA scan of an area in the slide, or snapshots of small areas in the slide. The image is then analyzed by a model that may include, but is not limited to, any algorithm, classifier, analytical system, computational approach, or decision-making framework capable of determining if the image is adequate for automatic cell classification with sufficiently accurate results. The model may be implemented through various approaches including, but not limited to: machine learning models (such as neural networks, convolutional neural networks, recurrent neural networks, or transformer-based models); statistical models (such as regression models, Bayesian models, or statistical classifiers); rule-based systems with predefined thresholds and criteria; computer vision algorithms (such as edge detection algorithms, feature extraction techniques, or image segmentation methods); hybrid approaches combining multiple analytical techniques; deep learning architectures trained on labeled datasets of adequately and inadequately stained samples; or any combination thereof. For example, a statistical model may include a logistic regression classifier that analyzes multiple image features (such as color intensity distributions, nuclear-cytoplasmic contrast ratios, and background staining levels) to calculate probability scores for each staining category (adequately stained, over-stained, or under-stained), with the highest probability score determining the final classification. The model may determine the adequacy by analyzing color patterns, cell level or area level contrasts, color saturation, hue, edge detection or other visible features in the image. These analyses may involve spectral characteristics, intensity distributions, texture features, morphological parameters, structural integrity of cellular components, or other image properties that correlate with staining quality. Determining the adequacy may happen before, during or after classification of cells in the image, and in certain embodiments, following determining the sample as inadequate, may lead to avoiding classification of cells in the image, which can save time, storage and inaccurate results, improving system reliability and raising trust in it. In the context of this embodiment, “for the purpose of” refers to what the model is testing, without imposing any order of actions, and without suggesting if the action is performed at all.

In some implementations of the method, the updated staining parameter comprises a quantitative adjustment of staining duration, specifying a reduction in staining time if the first cytological smear is over-stained, or an extension of the staining time if the first cytological smear is under-stained. Staining duration may directly influence the intensity of stain uptake by various cellular components, and the system may modify this parameter based on the specific nature of the staining inadequacy detected in the first cytological smear. When over-staining is detected, the system may implement a reduction in staining time by a first percent or duration, which may decrease the overall stain intensity and improve differentiation between cellular structures. Conversely, when under-staining is detected, the system may extend the staining time by a second percent or duration, which may enhance stain uptake and improve visualization of cellular components. These adjustments may be calibrated according to the degree of staining inadequacy detected, with more significant deviations prompting more substantial adjustments to staining duration.

In some implementations of the method, the cytological smear is a blood smear, and the updated staining parameter comprises a change to duration of at least one of first or second staining stages included in a staining process. Cytological smear staining protocols often involve multiple staining stages that utilize different components to stain various cellular structures, and the system may modify the durations of at least some of these staining stages to address specific staining inadequacies. For example, in protocols utilizing Romanowsky-type stains such as Wright or Wright-Giemsa, the ratio between the primary staining solution application and the buffer solution application may be adjusted. Increasing the proportion of primary stain may enhance overall staining intensity, while adjusting buffer exposure may affect cellular differentiation and contrast. The system may determine appropriate duration adjustments based on the specific staining characteristics observed in the first cytological smear, targeting the particular inadequacy while maintaining other staining qualities.

In some implementations of the method, the model was trained to determine whether the first cytological smear includes too many precipitates for the purpose of automatic cell classification, and responsive to determining that the first cytological smear includes too many precipitates, selecting the second staining protocol as a function of the precipitates. Precipitates in cytological smears may interfere with automatic cell classification by creating artifacts that may be misidentified as cellular structures or may obscure important cellular details. The model may be trained to identify various forms of precipitates including stain deposits, crystallized buffer components, or protein aggregates. When excessive precipitates are detected, the system may select a second staining protocol designed to minimize precipitate formation. Such protocol modifications may include adjustments to stain filtration processes, changes in buffer composition, modifications to staining temperature, a need to replace at least some of the stains, and/or alterations in washing procedures. The system may analyze the specific characteristics and distribution patterns of the precipitates to determine more appropriate protocol adjustments to address the underlying cause of precipitate formation.

In some implementations of the method, the model was trained to determine contrast ratios between different cellular elements within hematological cells, and to classify the staining as inadequate when the contrast ratios do not allow sufficient differentiation between cellular structures needed for accurate classification. Proper cellular classification depends on adequate contrast between different cellular components such as nuclei, cytoplasm, granules, and inclusions. The model may be trained to assess multiple contrast parameters including color intensity differentials, edge definition between structures, and spectral separation of stained components. The system may evaluate these contrast ratios against established thresholds required for reliable automated classification of specific cell types and their components. When contrast ratios fall below these thresholds, the system may identify this as a staining inadequacy even if overall staining intensity appears acceptable, and the subsequent protocol adjustments may target aspects of the staining process that influence contrast development, such as pH levels, staining component concentrations, and/or timing of different staining phases.

In some implementations of the method, the model was trained to determine intensities and contrast ratios between areas in an overview image of the smear, and to classify the staining as inadequate when the contrast ratios are predicted to not allow for accurate classification of cells in an image with sufficient resolution for cell level analysis. A preview camera or overview camera is often used to take images of large areas of the smear before taking high resolution images or scans of the smear, which allows for scan area choices, adequacy analysis and assisting reviewers in analysis. The overview image typically does not allow for analysis of details within cells. When a smear is overstained or under-stained, it will be visible in the overview image, and a model trained to detect these changes can find cases where the smear is too dark or too light, cases where the contrast between areas in the slide such as the monolayer, feathered edge or bulk is inadequate, or where gradients in the image are indicative of inadequate staining.

In some implementations, the method further comprises responsive to determining that the second cytological smear is inadequately stained: analyzing the second cytological smear to determine an improvement level relative to the first cytological smear; selecting a third staining protocol for preparing a third cytological smear, wherein parameter adjustments in the third staining protocol are smaller than corresponding adjustments between the first and second staining protocols; and requesting preparation of the third cytological smear utilizing the third staining protocol. The system may implement progressive refinement for protocol optimization that makes increasingly precise adjustments across successive iterations. For example, when addressing staining inadequacies, the first iteration might involve relatively large adjustments-such as increasing staining time by a first percentage if under-staining is detected. If the second cytological smear shows improvement but still exhibits under-staining, the system may implement a smaller additional increase by a second percentage (smaller than the first percentage). Conversely, if the second smear shows over-correction (now exhibiting over-staining), the system may decrease the staining time by a third percentage from the first adjustment. Additional iterations follow a similar pattern of increasingly fine-tuned adjustments, with the magnitude of a certain adjustment being smaller than that of a preceding adjustment. This progressive narrowing may be applied to various preparation parameters including staining time, stain concentration, smearing pressure, smearing speed, and/or smearing angle.

In some implementations of the method, selecting the second staining protocol comprises at least one of: transmitting instructions for the second staining protocol to the automated slide maker-stainer; transmitting an identifier of the second staining protocol to the automated slide maker-stainer, wherein the automated slide maker-stainer stores a plurality of predefined protocols including the second staining protocol; or transmitting adjustment instructions to the automated slide maker-stainer, wherein the adjustment instructions direct the automated slide maker-stainer to modify one or more parameters of the first staining protocol to produce the second staining protocol. The system may employ various approaches for implementing the selected second staining protocol on the automated slide maker-stainer. In one approach, the system may generate and transmit complete protocol instructions, providing a comprehensive set of parameters and procedures for preparing the second cytological smear. These instructions may include detailed specifications for each step of the staining process, encompassing parameters such as staining agent concentrations, application durations, temperature controls, and mechanical aspects of slide handling. Alternatively, when the automated slide maker-stainer maintains an internal repository of predefined protocols, the system may transmit an identifier that references the appropriate protocol within this repository. The identifier may include a protocol code, index number, or name that identifies the selected protocol within the slide maker-stainer's memory. In a third approach, rather than transmitting a complete protocol or selecting a predefined alternative, the system may instruct the automated slide maker-stainer to modify specific parameters of the first staining protocol. These adjustment instructions may indicate relative changes such as percentage increases or decreases to particular parameters, or may specify values for parameters that require precise modification. The slide maker-stainer may then internally generate the second staining protocol by applying these adjustments to the first protocol while maintaining other parameters unchanged.

In some implementations of the method, the model is a machine learning model, and analyzing the image utilizing the model comprises performing automatic cell detection and/or classification of at least hundreds of cells in the image of the first cytological smear. The machine learning model may incorporate various analytical approaches such as convolutional neural networks, random forests, or ensemble methods that have been trained on large datasets of cytological smears with varied staining characteristics. In some embodiments, detection and classification may represent distinct but potentially complementary analytical approaches. A detection model may identify the presence and location of cells it has been trained to recognize, potentially functioning to determine whether specific cellular entities exist within an image and where they may be situated. A classification model may determine the specific type or category of a cell, optionally applying distinctions between similar cell types based on morphological, colorimetric, or other features. Detectors may optionally be designed to consider the surrounding environment and contextual information to identify cell presence, while classification may typically focus on the intrinsic characteristics of the cellular object itself. The tasks of detection and classification may be combined in various possible embodiments. In one example, this combination may be achieved by integrating both detection and classification functions within the same neural network, or within a neural network architecture where certain layers may focus on localization while other layers may perform classification. In another example, the combination may be achieved by applying a unified approach that evaluates presence criteria and type-specific features according to rules or algorithmic patterns.

Moreover, analyzing large cell populations may provide statistical robustness for staining quality assessment, as individual cell variation is averaged across a comprehensive sample. For example, when the system analyzes a large number of cells it may detect subtle staining inadequacies that may not be apparent in smaller sample sizes. Classifying a large number of cells may also enable the system to identify staining patterns that affect specific cell populations or cellular structures while being less prominent in others. The high cell count may provide sufficient data for the model to determine with greater confidence whether staining inadequacies are present and to characterize their nature more precisely, which may lead to more accurate protocol adjustment decisions. Classifying a large number of cells further allows detecting differences that affect rare cell populations, such as the staining of the cytoplasm in plasma cells.

In some implementations of the method, analyzing the image utilizing the model comprises analyzing color properties of cells in the image, wherein the color properties comprise at least one of: color intensity, color distribution, or color aberrations indicative of inadequate staining. The model may evaluate multiple color properties to determine staining adequacy. Color intensity analysis may assess whether cellular components have absorbed sufficient stain to provide adequate visualization, with intensity measurements potentially calculated across different color channels or spectral ranges. Color distribution analysis may examine how evenly stain has been applied across the smear and within individual cells, potentially identifying regions of inconsistent staining that may affect classification accuracy. Analysis of color aberrations may identify inappropriate staining patterns such as non-specific background staining, inappropriate color shifts, or staining artifacts that deviate from expected patterns. The system may compare these color properties against references derived from properly stained samples to quantify deviations and determine whether these deviations are likely to impact the automatic cell classification performance.

In some implementations, the method further comprises analyzing results of preparing the second cytological smear according to the second staining protocol to determine an effectiveness of the second staining protocol; determining, based on analysis of patient-specific factors and protocol performance metrics, whether the staining inadequacy was caused by unique characteristics of the patient's blood or by limitations in the first staining protocol that would affect multiple patients. In some implementations, the method further comprises storing the second staining protocol with an association specifically to the patient when the determining indicates that the staining inadequacy was caused by unique characteristics of the patient's blood. In some implementations, the method further comprises updating a default protocol of the automated slide maker-stainer to incorporate at least some aspects of the second staining protocol when the determining indicates that the staining inadequacy was caused by limitations in the first staining protocol that would affect multiple patients. In various embodiments, the system employs an analytical determination process to distinguish between protocol modifications that should be patient-specific versus those that should be applied more broadly. When evaluating whether a protocol modification should be patient-specific, the system may analyze various data points such as: historical staining performance across multiple patients, comparative analysis of the patient's current and previous blood samples, known medical conditions or medications from the patient's electronic medical record that could affect blood properties, and/or statistical patterns of protocol adjustments across the patient population. For example, if analysis reveals that the second staining protocol improves staining quality for the specific patient but similar adjustments have not benefited other patients in the laboratory's historical data, the system classifies this as a patient-specific requirement. The system may further correlate this finding with medical record data indicating the patient is on medication known to alter blood characteristics. In this case, the modified protocol is tagged with a patient identifier and stored in association with that patient's record. When future samples from this patient arrive, the system may automatically retrieve and apply this customized protocol. Conversely, if the system detects that similar inadequacies have occurred across multiple patient samples, or if the staining issue correlates with a recent change in laboratory reagents or environmental conditions, it identifies the protocol improvement as having broader applicability. In these cases, after confirming that the second staining protocol successfully resolves the inadequacy, the system may update the default protocol used for other patients. By implementing this analytical determination process, the system creates an intelligent calibration framework that continuously refines both patient-specific and general protocols, improving overall staining quality while accommodating individual patient variations.

In some implementations, the method further comprises selecting the second staining protocol from a predefined set of staining protocols stored in a repository accessible by the automated slide maker-stainer. The system may maintain a repository of predefined staining protocols that address specific staining inadequacies identified by the model. Examples of repositories include a database, lookup table, table, stack, array, variables, text file, CSV file, neural network with encoded knowledge in hidden layers, deep learning model parameters, inference model weights, or any other suitable storage structure capable of maintaining and retrieving information. The repository may leverage networks that intrinsically store relevant information within their architecture for access and application in various analytical contexts. The selection of an appropriate protocol from this repository may be based on multiple factors including the type and severity of staining inadequacy, patient-specific factors, and/or historical performance of different protocols for similar staining issues.

In some implementations of the method, selecting the second staining protocol comprises: identifying a specific type of staining inadequacy in the first cytological smear, and selecting a specific staining parameter to modify based on the specific type of staining inadequacy identified. Optionally, the system may modify the protocol based on identifying specific inadequacy types and selecting corresponding preparation parameters to address the inadequacies, which focuses the adjustments on parameters related to the observed issue. For example, when under-staining is detected (characterized by insufficient color intensity in cellular components), the system may increase staining time by a first percentage or increase stain concentration by a second percentage. Conversely, when over-staining is identified (characterized by excessive color intensity that obscures cellular details), the system may decrease staining time by a third percentage or dilute the stain solution by a fourth percentage. For chemical issues such as excessive precipitates, the system may add a buffer washing step to the protocol or adjust the staining solution pH.

In some implementations of the method, selecting the specific staining parameter comprises: determining a severity level of the staining inadequacy, and determining adjustment magnitudes for the specific staining parameter as a function of the severity level. For example, for mild inadequacies, which slightly deviate from acceptable quality metrics, the system may select a single preparation parameter to modify with a relatively small adjustment; for moderate inadequacies, the system may select one or two parameters with medium-range adjustments; and for severe inadequacies, the system may adjust several preparation parameters to address the significant quality issues. The system may determine severity levels through quantitative analysis of the inadequacy's impact on cell classification accuracy, comparing measured quality metrics against reference standards for acceptable preparation. This process creates a flexible response system that matches the intervention levels to the specific needs of the preparation scenarios, allowing for minimally disruptive corrections for minor issues while providing comprehensive protocol revisions for more significant problems. In some implementations of the method, a non-transitory computer-readable medium comprising instructions that, when executed by a processor, cause the processor to perform the computer-implemented method.

In various implementations, a system for preparing additional slides based on staining inadequacy, comprising: a computer configured to: receive an image of a first cytological smear associated with a patient, wherein the first cytological smear was prepared utilizing a first staining protocol; analyze the image utilizing a model to determine whether the first cytological smear is adequately stained, over-stained, or under-stained for the purpose of automatic cell classification in the image; responsive to a determination that the first cytological smear is over-stained or under-stained, select a second staining protocol intended to produce an adequately stained cytological smear, wherein the second staining protocol utilizes the same stain type as the first staining protocol and comprises an updated staining parameter; and command an automated slide maker-stainer to prepare a second cytological smear associated with the patient, utilizing the second staining protocol.

In some implementations of the system, the updated staining parameter comprises a quantitative adjustment of staining duration, specifying a reduction in staining time if the first cytological smear is over-stained, or an extension of the staining time if the first cytological smear is under-stained. In some implementations of the system, the cytological smear is a blood smear, and the updated staining parameter comprises a change to duration of at least one of first or second staining stages included in a staining process. In some implementations of the system, the model was trained to determine whether the first cytological smear includes too many precipitates for the purpose of automatic cell classification, and wherein the computer is configured to, responsive to a determination that the first cytological smear includes too many precipitates, select the second staining protocol as a function of the precipitates.

In some implementations of the system, the model was trained to determine contrast ratios between different cellular elements within hematological cells, and to classify the staining as inadequate when the contrast ratios do not allow sufficient differentiation between cellular structures needed for accurate classification. In some implementations of the system, the model was trained to determine intensities and contrast ratios between areas in an overview image of the smear, and to classify the staining as inadequate when the contrast ratios are predicted to not allow for accurate classification of cells in an image with sufficient resolution for cell level analysis. In some implementations of the system, the computer is further configured to: responsive to a determination that the second cytological smear is inadequately stained: analyze the second cytological smear to determine an improvement level relative to the first cytological smear; select a third staining protocol for preparing a third cytological smear, wherein parameter adjustments in the third staining protocol are smaller than corresponding adjustments between the first and second staining protocols; and command the automated slide maker-stainer to prepare the third cytological smear utilizing the third staining protocol.

In some implementations of the system, the computer is configured to select the second staining protocol by at least one of: transmitting instructions for the second staining protocol to the automated slide maker-stainer; transmitting an identifier of the second staining protocol to the automated slide maker-stainer, wherein the automated slide maker-stainer stores a plurality of predefined protocols including the second staining protocol; or transmitting adjustment instructions to the automated slide maker-stainer, wherein the adjustment instructions direct the automated slide maker-stainer to modify one or more parameters of the first staining protocol to produce the second staining protocol. In some implementations of the system, the model is a machine learning model, and the computer is configured to analyze the image utilizing the model by performing automatic cell detection and/or classification of at least hundreds of cells in the image of the first cytological smear. In some implementations of the system, the computer is configured to analyze the image utilizing the model by analyzing color properties of cells in the image, wherein the color properties comprise at least one of: color intensity, color distribution, or color aberrations indicative of inadequate staining.

In some implementations of the system, the computer is further configured to: analyze results of preparing the second cytological smear according to the second staining protocol to determine an effectiveness of the second staining protocol; determine, based on analysis of patient-specific factors and protocol performance metrics, whether the staining inadequacy was caused by unique characteristics of the patient's blood or by limitations in the first staining protocol that would affect multiple patients. In some implementations of the system, the computer is further configured to store the second staining protocol with an association specifically to the patient when the determination indicates that the staining inadequacy was caused by unique characteristics of the patient's blood. In some implementations of the system, the computer is further configured to update a default protocol of the automated slide maker-stainer to incorporate at least some aspects of the second staining protocol when the determination indicates that the staining inadequacy was caused by limitations in the first staining protocol that would affect multiple patients.

In some implementations of the system, the computer is further configured to select the second staining protocol from a predefined set of staining protocols stored in a repository accessible by the automated slide maker-stainer. In some implementations of the system, the computer is configured to select the second staining protocol by: identifying a specific type of staining inadequacy in the first cytological smear, and selecting a specific staining parameter to modify based on the specific type of staining inadequacy identified. In some implementations of the system, the computer is configured to select the specific staining parameter by: determining a severity level of the staining inadequacy, and determining adjustment magnitudes for the specific staining parameter as a function of the severity level.

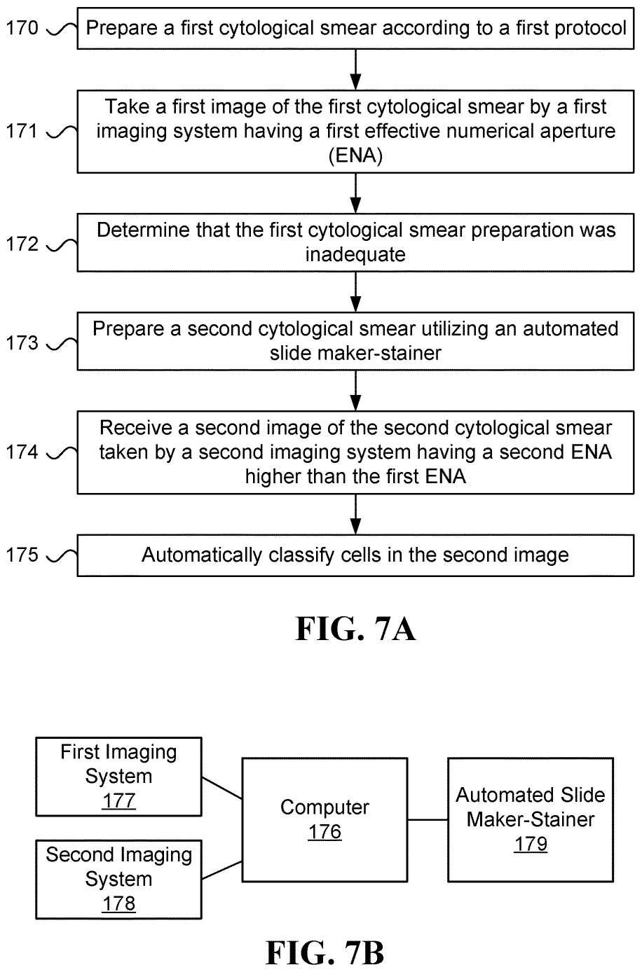

In various implementations, a computer-implemented method for preparing additional slides, comprising: preparing a first cytological smear associated with a patient according to a first protocol; taking, by a first imaging system having a first effective numerical aperture (ENA), a first image of the first cytological smear; determining that the preparing of the first cytological smear was inadequate; preparing, utilizing an automated slide maker-stainer, a second cytological smear associated with the patient; receiving a second image of the second cytological smear, wherein the second image was taken by a second imaging system having a second ENA higher than the first ENA; and automatically classifying cells in the second image. FIG. 7A illustrates an embodiment of a computer-implemented method for preparing additional slides. The method includes the following steps. In step 170, prepare a first cytological smear according to a first protocol. In step 171, take a first image of the first cytological smear by a first imaging system having a first effective numerical aperture (ENA). In step 172, determine that the first cytological smear preparation was inadequate. In step 173, prepare a second cytological smear utilizing an automated slide maker-stainer. In step 174, receive a second image of the second cytological smear taken by a second imaging system having a second ENA higher than the first ENA. And in step 175, automatically classify cells in the second image. In some implementations of the method, the preparing of the second cytological smear utilizes a second protocol expected to result in a more adequate cytological smear.

In some implementations of the method, the second imaging system comprises at least one of: a microscope with an objective lens designed to be immersed in a medium with refractive index above one, a microscope with an objective lens designed to work in air, a computational microscope where captured images are digitally processed to create a final image, such as in holography or diffractive imaging, a super-resolution imaging system configured to computationally combine multiple images to create a composite image with resolution exceeding the optical diffraction limit or aberrations limits of a single image; and wherein the first imaging system comprises an imaging camera configured to rapidly capture images, collectively comprising at least most of the slide area at lower resolution for initial quality assessment. The system may implement a multi-tiered imaging approach that optimizes both efficiency and analytical detail by employing different imaging technologies at different stages of the workflow. The first imaging system may utilize an imaging camera (such as a preview camera) that rapidly captures images which collectively comprise at least most of the slide area at lower resolution for initial quality assessment. It may be used to assess parameters such as staining intensity, smear distribution, and cellular density across a substantial portion of the slide surface in a time-efficient manner, creating an efficient screening mechanism that identifies obviously inadequate preparations early in the process. For slides meeting initial quality thresholds, the second imaging system may employ advanced imaging capabilities. Microscopes with objective lenses designed to be immersed in a medium with refractive index above one may enhance optical resolution by eliminating air gaps between the objective lens and the specimen, potentially increasing numerical aperture and reducing light refraction issues. These may allow visualization of subcellular structures and fine morphological details required for accurate cell classification and for distinguishing subtle nuclear features, granule characteristics, and inclusion bodies. Microscopes with objective lenses designed to work in air may offer different advantages in terms of workflow efficiency while still providing sufficient resolution for certain analyses. Alternatively or additionally, computational microscopes where captured images are digitally processed to create a final image, such as in holography or diffractive imaging, may enable alternative approaches to cell visualization. Super-resolution imaging systems may circumvent traditional optical limitations by computationally combining multiple images to create a composite image with resolution exceeding the optical diffraction limit or aberrations limits of a single image. This approach may involve capturing multiple at least partially overlapping images and applying computational algorithms to extract sub-pixel information, potentially resulting in images with effective resolution beyond what conventional optics alone could achieve. The multi-camera configuration may enable parallel image acquisition across different regions of the cytological smear, potentially accelerating the imaging process while maintaining high resolution. These complementary imaging approaches may create an efficient workflow that balances processing speed with analytical depth, optimizing system resources by reserving higher resolution imaging for slides that have been determined to have adequate preparation quality through initial lower resolution assessment.

In some implementations, the method further comprises determining that the first cytological smear exhibits severe preparation inadequacies meeting predetermined criteria for non-viability; and initiating the preparing of the second cytological smear without capturing images of the first cytological smear at the second ENA. The system may implement triage logic that identifies severe preparation inadequacies during initial lower-ENA assessment, potentially bypassing unnecessary high-resolution imaging of obviously inadequate specimens. Severe inadequacies that may trigger this bypass decision may include extreme smear length issues (excessively short or long smears), substantial physical damage to the slide such as significant scratches or cracks, staining problems such as extreme over-staining or under-staining that renders cellular details unrecognizable, absence of cellular material due to sampling failure or fluid flow issues during preparation, or major distribution problems such as extreme cell clumping that prevents proper monolayer assessment. When such severe inadequacies are detected, the system may determine that imaging at the second higher ENA would not yield meaningful additional information, and may therefore proceed directly to preparing a second cytological smear, optionally with a modified protocol specifically addressing the identified severe inadequacies. This approach may optimize system efficiency by conserving high-resolution imaging resources for specimens with higher likelihood of yielding useful analytical results. In other embodiments, the system may determine a need to order another smear, but continue to scan the first smear with the second higher ENA to see if there is potentially relevant information on it, which saves some time by ordering the additional smear early on.

In some implementations of the method, the determining that the preparing of the first cytological smear was inadequate comprises detecting an identification issue with a barcode associated with the first cytological smear, wherein the identification issue comprises at least one of: absence of a readable barcode, damaged barcode preventing proper reading, incorrect barcode information, or misalignment of barcode placement. Proper specimen identification through barcode systems may be essential for maintaining the integrity of the analytical workflow and ensuring that results are correctly associated with the appropriate patient. Various issues may affect barcode functionality, including printing failures that result in absent, faded, or partially printed barcodes; physical damage such as scratches or smudges that render portions of the barcode unreadable; software errors that generate incorrect barcode information or format; or mechanical alignment issues that place the barcode in positions where scanning systems cannot properly capture the complete code. When such identification issues are detected, the system may order another smear. In some embodiments, the system may further implement specific corrective measures in the second protocol such as adjusting printer settings to modify barcode darkness, size, or resolution; recalibrating barcode placement parameters to ensure proper positioning within scanner reading zones; or selecting alternative identification methods when primary approaches have failed. The system may maintain a log of barcode-related issues to facilitate identification of systematic problems that may require broader intervention, such as printer maintenance, barcode reader alignment, or software updates to barcode generation algorithms.

In some implementations of the method, the first ENA is below 0.4 and the second ENA is above 0.75 and potentially above 1. The use of different ENA levels between initial assessment and detailed classification may enable an efficient workflow that balances processing speed with analytical depth. Lower ENA imaging (below 0.4) during initial assessment may provide sufficient detail to identify preparation inadequacies while enabling rapid imaging of larger slide areas. Higher ENA imaging (above 0.75) may subsequently provide the cellular detail necessary for accurate automatic classification of cells and their morphological features. This tiered approach to ENA may optimize system resources by reserving higher resolution imaging for slides that have been determined to have adequate preparation quality through initial lower resolution assessment.

In some implementations of the method, the determining that the preparing of the first cytological smear was inadequate comprises determining that staining of the first cytological smear is inadequate for automatic cell classification. Staining quality impacts the system's ability to perform accurate automatic cell classification by affecting the visibility and differentiation of cellular structures. Inadequate staining may manifest in various forms including insufficient staining intensity, excessive staining that obscures cellular details, inappropriate color balance between nuclear and cytoplasmic components, and/or staining artifacts such as precipitates that interfere with proper visualization. The system may assess staining adequacy through analysis of color distributions, intensity patterns, and contrast ratios between different cellular structures. When these parameters deviate from established ranges associated with reliable classification, the system may determine that staining inadequacy necessitates preparation of an additional cytological smear with modified staining parameters.

In some implementations of the method, the second protocol comprises a modified staining parameter selected to address the inadequate staining determined in the first cytological smear. When staining inadequacies are identified in the first cytological smear, the second protocol may incorporate targeted modifications to specific staining parameters. These modifications may include adjustments to staining duration to address intensity issues, alterations in stain concentration to modify color saturation, changes in buffer composition or pH to affect stain specificity, or modifications to washing procedures to reduce background staining or precipitate formation. The system may select the specific parameters to modify based on the particular type of staining inadequacy detected, with different inadequacy patterns triggering adjustments to different aspects of the staining protocol. For example, under-staining may prompt increased staining time or concentration, while over-staining may lead to decreased staining time or dilution of staining reagents. These targeted modifications may efficiently address the specific staining issues observed rather than implementing generalized protocol changes.

In some implementations of the method, the determining that the preparing of the first cytological smear was inadequate is based on automatic analysis of the first image, and further comprising determining, based on automatic analysis of the second image, that the preparing of the second cytological smear is adequate, and refraining from preparing a third cytological smear associated with the patient. The system may implement decision-making logic that evaluates the adequacy of cytological smear preparations at multiple stages of the workflow. When analysis of the second cytological smear indicates that the preparation meets or exceeds quality requirements for reliable automatic cell classification, the system may terminate the preparation sequence for the specific patient, determining that no additional smears are necessary. This determination may be based on quantitative quality metrics such as staining intensity distributions, cell distribution patterns, monolayer region characteristics, and CCLs achieved during analysis of the second image. By refraining from preparing unnecessary additional smears when adequate quality has been achieved, the system may optimize resource utilization, reduce processing time, and minimize sample volume requirements.

In some implementations, the method further comprises determining, based on the automatic classification of the cells in the second image, that the preparing of the second cytological smear is inadequate, and preparing a third cytological smear associated with the patient according to a different protocol utilizing the automated slide maker-stainer. When the second cytological smear remains inadequate, possibly despite protocol adjustments, the system may initiate an additional preparation iteration with a different protocol modifications. The determination of inadequacy in the second cytological smear may be based on detailed cell classification analysis that reveals persistent issues affecting classification accuracy. These issues may include suboptimal staining characteristics, problematic cell distribution patterns, or other preparation artifacts that become apparent at higher ENA imaging. The different protocol may incorporate more substantial parameter adjustments or alternative preparation approaches based on cumulative learning from both previous preparation attempts. This iterative refinement process may continue until a cytological smear with adequate quality for reliable automatic cell classification is achieved, potentially with each iteration incorporating increasingly specialized protocol adjustments targeted to the specific preparation challenges identified in the particular patient sample.

In some implementations of the method, the determining that the preparing of the second cytological smear is inadequate is based on classification confidence level (CCL) associated with at least one cell type in the second image. CCLs may provide a quantitative metric for assessing whether preparation quality is sufficient for reliable automatic cell classification. For each classified cell and/or cell type, the system may generate a confidence score indicating the probability that the classification is correct based on how clearly the cell exhibits the expected morphological and staining characteristics of its assigned type. When CCLs for specific cell types consistently fall below predetermined thresholds, it may indicate that the preparation quality is insufficient for reliable classification of those cell types. Different cell populations may require different minimum CCL thresholds based on their clinical significance and the difficulty of their classification. For example, the system may require higher CCLs for accurate classification of immature cells or abnormal cells that may have diagnostic significance, while accepting somewhat lower CCLs for common mature cells with less clinical variability.

In some implementations, the method further comprises taking a third image of the first cytological smear at the second ENA after taking the first image and before determining that the preparing of the first cytological smear was inadequate, wherein the determining that the preparing of the first cytological smear was inadequate comprises analyzing a result calculated at least in part based on the third image. In some embodiments, after taking the first image of the first cytological smear, and before determining that the preparing of the first cytological smear was inadequate, a third image of the first cytological smear is taken at the second, higher ENA. One or more analysis algorithms are executed to analyze the first cytological smear, wherein the one or more analysis algorithms comprise at least one of a detector, a classifier, and/or an image processing algorithm configured to determine whether the first cytological smear is adequate. The determining that the preparing of the first cytological smear was inadequate is performed using data from the third image taken at the second ENA. The determining may additionally utilize results calculated from the first image in combination with the data from the third image to determine whether the preparing of the first cytological smear was inadequate. In some implementations of the method, a non-transitory computer-readable medium comprising instructions that, when executed by a processor, cause the processor to perform the computer-implemented method.

In various implementations, a system for preparing additional slides, comprising: a computer configured to: control preparation of a first cytological smear associated with a patient according to a first protocol; receive a first image of the first cytological smear taken by a first imaging system having a first effective numerical aperture (ENA); determine that the preparation of the first cytological smear was inadequate; command an automated slide maker-stainer to prepare a second cytological smear associated with the patient; receive a second image of the second cytological smear taken by a second imaging system having a second ENA higher than the first ENA; and automatically classify cells in the second image. FIG. 7B illustrates an embodiment of a block diagram of a system for preparing additional slides. The system includes a computer 176 that receives images from a first imaging system 177 having a first ENA, and from a second imaging system 178 having a second ENA higher than the first ENA. The computer is further coupled to an automated slide maker-stainer 179. In some implementations of the system, the computer is configured to command the automated slide maker-stainer to prepare the second cytological smear utilizing a second protocol expected to result in a more adequate cytological smear.

In some implementations of the system, the second imaging system comprises at least one of: a microscope with an objective lens designed to be immersed in a medium with refractive index above one, a microscope with an objective lens designed to work in air, a computational microscope where captured images are digitally processed to create a final image, such as in holography or diffractive imaging, a super-resolution imaging system configured to computationally combine multiple images to create a composite image with resolution exceeding the optical diffraction limit or aberrations limits of a single image; and wherein the first imaging system comprises an imaging camera configured to rapidly capture images, collectively comprising at least most of the slide area at lower resolution for initial quality assessment.

In some implementations of the system, the computer is further configured to: determine that the first cytological smear exhibits severe preparation inadequacies meeting predetermined criteria for non-viability; and command the automated slide maker-stainer to prepare the second cytological smear without capturing images of the first cytological smear at the second ENA. In some implementations of the system, the computer is configured to determine that the preparation of the first cytological smear was inadequate by detecting an identification issue with a barcode associated with the first cytological smear, wherein the identification issue comprises at least one of: absence of a readable barcode, damaged barcode preventing proper reading, incorrect barcode information, or misalignment of barcode placement. In some implementations of the system, the first ENA is below 0.4 and the second ENA is above 0.75 and potentially above 1.

In some implementations of the system, the computer is configured to determine that the preparation of the first cytological smear was inadequate by determining that staining of the first cytological smear is inadequate for automatic cell classification. In some implementations of the system, the second protocol comprises a modified staining parameter selected to address the inadequate staining determined in the first cytological smear. In some implementations of the system, the computer is configured to determine that the preparation of the first cytological smear was inadequate based on automatic analysis of the first image, and wherein the computer is further configured to: determine, based on automatic analysis of the second image, that the preparation of the second cytological smear is adequate; and refrain from commanding the automated slide maker-stainer to prepare a third cytological smear associated with the patient.

In some implementations of the system, the computer is further configured to: determine, based on the automatic classification of the cells in the second image, that the preparation of the second cytological smear is inadequate; and command the automated slide maker-stainer to prepare a third cytological smear associated with the patient according to a different protocol. In some implementations of the system, the computer is configured to determine that the preparation of the second cytological smear is inadequate based on classification confidence level (CCL) associated with at least one cell type in the second image. In some implementations of the system, the computer is further configured to: receive a third image of the first cytological smear at the second ENA after receiving the first image and before determining that the preparation of the first cytological smear was inadequate; and determine that the preparation of the first cytological smear was inadequate by analyzing a result calculated at least in part based on the third image.

The following implementation addresses the decision process for determining which cytological smear analyses require human expert review based on classification confidence levels. By selectively presenting cases with low confidence classifications for human review, the system may optimize the use of expert resources while maintaining diagnostic quality, which may reduce the workload on human reviewers by automating the handling of high-confidence classifications. The embodiment supports efficient allocation of human expertise to cases where it adds the most value. Additionally, in clinical scenarios where the number of cases is too numerous for comprehensive human review within reasonable time constraints, this selective routing approach enables focused human attention on the most diagnostically significant or uncertain classifications, rather than requiring review of all cases.

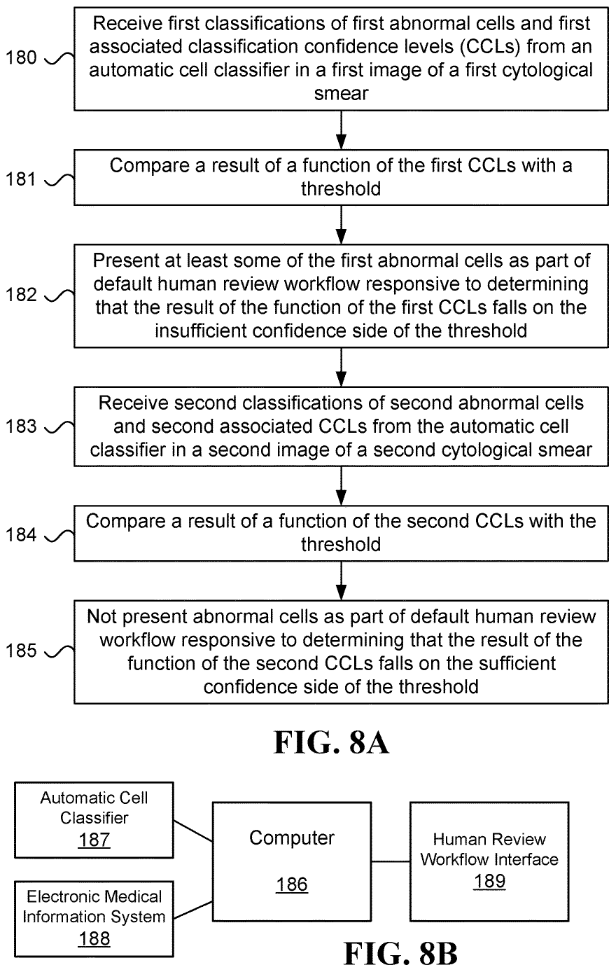

In various implementations, a computer-implemented method for preparing images for human review, comprising: receiving, from an automatic cell classifier, first classifications of first abnormal cells and first associated classification confidence levels (CCLs), wherein the first abnormal cells are in a first image of a first cytological smear associated with a first sample obtained from a first patient; comparing a result of a function of the first CCLs with a threshold; responsive to determining that the result of the function of the first CCLs falls on the insufficient confidence side of the threshold, presenting, as part of default human review workflow, at least some of the first abnormal cells; receiving, from the automatic cell classifier, second classifications of second abnormal cells and second associated CCLs, wherein the second abnormal cells are in a second image of a second cytological smear associated with a second sample obtained from a second patient; comparing a result of a function of the second CCLs with the threshold; and responsive to determining that the result of the function of the second CCLs falls on the sufficient confidence side of the threshold, not presenting, as part of default human review workflow, abnormal cells associated with the second sample. FIG. 8A illustrates an embodiment of a computer-implemented method for preparing images for human review. The method includes the following steps. In step 180, receive first classifications of first abnormal cells and first associated classification confidence levels (CCLs) from an automatic cell classifier in a first image of a first cytological smear. In step 181, compare a result of a function of the first CCLs with a threshold. In step 182, present at least some of the first abnormal cells as part of default human review workflow responsive to determining that the result of the function of the first CCLs falls on the insufficient confidence side of the threshold. In step 183, receive second classifications of second abnormal cells and second associated CCLs from the automatic cell classifier in a second image of a second cytological smear. In step 184, compare a result of a function of the second CCLs with the threshold. And in step 185, not present abnormal cells as part of default human review workflow responsive to determining that the result of the function of the second CCLs falls on the sufficient confidence side of the threshold.

In some implementations of the method, the not presenting, as part of default human review workflow, abnormal cells associated with the second sample comprises at least one of: preventing the second sample from entering human review queues, routing the second sample through pathways that bypass human review, or displaying the second sample in a manner distinguishable from the first sample included in the default human review workflow. In the context of this embodiment, “not presenting, as part of default human review workflow, abnormal cells associated with the second sample” refers to refraining from including the second sample or cells therefrom in the default review process that would otherwise route cytological smear images to human reviewers for examination and/or verification. This not presenting operation may be implemented through various technical mechanisms that prevent the second sample from entering the human review queue or workflow pathways that constitute the default review process established by the laboratory or clinical facility. The not presenting may involve the system bypassing the human review routing mechanisms for the second sample, wherein the second sample and its associated analysis results proceed through alternative pathways that may include direct transmission to electronic medical information systems, automatic report generation without human verification, storage in completed analysis repositories without human review flags, or other non-review processing channels. The not presenting operation may be distinguished from active rejection or deletion of the second sample, as the second sample and its associated data may remain accessible within the system for potential access through non-default mechanisms such as manual query, quality assurance sampling, or triggered review based on subsequent clinical information, while being excluded from the routine human review workflow that processes samples requiring human verification based on insufficient CCLs.

The implementation of not presenting may involve one or more of: marking the second sample with metadata flags indicating completion without human review requirement, marking the first sample and not marking the second sample in order to differentiate between the first and second samples, marking the first and second samples in different manners that are distinguishable through visual or textual indicators, routing the second sample through automated reporting pathways that bypass human review interfaces, excluding the second sample from review queue population algorithms that generate worklists for human reviewers, storing the second sample in database partitions designated for automatically completed analyses rather than pending review repositories, or presenting the second sample in a segregated display area below or separate from samples requiring default human review with visual and/or functional separators that prevent inadvertent review. For example, when the result of the function of the second CCLs falls on the sufficient confidence side of the threshold, the system may generate a completed analysis report for the second sample that proceeds directly to the laboratory information system without appearing in the human reviewer's assigned workload, review queue, or pending cases interface. In another example, the not presenting may involve applying routing rules that direct the second sample to an automated verification pathway where quality metrics are recorded and final results are transmitted to clinical systems without the second sample being displayed on review workstations or included in reviewer task lists. In a further example, the not presenting may involve presenting the final results for cells in the second sample to a human without an indication that the cells require review, whereas the final results for cells in the first sample are presented to a human with an indication that the cells require review. In yet another example, the not presenting may involve displaying the second sample below the first sample with a visual separator such as a distinct border, color coding, disabled interaction controls, and/or textual indicators that explicitly identify the second sample as excluded from default human review workflow, wherein the separator and/or interface design may prevent or discourage the human reviewer from examining or modifying the second sample as part of their default review activities. The not presenting operation may additionally involve maintaining audit records indicating that the second sample was processed through automated pathways based on sufficient classification confidence, while preserving the capability for the second sample to be accessed through review mechanisms outside the default workflow, such as when specifically requested by a physician, when flagged by subsequent quality assurance protocols, when laboratory policies require review, when a reviewer indicates a desire to review the second sample, and/or when clinical circumstances warrant retrospective examination of previously analyzed samples that bypassed initial human review based on their high CCLs falling on the sufficient confidence side of the threshold.

In some implementations, the method further comprises presenting, as part of default human review workflow, a subset of classified normal cells associated with the first sample; wherein the normal cells are associated with CCLs below a second threshold. In various embodiments, the systems may process and classify various cell types found in cytological smears. Normal cells may refer to cells that exhibit morphological characteristics, sizes, nuclear features, cytoplasmic ratios, staining properties, and/or optionally other parameters that fall within expected ranges for healthy individuals without pathological conditions. Normal cell types may include, but are not limited to, mature neutrophils, lymphocytes, monocytes, eosinophils, and basophils with typical morphological features and expected distribution patterns. Abnormal cells may refer to cells that exhibit deviations from these expected parameters, which may potentially indicate pathological conditions. These deviations may include atypical nuclear configurations, altered nuclear-to-cytoplasmic ratios, presence of inclusions, unusual staining characteristics, or morphological variations that differ from typical presentations. Abnormal cell types may include, but are not limited to, blast cells, immature forms of otherwise normal lineages, cells with dysplastic features, cells with reactive changes, cells with toxic granulation, and cells showing other morphological variations that may warrant further evaluation. The classification of cells as normal or abnormal may be context-dependent and may vary based on factors such as patient medical history, ongoing treatments, known genetic conditions, and/or other clinical parameters that may influence cellular presentation in cytological specimens. By further presenting normal cells with low CCL values, the system can bring to the attention of the human reviewer a broad spectrum of cases where the classifier results are uncertain, thus helping in preventing errors and improving the classifier.

In some implementations of the method, the first image comprises at least 100 white blood cells (WBCs), and wherein the presenting of the at least some of the first abnormal cells comprises at least one of (i) flagging the abnormal cells associated with CCLs below the threshold, whereby the flagged cells are visually distinguishable when presented for human review, or (ii) applying visual highlighting to the abnormal cells based on their CCLs, wherein different CCL ranges are associated with different highlighting styles.

In some implementations of the method, the presenting of the at least some of the first abnormal cells comprises extracting a subset of the first image containing the abnormal cells associated with CCLs that fall on the insufficient confidence side of the threshold, and combining the extracted subset into a review package.

In some implementations of the method, the first image comprises at least 100 white blood cells (WBCs), and the presenting of the at least some of the first abnormal cells further comprises at least one of (i) annotating the abnormal cells with contextual information derived from electronic health records of the patient, whereby at least some of the contextual information is expected to be relevant to interpreting the abnormal cells with CCLs below the threshold, or (ii) identifying, from patient records, medications or physiological conditions known to cause morphological changes that could affect cell classification; and annotating the abnormal cells that exhibit morphological features potentially attributable to the identified medications or physiological conditions.

In some implementations of the method, the presenting of the at least some of the first abnormal cells comprises assigning priority levels to different portions of the at least some of the first abnormal cells based on clinical significance of abnormal cell types and their corresponding CCLs, and further comprising guiding the human review of the first abnormal cells according to the assigned priority levels.

In some implementations of the method, the presenting of the at least some of the first abnormal cells comprises at least one of (i) formatting the abnormal cells for display within a review interface that presents the abnormal cells alongside reference comparison images of similar abnormal cell types, or (ii) presenting for human review at least two of: visual cellular features, the classifications, or the CCLs.

In some implementations of the method, the presenting of the at least some of the first abnormal cells comprises organizing the abnormal cells into a progressive presentation structure arranged as a function of clinical importance and CCL, such that abnormal cells associated with high clinical importance and lower CCLs are presented to the human reviewer first, followed by abnormal cells with lower clinical importance and relatively higher CCLs that are still below the threshold. In various embodiments, the system may present subsets of abnormal cells from cytological smear images for human review using multiple techniques that may enhance reviewer efficiency and diagnostic accuracy. Optionally, the system may flag abnormal cells with CCLs below predetermined thresholds using techniques such as colored borders, distinct icons, or opacity variations that make these cells distinguishable within the overall image context. For example, abnormal cells with CCLs below a first threshold may receive red borders, while those between the first and a second (higher) threshold may receive yellow borders. Alternatively, the system may extract a relevant image subset containing abnormal cells with low-confidence classifications to create focused review packages that eliminate the need for reviewers to scan through the entire image, which may reduce review time by consolidating abnormal cells requiring attention into a cohesive presentation. Additionally or alternatively, system may enhance review effectiveness by incorporating contextual information, such as recent chemotherapy treatments that could explain morphological variations in abnormal cells, or genetic conditions that predispose to certain abnormal cell presentations, which may provide reviewers with background that may influence their interpretation of borderline classifications. Optionally, prioritization capabilities may enable allocation of reviewer attention, with potentially malignant abnormal cells or those associated with acute conditions receiving higher priority than other abnormal cell variants. This prioritization may be implemented through numbered sequences, symbols, temporal changes such as blinking, color-coded urgency indicators, or structured review paths that guide reviewers through the subset of abnormal cells in order of clinical importance. Optionally, interface preparation features may further enhance review quality by providing reference comparisons that highlight subtle distinguishing features between similar abnormal cell types, or by presenting classifications alongside visual data to facilitate rapid verification of algorithmic decisions. Additionally or alternatively, progressive presentation structure may create an efficient workflow where reviewers first examine high-impact, low-confidence classifications of abnormal cells (such as potential blast cells with CCLs below a threshold), and subsequently review less critical classifications (such as minor variations in abnormal lymphocyte morphology with CCLs that are higher but still below the threshold) after addressing more urgent matters. These presentation techniques may reduce cognitive load on reviewers, focus attention on clinically significant decisions, and improve overall diagnostic efficiency while maintaining quality.

In some implementations, the method further comprises (i) determining, based on the CCLs, that a subset of the first abnormal cells requires human review; and presenting most of the first image along with the subset of the first abnormal cells to the human reviewer selected from available reviewers based on at least one of the CCLs and abnormal cell types present in the subset, wherein the subset is utilized to guide the reviewer's attention within the image, or (ii) generating aggregated analysis results based on the classifications and CCLs; and presenting the aggregated analysis results along with the subset of abnormal cells to the human reviewer selected from available reviewers based on at least one of the aggregated analysis results and abnormal cell types present in the first sample.

In some implementations, the method further comprises applying different CCL thresholds to different abnormal cell types when determining if the result of the function of the CCLs falls on the sufficient confidence side of the threshold or insufficient confidence side of the threshold, with higher CCL thresholds used for abnormal cell types associated with greater clinical significance. The system may implement abnormal cell type-specific CCL thresholds that reflect the varying clinical significance and morphological complexity of different abnormal cellular populations. Different abnormal cell types inherently vary in their clinical implications, with some abnormal cell types providing more diagnostic information than others. For example, blast cells, which may indicate serious conditions such as leukemia, may be included in the subset presented for human review even when their CCLs are relatively high but still below their type-specific threshold, as even small misclassification risks for these cells warrant human verification due to their significant clinical impact. Conversely, abnormal cell types with less severe clinical implications may be included in the subset when their CCLs fall below a much lower threshold. Rare abnormal cell types with uncertain diagnostic significance may receive intermediate thresholds that balance their relative rarity against their potential clinical significance. The differential threshold focuses human expertise on abnormal cells where misclassifications would have high potential clinical consequences, potentially improving diagnostic accuracy for critical abnormal cell populations while reducing unnecessary reviews of less clinically significant abnormal cell types with relatively higher CCLs.

In some implementations, the method further comprises receiving, from an Electronic Medical Information System, medical history information associated with the first patient to identify a medical condition relevant to the patient, and adjusting at least one threshold based on the medical condition for determining which of the abnormal cells to present to the human reviewer. Optionally, the abnormal cells presented may be adapted based on patient-specific medical history information to create more contextually relevant presentations for human review. By incorporating medical record data, the system can adjust which cells are included in the prepared subset based on conditions that affect expected cell morphology or distribution. For patients with known hematological disorders, the system may increase CCL thresholds for including specific cell lineages in the prepared subset, for example for a patient with chronic myeloid leukemia, immature granulocytes may be included in the subset even with relatively high CCLs to enable careful monitoring of disease progression. For patients undergoing chemotherapy, the system may adjust subset preparation criteria to highlight morphological variations in neutrophils that may otherwise be classified as abnormal but are expected side effects of treatment.

In some implementations, the method further comprises receiving information indicative of a certain disease progression stage for the first patient; and wherein the threshold is a function of the disease progression stage, whereby value of the threshold for an earlier stage of a disease differs from value of the threshold for a later stage of the disease. The system may implement disease progression stage-specific CCL thresholds that account for the different monitoring requirements and expected findings at various stages of disease progression. By receiving disease stage information from the patient's medical record, the system can apply appropriate CCL thresholds that reflect the clinical priorities associated with different stages when preparing review subsets. For example, for patients with newly diagnosed hematological malignancies, the system may implement higher CCL thresholds during the initial months following diagnosis, including cells in the prepared subset even when their CCLs are relatively high, to verify the baseline response patterns during this period. For patients with stable chronic conditions, the system may implement more selective subset preparation by lowering CCL thresholds, including cells for human review when CCLs fall below these lower thresholds or when significant changes from established baselines are detected. This may help optimize human review resources by creating more comprehensive review subsets during critical disease stages and producing more focused subsets during periods of disease stability.

In some implementations, the method further comprises determining a time period since initial diagnosis of a condition; and adjusting the threshold based on the time period, such that a first threshold is applied during an initial monitoring period following diagnosis, and a second threshold is applied as the time period increases for stable conditions; whereby applying the first and second thresholds generally decreases the rate of sending samples for human review with time and stability of the condition.

In some implementations, the method further comprises presenting to the human reviewer unclassified cells associated with the first sample, responsive to receiving from the automatic cell classifier an amount of unclassified cells that reaches a second threshold. Optionally, when the number of unclassified cells reaches a second threshold, it may indicate that the automatic cell classifier is encountering difficulties with the cytological smear that could affect overall classification reliability. The second threshold may be defined as an absolute number of unclassified cells (such as more than 10 unclassified WBCs), a percentage of the total cells analyzed (such as more than 5% unclassified WBCs cells), or a combination of factors incorporating both quantity and distribution patterns of unclassified cells. A relatively high number of unclassified cells may indicate various underlying issues, including unusual cellular morphologies, staining variations, preparation artifacts, and/or the presence of rare cell types not adequately represented in the automatic cell classifier's training data. By presenting at least a subset of the image containing these unclassified cells to the human reviewer, the system enables expert assessment of cells that may fall outside the reliable classification capabilities of the automatic cell classifier, potentially identifying clinically significant findings and providing valuable feedback for improving the classifier's performance with challenging cell types. Cells associated with the sample, may refer to any cells associated with the sample, may include cells classified according to any classification, and may relate to any image or any cytological smear.

In some implementations, the method further comprises receiving human reviewer classifications for at least some of the unclassified cells, and updating the automatic cell classifier based on the human reviewer classifications to improve future classifications for similar cells. Optionally, the system may implement a learning mechanism that utilizes human reviewer input on unclassified cells to improve the automatic cell classifier and/or to improve personal classification preferences for the specific human reviewer. When the human reviewer classifies cells that the automatic cell classifier marked as unclassified or cells that were classified but the human reviewer changed their classification, the system may capture these classifications and incorporate them into the automatic cell classifier's knowledge base. This process may include adding new reference examples for rare or unusual cell types, adjusting classification parameters for borderline morphologies, and/or expanding the automatic cell classifier's recognition capabilities for variant presentations of known cell types. This feedback loop between human expertise and the automatic cell classifier may also help the system to improve its classification capabilities for challenging cell presentations.

This embodiment may be integrated with human confirmation or oversight processes without departing from the automated decision-making framework for preparing images for human review based on CCLs. For example, the system may automatically compare a result of a function of the CCLs with a threshold while simultaneously providing laboratory personnel with user interface controls to monitor and adjust threshold settings for different abnormal cell types based on clinical requirements or laboratory-specific protocols. The system may provide a user interface that enables laboratory staff to review and approve CCL threshold configurations, establish custom threshold values for specific abnormal cell types with high clinical significance, and monitor the automated presenting and not presenting decisions. In clinical environments that may require human oversight, supervisors may utilize interface controls to review cases where the result of a function of the CCLs falls near threshold boundaries, approve modifications to default human review workflow parameters, and establish quality assurance protocols for validating automated presentation decisions. The user interface may display comparative analysis of CCLs across different abnormal cell types, enable manual override of automated presentation decisions when clinical judgment warrants deviation from standard threshold-based routing, and provide feedback mechanisms for laboratory staff to evaluate the effectiveness of threshold settings in optimizing human reviewer workload. Additionally, the system may integrate approval workflows where laboratory managers can review and confirm threshold adjustments, verify that abnormal cells associated with CCLs that fall on the insufficient confidence side of the threshold are appropriately presented as part of default human review workflow, and establish custom presentation criteria for specific patient populations or clinical contexts. Such human confirmation processes may operate in conjunction with the automated comparing of a result of a function of the CCLs with a threshold and presentation determination workflow, enhancing quality control while maintaining the efficiency gains provided by automated confidence-based routing of abnormal cells for human review. In some implementations of the method, a non-transitory computer-readable medium comprising instructions that, when executed by a processor, cause the processor to perform the computer-implemented method.

In various implementations, a system for preparing images for human review, comprising: a computer configured to: receive, from an automatic cell classifier, first classifications of first abnormal cells and first associated classification confidence levels (CCLs), wherein the first abnormal cells are in a first image of a first cytological smear associated with a first sample obtained from a first patient; compare a result of a function of the first CCLs with a threshold; responsive to determining that the result of the function of the first CCLs falls on the insufficient confidence side of the threshold, present, as part of default human review workflow, at least some of the first abnormal cells; receive, from the automatic cell classifier, second classifications of second abnormal cells and second associated CCLs, wherein the second abnormal cells are in a second image of a second cytological smear associated with a second sample obtained from a second patient; compare a result of a function of the second CCLs with the threshold; and responsive to determining that the result of the function of the second CCLs falls on the sufficient confidence side of the threshold, not present, as part of default human review workflow, abnormal cells associated with the second sample. FIG. 8B illustrates an embodiment of a block diagram of a system for preparing images for human review. The system includes a computer 186 configured to receive classifications and CCLs from an automatic cell classifier 187. Optionally, the computer 186 is also coupled to an electronic medical information system 188 for accessing patient medical history information. The computer 186 processes the CCLs by comparing results of functions of the CCLs with predetermined thresholds to determine whether abnormal cells should be presented for human review. When CCLs fall on the insufficient confidence side of the threshold, the computer 186 presents the abnormal cells through a human review workflow interface 189. The system selectively routes samples for human review based on confidence levels, with samples having sufficient confidence bypassing human review in the default workflow.

In some implementations of the system, the computer is further configured to present, as part of default human review workflow, a subset of classified normal cells associated with the first sample; wherein the normal cells are associated with CCLs below a second threshold. In some implementations of the system, the first image comprises at least 100 white blood cells (WBCs), and wherein the computer, when presenting the at least some of the first abnormal cells, is configured to perform at least one of (i) flagging the abnormal cells associated with CCLs below the threshold, whereby the flagged cells are visually distinguishable when presented for human review, or (ii) applying visual highlighting to the abnormal cells based on their CCLs, wherein different CCL ranges are associated with different highlighting styles.

In some implementations of the system, the computer, when presenting the at least some of the first abnormal cells, is configured to extract a subset of the first image containing the abnormal cells associated with CCLs that fall on the insufficient confidence side of the threshold, and combine the extracted subset into a review package. In some implementations of the system, the first image comprises at least 100 white blood cells (WBCs), and the computer, when presenting the at least some of the first abnormal cells, is further configured to perform at least one of (i) annotating the abnormal cells with contextual information derived from electronic health records of the first patient, whereby at least some of the contextual information is expected to be relevant to interpreting the abnormal cells with CCLs below the threshold, or (ii) identifying, from patient records, medications or physiological conditions known to cause morphological changes that could affect cell classification; and annotating the abnormal cells that exhibit morphological features potentially attributable to the identified medications or physiological conditions.

In some implementations of the system, the computer, when presenting the at least some of the first abnormal cells, is configured to assign priority levels to different portions of the at least some of the first abnormal cells based on clinical significance of abnormal cell types and their corresponding CCLs. In some implementations of the system, the computer is further configured to guide the human review of the first abnormal cells according to the assigned priority levels. In some implementations of the system, the computer, when presenting the at least some of the first abnormal cells, is configured to perform at least one of (i) formatting the abnormal cells for display within a review interface that presents the abnormal cells alongside reference comparison images of similar abnormal cell types, or (ii) presenting for human review at least two of: visual cellular features, the classifications, or the CCLs.

In some implementations of the system, the computer, when presenting the at least some of the first abnormal cells, is configured to organize the abnormal cells into a progressive presentation structure arranged as a function of clinical importance and CCL, such that abnormal cells associated with high clinical importance and lower CCLs are presented to the human reviewer first, followed by abnormal cells with lower clinical importance and relatively higher CCLs that are still below the threshold.

In some implementations of the system, the computer is further configured to perform at least one of (i) determining, based on the CCLs, that a subset of the first abnormal cells requires human review; and presenting most of the first image along with the subset of the first abnormal cells to the human reviewer selected from available reviewers based on at least one of the CCLs and abnormal cell types present in the subset, wherein the subset is utilized to guide the reviewer's attention within the image, or (ii) generating aggregated analysis results based on the classifications and CCLs; and presenting the aggregated analysis results along with the subset of abnormal cells to the human reviewer selected from available reviewers based on at least one of the aggregated analysis results and abnormal cell types present in the first sample.

In some implementations of the system, the computer is further configured to apply different CCL thresholds to different abnormal cell types when determining if the result of the function of the CCLs falls on the sufficient confidence side of the threshold or insufficient confidence side of the threshold, with higher CCL thresholds used for abnormal cell types associated with greater clinical significance.

In some implementations of the system, the computer is further configured to: receive, from an Electronic Medical Information System, medical history information associated with the first patient to identify a medical condition relevant to the first patient; and adjust at least one threshold based on the medical condition for determining which of the abnormal cells to present to the human reviewer. In some implementations of the system, the computer is further configured to receive information indicative of a certain disease progression stage for the first patient; and wherein the threshold is a function of the disease progression stage, whereby value of the threshold for an earlier stage of a disease differs from value of the threshold for a later stage of the disease.

In some implementations of the system, the computer is further configured to: determine a time period since initial diagnosis of a condition; and adjust the threshold based on the time period, such that a first threshold is applied during an initial monitoring period following diagnosis, and a second threshold is applied as the time period increases for stable conditions; whereby applying the first and second thresholds generally decreases the rate of sending samples for human review with time and stability of the condition.

In some implementations of the system, the computer is further configured to present to the human reviewer unclassified cells associated with the first sample, responsive to receiving from the automatic cell classifier an amount of unclassified cells that reaches a second threshold. In some implementations of the system, the computer is further configured to: receive human reviewer classifications for at least some of the unclassified cells; and update the automatic cell classifier based on the human reviewer classifications to improve future classifications for similar cells.

The following implementation focuses on differential presentation approaches for cytological smear analysis, including: (i) presenting to human reviewers the relevant subset of cytological smear analysis that require human review, and (ii) presenting the broader sample context with clear identification of the subset requiring review. By selectively presenting specific cells or data of interest to reviewers at a resolution sufficient for cell-level review, or by identifying these cells within the complete context, the system may reduce review time while maintaining diagnostic accuracy. The review process may be optimized by focusing expert attention on the first subset of classified cells that require human judgment, either by limiting the presented material to substantially the first subset or by guiding attention to the first subset within the complete sample. By refraining from presenting the second subset of classified cells at a resolution sufficient for cell-level review, unnecessary expert time is conserved. The embodiment supports more efficient use of human resources and potentially faster turnaround times for patient results while offering flexibility in presentation methods based on reviewer preferences, cell types of interest, or specific diagnostic requirements.

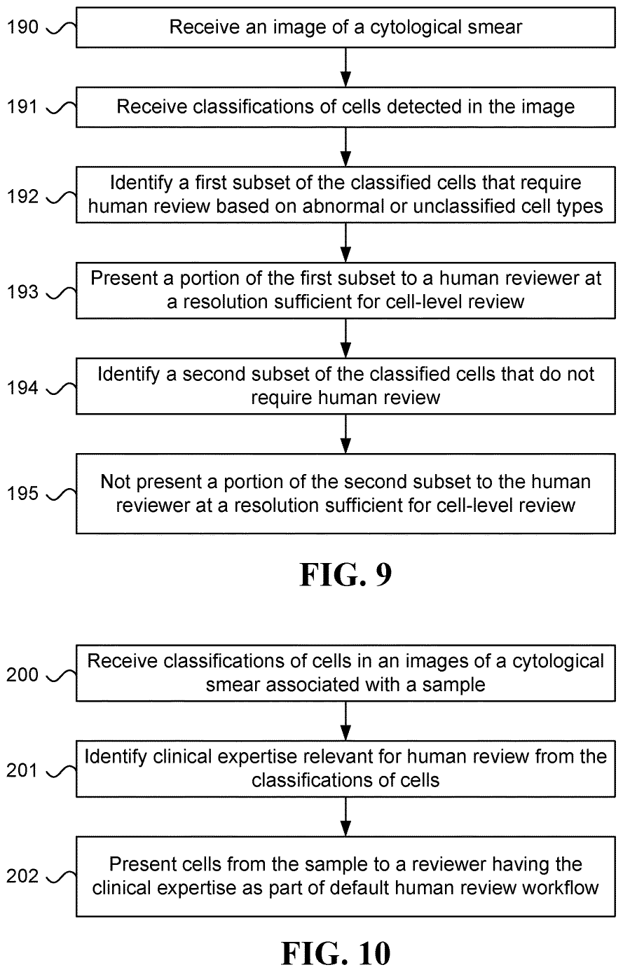

In various implementations, a computer-implemented method for differential human review of cytological smear analysis, comprising: receiving an image of a cytological smear associated with a patient; receiving, from an automatic cell classifier, classifications of cells detected in the image; identifying a first subset of the classified cells that require human review based on predetermined criteria comprising at least one of extent of cells classified as an abnormal cell type or extent of cells classified as an unclassified cell type; presenting, to a human reviewer, a portion of the first subset at a resolution sufficient for cell-level review; identifying a second subset of the classified cells that do not require human review based on the predetermined criteria; and not presenting to the human reviewer a portion of the second subset at a resolution sufficient for cell-level review, unless an indication is received from the human reviewer. FIG. 9 illustrates an embodiment of a computer-implemented method for differential human review of cytological smear analysis. The method includes the following steps. In step 190, receive an image of a cytological smear. In step 191, receive classifications of cells detected in the image. In step 192, identify a first subset of the classified cells that require human review based on abnormal or unclassified cell types. In step 193, present a portion of the first subset to a human reviewer at a resolution sufficient for cell-level review. In step 194, identify a second subset of the classified cells that do not require human review. And in step 195, not present a portion of the second subset to the human reviewer at a resolution sufficient for cell-level review.

The differential human review method may optimize the review process by directing human attention to cells requiring expert judgment while conserving review time by not presenting cells that have been reliably classified by the automatic cell classifier. The first subset may include cells that meet specific criteria indicating they warrant human verification, which may include cells classified as abnormal (such as blast cells, atypical lymphocytes, or cells showing morphological abnormalities), unclassified cells (which may represent either artifacts, cells that did not cross the threshold of any cell type in a predetermined group, or that have high confusion between cell types, rare cell types, or cells with ambiguous features), and/or cells with other characteristics indicating they benefit from human interpretation. The presentation of a portion of the first subset may involve various interface approaches including gallery views of individual cells, focused fields containing relevant cells, or highlighted regions within broader smear context. The identification of the second subset that does not require review may be based on high classification confidence, normal morphology, and/or established normal quantities, and may represent cells where the automatic cell classifier has demonstrated reliable performance. The not presenting to the human reviewer a portion of the second subset at a resolution sufficient for cell-level review may be implemented through various mechanisms including displaying the second subset at reduced resolution insufficient for diagnostic assessment, excluding the second subset from high-magnification viewing areas, presenting the second subset as statistical summaries or thumbnails rather than detailed cellular images, rendering the second subset in background layers with reduced visual prominence, and/or omitting the second subset entirely from the review interface unless the human reviewer provides an indication requesting access to these cells. The indication from the human reviewer may include cursor movements, interface selections, zoom requests, keyboard commands, or other explicit actions that signal intent to examine the second subset, at which point the system may present the requested portion of the second subset at a resolution sufficient for cell-level review. By not presenting the second subset at a resolution sufficient for cell-level review unless specifically requested, the system may reduce unnecessary visual information that could potentially distract from critical assessments, while maintaining the option for the reviewer to access this information when desired. The method may implement various presentation formats including split-screen views, toggled displays, hierarchical zoom functions, or interactive filtering to balance comprehensive context with focused review efficiency.

In some implementations of the method, the predetermined criteria are further based on classification confidence levels (CCLs) falling on the insufficient confidence side of type-specific thresholds, and further comprising determining the cell type-specific thresholds based on statistical confidence requirements calibrated according to clinical significance of their respective cell types and statistical sampling adequacy. The CCL-based subset selection may incorporate variable confidence thresholds that reflect the differential diagnostic significance of various cell types. Cell types with higher clinical significance and/or greater potential impact on diagnosis and treatment decisions may be associated with more stringent confidence thresholds, potentially resulting in more cells of these types being included in the first subset requiring human review. These type-specific thresholds may be established through various methods, including analysis of expert consensus datasets, statistical evaluation of classification performance across diverse sample populations, correlation with clinical outcomes, and/or consideration of established medical guidelines regarding diagnostic certainty requirements. The calibration process may take into account both the clinical significance of potential misclassification (with higher-stakes classifications requiring greater confidence) and statistical sampling adequacy (ensuring sufficient cells are available to support reliable classification). The threshold determination may incorporate factors such as the rarity of the cell type, the similarity to other cell types that may cause classification confusion, the stability of morphological features under various preparation conditions, and/or the diagnostic weight carried by the particular cell type in various clinical contexts. This tailored approach to confidence thresholds may help balance review efficiency with diagnostic thoroughness by adjusting the selectivity of human review requirements based on clinically meaningful parameters.

In some implementations, the method further comprises presenting to the human reviewer a portion of abnormal cells from the first subset associated with classification confidence levels (CCLs) falling on the insufficient-confidence side of a threshold; and refraining from presenting to the human reviewer a portion of abnormal cells from the second subset associated with CCLs falling on the sufficient-confidence side of the threshold, unless a relevant indication is received from the human reviewer. The classification confidence threshold approach for abnormal cells may implement a more nuanced review strategy for cells with potentially significant clinical implications. Abnormal cells with CCLs falling on the insufficient-confidence side of the threshold may include those with borderline or ambiguous morphological features, those belonging to cell types that commonly present classification challenges, or those with characteristics that match multiple possible cell type patterns. The presentation of these lower-confidence abnormal cells may prioritize cases where human judgment may add high diagnostic value, potentially improving overall classification accuracy for clinically significant findings. Conversely, abnormal cells with CCLs falling on the sufficient-confidence side of the threshold may represent cells with clear morphological features that align strongly with established abnormal cell patterns and can be reliably classified by the automatic cell classifier. By refraining from presenting these high-confidence abnormal cells unless specifically requested, the system may further streamline the review process while maintaining the option for comprehensive verification when desired. The threshold implementation may also incorporate adaptive elements that adjust based on classifier performance validation, laboratory-specific requirements, or individual reviewer preferences while maintaining underlying clinical standards. The system may further implement different thresholds for different categories of abnormal cells based on their respective diagnostic implications and classifier performance characteristics.

In some implementations of the method, the predetermined criteria are further based on cells of a particular classification, wherein the particular classification corresponds to cell types requiring additional verification due to their clinical significance. The classification-based subset selection may prioritize specific cell types that carry heightened diagnostic importance regardless of their classification confidence level. These particular classifications may include cell types associated with serious pathological conditions (such as blast cells potentially indicating leukemia), cell types that serve as key diagnostic indicators for specific disorders (such as plasma cells in multiple myeloma evaluations), rare cell types whose presence may trigger specific clinical pathways, cell types that may influence immediate treatment decisions, and/or cell types that frequently require morphological interpretation beyond automated classification capabilities. The selection of these particular classifications for human review may be based on established clinical guidelines, institutional protocols, current research on diagnostic significance, and/or regulatory requirements for specific testing contexts. The implementation may include tiered classification systems where certain high-impact cell types automatically trigger review regardless of confidence level, while others require review when additional factors such as quantity or distribution patterns suggest potential clinical significance. This approach may help ensure that human expertise is consistently applied to cell classifications with high potential impact on patient care, even when the automatic cell classifier demonstrates generally high confidence in these classifications. The methodology may further include mechanisms to periodically review and update the list of particular classifications requiring verification based on evolving medical knowledge, classifier performance improvements, or specific patient population characteristics.

In some implementations of the method, the predetermined criteria are further based on cells from specific regions of the cytological smear, wherein the specific regions are determined based on known distribution patterns of certain cell types within cytological smears. The region-based subset selection may leverage knowledge about non-random cell distribution patterns within cytological smears to enhance review efficiency and diagnostic accuracy. Various cell types may exhibit predictable distribution tendencies within properly prepared cytological smears due to factors such as cell size, density, adhesion properties, and fluid dynamics during smear preparation. For example, in peripheral blood smears, certain cell types may be more commonly found in the feathered edge, body, or thick regions of the smear. The specific regions selected for focused review may be determined through various approaches, including: analysis of historical smear preparation patterns specific to the laboratory's techniques, established knowledge about optimal observation zones for particular cell types, computerized mapping of cell distribution across multiple samples, and/or adaptive learning based on where diagnostically significant cells have been previously identified. This approach may help concentrate human review on regions with higher probability of containing diagnostically important cells while potentially reducing time spent on regions with lower diagnostic yield. The implementation may include dynamic region selection that adapts based on the specific cell types of interest for a particular analysis, the preparation quality of the individual smear, detected distribution anomalies that may signal pathological conditions, and/or hybrid approaches that combine region-based prioritization with other selection criteria such as classification confidence or morphological features. This may facilitate more effective allocation of human review resources while maintaining diagnostic thoroughness.

In some implementations of the method, the predetermined criteria are further based on cells exhibiting particular morphological features, wherein the particular morphological features are associated with diagnostic ambiguity or clinical significance. The morphology-based subset selection may identify cells that exhibit specific structural, textural, or compositional characteristics warranting expert interpretation, even when the automatic cell classifier has assigned them to particular categories with some confidence. These particular morphological features may include nuclear irregularities (such as hypersegmentation, hypochromasia, or unusual chromatin patterns), cytoplasmic abnormalities (such as unusual granulation patterns, vacuolation, or inclusion bodies), size variations outside typical ranges, unusual nuclear-to-cytoplasm ratios, membrane abnormalities, or morphological features that are shared across multiple cell types creating potential classification ambiguity. The selection may focus on morphological features associated with diagnostic “gray areas” where subtle distinctions may carry significant clinical implications, features that may indicate early manifestations of pathological processes, features that represent transitional or intermediate forms between cell types, and/or rare morphological variants that merit specialist confirmation. The implementation may incorporate quantitative morphometric analysis that flags cells with measurements falling into ambiguous ranges, pattern recognition algorithms that identify unusual or borderline morphological signatures, and/or comparison against reference databases of morphological variations with established diagnostic correlations. This approach may enhance diagnostic value by ensuring human expertise is applied to cells with morphological characteristics that may be challenging for automated classification systems, potentially improving detection of subtle pathological changes while maintaining efficient workflow for cells with straightforward morphological presentations.

In some implementations of the method, the predetermined criteria are further based on relations between at least some of the classified cells and respective normal ranges of clinical thresholds, comprising at least one of: category statistics indicating distribution of different cell types within the cytological smear, or cell statistics indicating morphological parameter variations within specific cell populations. The normal range criteria approach may incorporate reference ranges to identify cells or cell populations that merit human review based on their deviation from established clinical patterns. These criteria may include analysis of category statistics, such as relative percentages of specific cell types compared to typical distributions, ratios between related cell types that may indicate physiological responses or pathological processes, absolute counts of certain cell types that approach or exceed clinical action thresholds, and/or distribution patterns that suggest potential shift reactions or lineage abnormalities. Additionally, the criteria may evaluate cell statistics within specific populations, examining morphological parameter variations such as size distribution curves that show unusual clustering or skewing, nuclear chromatin pattern variations that exceed normal heterogeneity for a given cell type, granulation density distributions that suggest potential functional abnormalities, and/or other quantifiable morphological characteristics with established normal ranges. The implementation may involve multi-parameter statistical analysis that identifies combinations of values falling outside typical correlation patterns, comparison against reference datasets stratified by relevant factors such as age, sex, or medical condition, and/or temporal comparison with the patient's own historical values when available to identify significant changes that may warrant human verification. This approach may help focus human review on cells and cell populations that represent potential deviations from physiological norms, even when individual cells may be classified with high confidence, potentially enhancing detection of clinically significant patterns while maintaining efficient review processes for results falling within expected parameters.

In some implementations of the method, the predetermined criteria are further based on sample preparation quality parameters comprising at least one of: staining adequacy, cell distribution uniformity, presence of preparation artifacts, smear thickness, or monolayer formation characteristics. The sample preparation criteria may identify areas requiring human review based on technical factors that may influence classification reliability. These quality parameters may include evaluation of staining adequacy, such as stain intensity, color differentiation, contrast between cellular components, consistency across the smear, and/or presence of staining artifacts that may affect morphological interpretation. Cell distribution uniformity assessment may identify regions with suboptimal spreading, cell clustering, excessive overlap, or distribution patterns that deviate from expected monolayer formations. The criteria may detect preparation artifacts including smudge cells, mechanical damage to cells during preparation, drying artifacts that alter morphology, precipitation of staining reagents, air bubbles, contaminants from collection materials, or other technical factors that may compromise automated classification accuracy. Smear thickness evaluation may identify regions where excessive thickness may obscure cellular details or areas where insufficient material may limit statistical validity. Monolayer formation characteristics assessment may examine the achievement of appropriate cell separation while maintaining cellular integrity, which may assist in accurate morphological analysis. The implementation may incorporate multi-parameter quality assessment algorithms that weigh various preparation aspects against established quality benchmarks, differential criteria for various cell types based on their specific preparation sensitivities, and/or adaptive thresholds that consider the overall preparation quality in relation to the specific analytical goals. This approach may enhance classification reliability by ensuring human expertise is applied to areas where preparation factors may influence automated classification accuracy, while potentially reducing unnecessary review of technically optimal regions.

In some implementations, the method further comprises providing location information for the portion of the first subset presented to the human reviewer, wherein the location information indicates positions of cells within the overall cytological smear to facilitate spatial interpretation. Location information may enhance diagnostic context by preserving the spatial relationships between presented cells and the broader smear environment. This contextual information may be implemented through various approaches, including: miniature overview maps with highlighted regions indicating where presented cells are located within the complete smear, coordinate systems that reference standardized smear regions such as feathered edge, body, or monolayer zones, navigational tools that allow reviewers to seamlessly transition between focused cell views and broader contextual perspectives, and/or heat maps that indicate cell distribution patterns with markers for reviewed cells. The location information may be particularly valuable for interpretation of certain cell types whose diagnostic significance may be influenced by their spatial distribution, such as cell clusters that may suggest colony formation, gradients of maturation stages that follow specific distribution patterns, or cell types that typically exhibit non-random positioning within properly prepared smears. The implementation may include interactive features that enable reviewers to access location context on demand, persistent location markers that remain visible regardless of magnification level, and/or comparative location information that shows distribution patterns from multiple samples when relevant for progressive monitoring. This spatial context may help reviewers interpret cellular findings within their physical distribution context, potentially enhancing detection of meaningful distribution patterns while maintaining focus on individual cells requiring detailed examination. The system may also track reviewed locations to ensure comprehensive coverage of relevant areas and prevent inadvertent duplication of review effort.

In some implementations, the method further comprises providing information about cells adjacent to cells in the portion of the first subset presented to the human reviewer, wherein the adjacent cells provide contextual information that assists in interpreting morphological variations and distinguishing between similar cells. The adjacent cell contextual information may provide microenvironmental context that can assist in accurate interpretation of certain cell types and morphological features. This information may be presented through various mechanisms, including expanded field views that show surrounding cells while maintaining focus on the primary cells of interest, supplementary images that display adjacent fields upon request, interactive zooming capabilities that allow seamless transition between focused and contextual views, and/or annotated information about nearby cell populations without necessarily displaying their images. The adjacent cell information may be particularly valuable in various interpretive contexts, such as: distinguishing reactive lymphocytes from abnormal lymphoid cells by examining the cellular environment in which they appear, evaluating red blood cell morphology variations that may present differently in different regions of the smear, assessing whether morphological features represent genuine cellular characteristics or artifacts by examining patterns in neighboring cells, and/or identifying cellular interactions or relationships that may have diagnostic significance. The implementation may employ selection algorithms that prioritize adjacency information with potential interpretive value, customizable display options that allow reviewers to adjust the extent of contextual information based on their preferences and the specific review requirements, and/or filtered adjacency information that emphasizes certain cell types within the microenvironment based on their relevance to the primary cells being reviewed. This contextual approach may enhance diagnostic accuracy by providing information about the cellular microenvironment that may influence interpretation of morphological features, potentially improving discrimination between similar-appearing cell types while maintaining review efficiency.

In some implementations, the method further comprises providing statistical summaries of cell distributions alongside the portion of the first subset presented to the human reviewer, wherein the statistical summaries establish population-level context for interpreting individual cells. The statistical contextual information may provide quantitative landscape information that establishes broader patterns against which individual cells can be interpreted. These statistical summaries may include various elements such as: differential counts showing proportional relationships between cell types within the sample, population distribution graphs depicting morphological parameter ranges for relevant cell types, comparative statistics showing how current sample metrics relate to patient-specific historical values when available, reference range indicators that position current findings within established clinical parameters, and/or flagged deviations that highlight statistically significant variations from expected patterns. The implementation may incorporate various visualization approaches including numerical summaries, graphical representations such as histograms or scatter plots, color-coded indicators that intuitively communicate statistical significance, and/or comparative displays that facilitate pattern recognition across multiple parameters. The statistical information may be valuable for various interpretive tasks, including: distinguishing between reactive changes and pathological processes by evaluating overall population shifts, identifying subtle distribution abnormalities that may not be apparent from examining individual cells, establishing whether borderline morphological features represent statistically significant variations or expected diversity within a population, and/or providing quantitative context for rare findings whose significance may depend on their frequency within the overall sample. This population-level perspective may enhance diagnostic accuracy by allowing reviewers to interpret individual cellular features within the context of broader distribution patterns, potentially improving detection of clinically significant variations while facilitating efficient interpretation of findings that, while perhaps morphologically interesting, represent expected statistical variations within normal ranges.

In some implementations of the method, presenting the portion of the first subset comprises providing a combination of cell images and textual information, wherein certain cell types are presented as images while information about other cell types is provided as text. The combined presentation approach may leverage different information modalities to optimize review efficiency while maintaining comprehensive diagnostic information. This hybrid approach may implement various presentation methods, such as: displaying full-resolution images for cell types requiring detailed morphological assessment while providing text summaries for cell types with straightforward or confidently classified features, presenting images for rare or abnormal cells while summarizing normal populations textually, creating tiered presentation formats where primary findings appear as images while secondary or supportive information appears as text, and/or implementing interactive designs where textual information can be expanded into visual information based on reviewer request. The determination of which cell types to present as images versus text may be based on multiple factors, including the clinical significance of the cell type, the confidence level of the automatic classification, the complexity of morphological features requiring interpretation, the rarity of the finding, and/or the reviewer's stated preferences or specialized expertise. The textual information may include various elements such as quantitative data about cell populations, classification confidence metrics, morphometric measurements of key cellular features, comparative information against reference ranges, and/or system-generated interpretive suggestions based on recognized patterns. This multimodal presentation may optimize the review process by directing visual attention to cells requiring morphological assessment while efficiently communicating supporting information that may not require visual inspection, potentially reducing review time while maintaining diagnostic thoroughness. The implementation may incorporate customizable formats that adapt to individual reviewer workflows, laboratory protocols, or specific diagnostic contexts.

In some implementations, the method further comprises selecting the extent from a count or a percentage of cells, and wherein the extent of unclassified cells refers to at least one specific cell type comprising unclassified white blood cells. The extent selection between count-based or percentage-based extent measures may depend on various factors, including the typical abundance of the cell type, clinical significance thresholds established in medical literature or guidelines, statistical considerations related to sampling adequacy, and/or compatibility with reference ranges used in clinical interpretation. For relatively abundant cell populations, percentage-based extents may often provide more stable measures across samples with different cellularity levels, while for rare cell types, absolute counts may sometimes more clearly communicate clinical significance when percentages might appear misleadingly small. The specific reference to unclassified white blood cells may address a particularly important category that often requires human interpretation, as these cells may represent either technical limitations in the classification algorithm, cells with ambiguous or transitional features, abnormal cells that fall outside established classification parameters, or rare subtypes with diagnostic significance. The implementation may incorporate different extent thresholds for different cell types based on their clinical significance, expected frequency in normal samples, and/or classification reliability characteristics. This approach to extent definition may provide appropriate flexibility in determining review thresholds while maintaining focus on clinically meaningful quantification methods.

In some implementations of the method, the resolution sufficient for cell-level review comprises an effective numerical aperture and image quality that enables visualization of cellular morphological features necessary for human classification and verification of white blood cells. For WBCs, the resolution sufficient for cell-level review enables the human reviewer to clearly distinguish nuclear details, cytoplasmic characteristics, and granulation patterns that differentiate cell types such as neutrophils, lymphocytes, and monocytes. For RBCs, the resolution sufficient for cell-level review enables the human reviewer to identify cell shape, hemoglobinization, and inclusion bodies such as Howell-Jolly bodies or Pappenheimer bodies that may indicate pathological conditions.

In some implementations, the method further comprises implementing a tiered review process wherein an initial reviewer receives the portion of the first subset and determines whether to escalate specific elements to a specialist reviewer based on analysis of the portion of the first subset. The hierarchical review process may implement a multi-level approach that optimizes the application of specialized expertise while maintaining workflow efficiency. This tiered structure may involve various personnel roles, including: laboratory technicians or generalist reviewers who conduct initial assessments and handle cases with straightforward findings, specialist pathologists or hematologists who review escalated cases requiring advanced expertise, subspecialists with expertise in specific areas such as pediatric hematopathology or rare disorders who receive further escalations for particularly challenging cases, and/or supervisory reviewers who conduct quality assurance checks on a sampling of cases across different escalation levels. The escalation criteria may include various triggers such as: detection of specific cell types or morphological features associated with serious conditions, reviewer indication, large discrepancy between automatic results and human review results, identification of patterns requiring specialized interpretation, findings that conflict with prior diagnoses or current clinical presentation, uncertainty in interpretation of borderline or ambiguous features, and/or qualification-based routing to ensure appropriate credentials for specific diagnostic determinations. The implementation may incorporate workflow management tools that track escalation status, documentation systems that maintain records of review decisions at each level, communication mechanisms that facilitate consultation between reviewers at different tiers, and/or analytics that monitor escalation patterns to identify potential training opportunities or classification algorithm improvements. This tiered approach may enhance diagnostic quality by ensuring that challenging cases receive appropriate levels of expertise while optimizing resource utilization by directing routine cases to appropriate personnel, potentially improving both efficiency and diagnostic accuracy. The system may also implement automated pre-escalation of certain findings based on established criteria, bypassing initial review tiers for findings that consistently require specialist interpretation.

In some implementations of the method, the predetermined indication from the human reviewer comprises at least one of: a cursor movement operation, a scrolling operation, a link activation operation, a content highlighting operation, an area marking operation, a text command input, a voice command input, a keyboard stroke operation corresponding to a predetermined action, or an eye movement pattern detected by an eye tracking device. The human indication detection may implement diverse interaction mechanisms that accommodate different reviewer preferences, workstation configurations, and laboratory environments. These interaction methods may be implemented through various technical approaches, including: traditional input devices such as mice, trackpads, or keyboards that detect explicit user actions, touch-sensitive interfaces that allow direct interaction with displayed content, voice recognition systems that process verbal commands for hands-free operation, eye tracking technologies that monitor gaze patterns to interpret viewing intentions, and/or hybrid systems that integrate multiple input modalities to enhance interaction flexibility. The specific indications may be customized based on workflow analysis, ergonomic considerations, reviewer feedback, and/or laboratory-specific protocols. Cursor movement operations may include hovering over specific areas to trigger additional information display, clicking on cells or regions to request expanded views, or dragging actions to select multiple elements for comparison. Scrolling operations may navigate through cell galleries, extended fields of view, or additional contextual information. Link activation operations may connect to reference materials, historical records, jump between sections, open hidden sections, or connect to related case information. In some implementations of the method, a non-transitory computer-readable medium comprising instructions that, when executed by a processor, cause the processor to perform the computer-implemented method.

In various implementations, a system for differential human review of cytological smear analysis, comprising: a computer configured to: receive an image of a cytological smear associated with a patient; receive, from an automatic cell classifier, classifications of cells detected in the image; identify a first subset of the classified cells that require human review based on predetermined criteria comprising at least one of extent of cells classified as an abnormal cell type or extent of cells classified as an unclassified cell type; cause a portion of the first subset to be presented to a human reviewer at a resolution sufficient for cell-level review; identify a second subset of the classified cells that do not require human review based on the predetermined criteria; and exclude a portion of the second subset from presentation to the human reviewer at a resolution sufficient for cell-level review, unless an indication is received from the human reviewer.

In some implementations of the system, the predetermined criteria are further based on classification confidence levels (CCLs) falling on the insufficient confidence side of type-specific thresholds, and wherein the computer is further configured to determine the cell type-specific thresholds based on statistical confidence requirements calibrated according to clinical significance of their respective cell types and statistical sampling adequacy. In some implementations of the system, the computer is further configured to: present to the human reviewer a portion of abnormal cells from the first subset associated with classification confidence levels (CCLs) falling on the insufficient-confidence side of a threshold; and refrain from presenting to the human reviewer a portion of abnormal cells from the second subset associated with CCLs falling on the sufficient-confidence side of the threshold, unless a relevant indication is received from the human reviewer.

In some implementations of the system, the predetermined criteria are further based on cells of a particular classification, wherein the particular classification corresponds to cell types requiring additional verification due to their clinical significance. In some implementations of the system, the predetermined criteria are further based on cells from specific regions of the cytological smear, wherein the specific regions are determined based on known distribution patterns of certain cell types within cytological smears.

In some implementations of the system, the predetermined criteria are further based on cells exhibiting particular morphological features, wherein the particular morphological features are associated with diagnostic ambiguity or clinical significance. In some implementations of the system, the predetermined criteria are further based on relations between at least some of the classified cells and respective normal ranges of clinical thresholds, comprising at least one of: category statistics indicating distribution of different cell types within the cytological smear, or cell statistics indicating morphological parameter variations within specific cell populations. In some implementations of the system, the predetermined criteria are further based on sample preparation quality parameters comprising at least one of: staining adequacy, cell distribution uniformity, presence of preparation artifacts, smear thickness, or monolayer formation characteristics.

In some implementations of the system, the computer is further configured to provide location information for the portion of the first subset presented to the human reviewer, wherein the location information indicates positions of cells within the overall cytological smear to facilitate spatial interpretation. In some implementations of the system, the computer is further configured to provide information about cells adjacent to cells in the portion of the first subset presented to the human reviewer, wherein the adjacent cells provide contextual information that assists in interpreting morphological variations and distinguishing between similar cells. In some implementations of the system, the computer is further configured to provide statistical summaries of cell distributions alongside the portion of the first subset presented to the human reviewer, wherein the statistical summaries establish population-level context for interpreting individual cells.

In some implementations of the system, presenting the portion of the first subset comprises providing a combination of cell images and textual information, wherein certain cell types are presented as images while information about other cell types is provided as text. In some implementations of the system, the computer is further configured to select the extent from a count or a percentage of cells, and wherein the extent of unclassified cells refers to at least one specific cell type comprising unclassified white blood cells. In some implementations of the system, the resolution sufficient for cell-level review comprises an effective numerical aperture and image quality that enables visualization of cellular morphological features necessary for human classification and verification of white blood cells.

In some implementations of the system, the computer is further configured to implement a tiered review process wherein an initial reviewer receives the portion of the first subset and determines whether to escalate specific elements to a specialist reviewer based on analysis of the portion of the first subset. In some implementations of the system, the predetermined indication from the human reviewer comprises at least one of: a cursor movement operation, a scrolling operation, a link activation operation, a content highlighting operation, an area marking operation, a text command input, a voice command input, a keyboard stroke operation corresponding to a predetermined action, or an eye movement pattern detected by an eye tracking device.

The following implementation addresses the routing of cytological smear analysis to appropriate human reviewers based on the relevance of identified cell types to different clinical expertise areas. By matching reviewers with relative experience to the specific cell types identified through automated classification, the system may improve diagnostic accuracy and efficiency, and increase the percentage of cases reviewed by human reviewers with the relevant knowledge for the particular findings. The embodiment supports improved allocation of review resources and potentially improved patient outcomes through targeted routing of cases to reviewers with appropriate clinical expertise.

In various implementations, a computer-implemented method for presenting images of cytological smears to human reviewers, comprising: receiving, from an automatic cell classifier, first and second classifications of cells in first and second images of first and second cytological smears associated with first and second samples obtained from first and second patients, respectively; identifying, from the first classifications of cells, a first clinical expertise relevant for human review of cells associated with the first sample, and from the second classifications of cells, a second clinical expertise relevant for human review of cells associated with the second sample; responsive to the identification of the relevance of the first clinical expertise to the first sample, presenting, as part of default human review workflow of a first reviewer having the first clinical expertise, cells associated with the first sample; and responsive to the identification of the relevance of the second clinical expertise to the second sample, presenting, as part of default human review workflow of a second reviewer having the second clinical expertise, cells associated with the second sample. FIG. 10 illustrates an embodiment of a computer-implemented method for presenting images of cytological smears to human reviewers. The method includes the following steps. In step 200, receive classifications of cells in images of a cytological smear associated with a sample. In step 201, identify clinical expertise relevant for human review from the classifications of cells. And in step 202, present cells from the sample to a reviewer having the clinical expertise as part of default human review workflow.

The computer-implemented method may enhance diagnostic efficiency and accuracy by routing cytological smear images to human reviewers with appropriate clinical expertise based on the automatic cell classifications. The automated routing system may analyze the classifications of cells to identify patterns, abnormalities, and/or cell types that correspond to specific areas of clinical expertise. The system may employ various algorithms and/or rule sets to match cell classification patterns with relevant clinical specialties, such as oncology, hematology, immunology, and/or pediatric subspecialties. This targeted routing may reduce review time, enhance diagnostic accuracy, and/or optimize utilization of specialized human expertise. The presenting of cells as part of a default human review workflow may include, for example, automatically adding the cases to specialized review queues, prioritizing cases based on urgency and/or clinical significance, and/or providing tailored interfaces that highlight relevant cellular features. The system may integrate with laboratory information systems and/or electronic health records to facilitate seamless workflow integration. By directing samples to reviewers with relevant expertise, the system may help ensure that nuanced morphological features and/or rare cell types are evaluated by reviewers most experienced in their interpretation. Cells associated with the sample, may refer to any cells associated with the sample, may include cells classified according to any classification, and may relate to any image or any cytological smear.

In some implementations, the method further comprises tracking workload of the first and second human reviewers and availability of additional human reviewers, and presenting to a reviewer in response to changes in at least one of the workload or the availability. The system may implement workload management features that dynamically adjust routing decisions based on real-time workload metrics and/or reviewer availability. This tracking and adjustment functionality may include monitoring active case counts per reviewer, time spent on reviews, queue lengths, reviewer schedules, and/or unexpected absences. When the system detects imbalances in workload distribution or changes in reviewer availability, it may implement various adjustment methods, such as re-routing pending cases to available reviewers with appropriate expertise, adjusting routing thresholds to balance workloads, or temporarily broadening expertise matching criteria to maintain throughput during high-volume periods. The workload tracking may incorporate different weighting factors for cases of varying complexity, recognizing that some cell patterns require more intensive review time than others. The system may also incorporate predictive algorithms that anticipate workload peaks based on historical patterns and proactively adjust routing parameters. These dynamic adjustments may help laboratories maintain consistent turnaround times, prevent bottlenecks in specialized review queues, and/or optimize utilization of available clinical expertise while preserving the quality benefits of expertise-based routing.

In some implementations of the method, identifying the first clinical expertise relevant for human review of cells associated with the first sample and identifying the second clinical expertise relevant for human review of cells associated with the second sample is based on at least one of clinical implications or morphological characteristics of the first classifications of cells and the second classifications of cells, respectively. The identification of a first clinical expertise relevant for human review of cells associated with the first sample and a second clinical expertise relevant for human review of cells associated with the second sample is derived from automatic cell classification, which may reveal findings that require specific expertise for accurate interpretation. The system may evaluate the cell types identified in the first classifications of cells and the second classifications of cells and determine which specialized knowledge areas are needed for proper review. Based on this evaluation, the system may present cells associated with the first sample to the first human reviewer having the first clinical expertise and cells associated with the second sample to the second human reviewer having the second clinical expertise.

In some implementations of the method, the first cell type is more relevant to potential cancerous conditions, and wherein the first human reviewer is relatively experienced in oncology. The system may route cell images showing characteristics associated with potential cancerous conditions to reviewers with specialized oncology expertise. Human reviewers with oncology expertise may include, but are not limited to, medical oncologists, surgical oncologists, radiation oncologists, hematologic oncologists, pediatric oncologists, oncologic pathologists, cytopathologists with oncology specialization, clinical laboratory specialists with oncology certification, and/or research oncologists with cytology experience. The identification of cell characteristics relevant to cancerous conditions may include recognition of features such as abnormal nuclear-to-cytoplasmic ratios, irregular nuclear membranes, prominent nucleoli, abnormal chromatin patterns, mitotic figures, blasts, smudge cells, blasts with Auer-rods, cellular pleomorphism, and/or specific cancer cell morphologies associated with particular cancer types. The routing system may incorporate varying thresholds for different cancer-associated features, potentially triggering oncology routing when certain high-specificity features are present even in small numbers, while requiring higher counts for features with lower specificity. The system may also consider contextual factors such as anatomical site, patient history, and/or prior diagnoses when determining oncology routing relevance.

In some implementations, the method further comprises real-time redistribution of pending human review tasks among human reviewers when workload of at least one of the first and second human reviewers exceeds a predetermined threshold or when at least one of the first and second human reviewers becomes unavailable. The system may implement an adaptive queue management functionality that monitors reviewer workloads and availability in real-time and dynamically redistributes pending review tasks to maintain workflow efficiency. This redistribution may be triggered by various conditions, such as when a reviewer's queue length exceeds a predetermined threshold (which may be defined in terms of absolute case counts, estimated review time, or relative comparison to other reviewers' workloads), when system analytics detect a significant delay in expected completion times, or when a reviewer becomes unavailable due to scheduled absences, unexpected illness, or other factors. The redistribution algorithm may consider multiple factors when reallocating cases, including but not limited to: the expertise match between the case requirements and available reviewers' specializations, current and projected workloads of alternative reviewers, case priorities and urgency levels, regulatory or institutional requirements for review timeliness, and historical performance metrics for different reviewer-case type combinations. The system may implement graduated response strategies, potentially beginning with minor workload shifts when thresholds are slightly exceeded and progressing to more substantial redistribution when significant imbalances occur. The predetermined thresholds that trigger redistribution may be customizable based on laboratory preferences, staffing levels, and/or expected throughput requirements, and may vary by case type, reviewer expertise level, or time of day.

In some implementations, the method further comprises monitoring for the first and second human reviewers at least one of: review completion times, quality metrics, or review queues; and utilizing this information to refine future assignments to reviewers. The system may implement a continuous improvement mechanism that monitors reviewer performance metrics and leverages this data to optimize future case assignments. The monitoring may track various parameters including, but not limited to: average review completion times for different case types and complexity levels, concordance rates with secondary reviews or final diagnoses, frequency of reclassifications or diagnostic adjustments following review, number and types of annotations or comments added during review, consistency of interpretations for similar case types, length of review queues over time, and/or patterns in case acceptance or deferral. This collected information may be analyzed to identify reviewer-specific strengths, preferences, and efficiency patterns, which may then inform refinement of the assignment algorithms. For example, the system may adjust expertise matching parameters to route more complex cases of a particular type to reviewers who consistently demonstrate high accuracy with reasonable completion times for those cases, or modify workload distribution patterns to account for observed differences in review speeds for different case types. The refinement process may incorporate machine learning techniques that identify subtle patterns in the relationship between case characteristics and reviewer performance metrics, potentially improving routing efficiency beyond what could be achieved with static rule-based assignments. This adaptive approach may help laboratories optimize both diagnostic quality and throughput by continually tuning the alignment between case requirements and reviewer capabilities based on empirical performance data.

In some implementations of the method, the relevance to the first and second clinical expertise comprises at least one of clinical implications or morphological characteristics of the first and second cell classifications, respectively. The relevance to clinical expertise derived from automatic cell classification may suggest findings that require specific expertise for accurate interpretation. The system may evaluate the cell types identified in the classifications and determine their relevance to specialized knowledge areas. Based on this evaluation, the system may present subsets of images to human reviewers whose experience corresponds to the relevant clinical expertise. To enhance the efficiency of reviews, the system may provide tailored indications within the image subsets, such as annotations, markers, or highlights that direct the reviewer's attention to specific cells or areas that are most relevant to their specialty. This may help the reviewer to quickly and effectively assess the aspects of the image that pertain to their domain, enhancing overall diagnostic accuracy and speed.

In some implementations of the method, identifying the first clinical expertise relevant for human review of cells associated with the first sample and identifying the second clinical expertise relevant for human review of cells associated with the second sample comprises applying predetermined rules about patient age; and wherein the first clinical expertise is identified as pediatric oncology when the first classifications of cells indicate oncology-related characteristics and the first patient is a child. The system may incorporate age-specific routing rules that recognize the specialized expertise required for interpreting cytological findings in different age groups. These predetermined rules may reflect the understanding that cellular presentations and diagnostic implications can vary significantly across age ranges, potentially requiring specialized knowledge of age-related variations in cellular morphology, development-associated changes, and/or age-specific disease presentations. For pediatric patients, the system may implement specialized routing logic that recognizes the unique aspects of childhood cellular development and pathology. When oncology-related cellular characteristics are identified in a child's sample, the system may specifically route these cases to reviewers with pediatric oncology expertise, recognizing that childhood cancers often have distinct cellular presentations, progression patterns, and treatment implications compared to adult malignancies. The age thresholds for pediatric classification may vary based on institutional definitions, specific conditions being evaluated, and/or regulatory guidelines, potentially using different age cutoffs for different cell types or suspected conditions. The system may incorporate graduated expertise matching for adolescent patients, potentially considering both pediatric and adult expertise depending on the specific cell types and patterns identified. Additional age-related routing rules may be implemented for geriatric patients, where cellular presentations may be affected by aging processes, comorbidities, and/or medication effects, potentially routing these cases to reviewers with expertise in geriatric-specific cellular changes.

In some implementations, the method further comprises receiving information indicative of a disease progression stage for the first patient; and applying a relevance threshold that varies as a function of the disease progression stage, whereby the relevance threshold for an earlier stage of a disease differs from the relevance threshold for a later stage of the disease. The system may implement disease progression stage-specific routing criteria that account for different monitoring requirements and expected findings at various stages of disease progression. By receiving disease stage information from the patient's medical record, the system can apply appropriate routing criteria that reflect the clinical priorities associated with different stages. For example, for patients with newly diagnosed hematological malignancies, the system may route cytological smear analyses to reviewers experienced in early-stage malignancies during the initial months following diagnosis to verify baseline response patterns during this period. For patients with stable chronic conditions, the system may route to reviewers experienced in general hematology or clinical pathology, triggering specialized review when significant changes from established baselines are detected, such as when lymphocyte morphology changes exceed a first percentage from baseline. For patients with conditions showing active progression, the system may route to reviewers experienced in advanced disease management as the disease advances, helping allocate reviewer resources most effectively while maintaining appropriate monitoring intensity appropriate to different disease stages.

In some implementations, the method further comprises determining a time period since initial diagnosis for the first patient; applying a lower relevance threshold for presenting cells associated with the first sample to the first reviewer during an initial monitoring period following the initial diagnosis, wherein the lower relevance threshold leads to a higher human review rate; and increasing the relevance threshold for presenting cells associated with future samples of the first patient to a human reviewer as the time period increases and the first patient demonstrates relative stability in condition. For newly diagnosed conditions, establishing baseline patterns and treatment response characteristics typically requires more intensive monitoring from reviewers with expertise in early disease management during an initial period. A temporal weighting may be applied to increase routing sensitivity to these reviewers during this initial monitoring period. For example, following a new diagnosis of a condition that manifests in cells, the system may route all or a significant subset of analyses to reviewers experienced in disease onset patterns during the first few months to help establish baseline patterns and support appropriate treatment response. As the time since diagnosis increases, if the condition shows stability, the system may decrease routing sensitivity by adjusting routing thresholds based on a temporal function and shift routing toward reviewers experienced in general monitoring. This temporal adjustment may consider factors such as the type of condition, treatment protocol, and observed stability of cellular parameters over time. For some conditions with established stability indicators, the routing sensitivity to specialized reviewers may decrease according to a predetermined schedule, while for other conditions, the adjustment may depend on quantitative measures of parameter stability across sequential analyses. It may help allocate more intensive expert monitoring when it provides the most clinical value, while transitioning to more selective expert involvement as baseline patterns become established.

In some implementations of the method, the first cell type is more relevant to blood disorders, and wherein the first human reviewer is relatively experienced in hematology or hematopathology with specialization in the analysis of blood cell abnormalities. The system may implement specialized routing for cytological findings associated with blood disorders, directing these cases to reviewers with advanced training and experience in hematology or hematopathology. Cell types that may trigger hematology routing include, but are not limited to, abnormal erythrocyte morphologies (such as sickle cells, spherocytes, or schistocytes), leukocyte abnormalities (such as hypersegmented neutrophils, atypical lymphocytes, or blast cells), platelet anomalies, and/or cellular indicators of bone marrow disorders. The system may evaluate various morphological features and quantitative parameters to determine relevance to blood disorders, potentially incorporating complex pattern recognition that weighs multiple factors simultaneously. Reviewers with appropriate hematological expertise may include hematologists, hematopathologists, laboratory medicine specialists with hematology certification, clinical pathologists with specialized hematology training, and/or cytotechnologists with advanced hematology qualifications. These specialists may have particular skills in evaluating cellular features indicative of benign versus malignant hematological processes, distinguishing reactive changes from pathological abnormalities, and/or identifying subtle morphological changes associated with specific hematological disorders. The routing threshold for hematology specialists may vary based on the specific abnormalities detected, potentially implementing lower thresholds for high-risk findings such as potential leukemic blasts while using higher thresholds for more common abnormalities that could be adequately interpreted by generalists in many circumstances.

In some implementations of the method, the first cell type is more relevant to conditions requiring emergency review, and wherein the first human reviewer is relatively experienced in emergency medicine. The system may incorporate emergency routing protocols that identify cytological findings associated with urgent clinical conditions requiring prompt intervention. This emergency routing may be triggered by detection of cell types or patterns associated with time-sensitive diagnoses, such as malignant cells in cerebrospinal fluid suggesting leptomeningeal spread, blast cells in peripheral blood suggesting acute leukemia, findings indicating sepsis, certain pathogenic microorganisms indicating severe infection, or cellular changes consistent with rapidly progressive inflammatory conditions. The system may apply different urgency thresholds to different emergency-associated findings, potentially implementing multiple tiers of urgency that influence both routing destinations and notification protocols. Reviewers designated for emergency routing may include laboratory medicine specialists with emergency response training, pathologists with certain experience, clinical consultants designated for urgent case review, and/or specialists in rapid diagnosis of time-sensitive conditions. The emergency routing functionality may include additional features beyond standard expertise matching, such as pushing high-priority notifications to designated reviewers, initiating backup reviewer protocols when primary emergency reviewers are unavailable, activating expedited reporting pathways, and/or triggering automatic clinical team alerts for potentially life-threatening findings. The system may incorporate contextual information such as the clinical setting (e.g., intensive care unit versus outpatient clinic) when determining emergency routing thresholds, potentially lowering thresholds for patients already identified as critically ill.

In some implementations of the method, the first cell type exhibits relatively complex morphological patterns, and wherein the first human reviewer is relatively experienced in laboratory morphology. The system may route cytological samples presenting complex or challenging morphological patterns to reviewers with advanced training and experience in cellular morphology interpretation. Complex morphological patterns may include, but are not limited to, cells with atypical or ambiguous features, unusual cellular arrangements, rare cell types, cells showing features intermediate between diagnostic categories, cells with dual or mixed lineage characteristics, cells affected by treatment effects that alter typical morphology, and/or cells with subtle distinctions between reactive and neoplastic changes. The routing criteria may incorporate complexity scores derived from multiple morphological parameters, pattern recognition algorithms that identify challenging morphological presentations, and/or statistical measures of classification uncertainty. Reviewers designated for morphology routing may include cytomorphologists, specialists in diagnostic cytopathology, researchers with expertise in unusual cell types, reference laboratory consultants for difficult cases, and/or senior laboratory scientists with extensive morphological interpretation experience. The morphology routing threshold may be adjustable based on laboratory resources, case volumes, and/or institutional quality assurance protocols, potentially allowing for different complexity thresholds under different operational conditions. The system may incorporate continuous feedback mechanisms that track concordance rates for complex morphology cases, potentially refining routing thresholds and specialist assignments based on outcome analysis and diagnostic agreement metrics.

In some implementations of the method, the first cell type relates to results within a normal range that require validation for quality assurance, and wherein the first human reviewer is a laboratory technician. The system may implement quality assurance routing protocols that direct a subset of cases with apparently normal or within-range results to appropriate reviewers for validation purposes. This quality assurance routing may support laboratory quality management programs by facilitating systematic review of normal cases to verify algorithm performance, maintain reviewer proficiency across the full spectrum of findings, detect subtle deviations that may be missed by automated systems, and/or fulfill regulatory requirements for human verification of normal results. The selection of normal-range cases for quality assurance review may follow various methodologies, such as random sampling at predefined percentages, targeted selection based on algorithm confidence levels, stratified sampling across different cell types and patient demographics, and/or periodic validation of specific normal patterns. Laboratory technicians selected for quality assurance routing may include cytotechnologists, clinical laboratory scientists, quality assurance specialists, and/or laboratory staff with appropriate certification for routine cytology review. The quality assurance routing parameters may be configurable based on laboratory protocols, accreditation requirements, and/or institutional quality management strategies, potentially with different validation rates for different normal cell types or classifier confidence levels. The system may incorporate documentation features specifically designed for quality assurance purposes, such as structured verification forms, integrated audit trails of validation activities, and/or aggregated reporting of quality metrics derived from normal case reviews.

In some implementations, the method further comprises providing to the first human reviewer, in addition to the cells associated with the first sample, a report comprising numerical values related to at least one of a number of cells or a percentage of cells related to the cells associated with the first sample. The system may enhance the review process by providing contextual numerical data alongside the presented cell images, giving reviewers quantitative information that may support more informed interpretation. This numerical data may include, but is not limited to, absolute counts of different cell types identified in the sample, percentages of specific cell populations relative to total cells counted, normative reference ranges for the reported cell types, statistical confidence intervals for the reported values, deviation metrics comparing the current values to patient-specific baselines or population averages, trend analysis showing changes from previous samples when available, and/or correlation coefficients between related cell parameters. The presentation format for this numerical data may be customizable, potentially including tabular displays, embedded annotations within image views, graphical representations such as histograms or scatterplots, color-coded indicators highlighting values outside reference ranges, and/or interactive elements allowing reviewers to explore relationships between different parameters. The system may implement context-sensitive data presentation that highlights different numerical metrics based on the specific clinical expertise of the reviewer, the cell types being evaluated, and/or the suspected clinical conditions, potentially emphasizing different aspects of the quantitative data for different specialist reviewers. This integration of images with relevant numerical data may help reviewers more efficiently contextualize morphological findings within the overall cellular distribution pattern, potentially improving both accuracy and review speed.

In some implementations, the method further comprises providing to the first human reviewer, in addition to the cells associated with the first sample, information indicative of reasons for presenting those cells or that sample to the first human reviewer. The system may provide transparency in its routing decisions by communicating to reviewers the specific factors that led to their assignment to particular cases. This routing rationale information may include, but is not limited to, identification of the specific cell types or morphological features that triggered expertise matching, quantitative metrics or threshold exceedances that influenced routing, patient demographic or clinical factors incorporated into the routing decision, prioritization factors that affected case sequencing, quality assurance protocols that selected the case for review, and/or specific institutional policies that mandated particular routing pathways. The presentation of this rationale may be implemented through various mechanisms, such as standardized notification codes, textual explanations, visual highlights of triggering features, graphical indicators of threshold exceedances, or interactive elements allowing reviewers to explore the decision criteria in greater detail. The system may adjust the level of detail in routing rationale based on reviewer preferences, institutional protocols, and/or the complexity of the routing decision, potentially providing more comprehensive explanations for complex multifactorial routing decisions while using simplified indicators for straightforward routing cases. This transparency in routing logic may help reviewers focus their attention on the most relevant aspects of each case, potentially improving review efficiency by highlighting the specific cellular features or patterns that warranted their expertise, while also facilitating ongoing quality improvement by allowing reviewers to provide feedback on routing appropriateness.

In some implementations, the method further comprises receiving, from the automatic cell classifier, a third classification of cells in a third image of a third cytological smear associated with a third sample obtained from a third patient; receiving patient data associated with the third patient, wherein the patient data comprises at least one of patient age or patient medical history; and identifying, from a combination of the third classification of cells and the patient data, a third clinical expertise relevant for human review of cells associated with the third sample. The system may incorporate patient-specific contextual data to refine expertise matching, recognizing that the same cellular findings may have different clinical implications depending on patient factors. This contextual routing approach may modify the selection criteria applied to the cell classifications based on various patient attributes. For age-based modifications, the system may adjust routing thresholds, with potentially lower thresholds for findings that would be considered more unusual or significant in certain age groups. For example, blast cells that might trigger standard hematology routing in adults could be directed to pediatric hematology specialists when found in children, while certain lymphocyte patterns might require specialized review in elderly populations where they carry different diagnostic implications. For medical history-based modifications, the system may apply different interpretive frameworks based on documented conditions, treatments, or risk factors. The system may implement lower routing thresholds for patients with known malignancies, potentially directing even subtle abnormalities to oncology specialists for evaluation in the context of disease monitoring. Conversely, the system may adjust routing criteria for patients with known benign conditions that affect cellular appearance, potentially avoiding unnecessary specialist review for expected morphological variations. This integrated approach may incorporate decision matrices that weigh multiple patient factors simultaneously, potentially using different weighting schemes for different cell types based on their relationship to various clinical contexts. The contextual routing may be valuable for findings with ambiguous significance, where patient factors substantially affect the appropriate interpretive framework and specialist expertise requirements.

In some implementations, the method further comprises responsive to the identification of the relevance of the third clinical expertise to the third sample, presenting, as part of default human review workflow of a third reviewer having the third clinical expertise, cells associated with the third sample; wherein when the third classification of cells indicates presence of atypical erythrocytes and the patient data indicates the third patient is under twelve years of age, the third clinical expertise is identified as pediatric hematology. The system may implement condition-specific expertise matching that recognizes patterns suggestive of particular clinical scenarios requiring specialized knowledge. For the combination of atypical erythrocytes in pediatric patients, the system may route to pediatric hematology specialists who have expertise in age-specific erythrocyte disorders and developmental variations. This routing may recognize that erythrocyte abnormalities in children may represent different conditions than similar findings in adults, potentially including hereditary erythrocyte disorders that typically present in childhood, developmental variations in erythrocyte morphology, nutritional deficiencies affecting pediatric erythropoiesis, and/or erythrocyte changes associated with childhood systemic diseases. The age threshold for pediatric routing may be adjustable based on institutional protocols and specific condition parameters, with different age cutoffs potentially applied to different erythrocyte abnormalities based on their typical presentation patterns. The system may implement additional combination rules beyond these examples, potentially covering numerous clinical scenarios where specific combinations of cellular findings and patient factors suggest particular expertise requirements. In some implementations of the method, a non-transitory computer-readable medium comprising instructions that, when executed by a processor, cause the processor to perform the computer-implemented method.

In various implementations, a system for presenting images of cytological smears to human reviewers, comprising: a computer configured to: receive, from an automatic cell classifier, first and second classifications of cells in first and second images of first and second cytological smears associated with first and second samples obtained from first and second patients, respectively; identify, from the first classifications of cells, a first clinical expertise relevant for human review of cells associated with the first sample, and from the second classifications of cells, a second clinical expertise relevant for human review of cells associated with the second sample; responsive to the identification of the relevance of the first clinical expertise to the first sample, cause cells associated with the first sample to be presented, as part of default human review workflow of a first reviewer having the first clinical expertise; and responsive to the identification of the relevance of the second clinical expertise to the second sample, cause cells associated with the second sample to be presented, as part of default human review workflow of a second reviewer having the second clinical expertise.

In some implementations of the system, the computer is further configured to: track workload of the first and second human reviewers and availability of additional human reviewers, and present to a reviewer in response to changes in at least one of the workload or the availability. In some implementations of the system, identifying the first clinical expertise relevant for human review of cells associated with the first sample and identifying the second clinical expertise relevant for human review of cells associated with the second sample is based on at least one of clinical implications or morphological characteristics of the first classifications of cells and the second classifications of cells, respectively. In some implementations of the system, the first classifications of cells indicate a first cell type more relevant to potential cancerous conditions, and wherein the first human reviewer is relatively experienced in oncology.

In some implementations of the system, the computer is further configured to: perform real-time redistribution of pending human review tasks among human reviewers when workload of at least one of the first and second human reviewers exceeds a predetermined threshold or when at least one of the first and second human reviewers becomes unavailable. In some implementations of the system, the computer is further configured to: monitor for the first and second human reviewers at least one of: review completion times, quality metrics, or review queues; and utilize this information to refine future assignments to reviewers. In some implementations of the system, the relevance to the first and second clinical expertise comprises at least one of clinical implications or morphological characteristics of the first and second classifications of cells, respectively.

In some implementations of the system, identifying the first clinical expertise relevant for human review of cells associated with the first sample and identifying the second clinical expertise relevant for human review of cells associated with the second sample comprises applying predetermined rules about patient age; and wherein the first clinical expertise is identified as pediatric oncology when the first classifications of cells indicate oncology-related characteristics and the first patient is a child.

In some implementations of the system, the computer is further configured to: receive information indicative of a disease progression stage for the first patient; and apply a relevance threshold that varies as a function of the disease progression stage, whereby the relevance threshold for an earlier stage of a disease differs from the relevance threshold for a later stage of the disease. In some implementations of the system, the computer is further configured to: determine a time period since initial diagnosis for the first patient; apply a lower relevance threshold for presenting cells associated with the first sample to the first reviewer during an initial monitoring period following the initial diagnosis, wherein the lower relevance threshold leads to a higher human review rate; and increase the relevance threshold for presenting cells associated with future samples of the first patient to a human reviewer as the time period increases and the first patient demonstrates relative stability in condition.

In some implementations of the system, the first classifications of cells indicate a first cell type more relevant to blood disorders, and wherein the first human reviewer is relatively experienced in hematology or hematopathology with specialization in the analysis of blood cell abnormalities. In some implementations of the system, the first classifications of cells indicate a first cell type more relevant to conditions requiring emergency review, and wherein the first human reviewer is relatively experienced in emergency medicine. In some implementations of the system, the first classifications of cells indicate a first cell type that exhibits relatively complex morphological patterns, and wherein the first human reviewer is relatively experienced in laboratory morphology.

In some implementations of the system, the first classifications of cells indicate a first cell type that relates to results within a normal range that require validation for quality assurance, and wherein the first human reviewer is a laboratory technician. In some implementations of the system, the computer is further configured to: provide to the first human reviewer, in addition to the cells associated with the first sample, a report comprising numerical values related to at least one of a number of cells or a percentage of cells related to the cells associated with the first sample. In some implementations of the system, the computer is further configured to: provide to the first human reviewer, in addition to the cells associated with the first sample, information indicative of reasons for presenting those cells or that sample to the first human reviewer.

In some implementations of the system, the computer is further configured to: receive, from the automatic cell classifier, a third classification of cells in a third image of a third cytological smear associated with a third sample obtained from a third patient; receive patient data associated with the third patient, wherein the patient data comprises at least one of patient age or patient medical history; and identify, from a combination of the third classification of cells and the patient data, a third clinical expertise relevant for human review of cells associated with the third sample. In some implementations of the system, the computer is further configured to: responsive to the identification of the relevance of the third clinical expertise to the third sample, present, as part of default human review workflow of a third reviewer having the third clinical expertise, cells associated with the third sample; wherein when the third classification of cells indicates presence of atypical erythrocytes and the patient data indicates the third patient is under twelve years of age, the third clinical expertise is identified as pediatric hematology.

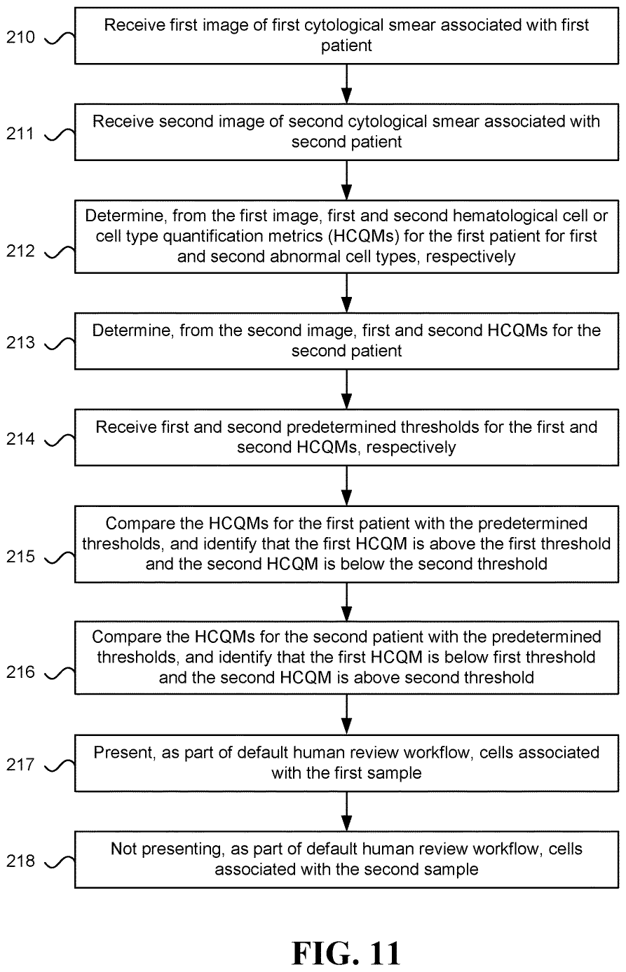

In various implementations, a computer-implemented method for cytological smear human review workload management, comprising: receiving a first image of a first cytological smear associated with a first sample obtained from a first patient; receiving a second image of a second cytological smear associated with a second sample obtained from a second patient; determining, from the first image, first and second hematological cell or cell type quantification metrics (HCQMs) for the first patient, wherein the first and second HCQMs are for first and second abnormal cell types, respectively; determining, from the second image, the first and second HCQMs for the second patient; receiving first and second predetermined thresholds associated with the first and second HCQMs, respectively; comparing the first and second HCQMs for the first patient with the first and second predetermined thresholds, respectively, and identifying that the first HCQM is above the first threshold and the second HCQM is below the second threshold; comparing the first and second HCQMs for the second patient with the first and second predetermined thresholds, respectively, and identifying that the first HCQM is below the first threshold and the second HCQM is above the second threshold; presenting, as part of default human review workflow, cells associated with the first sample; and not presenting, as part of default human review workflow, cells associated with the second sample. FIG. 11 illustrates an embodiment of a computer-implemented method for cytological smear human review workload management. The method includes the following steps. In step 210, receive first image of first cytological smear associated with first patient. In step 211, receive second image of second cytological smear associated with second patient. In step 212, determine, from the first image, first and second hematological cell or cell type quantification metrics (HCQMs) for the first patient for first and second abnormal cell types, respectively. In step 213, determine, from the second image, first and second HCQMs for the second patient. In step 214, receive first and second predetermined thresholds for the first and second HCQMs, respectively. In step 215, compare, optionally by a processor, the HCQMs for the first patient with the predetermined thresholds, and identify that the first HCQM is above the first threshold and the second HCQM is below the second threshold. In step 216, compare, optionally by the processor, the HCQMs for the second patient with the predetermined thresholds, and identify that the first HCQM is below first threshold and the second HCQM is above second threshold. In step 217, present, as part of default human review workflow, cells associated with the first sample. And in step 218, not presenting, as part of default human review workflow, cells associated with the second sample.

The computer-implemented method may utilize selective routing of cytological smear images for human review based on the comparative analysis of multiple HCQMs for different abnormal cell types against their respective thresholds. This approach may optimize laboratory workflow by prioritizing cases with specific patterns of abnormality for human expert attention while allowing cases with different patterns to proceed through automated workflows without human review, even when their results fall outside normal ranges. Multiple implementation approaches may be employed for the threshold comparisons. In a first implementation, the thresholds may be static values established based on clinical guidelines or laboratory protocols. In a second implementation, the thresholds may be dynamically adjusted based on factors such as laboratory workload, clinical context, or historical performance metrics. The selection of which sample to present for human review may be based on the specific pattern of threshold exceedances, recognizing that different combinations of abnormal cell types may have varying clinical significance and/or classification reliability. For example, samples with elevated levels of highly significant abnormal cell types may be prioritized for expert review, while samples with elevations only in less significant or more reliably classified abnormal cell types may bypass routine human review. This pattern-based routing may optimize the allocation of expert resources, potentially reducing overall review workload while maintaining analytical quality for clinically significant findings. The method may be implemented across various hardware configurations including standalone systems, networked environments, and/or cloud-based infrastructures, potentially allowing for scalable deployment across different laboratory settings with varying throughput requirements and staffing resources. Cells associated with the sample, may refer to any cells associated with the sample, may include cells classified according to any classification, and may relate to any image or any cytological smear.

In the context of this embodiment, “not presenting, as part of default human review workflow, cells associated with the second sample” refers to excluding the second sample from the standard review process based on the specific pattern of HCQM threshold comparisons for the second patient. While the first sample is presented for human review because the first patient exhibits a pattern where the first HCQM exceeds the first threshold (potentially indicating a clinically significant finding for the first abnormal cell type), the second sample is not presented because the second patient exhibits a different pattern where the first HCQM remains below the first threshold but the second HCQM exceeds the second threshold. This pattern-based routing recognizes that different combinations of abnormal cell type presentations may have varying clinical significance and/or classification reliability, and the system selectively routes those samples with specific HCQM patterns requiring human verification. The not presenting may be implemented through various mechanisms including: excluding the second sample from review queues, routing the second sample directly to automated reporting pathways, preventing the second sample from appearing in reviewer worklists, marking the second sample with metadata flags indicating completion without human review requirement while marking the first sample with flags indicating review requirement, applying different marking schemes to the first and second samples that are distinguishable through visual or textual indicators, storing the second sample in repositories designated for completed analyses that bypassed human review based on their specific HCQM threshold pattern, presenting the HCQM results for the second sample to a human without an indication that review is required whereas the HCQM results for the first sample are presented with an indication that review is required, and/or displaying the second sample in a manner distinguishable from the first sample in the default human review workflow, wherein such distinguishing manners may include distinct borders, different color coding, modified interaction controls, presence or absence of review indicators, different positioning in the interface, or textual labels that identify the second sample as excluded from default human review workflow based on its HCQM pattern. By not presenting the second sample while presenting the first sample, the system may optimize human review resources by focusing expert attention on samples with HCQM patterns identified as requiring human verification, while allowing samples with different HCQM patterns to proceed through automated workflows without human review as part of the default workflow. The system may maintain audit records indicating that the second sample was processed through automated pathways based on its specific HCQM pattern, while preserving the capability for the second sample to be accessed through review mechanisms outside the default workflow, such as when specifically requested by a physician, when flagged by subsequent quality assurance protocols, when laboratory policies require review of samples with certain HCQM patterns, when a reviewer indicates a desire to review the second sample despite its HCQM pattern, and/or when clinical circumstances warrant retrospective examination of samples that initially bypassed human review based on their HCQM threshold patterns.

In some implementations of the method, the first abnormal cell type is selected from: blast cell, aberrant lymphocyte, promyelocyte, atypical lymphocyte, Sézary cell, hairy cell, large granular lymphocyte, Pelger-Huet cell. The specified abnormal cell types may represent cells with high clinical significance that often warrant careful evaluation. Blast cells may indicate potential hematological malignancies such as leukemia, while aberrant lymphocytes may suggest lymphoproliferative disorders or viral infections. Promyelocytes, particularly when abnormal, may be associated with acute promyelocytic leukemia, a medical emergency requiring rapid intervention. Atypical lymphocytes may indicate viral infections or other immunological responses. Sézary cells may be found in cutaneous T-cell lymphoma, particularly Sezary syndrome. Hairy cells may indicate hairy cell leukemia, a rare chronic lymphoproliferative disorder. Large granular lymphocytes in abnormal quantities may suggest large granular lymphocyte leukemia or reactive processes. Pelger-Huet cells may be associated with myelodysplastic syndromes or may represent inherited anomalies. The selection of these particular cell types may reflect their challenging morphological characteristics that may benefit from expert human review, potentially including subtle distinctions from reactive or normal cells that automated systems may find difficult to reliably classify. The system may prioritize these cell types based on their potential diagnostic significance and/or the established performance characteristics of the automatic cell classifier for each cell type. The list may not be exhaustive, and additional abnormal cell types with similar characteristics and/or clinical significance may be included in various implementations of the invention.

In some implementations of the method, the second abnormal cell type is selected from: blast cell, aberrant lymphocyte, promyelocyte, atypical lymphocyte, Sézary cell, hairy cell, large granular lymphocyte, Pelger-Huet cell, Plasma cell, schistocyte, smudge cell, helmet cell, bite cell, blister cell or cell containing a microorganism. The second abnormal cell type may be selected from a list that includes both clinically significant immature or malignant white blood cells and various morphologically abnormal red blood cells or other cellular elements. Plasma cells in peripheral blood may indicate plasma cell disorders such as multiple myeloma or severe inflammatory conditions. Schistocytes may suggest microangiopathic hemolytic anemias or mechanical damage to red blood cells. Smudge cells, while often artifacts, may be associated with chronic lymphocytic leukemia when present in significant numbers. Helmet cells may indicate severe mechanical trauma to red blood cells or specific types of hemolytic anemia. Bite cells may suggest oxidative hemolysis such as in G6PD deficiency. Blister cells may be seen in conditions like unstable hemoglobinopathies or after certain drug exposures. Cells containing microorganisms may indicate infectious processes such as malaria, babesiosis, or bacterial infections. This expanded list may include cell types with varying degrees of clinical urgency and morphological distinctiveness, potentially allowing the system to prioritize human review based on the specific combination of abnormalities detected. The inclusion of the same cell types from the first abnormal cell type list may provide flexibility in implementing different threshold schemes for the same cell type depending on the specific clinical context or laboratory protocols. The system may apply different thresholds to identical cell types based on their co-occurrence with other abnormalities or based on quantitative considerations that may affect their clinical significance. This further reflects the ability of the system to improve over time, allowing a growing set of abnormal cell types to be sent automatically based on system results without requiring human review.

In some implementations of the method, the first abnormal cell type is selected from: myelocyte, metamyelocyte Plasma cell, schistocyte, smudge cell, helmet cell, bite cell, nucleated RBC, blister cell or cell containing a microorganism. The first abnormal cell type may include immature granulocytic precursors and various abnormal red blood cell forms. Myelocytes and metamyelocytes in peripheral blood may indicate bone marrow stress, infection, inflammation, myeloproliferative disorders, or leukemic processes. Nucleated RBCs in adult peripheral blood may suggest bone marrow stress, infiltrative processes, or severe hypoxia. The combination of these immature white cell precursors and abnormal red cell forms may provide a comprehensive approach to detecting various hematological disorders, potentially including both acute and chronic conditions. Different implementations of the system may assign varying priorities to these cell types based on their established clinical significance in specific patient populations or clinical settings. For example, nucleated RBCs may receive higher priority in critical care settings where they may indicate acute physiological stress, while myelocytes and metamyelocytes may receive higher priority in oncology settings where they may suggest possible progression of myeloproliferative disorders. The system may also consider the relative difficulty of accurately classifying each cell type when determining which samples to route for human review, potentially prioritizing cell types that have demonstrated lower automatic classification reliability in validation studies or ongoing quality assessments. This approach may optimize the use of human expertise for cell types where automated analysis faces greater challenges.

In some implementations of the method, the second abnormal cell type is selected from: myelocyte, band neutrophil, metamyelocyte, nucleated RBC, sickle cell, polychromatic cell or cell with Howell-jolly body. The second abnormal cell type may include cells that indicate various physiological responses or conditions. Band neutrophils in elevated numbers may suggest acute bacterial infections or inflammatory conditions, representing a “left shift” in granulocyte maturation. Sickle cells may indicate sickle cell disease or sickle cell trait, particularly important during acute sickling crises. Polychromatic cells (reticulocytes) may suggest increased red blood cell production in response to blood loss, hemolysis, or recovery from anemia. Cells with Howell-Jolly bodies may indicate splenic dysfunction or asplenia, which may have implications for immune function. The combination of these cell types with the first abnormal cell types may create a comprehensive detection system for a wide range of hematological conditions, potentially including both genetic disorders and acquired conditions. The thresholds for these cell types may be adjusted based on patient demographics, clinical history, or specific clinical settings, recognizing that certain cell types may have different significance in different patient populations. For example, the threshold for band neutrophils may be higher in pediatric patients who often demonstrate more pronounced left shifts during infection compared to adult patients. Similarly, thresholds for sickle cells may be adjusted based on known patient genotype or history. This adaptive approach may optimize the system's sensitivity and specificity for clinically significant findings across diverse patient populations. While these cells are abnormal and may reflect health risks, the system may be confident enough in its result to not require human review, and the guidelines may not mandate human review before automatically sending the results to a physician, who can then review them and decide on a course of action for the patient.

In some implementations, the method further comprises generating a first report based at least on the first and second HCQMs for the first patient; generating a second report based at least on the first and second HCQMs for the second patient; and automatically transmitting the second report to an electronic medical information system. The report generation and transmission functionality may streamline the integration of analytical results into clinical workflows. Reports may be generated in various formats depending on the specific requirements of the healthcare setting, potentially including structured data formats for electronic health record integration, PDF documents for clinical review, or specialized formats compatible with laboratory information systems. The first report for the first patient (whose cells are presented for human review) may include additional annotations, confidence indicators, or detailed morphological descriptions resulting from the human review process. The second report (automatically transmitted without default human review) may include appropriate indicators of the automated nature of the analysis, potentially including confidence metrics or quality indicators that justify the bypassing of human review. The automatic transmission to electronic medical information systems may occur through various secure communication protocols such as HL7, FHIR, or proprietary APIs, potentially facilitating seamless integration with diverse healthcare IT infrastructures. The timing of the automatic transmission may be configurable based on laboratory workflows, potentially including options for immediate transmission upon completion of automated analysis, batched transmission at scheduled intervals, or transmission triggered by specific events such as verification steps or quality checks. This automated reporting pipeline may reduce manual data entry errors, decrease turnaround time for results, and improve the accessibility of hematological findings for clinical decision-making, while maintaining appropriate distinctions between results that have undergone different levels of human expert involvement.

In some implementations, the method further comprises receiving a third image of a third cytological smear associated with a third sample obtained from a third patient; receiving a fourth image of a fourth cytological smear associated with a fourth sample obtained from a fourth patient; determining, from the third image, a quantity of unclassified cells for the third patient; determining, from the fourth image, a quantity of unclassified cells for the fourth patient; receiving a predetermined unclassified cell threshold; comparing the quantity of unclassified cells for the third patient with the predetermined unclassified cell threshold and identifying that the quantity of unclassified cells for the third patient is above the predetermined unclassified cell threshold; comparing the quantity of unclassified cells for the fourth patient with the predetermined unclassified cell threshold and identifying that the quantity of unclassified cells for the fourth patient is below the predetermined unclassified cell threshold; presenting, as part of default human review workflow, cells associated with the third sample; and not presenting, as part of default human review workflow, cells associated with the fourth sample. The unclassified cell threshold-based routing mechanism may address samples with ambiguous cellular elements that resist confident automated classification. Unclassified cells may result from various factors including unusual morphological features, technical artifacts, rare cell types not well-represented in the classifier's training data, or cells in transitional states between established morphological categories. The predetermined unclassified cell threshold may be established through various approaches, potentially including statistical analysis of historical laboratory data, alignment with clinical guidelines for morphological assessment, lab policy, or validation studies that correlate unclassified cell quantities with diagnostic outcomes. The implementation may include different threshold levels based on patient demographics, clinical context, or sample characteristics, recognizing that certain patient populations or clinical scenarios may naturally present with higher levels of morphologically ambiguous cells. The presentation of samples with above-threshold unclassified cells for human review may leverage expert morphological expertise to resolve classification ambiguities, potentially improving diagnostic accuracy for challenging cases while maintaining efficient workflows for samples with more typical cellular presentations. The system may implement various visualization approaches for presenting unclassified cells during human review, potentially including side-by-side comparisons with similar classified cells, highlighting of specific morphological features that contributed to classification uncertainty, or presentation of confidence metrics for potential alternative classifications. This focused review approach may optimize expert time allocation by concentrating attention on cells with the highest diagnostic uncertainty.

In some implementations, the method further comprises monitoring human review results over time for multiple cytological smears; adjusting at least the first predetermined threshold based on the monitoring, wherein the adjusting comprises increasing the at least first predetermined threshold to reduce a number of smears presented for human review. The adaptive threshold adjustment mechanism may create a self-improving system that evolves based on accumulated performance data. The monitoring of human review results may encompass various metrics including correction rates, agreement percentages between automated and human classifications, identification of systematic classification challenges, or temporal trends in review outcomes. This monitoring may occur across multiple timescales, potentially including real-time tracking, daily aggregation, weekly trend analysis, or longitudinal studies over months or years to identify seasonal or gradually evolving patterns. The threshold adjustment process may implement various mathematical approaches including incremental step changes, statistical confidence-based adjustments, machine learning optimization algorithms, or rule-based modifications based on predefined performance targets. The specific amount of threshold increase may vary based on factors such as the observed agreement rates, the clinical significance of the cell type, or resource optimization goals, potentially implementing more aggressive adjustments for cell types with consistently high agreement rates and more conservative adjustments for clinically critical classifications. This adaptive approach may balance workload optimization with quality assurance, potentially establishing a gradual refinement process that maintains appropriate safety margins while systematically reducing unnecessary human review. The system may include appropriate oversight mechanisms such as periodic validation checks, statistical quality control, or randomized audit samples to ensure that threshold adjustments maintain appropriate sensitivity for clinically significant findings while successfully reducing review volume for cases where automated analysis demonstrates consistent reliability.

In some implementations of the method, the monitoring comprises: tracking a rate of agreement between automated analysis results and human review results; and wherein the adjusting comprises automatically increasing the at least first predetermined threshold when the rate of agreement exceeds a predetermined agreement threshold. The agreement rate-based threshold adjustment approach may provide an objective, data-driven mechanism for optimizing human review rates. The agreement rate may be calculated through various methodologies, potentially including simple percentage agreement on final classifications, weighted agreement metrics that prioritize clinically significant cell types, kappa statistics that account for chance agreement, or confidence-adjusted measures that incorporate the certainty levels of both human and automated assessments. The predetermined agreement threshold that triggers automatic increases may be established through various approaches, potentially including statistical analyses that identify inflection points where agreement rates stabilize, validation studies that correlate agreement rates with diagnostic outcomes, or iterative optimization processes that balance workload reduction with quality maintenance. Different agreement thresholds may be implemented for different cell types, potentially setting higher agreement requirements for clinically critical classifications and more moderate requirements for findings with lower clinical urgency. The automatic threshold increase may occur through various mechanisms including fixed increments, proportional adjustments based on the margin by which agreement exceeds the threshold, or adaptive algorithms that accelerate or decelerate adjustment rates based on accumulated performance data. This approach may create a feedback loop wherein consistently reliable automated classifications are progressively trusted with less human verification, potentially allowing laboratory resources to be focused on cases with higher classification complexity or clinical significance. The system may implement appropriate safeguards including periodic revalidation, monitoring for sudden performance changes, or manual override capabilities to maintain quality assurance throughout the adaptive process.

In some implementations of the method, the adjusting is performed periodically according to a predetermined schedule to progressively reduce human review workload over time while maintaining analysis quality. The scheduled progressive adjustment approach may provide a structured, controlled methodology for gradually optimizing laboratory workflows. The predetermined schedule may be implemented through various timeframes and mechanisms, potentially including regular calendar-based adjustments (monthly, quarterly, or annually), throughput-based milestones (after processing specific numbers of samples), performance-based triggers (after achieving stability at current thresholds), or hybrid approaches that combine multiple scheduling factors. The progressive nature of the adjustments may follow various patterns, potentially including linear increments, diminishing-returns models that implement larger initial adjustments followed by more conservative later adjustments, or context-sensitive patterns that accelerate or decelerate based on performance metrics. The maintenance of analysis quality throughout this progressive process may be ensured through various mechanisms including parallel quality tracking, statistical process control methodologies, randomized verification samples, or periodic comprehensive validations. These quality assurance mechanisms may track multiple metrics including clinical outcome correlations, comparison with reference methods, internal consistency measures, or specific performance indicators for high-risk sample categories. The system may implement appropriate circuit-breakers or adjustment reversals if quality metrics show concerning trends, potentially including automatic threshold restoration if agreement rates decline, targeted reviews of specific cell types showing performance changes, or temporary suspension of further adjustments until stability is reestablished. This gradual, controlled approach may allow laboratories to find optimal operational equilibrium between automation and human expertise while maintaining confidence in analytical reliability through measured, validated steps rather than abrupt transitions. This methodology may be useful during onboarding of a new system or software update, where there is a desire to gain confidence in the performance.

In some implementations of the method, the adjusting comprises: determining cell type-specific agreement rates between automated analysis results and human review results for different abnormal cell types; and selectively increasing thresholds for specific abnormal cell types based on their respective agreement rates. The cell type-specific threshold tuning approach may recognize the varying classification challenges presented by different abnormal cell types. Different cell types may demonstrate different automated classification performance characteristics based on factors such as morphological distinctiveness, prevalence in training data, technical factors affecting visualization, or biological variability within the cell type. The determination of cell type-specific agreement rates may involve various analytical approaches, potentially including confusion matrix analysis to identify specific misclassification patterns, confidence distribution analysis to assess classification certainty, boundary case studies to evaluate challenging morphologies, or temporal trend analysis to identify evolving performance characteristics. The selective threshold increases may follow various methods, potentially including prioritizing adjustments for high-volume cell types to improve efficiency gains, focusing on clinically less critical cell types to maintain conservative approaches for significant findings, or targeting cell types with the highest agreement rates to build on demonstrated strengths of the automated system. The implementation may include interdependency considerations, recognizing that some cell types may share morphological features or exist along biological continua, potentially requiring coordinated threshold adjustments across related cell categories to maintain classification coherence. This granular approach may optimize workflow efficiency by allowing more aggressive automation for cell types with demonstrated classification reliability while maintaining appropriate human oversight for cell types with more challenging classification characteristics or higher clinical significance. The system may implement appropriate monitoring mechanisms to track the impact of these differentiated thresholds, potentially including periodic comprehensive reviews, trend analysis for specific cell types, or targeted validation studies for categories undergoing significant threshold changes.

This embodiment may be integrated with human confirmation or oversight processes without departing from the automated decision-making framework for cytological smear human review workload management based on HCQMs. For example, the system may automatically determine HCQMs for abnormal cell types from cytological smear images while simultaneously providing laboratory personnel with user interface controls to monitor and validate HCQM calculations, review predetermined threshold settings for different abnormal cell types, and approve threshold modifications based on laboratory-specific protocols or clinical requirements. The system may provide a user interface that enables laboratory staff to review comparative HCQM results across multiple patients, establish custom predetermined thresholds for specific abnormal cell types based on clinical significance, and monitor the automated presenting and not presenting decisions for quality assurance purposes. In clinical environments requiring human oversight, supervisors may utilize interface controls to review cases where HCQMs fall near predetermined threshold boundaries, approve modifications to default human review workflow parameters for specific abnormal cell types, and establish validation protocols for automated workload management decisions. The user interface may display real-time analysis of HCQM patterns across different abnormal cell types, enable manual override of automated presentation decisions when clinical judgment warrants deviation from standard threshold-based routing, and provide feedback mechanisms for laboratory staff to evaluate the effectiveness of predetermined thresholds in optimizing human reviewer workload distribution. Additionally, the system may integrate approval workflows where laboratory managers can review and confirm predetermined threshold adjustments, verify that samples with appropriate HCQM patterns are correctly presented or not presented as part of default human review workflow, and establish custom workload management criteria for specific patient populations or clinical contexts. Such human confirmation processes may operate in conjunction with the automated HCQM determination and threshold comparison workflow, enhancing quality control while maintaining the efficiency gains provided by automated pattern-based routing of cytological smears for human review. In some implementations of the method, a non-transitory computer-readable medium comprising instructions that, when executed by a processor, cause the processor to perform the computer-implemented method.

In various implementations, a system for cytological smear human review workload management, comprising: a computer configured to: receive a first image of a first cytological smear associated with a first sample obtained from a first patient; receive a second image of a second cytological smear associated with a second sample obtained from a second patient; determine, from the first image, first and second hematological cell or cell type quantification metrics (HCQMs) for the first patient, wherein the first HCQM is for a first abnormal cell type and the second HCQM is for a second abnormal cell type; determine, from the second image, the first and second HCQMs for the second patient; receive first and second predetermined thresholds associated with the first and second HCQMs, respectively; compare the first and second HCQMs for the first patient with the first and second predetermined thresholds, respectively, and identify that the first HCQM is above the first threshold and the second HCQM is below the second threshold; compare the first and second HCQMs for the second patient with the first and second predetermined thresholds, respectively, and identify that the first HCQM is below the first threshold and the second HCQM is above the second threshold; cause cells associated with the first sample to be presented as part of default human review workflow; and cause cells associated with the second sample to be excluded from presentation as part of default human review workflow.

In some implementations of the system, the first abnormal cell type is selected from: blast cell, aberrant lymphocyte, promyelocyte, atypical lymphocyte, Sézary cell, hairy cell, large granular lymphocyte, Pelger-Huet cell. In some implementations of the system, the second abnormal cell type is selected from: blast cell, aberrant lymphocyte, promyelocyte, atypical lymphocyte, Sézary cell, hairy cell, large granular lymphocyte, Pelger-Huet cell, Plasma cell, schistocyte, smudge cell, helmet cell, bite cell, blister cell or cell containing a microorganism.

In some implementations of the system, the first abnormal cell type is selected from: myelocyte, metamyelocyte Plasma cell, schistocyte, smudge cell, helmet cell, bite cell, nucleated RBC, blister cell or cell containing a microorganism; and wherein the second abnormal cell type is selected from: myelocyte, band neutrophil, metamyelocyte, nucleated RBC, sickle cell, polychromatic cell or cell with Howell-jolly body. In some implementations of the system, the computer is further configured to: monitor human review results over time for multiple cytological smears; adjust at least the first predetermined threshold based on the monitoring, wherein the adjusting comprises increasing the at least first predetermined threshold to reduce a number of smears presented for human review. In some implementations of the system, the computer is further configured to: generate a first report based at least on the first and second HCQMs for the first patient; generate a second report based at least on the first and second HCQMs for the second patient; and automatically transmit the second report to an electronic medical information system.

In some implementations of the system, the computer is further configured to: receive a third image of a third cytological smear associated with a third sample obtained from a third patient; receive a fourth image of a fourth cytological smear associated with a fourth sample obtained from a fourth patient; determine, from the third image, a quantity of unclassified cells for the third patient; determine, from the fourth image, a quantity of unclassified cells for the fourth patient; receive a predetermined unclassified cell threshold; compare the quantity of unclassified cells for the third patient with the predetermined unclassified cell threshold and identify that the quantity of unclassified cells for the third patient is above the predetermined unclassified cell threshold; compare the quantity of unclassified cells for the fourth patient with the predetermined unclassified cell threshold and identify that the quantity of unclassified cells for the fourth patient is below the predetermined unclassified cell threshold; cause cells associated with the third sample to be presented as part of default human review workflow; and cause cells associated with the fourth sample to be excluded from presentation as part of default human review workflow.

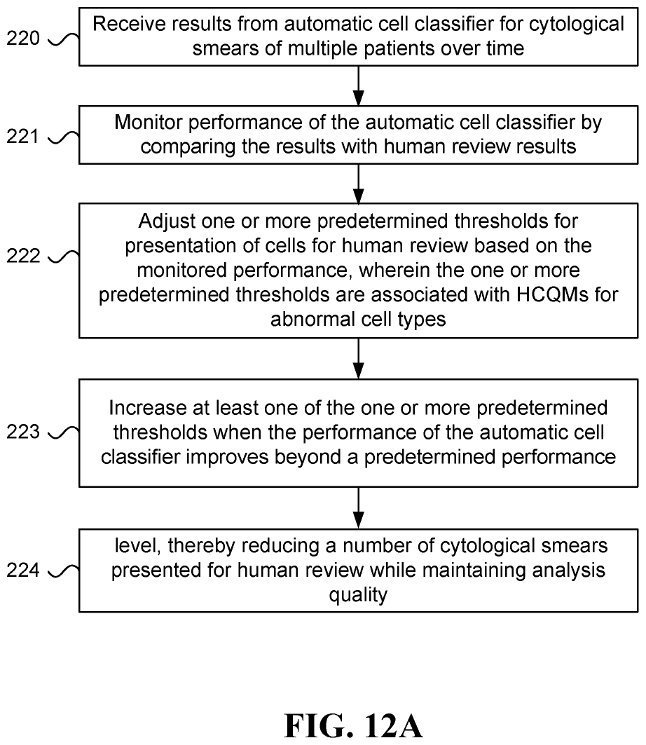

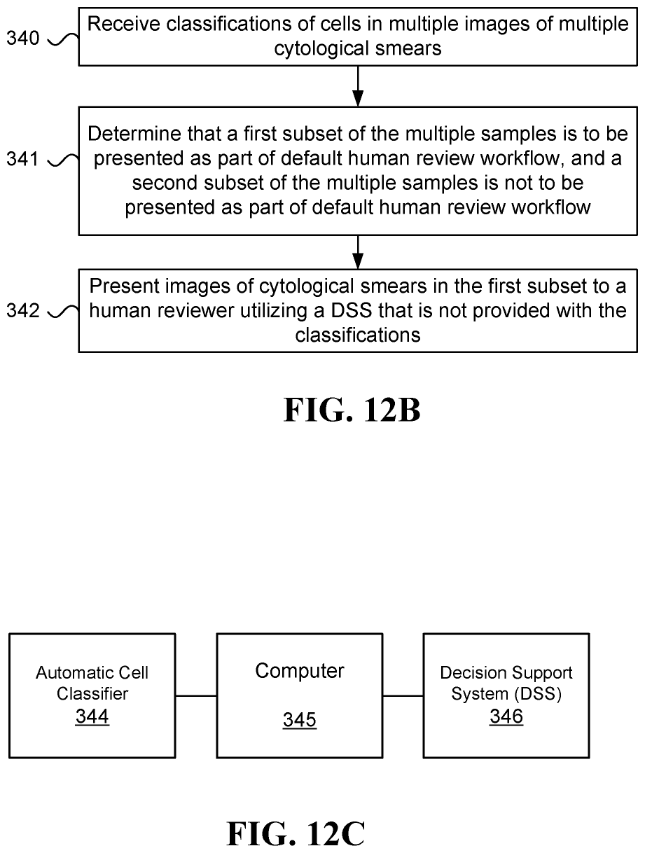

In various implementations, a computer-implemented method for adaptive management of cytological smear human review workload, comprising: receiving results from an automatic cell classifier for cytological smears of multiple patients over time; monitoring performance of the automatic cell classifier by comparing the results with human review results; adjusting one or more predetermined thresholds for presentation of cells for human review based on the monitored performance, wherein the one or more predetermined thresholds are associated with hematological cell or cell type quantification metrics (HCQMs) for abnormal cell types; and increasing at least one of the one or more predetermined thresholds when the performance of the automatic cell classifier improves beyond a predetermined performance level, thereby reducing a number of cytological smears presented for human review while maintaining analysis quality. FIG. 12A illustrates an embodiment of a computer-implemented method for adaptive management of cytological smear human review workload. The method includes the following steps. In step 220, receive results from an automatic cell classifier for cytological smears of multiple patients over time. In step 221, monitor performance of the automatic cell classifier by comparing the results with human review results. In step 222, adjust one or more predetermined thresholds for presentation of cells for human review based on the monitored performance, wherein the one or more predetermined thresholds are associated with HCQMs for abnormal cell types. And in step 223, increase at least one of the one or more predetermined thresholds when the performance of the automatic cell classifier improves beyond a predetermined performance level, thereby reducing a number of cytological smears presented for human review while maintaining analysis quality.

The adaptive management method may establish a systematic framework for continuous optimization of laboratory resource allocation. The receipt of results from the automatic cell classifier may occur through various integration mechanisms, potentially including direct system-to-system interfaces, database queries, structured file exchanges, or API interactions, depending on the specific hardware and software infrastructure of the laboratory environment. The monitoring process may implement various methodologies for performance assessment, potentially including statistical quality control approaches, error tracking mechanisms, agreement analysis tools, or machine learning performance metrics. The predetermined performance level that triggers threshold increases may be established through various approaches, potentially including validation studies that correlate performance metrics with diagnostic outcomes, statistical analyses that identify stability inflection points, industry benchmark comparisons, or iterative optimization processes. Different performance metrics may be prioritized in different implementation contexts, potentially including sensitivity and specificity for clinically significant findings, overall classification accuracy, confidence level distributions, or throughput efficiency measures. The threshold adjustments may follow various mathematical models, potentially including stepwise increments, proportional adjustments based on performance margins, or adaptive algorithms that accelerate or decelerate adjustment rates based on historical outcome data. The maintenance of analysis quality may be ensured through various oversight mechanisms, potentially including randomized verification samples, periodic complete validation studies, statistical process control monitoring, or targeted reviews of high-risk categories. This adaptive approach may create a continuous improvement ecosystem that systematically optimizes the distribution of human expertise while maintaining appropriate quality assurance throughout the evolutionary process.

In some implementations, the method further comprises tracking a rate of agreement between the results from the automatic cell classifier and the human review results; and automatically increasing or suggest increasing the at least one of the one or more predetermined thresholds when the rate of agreement exceeds a predetermined agreement threshold. The agreement rate monitoring approach may provide quantifiable metrics to guide threshold adjustment decisions. The tracking of agreement rates may involve various analytical methodologies, potentially including cell-by-cell concordance analysis, case-level outcome agreement assessment, weighted agreement calculations that prioritize clinically significant findings, or confidence-adjusted measures that incorporate certainty levels from both human and automated sources. The agreement tracking may be implemented across various timeframes and aggregation levels, potentially including real-time continuous monitoring, periodic batch analyses, rolling time-window calculations, or stratified tracking across different sample categories or patient demographics. The predetermined agreement threshold may be established through various approaches, potentially including statistical analyses of historical laboratory data, correlation studies linking agreement rates to diagnostic outcomes, or progressive validation protocols that systematically evaluate performance at different threshold levels. The system may implement either automatic threshold increases or suggestion mechanisms depending on laboratory preferences and regulatory considerations, potentially including fully automated adjustments with appropriate documentation, semi-automated suggestions requiring human confirmation, or hybrid approaches with automatic adjustments for certain cell types and suggested adjustments for others based on their clinical significance. The suggestion mechanism may present various supporting data to inform human decision-making, potentially including trend visualizations, statistical significance indicators, predicted workload impact analyses, or comparison benchmarks from similar laboratory environments. This data-driven approach may provide objective criteria for workflow optimization decisions, potentially reducing subjective factors in resource allocation while maintaining appropriate quality assurance mechanisms throughout the adjustment process.

In some implementations of the method, the adjusting is performed periodically according to a predetermined schedule to progressively reduce human review workload over time while maintaining analysis quality. The predetermined schedule approach may provide a structured framework for systematic workflow evolution. The schedule may be implemented through various temporal patterns, potentially including fixed calendar intervals (monthly, quarterly, or annually), sample volume milestones (after processing specific numbers of cases), performance stability periods (after achieving consistent metrics at current settings), or hybrid approaches that combine multiple triggering criteria. The progressive reduction may follow various trajectories, potentially including linear step reductions, front-loaded approaches that implement larger initial changes followed by smaller refinements, or variable patterns that adjust the magnitude of changes based on performance metrics or resource considerations. Different implementation approaches may establish the predetermined schedule through various mechanisms, potentially including fixed schedules established during system implementation, dynamically adjusted schedules that respond to performance trends, or adaptive schedules that accelerate or decelerate based on outcome monitoring. The maintenance of analysis quality throughout this progressive process may be ensured through various verification mechanisms, potentially including statistical quality control approaches, randomized validation samples, targeted reviews of boundary cases, or periodic comprehensive assessments across all cell types and sample categories. These verification approaches may track multiple quality indicators, potentially including correlation with reference methods, internal consistency measures, clinical outcome associations, or specific performance metrics for high-risk categories. The system may implement appropriate safeguards if quality concerns arise, potentially including temporary suspension of further adjustments, targeted threshold reversals for specific cell types, or comprehensive review of classification models. This measured, incremental approach may allow laboratories to optimize resource allocation through controlled evolution rather than abrupt transitions, potentially balancing efficiency improvements with appropriate quality assurance throughout the optimization process.

In some implementations of the method, the adjusting comprises: determining cell type-specific agreement rates between the results from the automatic cell classifier and the human review results for different abnormal cell types; and selectively increasing thresholds for specific abnormal cell types based on their respective agreement rates. The cell type-specific adjustment approach may provide granular optimization that recognizes the varying classification challenges and clinical significance of different abnormal cell types. The determination of cell type-specific agreement rates may involve various analytical methodologies, potentially including confusion matrix analysis to identify specific misclassification patterns, boundary case studies to evaluate challenging morphologies, confidence distribution analysis to assess classification certainty across the cell type population, or temporal trend analysis to identify evolving performance characteristics. The agreement analysis may incorporate various nuanced considerations, potentially including morphological subtypes within broader cell categories, technical factors that may affect visualization of specific features, prevalence considerations that may impact statistical confidence, or clinical significance weighting that prioritizes diagnostic impact over simple numerical agreement. The selective threshold increases may follow various approaches, potentially including prioritizing adjustments for high-volume cell types to improve efficiency impact, focusing on clinically less critical classifications to maintain conservative approaches for significant findings, targeting cell types with consistently high agreement rates to build on demonstrated performance strengths, or addressing cell types with the largest divergence between current performance and threshold levels to optimize adjustment impact. The implementation may include appropriate interdependency considerations, recognizing that some cell types share morphological features or exist along biological continua, potentially requiring coordinated threshold management across related categories. This differentiated approach may optimize resource allocation by allowing more aggressive automation for cell types with demonstrated classification reliability while maintaining appropriate human oversight for more challenging or clinically significant classifications, potentially creating a nuanced balance between efficiency and quality assurance across the full spectrum of hematological findings.

In some implementations of the method, the number of cytological smears presented for human review constitutes less than 15% of the cytological smears analyzed by the automatic cell classifier. The specific ratio threshold may represent an operational efficiency target that reflects advanced automation capabilities. The ratio of less than 15% may be achieved through various implementation approaches, potentially including progressive threshold optimization, classifier performance improvements, routing algorithms that consider multiple parameters beyond simple thresholds, or combinations of these approaches working synergistically. Different laboratory environments may implement various paths toward achieving this ratio, potentially including gradual progression from higher review percentages, targeted focus on high-volume normal cases to improve efficiency impact, or stratified approaches that vary review percentages across different sample categories or clinical contexts. The maintenance of appropriate quality assurance with this limited review ratio may be ensured through various mechanisms, potentially including selection of which samples receive human review, enhanced quality control processes for automated-only analyses, statistical monitoring of system performance, or compensatory increases in targeted quality assurance activities. The specific percentage target may vary across implementation environments based on factors such as patient population characteristics, regulatory requirements, laboratory accreditation standards, or specific clinical applications. This efficient resource allocation may provide various operational benefits, potentially including increased laboratory throughput, reduced turnaround times, more focused specialist attention on challenging cases, or improved cost-effectiveness while maintaining appropriate diagnostic quality. The system may implement various tracking mechanisms to monitor the actual achieved ratio against the target, potentially including real-time dashboards, periodic performance reports, trend analyses across time, or benchmarking comparisons across similar laboratory environments. In some implementations of the method, a non-transitory computer-readable medium comprising instructions that, when executed by a processor, cause the processor to perform the computer-implemented method.

In various implementations, a system for adaptive management of cytological smear human review workload, comprising: a computer configured to: receive results from an automatic cell classifier for cytological smears of multiple patients over time; monitor performance of the automatic cell classifier by comparing the results with human review results; adjust one or more predetermined thresholds for presentation of cells for human review based on the monitored performance, wherein the one or more predetermined thresholds are associated with hematological cell or cell type quantification metrics (HCQMs) for abnormal cell types; and increase at least one of the one or more predetermined thresholds when the performance of the automatic cell classifier improves beyond a predetermined performance level, thereby reducing a number of smears presented for human review while maintaining analysis quality.

In some implementations of the system, the computer is further configured to: track a rate of agreement between the results from the automatic cell classifier and the human review results; and automatically increase or suggest to increase the at least one of the one or more predetermined thresholds when the rate of agreement exceeds a predetermined agreement threshold. In some implementations of the system, the computer is further configured to adjust or suggest adjusting the one or more predetermined thresholds periodically according to a predetermined schedule to progressively reduce human review workload over time while maintaining analysis quality.

In some implementations of the system, the computer is further configured to: determine cell type-specific agreement rates between the results from the automatic cell classifier and the human review results for different abnormal cell types; and selectively increase thresholds or suggest increasing the thresholds for specific abnormal cell types based on their respective agreement rates. In some implementations of the system, the computer is further configured to: reduce the number of cytological smears presented for human review such that they constitute less than 15% of the cytological smears analyzed by the automatic cell classifier.

The following implementation may address two operational concerns that may arise when an automatic cell classifier is utilized in combination with a downstream decision support system operated by a human reviewer. First, when a human reviewer interacts with a decision support system that has received outputs of an automatic cell classifier, the reviewer's subsequent assessment may be influenced by those outputs, which may affect the independence of the human judgment, and in some cases may increase the likelihood of anchoring to the results of the automatic cell classifier. Second, when a decision support system itself performs analytical functions on cytological smear images, providing that decision support system with outputs of the automatic cell classifier may reduce the utility of the decision support system as a second-opinion tool, since its own analysis may be influenced by, or duplicative of, the outputs of the automatic cell classifier. By automatically triaging samples using an automatic cell classifier, and then presenting images of cytological smears associated with a relatively small subset of those samples to a human reviewer utilizing a decision support system that is not provided, for all cells in the presented images, with classifications from the automatic cell classifier, the embodiment may support independent downstream analysis and may reduce classifier-induced bias in the human review, while still benefiting from the efficiency gains of automated triage. The embodiment may be deployed in clinical environments where most samples are handled autonomously by the automatic cell classifier, and only a relatively small portion is presented to a human reviewer utilizing the decision support system, which may reduce reviewer workload while preserving human oversight of the cases that may benefit most from independent assessment.

In various implementations, a computer-implemented method for preparing images for human review, comprising: receiving, from an automatic cell classifier, classifications of cells in multiple images of multiple cytological smears, wherein each of the multiple cytological smears is associated with one of multiple samples, and wherein the multiple samples are obtained from multiple patients; determining, based on the classifications, that a first subset of the multiple samples is to be presented as part of default human review workflow, and that a second subset of the multiple samples is not to be presented as part of default human review workflow; wherein the first subset comprises less than 10% of the multiple samples, and the second subset comprises more than 90% of the multiple samples; and presenting images of cytological smears to a human reviewer utilizing a decision support system (DSS), wherein the images presented utilizing the DSS are of cytological smears associated with samples in the first subset, and wherein the DSS is not provided with classifications produced by the automatic cell classifier for all cells in the images presented utilizing the DSS. FIG. 12B illustrates an embodiment of a computer-implemented method for preparing images for human review utilizing a decision support system (DSS). The method includes the following steps. In step 340, receive, from an automatic cell classifier, classifications of cells in multiple images of multiple cytological smears, wherein each of the multiple cytological smears is associated with one of multiple samples, and wherein the multiple samples are obtained from multiple patients. In step 341, determine, based on the classifications, that a first subset of the multiple samples is to be presented as part of default human review workflow, and that a second subset of the multiple samples is not to be presented as part of default human review workflow, wherein the first subset comprises less than 10% of the multiple samples, and the second subset comprises more than 90% of the multiple samples. And in step 342, present images of cytological smears to a human reviewer utilizing a DSS, wherein the images presented utilizing the DSS are of cytological smears associated with samples in the first subset, and wherein the DSS is not provided with classifications produced by the automatic cell classifier for all cells in the images presented utilizing the DSS.

“Decision support system” (DSS) refers to a computerized system utilized by a human reviewer to facilitate review, interpretation, and/or diagnosis of one or more images of cytological smears. The DSS may include hardware, software, firmware, or any combination thereof, and may execute on a general-purpose processor, a dedicated workstation, a distributed computing environment, a cloud-hosted service, or any other computing arrangement suitable for the functions described herein. The DSS may reside on the same computing environment as the automatic cell classifier or on a different computing environment, and may be provided by the same vendor as the automatic cell classifier or by a different vendor. The DSS may include, by way of non-limiting example, an image viewing and annotation application supporting human assessment, an image analysis application performing detection, classification, measurement, and/or segmentation of cells in the images, and/or any combination thereof. Outputs produced utilizing the DSS may include review findings, annotations, measurements, counts, classifications provided or confirmed by the human reviewer, aggregated analysis results, textual reports, or any other data useful for downstream clinical decision-making.

Each of the multiple samples is associated with at least one cytological smear, and may be associated with one or more cytological smears. The triage based on the classifications allocates each of the multiple samples into the first subset or the second subset. Because the first subset is defined as a subset of samples, the images of cytological smears associated with samples in the first subset that are presented to the human reviewer utilizing the DSS may include images of the specific cytological smears that were analyzed by the automatic cell classifier, images acquired by additional scans of those cytological smears, images of further cytological smears prepared from samples in the first subset (associate with this the relevant patients), and/or any combination thereof.

Phrases in the form of “the DSS is not provided with classifications produced by the automatic cell classifier for all cells in the images presented utilizing the DSS” refer to refraining from making available to the DSS, for at least some cells among the cells in the images presented utilizing the DSS, the classifications produced by the automatic cell classifier. In this context, “making available to the DSS” and “provided with” refer to arrangements in which the classifications are not functionally available to the DSS for producing its outputs or for presenting information to the human reviewer, regardless of whether the classifications are nominally present in data that reaches the DSS. Accordingly, an arrangement in which the classifications are transmitted to or stored on a component of the DSS but the DSS is configured to disregard, not access, not apply, not display, not process, or otherwise not functionally employ the classifications in producing its outputs, falls within the scope of “the DSS is not provided with the classifications” for the corresponding cells. Under this phrasing, the DSS may operate on images in which the classifier's classifications are not provided for any cell, or may operate on images in which the classifier's classifications are provided for some cells but not all (such as when the human reviewer retrieves classifications for specific cells or for a specific area through a user-initiated request). This operation may be implemented through various technical mechanisms, which may be utilized individually or in combination, including, but not limited to: not transmitting the classifications to the DSS over a data interface; not storing the classifications in a storage accessible to the DSS; excluding the classifications from data packages sent to the DSS; not loading the classifications into database, cache, or memory location from which the DSS retrieves data for its analysis; not rendering the classifications in user interface element of the DSS; not pre-populating fields or controls of the DSS with the classifications; removing the classifications from metadata associated with the images before the images are provided to the DSS; configuring the DSS to disregard the classifications when present in data received by the DSS; configuring the DSS to refrain from applying the automatic cell classifier to cells in the images; and/or any other mechanism that prevents the classifications from reaching or being utilized by the DSS in a manner that would influence its outputs or the interaction of the human reviewer therewith with respect to cells for which the classifications are withheld. The operation may coexist with maintaining the classifications separately within the system, such that the classifications remain accessible for audit, traceability, or other non-DSS purposes, without being provided to the DSS during its operation on the corresponding cells.

The images of cytological smears associated with samples in the first subset that are presented utilizing the DSS may be the same images that were previously analyzed by the automatic cell classifier, may be different images of the same cytological smears acquired in one or more additional scans, may be images of further cytological smears prepared from samples in the first subset, or may include any combination thereof. When previously acquired images are utilized, no additional scanning operation may be required, which may reduce operational time. When additional scans or new smears are utilized, imaging parameters may be selected for a different purpose than the initial scan, such as a higher effective numerical aperture (ENA), a larger imaged area, a different focus technique, different illumination conditions, and/or different magnification, which may produce images more suitable for independent analysis by the DSS and/or by the human reviewer.

The determining of the first subset and the second subset may be implemented through various computational approaches that operate on the classifications. Non-limiting examples of such approaches include: comparing a result of a function of CCLs associated with the classifications with one or more thresholds; comparing HCQM derived from the classifications with one or more predetermined thresholds; evaluating combinations of parameters derived from the classifications against a screening function; comparing analysis results derived from the classifications with historical analysis results for the patient; evaluating deviations of the classifications from expected medication-induced or condition-induced cellular patterns; applying rule-based decision logic to the classifications; and/or applying machine learning models trained to distinguish between samples that merit human review and samples that do not. The determining step may also incorporate additional inputs beyond the classifications, such as patient medical history, laboratory protocols, reviewer availability, and/or quality indicators associated with the cytological smears. The specific proportion of the first and second subsets, which in the main claim is less than 10% and more than 90% of the multiple samples, respectively, may be governed by the selection of these thresholds, functions, or models, and may vary with classifier performance, patient population characteristics, clinical setting, and/or regulatory context. In non-limiting examples, the first subset may include less than 10%, less than 5%, or less than 1% of the multiple samples, and the second subset may correspondingly comprise more than 90%, more than 95%, or more than 99% of the multiple samples.

The receiving of classifications of cells in multiple images of multiple cytological smears may occur in a single event, in multiple events over time, continuously, or in any combination thereof. Accordingly, the multiple samples may be received simultaneously, sequentially over time, or in any combination thereof, and the determining of the first subset and the second subset may be performed on samples received together, on samples received at different times, or on any combination thereof. The percentages defining the first and second subsets represent a design or statistical property of the implementation, and are not limited to any particular temporal or batch-size arrangement. In non-limiting examples, the percentages may be evaluated over a single receipt event, over samples received within a predefined time window (such as a work shift, a day, a week, or a month), over samples received from a specified set of sources (such as one or more laboratories, one or more imaging systems, or one or more clinical departments), over a rolling population of samples, over a sliding window of recently received samples, or over any other aggregation suitable for the operational setting.

Phrases in the form of “presenting images of cytological smears to a human reviewer utilizing a DSS” may refer to various methods of making the images available to the human reviewer through the DSS, including, but not limited to: loading the images into the DSS for viewing and analysis by the human reviewer, placing the images in a worklist or queue accessed through the DSS, routing the images to a reviewer workstation running the DSS, transmitting the images to a server that serves the DSS interface to the human reviewer, and/or any other mechanism by which the human reviewer accesses the images through the DSS. The operation may occur in real time, in batch, asynchronously, or according to any scheduling arrangement suitable for the laboratory workflow. Determining that the second subset is “not to be presented as part of default human review workflow” may be implemented as described elsewhere in this disclosure for the corresponding operation, and may involve preventing the samples in the second subset from entering human review queues, routing them through pathways that bypass human review, marking them with metadata indicating completion without human review, storing them in repositories for completed analyses, and/or any combination thereof.

In a non-limiting implementation, the method may be carried out by a computer communicatively coupled to an automatic cell classifier (which may include a machine learning model, a deep neural network, an ensemble classifier, or any other model suitable for cell classification), to an imaging system capable of acquiring images of cytological smears, and to a DSS operable by a human reviewer. The computer may include one or more processors configured to execute instructions that perform the receiving, determining, and presenting operations. The computer may maintain a data pathway for the classifications that is separate from the data pathway provided to the DSS, such that the classifications are processed for the determining operation but not forwarded to the DSS for the presenting operation, at least with respect to those cells for which the classifications are withheld. The DSS may include a client application running on a reviewer workstation, a web-based application served to the reviewer through a browser, a mobile application, and/or any other user-facing software capable of receiving the images and accepting input from the human reviewer.

In operation, the automatic cell classifier analyzes multiple images of multiple cytological smears associated with samples obtained from multiple patients, and produces classifications of cells in those images. Based on the classifications, the computer determines that less than 10% of the multiple samples belong to the first subset and are to be presented as part of default human review workflow, and that more than 90% belong to the second subset and are not to be presented. The computer then provides images of cytological smears associated with samples in the first subset to the DSS, while withholding, for all cells in the presented images, the classifications produced by the automatic cell classifier. The human reviewer operates the DSS on the received images, which may include performing image analysis with the DSS, annotating findings, selecting cells or regions of interest, confirming or modifying DSS-generated results, and producing a review output. The review output may be utilized for downstream clinical purposes, such as reporting to treating physicians, integration with an electronic medical information system, or further investigations. The arrangement may support independent assessment by the human reviewer and the DSS on the presented images, and may reduce the likelihood that the review output is anchored to the classifications produced by the automatic cell classifier.

The determining of the first and second subsets may be implemented, in non-limiting examples, by algorithms including: threshold comparison algorithms that evaluate CCLs or functions thereof against predetermined thresholds; statistical classifiers such as logistic regression, support vector machines, random forests, or gradient-boosted decision trees that produce a triage decision from classifier-derived features; deep learning models such as convolutional neural networks or transformer-based architectures that operate on the classifications and/or on intermediate features extracted from the images; Bayesian inference algorithms that combine classifier outputs with prior probabilities derived from patient data and/or population statistics; rule-based expert systems that encode laboratory protocols or clinical guidelines; and/or ensemble methods that combine outputs of multiple such algorithms. The DSS may implement, in non-limiting examples, image processing algorithms such as edge detection, segmentation algorithms (for example, watershed, region growing, U-Net, Mask R-CNN), cell detection algorithms, morphological analysis algorithms, color analysis algorithms, and/or classification algorithms, any of which may operate on the images provided to the DSS without utilizing the classifications produced by the automatic cell classifier for cells for which the classifications are withheld.

In some implementations of the method, the images presented utilizing the DSS are selected from the multiple images. The images presented utilizing the DSS for the samples in the first subset are selected from the same multiple images that were previously analyzed by the automatic cell classifier. The selection may include retrieving the previously acquired images from a storage medium accessible to the computer, such as a file system, database, object store, or image management repository, and providing the retrieved images to the DSS. The selection may encompass all previously acquired images for a given cytological smear associated with a sample in the first subset, one or more image fields selected from the previously acquired images, one or more regions of interest extracted from the previously acquired images, or any other subset of the previously acquired images suitable for the human reviewer's analysis utilizing the DSS. Utilizing the previously acquired images may avoid additional scanning operations, may reduce operational time and resource consumption, and may maintain consistency between the data analyzed by the automatic cell classifier and the data available to the human reviewer through the DSS. The selection may be performed by the same computer that performed the determining step, by a separate component, or by any combination thereof.

In some implementations of the method, the images presented utilizing the DSS are acquired, after determining that data related to a sample belonging to the first subset is to be presented as part of default human review workflow, by at least one of: re-scanning one or more of the cytological smears associated with samples in the first subset, or scanning one or more new cytological smears prepared from samples in the first subset. The images of cytological smears associated with samples in the first subset are acquired through an operation performed after the determining step has identified the first subset. The operation may include re-scanning of cytological smears previously analyzed by the automatic cell classifier, scanning of new cytological smears prepared from samples in the first subset, or any combination thereof. The re-scanning or the scanning of new cytological smears may be performed by the same imaging system that acquired the multiple images analyzed by the automatic cell classifier, by a different imaging system, or by any combination thereof. The acquiring may utilize imaging parameters selected for the purpose of human review and/or independent analysis by the DSS, which may differ from the imaging parameters utilized for the initial acquisition of the multiple images. Non-limiting examples of such imaging parameters include effective numerical aperture (ENA), magnification, field of view, imaged area, focus strategy (such as single-plane or multi-plane capture), illumination conditions (such as brightfield, darkfield, phase contrast, or differential interference contrast), spectral characteristics, exposure time, gain, image resolution, and/or computational image reconstruction methods. The acquiring may be initiated automatically by the computer upon identifying the first subset, may be initiated upon receiving an operator command, or may be initiated according to a scheduling mechanism that coordinates acquiring with other laboratory activities. Applying the acquiring selectively to the first subset may confine the additional scanning time, storage, and computational overhead, as well as any additional smear preparation, to cases where the additional cost may be justified by the clinical value of the subsequent human review, which may reduce the overall operational cost per sample. Preparing one or more new cytological smears from samples in the first subset may be beneficial when the original cytological smears are inadequate for human review or for independent analysis by the DSS, or when additional preparation methods (such as alternative staining protocols or alternative smearing parameters) may improve morphological visualization of cells relevant to the human reviewer.

In some implementations of the method, the acquiring of the images presented utilizing the DSS is performed by an imaging system having an effective numerical aperture (ENA) higher than an ENA of an imaging system that acquired the multiple images. A higher ENA may provide higher resolving power, which may enable finer morphological details of cells to be visualized in the images acquired after the determining step. Non-limiting examples of higher-ENA imaging systems include microscopes with objective lenses designed to be immersed in a medium with refractive index above one (such as oil-immersion objectives), microscopes with high-NA dry objectives, computational microscopes configured to reconstruct images from a plurality of captured images taken under different illumination conditions such that the reconstructed images correspond to an ENA higher than the NA of the physical objective lens, super-resolution imaging systems configured to computationally combine multiple images to create composite images with resolution exceeding the optical diffraction limit, holographic or diffractive imaging systems, and/or any other imaging arrangement capable of producing images with resolving power exceeding that of the imaging system utilized for acquiring the multiple images.

In some implementations of the method, the DSS is configured to operate interactively with the human reviewer, such that at least some outputs of the DSS depend on input received from the human reviewer, and wherein the automatic cell classifier produces the classifications without input from the human reviewer. This embodiment characterizes the DSS as a system whose operation is interactive with the human reviewer. At least some outputs produced utilizing the DSS depend on input received from the human reviewer, which distinguishes the DSS from the automatic cell classifier, the latter producing the classifications without input from the human reviewer during the classification operation. The input received from the human reviewer may be provided at various points in the DSS workflow, including before the DSS performs its analysis (such as parameter selection, region of interest definition, or query formulation), during the DSS analysis (such as interactive guidance, iterative refinement, or selective triggering of analytical operations), and/or after initial DSS analysis (such as confirmation, modification, or correction of preliminary analysis results produced utilizing the DSS). The interactive nature of the DSS may permit the human reviewer to incorporate clinical judgment, contextual knowledge, and individualized expertise into the analysis, while leveraging the computational capabilities of the DSS for image processing, measurement, quantification, and/or presentation. The automatic cell classifier, by contrast, may operate autonomously on the multiple images once initiated, producing the classifications based solely on the classifier's trained parameters and the input images, without real-time input from the human reviewer.

In some implementations of the method, the DSS is configured to perform image analysis on the images presented to the human reviewer utilizing the DSS, the image analysis comprising at least one of: cell detection, cell classification, cell measurement, cell segmentation, cell counting, analysis of cell morphology, analysis of cell staining characteristics, or analysis of spatial distribution of cells; and wherein the input received from the human reviewer comprises at least one of: an annotation applied to at least one of the images, a selection of a cell or an area within at least one of the images, a confirmation or correction of an analysis result produced by the DSS, a classification provided by the human reviewer for a cell or a group of cells, a measurement provided by the human reviewer, a query requesting additional information, a text input, or a voice input. The image analysis performed utilizing the DSS may include one or more operations that process the images presented to the human reviewer utilizing the DSS to produce analytical outputs. Cell detection may include identifying the presence and location of cells within the images, which may be implemented by object detection algorithms such as region-based convolutional neural networks, you-only-look-once architectures, feature pyramid networks, and/or other detection models. Cell classification performed by the DSS may include assigning cells to cell types or categories; when such classification is performed, it may be implemented without utilizing the classifications received from the automatic cell classifier for the cells for which the classifications are withheld, such that the classification produced utilizing the DSS may provide an independent second-opinion output. Cell measurement may include determining quantitative parameters of cells, such as size, diameter, area, perimeter, nuclear-to-cytoplasmic ratio, staining intensity, or texture metrics. Cell segmentation may include delineating boundaries of cells or cellular components, which may be implemented by semantic or instance segmentation algorithms. Cell counting may include determining numbers of cells of various types within the images or within selected regions. Analysis of cell morphology may include assessing shape features, structural features, or morphological patterns of cells. Analysis of cell staining characteristics may include evaluating color properties, color intensity, color distribution, and/or staining artifacts within cells. Analysis of spatial distribution of cells may include evaluating how cells are distributed across the cytological smear, including clustering patterns, density variations, and/or positional relationships between different cell types.

The input received from the human reviewer may be provided through various interaction mechanisms implemented by the DSS. An annotation applied to at least one of the images may include a text label, a graphical marker, a shape such as a rectangle, circle, or polygon, a freehand drawing, a classification tag, a comment, and/or any other visible or associated indicator placed on or associated with a portion of the image. A selection of a cell or an area within at least one of the images may be provided through pointer operations (such as clicks or taps), region drawing operations, area marking operations, lasso selection operations, and/or other selection mechanisms. A confirmation or correction of an analysis result produced by the DSS may include accepting a DSS-proposed finding as-is, modifying a value, changing a classification, removing a proposed finding, adding a finding that the DSS did not propose, and/or any combination thereof. A classification provided by the human reviewer for a cell or a group of cells may include assigning a cell type, category, or descriptive label from a predetermined list or from a free-form vocabulary. A measurement provided by the human reviewer may include entering a numerical value for a measurement parameter, drawing a measurement reference (such as a caliper or ruler tool), and/or selecting a predefined measurement option. A query requesting additional information may include a request for comparison with reference images, retrieval of patient history, contextual information, or other data accessible to the DSS. A text input may be provided through a keyboard, on-screen keyboard, stylus, or any other text entry mechanism. A voice input may be provided through a microphone coupled to a voice recognition module of the DSS. The DSS may support any combination of these interaction mechanisms, and may adapt the available interaction mechanisms to the reviewer's workflow, workstation configuration, and/or laboratory preferences.

In some implementations of the method, the images presented to the human reviewer utilizing the DSS are provided to the DSS without any output of the automatic cell classifier. The DSS may receive the images presented to the human reviewer without receiving any output of the automatic cell classifier for those images. No classifications, no CCLs, no HCQMs, no aggregated analysis results, no detection locations, and no other classifier-derived data are provided to the DSS. This arrangement may provide the highest degree of independence of the DSS operation and of the human reviewer's assessment from the outputs of the automatic cell classifier, and may be selected in deployments where complete decoupling of the two analytical pathways is desired. The arrangement may be implemented by a data pipeline that isolates the output stream of the automatic cell classifier from the input stream of the DSS, such that only raw image data, optionally with image-level metadata not derived from the automatic cell classifier (such as patient identifier, acquisition timestamp, imaging parameters, or quality indicators computed independently of the automatic cell classifier), reaches the DSS. This implementation may be compatible with images selected from the multiple images, images acquired by re-scanning, and images of new cytological smears prepared from samples in the first subset.

In some implementations of the method, the images presented to the human reviewer utilizing the DSS comprise at least a portion of the multiple images, and wherein the DSS is provided with the at least a portion of the multiple images and with data indicative of locations of cells detected by the automatic cell classifier in the at least a portion of the multiple images, and wherein the classifications of cells at the locations are withheld from the DSS. The DSS may receive, in addition to at least a portion of the multiple images, data indicative of locations of cells detected by the automatic cell classifier in those images, while the classifications of the cells at those locations are withheld from the DSS. Because the automatic cell classifier operates on the multiple images, the detection locations are available for those multiple images. The data indicative of locations may include bounding boxes enclosing detected cells, coordinate lists identifying cell centers, segmentation masks delineating cell boundaries, region-of-interest identifiers associated with image patches containing cells, and/or any other representation that indicates where cells have been detected without indicating the classifications assigned to those cells. Providing the locations may enable the DSS and/or the human reviewer to focus on regions of the images where cells have been identified, which may improve efficiency of the review, while withholding the classifications may preserve the independence of any classification, categorization, or interpretation performed utilizing the DSS or by the human reviewer. In some implementations, the data indicative of locations may be anonymized, such that any labels or identifiers associated with the locations carry no information about the cell type (for example, the locations may be represented with generic identifiers such as numerical indices rather than with cell-type-specific labels).

In some implementations of the method, the DSS is not provided with at least one of: (i) classification confidence levels (CCLs) associated with the classifications of cells for which the classifications are withheld; (ii) hematological cell or cell type quantification metrics (HCQMs) derived from the classifications of cells associated with samples in the first subset; (iii) aggregated analysis results derived from the classifications of cells associated with samples in the first subset; or (iv) any indication of a basis on which the first subset was determined to be presented as part of default human review workflow. This embodiment specifies further categories of data derived from or associated with the operation of the automatic cell classifier that may be withheld from the DSS, in addition to the classifications themselves. CCLs may represent measures of certainty associated with the classifications, and withholding the CCLs may prevent the DSS and/or the human reviewer from being influenced by the classifier's self-assessment of its outputs. HCQMs may represent values derived from the classifications that describe the presence, abundance, concentration, density, or relative proportion of specific cell types associated with samples in the first subset; withholding HCQMs may prevent the DSS and/or the human reviewer from being influenced by aggregated cell-type statistics derived from the classifications. Aggregated analysis results may include any other values or interpretations computed from the classifications, such as differential counts, flag indications, scoring outputs, or derived diagnostic suggestions; withholding such results may maintain the independence of the analysis performed utilizing the DSS. Withholding any indication of the basis on which the first subset was determined to be presented may prevent the DSS and/or the human reviewer from inferring, from context or hints provided by the system, the nature of the finding that triggered the presentation, which may further preserve the independence of the DSS analysis and of the human reviewer's assessment. Various combinations of these categories may be withheld, depending on the degree of independence desired in a particular deployment, and the selection of categories to be withheld may be made configurable through system settings operable by laboratory personnel.

In some implementations of the method, the DSS not being provided with the classifications for all cells in the images presented utilizing the DSS is implemented by at least one of: not transmitting the classifications to the DSS, not storing the classifications in a storage accessible to the DSS, excluding the classifications from data sent to the DSS, removing the classifications from metadata associated with the images before the images are provided to the DSS, configuring the DSS to disregard the classifications when present in data received by the DSS, or configuring the DSS to refrain from applying the automatic cell classifier to the images presented utilizing the DSS. The withholding of the classifications from the DSS may be realized through various technical implementations, which may be utilized alone or in combination. In one implementation, the classifications are not transmitted to the DSS, such that no communication channel carries the classifications from the computer implementing the determining step to the DSS. In another implementation, the classifications are not stored in any storage location accessible to the DSS, which may include configuring access control mechanisms, database permissions, or file system permissions such that the DSS cannot retrieve the classifications from any shared storage. In a further implementation, the classifications are excluded from data packages (such as messages, structured documents, or binary payloads) sent to the DSS, such that the data packages contain the images and optionally other non-classification data but do not contain the classifications. In another implementation, the classifications, if present in metadata associated with the images, are removed from the metadata before the images are provided to the DSS, such as by stripping, rewriting, or replacing the relevant metadata fields. In a further implementation, the DSS is configured (such as through configuration parameters, policy settings, or source-level modifications) to disregard the classifications when the classifications are nevertheless present in data received by the DSS, such that the DSS neither displays, processes, nor utilizes the classifications in producing its outputs or in presenting information to the human reviewer. In yet a further implementation, the DSS is configured to refrain from applying the automatic cell classifier to the images presented utilizing the DSS, such that the DSS does not produce, through application of the automatic cell classifier, classifications for cells in those images. The selection among these implementations may be governed by technical constraints of the existing system architecture, integration requirements with external systems, privacy and audit considerations, and/or operational preferences. The system may further maintain audit records that document which implementation is applied for each case, and may provide mechanisms for verifying that the classifications have not been provided to the DSS.

In some implementations, the method further comprises, responsive to receiving from the human reviewer an indication identifying a specific cell in one of the images presented to the human reviewer utilizing the DSS, providing the human reviewer with the classification of the specific cell. The system may permit the human reviewer to retrieve, on a targeted basis, the classification associated with a specific cell in an image presented to the human reviewer utilizing the DSS, notwithstanding that the DSS is not provided with classifications produced by the automatic cell classifier for all cells in the presented images. The indication identifying the specific cell may be received through any interaction mechanism supported by the DSS, the computer, or an adjunct interface, including, by way of non-limiting example: a pointer-based selection such as a click or tap on the specific cell, a drawing of a shape that encloses the specific cell, a keyboard-based selection, a voice command identifying the specific cell, a gaze indication detected by an eye-tracking device, and/or a textual identifier of the specific cell entered by the human reviewer. Upon receiving the indication, the system retrieves the classification of the specific cell from a data repository maintained separately from the DSS, and provides the classification to the human reviewer through an appropriate output channel, which may include a display element within the DSS interface, a separate display window, an audio output, a printout, a message delivered to a reviewer's device, and/or any combination thereof. The retrieval and provision of the classification may be logged for audit and quality-assurance purposes, which may enable post-hoc analysis of override patterns, reviewer behavior, and/or correlation between override use and final review outcomes. The arrangement may permit the human reviewer to consult the classification of a specific cell when the reviewer seeks a second opinion on that cell, while the review of other cells in the image may proceed utilizing the DSS without the influence of the classifications.

In some implementations, the method further comprises, responsive to receiving from the human reviewer an indication identifying a specific area within one of the images presented to the human reviewer utilizing the DSS, providing the human reviewer with classifications of cells in the specific area. The system may permit the human reviewer to retrieve, for cells located within a specific area identified by the human reviewer, the classifications associated with those cells, notwithstanding that the DSS is not provided with classifications produced by the automatic cell classifier for all cells in the presented images. The indication identifying the specific area may be received through any interaction mechanism, including, by way of non-limiting example: drawing a bounding box around the area, drawing a freehand region around the area, selecting a grid cell or predefined region from the image, identifying the area by coordinates, identifying a region centered on a cell or landmark with a specified radius or extent, and/or any combination thereof. Upon receiving the indication, the system identifies those cells whose locations (as determined by the automatic cell classifier or by another detection mechanism) fall within the specific area, retrieves the classifications of those cells from a data repository maintained separately from the DSS, and provides the retrieved classifications to the human reviewer through an appropriate output channel. The retrieval and provision of the classifications for a specific area may be logged for audit and quality-assurance purposes. The arrangement may permit the human reviewer to consult the classifications of cells in a region of interest when the reviewer seeks a second opinion on that region, while the review of other regions of the image may proceed utilizing the DSS without the influence of the classifications.

In some implementations of the method, the determining is further based on a biomarker derived from the classifications, wherein the biomarker is correlated with a condition of one of the multiple patients, and wherein the condition comprises at least one of: a hematological malignancy, an infection, an inflammation, an anemia, a treatment response, a treatment toxicity, a disease progression, a disease remission, or a genetic condition. “Biomarker” in the context of this embodiment refers to a measurable indicator derived from the classifications that is correlated with a biological or clinical state of a patient. The biomarker may be computed from the classifications through various operations, including, by way of non-limiting example: counts of cells of specific types, ratios between counts of different cell types, proportions of cell types within defined populations, distributions of cell types, patterns of co-occurrence of cell types, and/or composite scores computed by weighting or combining multiple such values. The biomarker may be compared with one or more reference values, ranges, or thresholds to yield an indication that contributes to the determining of the first and second subsets. For example, a biomarker may indicate presence of a hematological malignancy when specific cell types (such as blast cells or aberrant lymphocytes) exceed defined proportions; a biomarker may indicate an infection when neutrophil counts or distributions depart from expected ranges; a biomarker may indicate inflammation when reactive cellular features are detected in specific proportions; a biomarker may indicate an anemia when red blood cell quantification metrics fall outside normal ranges; a biomarker may indicate a treatment response when cellular changes align with expected pharmacological effects; a biomarker may indicate a treatment toxicity when cellular changes exceed expected pharmacological effects; a biomarker may indicate disease progression or disease remission through changes in cellular patterns compared with historical cellular patterns for the same patient; and a biomarker may indicate a genetic condition when cellular features characteristic of specific genetic conditions are detected. The correlation between the biomarker and the condition may be established through clinical literature, retrospective studies, prospective validation, or machine learning training on labeled datasets. The determining of the first and second subsets may be based on the biomarker alone, on the biomarker in combination with other criteria (such as CCLs, HCQMs, screening functions, or historical comparisons), or on any other combination that incorporates the biomarker.

In some implementations of the method, the determining is further based on a biomarker derived from data extracted from the multiple images, wherein the biomarker is correlated with a condition of one of the multiple patients, wherein the data comprises at least one of: the classifications, morphological features of cells detected in the multiple images, staining characteristics of cells detected in the multiple images, spatial distribution of cells detected in the multiple images, or aggregated values computed over cells detected in the multiple images; and wherein the condition comprises at least one of: a hematological malignancy, an infection, an inflammation, an anemia, a treatment response, a treatment toxicity, a disease progression, a disease remission, or a genetic condition. Morphological features of cells may include size, shape, nuclear features, nuclear-to-cytoplasmic ratio, presence of inclusions, granulation patterns, chromatin patterns, and/or other shape-based and structure-based descriptors derivable from the images. Staining characteristics of cells may include color intensity, color distribution, color ratios between different cellular components, staining artifacts, and/or other color-based descriptors. Spatial distribution of cells may include clustering patterns, density variations, positional relationships between cell types, distribution across smear regions (such as feathered edge, body, or monolayer zones), and/or distribution anomalies. Aggregated values computed over cells may include statistical summaries, histograms, population-level metrics, and/or composite scores computed over subsets of cells or over all detected cells. The biomarker may be computed from any combination of these data categories. Utilizing data extracted from the images beyond the classifications may permit biomarkers that capture features the automatic cell classifier does not explicitly report, such as morphological or staining features that are informative independently of cell type assignment. The determining of the first and second subsets may be based on the biomarker alone, on the biomarker in combination with other criteria, or on any other combination that incorporates the biomarker.

In some implementations of the method, the first subset comprises less than 5% of the multiple samples, and the second subset comprises more than 95% of the multiple samples; and wherein the DSS performs, on the images presented to the human reviewer utilizing the DSS, at least one of cell detection, cell classification, cell measurement, or cell segmentation, without utilizing the classifications for cells for which the classifications are withheld. The triage applied by the automatic cell classifier yields a first subset comprising less than 5% of the multiple samples and a second subset comprising more than 95% of the multiple samples, which may be suited to deployments where the automatic cell classifier exhibits high autonomy over routine cases and routes only a small proportion of cases for human review utilizing the DSS. In addition, the DSS performs, on the images presented to the human reviewer utilizing the DSS, at least one of cell detection, cell classification, cell measurement, or cell segmentation, and does so without utilizing the classifications produced by the automatic cell classifier for cells for which the classifications are withheld. The DSS may thereby produce an independent analytical output for the presented images, which may serve as a second-opinion analysis, may be compared against the outputs of the automatic cell classifier in post-hoc quality-assurance processes, and/or may be utilized by the human reviewer as a starting point for interactive review. The analytical operations performed by the DSS may be implemented by algorithms including, by way of non-limiting example, convolutional neural networks for detection and classification, instance or semantic segmentation models, morphological operators, color analysis algorithms, and/or rule-based analysis modules. Because these operations are performed without utilizing the withheld classifications, the DSS outputs may be generated independently of any influence from the automatic cell classifier's assignments for those cells, which may support the independence goals described for the main claim.

In some implementations of the method, the first subset comprises less than 5% of the multiple samples, and the second subset comprises more than 95% of the multiple samples; and wherein the images presented to the human reviewer utilizing the DSS are acquired utilizing at least one imaging parameter different from a corresponding imaging parameter utilized for acquiring at least some of the multiple images analyzed by the automatic cell classifier. The triage applied by the automatic cell classifier yields a first subset comprising less than 5% of the multiple samples and a second subset comprising more than 95% of the multiple samples, and the images presented to the human reviewer utilizing the DSS are acquired utilizing at least one imaging parameter that differs from a corresponding imaging parameter utilized for the initial acquisition. The differing imaging parameter may include, by way of non-limiting example, a higher or lower ENA, a different magnification, a different field of view, a different focus strategy (for example, acquisition of a z-stack of images at multiple focal planes for samples in the first subset, as compared with single-plane acquisition for the initial scan), different illumination conditions (such as additional illumination wavelengths, different illumination angles, or use of phase-contrast or differential interference contrast as opposed to brightfield), different spectral characteristics, different exposure times, different gain settings, or different computational reconstruction methods. The differing imaging parameter may be selected to produce images more suitable for human review and/or for analysis performed by the DSS. Applying the differing imaging parameter selectively to the first subset may reduce the time and resource cost that would be incurred if the differing parameter were applied to all of the multiple samples, and may be compatible with the low-percentage triage defined in the claim. The acquisition of images with the differing imaging parameter may be performed in a re-scanning operation, a new-smear scanning operation, or any other arrangement that associates differing imaging parameters with the images presented utilizing the DSS. In some implementations of the method, a non-transitory computer-readable medium comprising instructions that, when executed by a processor, cause the processor to perform the computer-implemented method.

In various implementations, a system for preparing images for human review, comprising: a computer configured to: receive, from an automatic cell classifier, classifications of cells in multiple images of multiple cytological smears, wherein each of the multiple cytological smears is associated with one of multiple samples, and wherein the multiple samples are obtained from multiple patients; determine, based on the classifications, that a first subset of the multiple samples is to be presented as part of default human review workflow, and that a second subset of the multiple samples is not to be presented as part of default human review workflow; wherein the first subset comprises less than 10% of the multiple samples, and the second subset comprises more than 90% of the multiple samples; and present images of cytological smears to a human reviewer utilizing a decision support system (DSS), wherein the images presented utilizing the DSS are of cytological smears associated with samples in the first subset, and wherein the DSS is not provided with classifications produced by the automatic cell classifier for all cells in the images presented utilizing the DSS. FIG. 12C illustrates an embodiment of a block diagram of a system for preparing images for human review utilizing a DSS. The system includes a computer configured to receive classifications from an automatic cell classifier, determine first and second subsets of multiple samples based on the classifications, and present images of cytological smears associated with samples in the first subset to a human reviewer utilizing a DSS that is not provided, for all cells in the presented images, with the classifications. The computer 345 may be communicatively coupled to the automatic cell classifier 344, to an imaging system, and to the DSS 346, and may be implemented as any suitable computing arrangement described throughout this disclosure.

In some implementations of the system, the images presented utilizing the DSS are selected from the multiple images. The computer may select, from the multiple images previously analyzed by the automatic cell classifier, the images corresponding to cytological smears associated with samples in the first subset, and provide the selected images to the DSS.

In some implementations of the system, the images presented utilizing the DSS are acquired, after determining that the first subset is to be presented as part of default human review workflow, by at least one of: re-scanning one or more of the cytological smears associated with samples in the first subset, or scanning one or more new cytological smears prepared from samples in the first subset. The computer may command an imaging system to re-scan cytological smears associated with samples in the first subset and/or to scan new cytological smears prepared from samples in the first subset after the first subset is determined, and may provide the resulting images to the DSS.

In some implementations of the system, the acquiring of the images presented utilizing the DSS is performed by an imaging system having an effective numerical aperture (ENA) higher than an ENA of an imaging system that acquired the multiple images. The computer may coordinate imaging operations across multiple imaging systems of different ENAs, with the imaging system of higher ENA utilized for the acquiring of images of cytological smears associated with samples in the first subset.

In some implementations of the system, the DSS is configured to operate interactively with the human reviewer, such that at least some outputs of the DSS depend on input received from the human reviewer, and wherein the automatic cell classifier produces the classifications without input from the human reviewer. The computer may interface with a DSS implemented as an interactive system whose outputs depend on human reviewer input, as distinguished from the automatic cell classifier which operates autonomously.

In some implementations of the system, the DSS is configured to perform image analysis on the images presented to the human reviewer utilizing the DSS, the image analysis comprising at least one of: cell detection, cell classification, cell measurement, cell segmentation, cell counting, analysis of cell morphology, analysis of cell staining characteristics, or analysis of spatial distribution of cells; and wherein the input received from the human reviewer comprises at least one of: an annotation applied to at least one of the images, a selection of a cell or an area within at least one of the images, a confirmation or correction of an analysis result produced by the DSS, a classification provided by the human reviewer for a cell or a group of cells, a measurement provided by the human reviewer, a query requesting additional information, a text input, or a voice input. The DSS may perform any combination of the enumerated image analyses and may accept any combination of the enumerated interaction modes from the human reviewer.

In some implementations of the system, the computer, when presenting the images to the human reviewer utilizing the DSS, is configured to provide the DSS with the images without any output of the automatic cell classifier. The computer may isolate its data output stream to the DSS such that no output of the automatic cell classifier reaches the DSS.

In some implementations of the system, the images presented to the human reviewer utilizing the DSS comprise at least a portion of the multiple images, and wherein the computer, when presenting the images to the human reviewer utilizing the DSS, is configured to provide the DSS with the at least a portion of the multiple images and with data indicative of locations of cells detected by the automatic cell classifier in the at least a portion of the multiple images, and wherein the classifications of cells at the locations are withheld from the DSS. The computer may extract and forward location data (such as bounding boxes or coordinate lists) derived from the classifier's analysis of the multiple images while filtering out the classifications associated with the same locations.

In some implementations of the system, the DSS is not provided with at least one of: (i) classification confidence levels (CCLs) associated with the classifications of cells for which the classifications are withheld; (ii) hematological cell or cell type quantification metrics (HCQMs) derived from the classifications of cells associated with samples in the first subset; (iii) aggregated analysis results derived from the classifications of cells associated with samples in the first subset; or (iv) any indication of a basis on which the first subset was determined to be presented as part of default human review workflow. The computer may be configured, through data-filtering and routing logic, to withhold any combination of CCLs, HCQMs, aggregated analysis results, and/or basis indicators from the DSS.

In some implementations of the system, the DSS not being provided with the classifications for all cells in the images presented utilizing the DSS is implemented by at least one of: not transmitting the classifications to the DSS, not storing the classifications in a storage accessible to the DSS, excluding the classifications from data sent to the DSS, removing the classifications from metadata associated with the images before the images are provided to the DSS, configuring the DSS to disregard the classifications when present in data received by the DSS, or configuring the DSS to refrain from applying the automatic cell classifier to the images presented utilizing the DSS. The computer may implement the withholding through any combination of transmission control, access control, data exclusion, metadata stripping, DSS configuration, and/or restriction on applying the automatic cell classifier within the DSS.

In some implementations of the system, the computer is further configured to, responsive to receiving from the human reviewer an indication identifying a specific cell in one of the images presented to the human reviewer utilizing the DSS, provide the human reviewer with the classification of the specific cell. The computer may provide the classification of the specific cell to the human reviewer upon receiving a targeted indication, while otherwise maintaining the withholding from the DSS for cells not specifically indicated.

In some implementations of the system, the computer is further configured to, responsive to receiving from the human reviewer an indication identifying a specific area within one of the images presented to the human reviewer utilizing the DSS, provide the human reviewer with classifications of cells in the specific area. The computer may provide the classifications of cells located within the specific area upon receiving an area-based indication, while otherwise maintaining the withholding from the DSS for cells outside the specific area.

In some implementations of the system, the computer is configured to determine the first subset and the second subset further based on a biomarker derived from the classifications, wherein the biomarker is correlated with a condition of one of the multiple patients, and wherein the condition comprises at least one of: a hematological malignancy, an infection, an inflammation, an anemia, a treatment response, a treatment toxicity, a disease progression, a disease remission, or a genetic condition. The computer may compute a biomarker from the classifications and utilize the biomarker in determining the first and second subsets, based on correlation with the enumerated conditions.

In some implementations of the system, the computer is configured to determine the first subset and the second subset further based on a biomarker derived from data extracted from the multiple images, wherein the biomarker is correlated with a condition of one of the multiple patients, wherein the data comprises at least one of: the classifications, morphological features of cells detected in the multiple images, staining characteristics of cells detected in the multiple images, spatial distribution of cells detected in the multiple images, or aggregated values computed over cells detected in the multiple images; and wherein the condition comprises at least one of: a hematological malignancy, an infection, an inflammation, an anemia, a treatment response, a treatment toxicity, a disease progression, a disease remission, or a genetic condition. The computer may compute a biomarker from broader image-extracted data, including the classifications and/or other data categories, and utilize the biomarker in determining the first and second subsets.

In some implementations of the system, the first subset comprises less than 5% of the multiple samples, and the second subset comprises more than 95% of the multiple samples; and wherein the DSS performs, on the images presented to the human reviewer utilizing the DSS, at least one of cell detection, cell classification, cell measurement, or cell segmentation, without utilizing the classifications for cells for which the classifications are withheld. The computer may apply narrower triage percentages and interface with a DSS that performs one or more independent analytical operations on the presented images without utilizing the withheld classifications.

In some implementations of the system, the first subset comprises less than 5% of the multiple samples, and the second subset comprises more than 95% of the multiple samples; and wherein the images presented to the human reviewer utilizing the DSS are acquired utilizing at least one imaging parameter different from a corresponding imaging parameter utilized for acquiring at least some of the multiple images analyzed by the automatic cell classifier. The computer may apply narrower triage percentages and coordinate acquisition of images for samples in the first subset with at least one imaging parameter differing from the corresponding parameter used for the initial acquisition.

The following implementation addresses the optimization of classification or decision thresholds based on various contextual factors and their impact on human review decisions. By dynamically adjusting thresholds rather than using fixed values, the system may determine with greater precision which cells or samples require human expert attention, which may increase overall system efficiency by intelligently allocating human review resources based on contextually-appropriate confidence levels rather than static cutoffs. The embodiment supports a more personalized review process that considers the specific characteristics of the scenarios, potentially reducing unnecessary reviews while enhancing the likelihood of clinically significant findings to receive appropriate expert validation. The dynamic nature of these thresholds may adapt to changing conditions, emerging patterns, and varying clinical requirements, creating a more responsive and adaptive cytological analysis workflow.

For example, the system may initially use standard thresholds for blast cells in all or most patients, and over time may recognize that patients with certain medication profiles consistently show slightly different patterns that require adjusted thresholds for optimal classification. Rather than waiting for these patterns to be formally documented in medical literature, the dynamic threshold system can adapt to these emerging patterns as they're detected. This capability is useful in hematology where subtle changes in cell morphology or distribution are associated with significant diagnostic implications. The system does not just apply static knowledge but can evolve its threshold adjustment as new patterns emerge from ongoing clinical practice and analysis, where some changes may be applied automatically while other may require human confirmation.

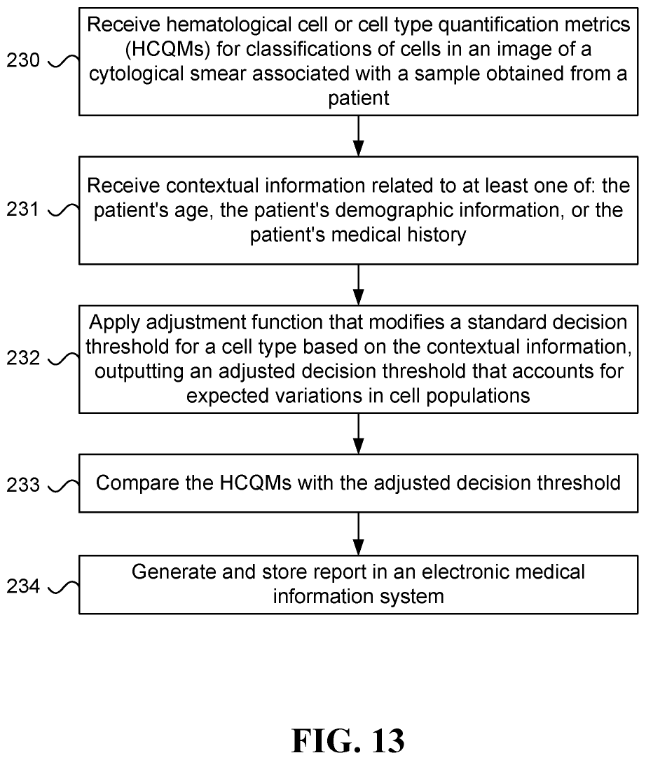

In various implementations, a computer-implemented method for threshold adjustment according to contextual information, comprising: receiving hematological cell or cell type quantification metrics (HCQMs) for classifications of cells in an image of a cytological smear associated with a sample obtained from a patient; receiving contextual information related to at least one of: the patient's age, the patient's demographic information, or the patient's medical history; applying an adjustment function that modifies a standard decision threshold for a cell type based on the contextual information, wherein the adjustment function outputs an adjusted decision threshold that accounts for expected variations in cell populations specific to the patient's context; comparing the HCQMs with the adjusted decision threshold; and generating and storing a report in an electronic medical information system, wherein the report presents analysis results according to the adjusted decision threshold. FIG. 13 illustrates an embodiment of a computer-implemented method for threshold adjustment according to contextual information. The method includes the following steps. In step 230, receive hematological cell or cell type quantification metrics (HCQMs) for classifications of cells in an image of a cytological smear associated with a sample obtained from a patient. In step 231, receive contextual information related to at least one of: the patient's age, the patient's demographic information, or the patient's medical history. In step 232, apply adjustment function that modifies a standard decision threshold for a cell type based on the contextual information, wherein the adjustment function outputs an adjusted decision threshold that accounts for expected variations in cell populations specific to the patient's context. In step 233, compare the HCQMs with adjusted decision threshold. And in step 234, generate and store report in an electronic medical information system, wherein the report presents analysis results according to the adjusted decision threshold.

The system may implement threshold adjustment mechanisms that modify standard decision thresholds based on contextual information specific to individual patients. By receiving hematological cell or cell type quantification metrics (HCQMs) and relevant contextual information such as patient age, demographic information, or medical history, the system may apply adjustment functions that account for expected variations in cell populations associated with these contextual factors. These adjustment functions may transform standard thresholds into patient-specific adjusted decision thresholds, potentially improving classification accuracy by incorporating individual patient context. The comparison between HCQMs and these adjusted thresholds may produce analysis results that better reflect the patient's specific physiological state rather than applying population-wide standards uniformly. The report generated and stored in the electronic medical information system may present these contextualized analysis results, potentially enabling more personalized interpretation of cytological findings. This approach may recognize that what constitutes “normal” or “abnormal” cellular presentations can vary significantly based on patient-specific factors, which may help improve diagnostic precision and reduce both false positives and false negatives that might otherwise occur when applying standardized thresholds without contextual adjustment.

Optionally, the standard decision thresholds and the contextual information utilized by the system may be derived from a variety of sources and may possess different characteristics. For example, a standard decision threshold may be a fixed value established by clinical guidelines, regulatory bodies, or laboratory-specific validation studies. In other embodiments, the threshold may be dynamic, possibly adjusting over time based on system learning, population statistics, or other performance metrics. Similarly, the contextual information may be received from various integrated systems, such as a Laboratory Information System (LIS) or an Electronic Health Record (EHR), and may be processed in real-time or retrieved from a stored patient profile. The adjustment function itself may be a simple mathematical formula, a set of rule-based conditions, or a more complex machine learning model trained to correlate specific contextual factors with expected hematological variations. Optionally, the cytological smear may be prepared by various entities and at various times relative to the analysis. For example, the smear may be prepared by automated machinery, by laboratory personnel such as a technician, or through a combination of automated and manual processes. The preparation may occur at the same facility where the analysis is performed, or may take place at a separate location with the prepared smear subsequently being transported to the analysis facility.

In some implementations of the method, the report indicates cellular abnormalities based on the comparison of the HCQMs with the adjusted decision threshold. The report may include specific indications of cellular abnormalities identified through the comparison of HCQMs with the adjusted decision thresholds. These indications may highlight cell populations or characteristics that deviate from the patient-specific expected ranges established by the adjusted thresholds. By incorporating these abnormality indications within the report, the system may provide focused attention on findings that warrant clinical consideration within the context of the patient's specific profile. The abnormality indications may take various forms, such as highlighted text, color-coded indicators, numerical deviations from expected ranges, or graphical representations that visualize the degree of deviation from adjusted thresholds. This contextual presentation of abnormalities may help physicians interpret results in light of relevant patient factors rather than against standardized thresholds that may not account for the patient's unique circumstances.

In some implementations, the method further comprises upon identifying that the HCQMs cross the adjusted decision threshold, presenting, as part of default human review workflow, cells associated with the sample. When HCQMs cross the adjusted decision thresholds, the system may automatically present cells associated with the sample as part of a default human review workflow. This threshold-crossing trigger may create an intelligent routing mechanism that directs human attention to cases where contextually adjusted thresholds indicate potential abnormalities requiring expert assessment. The presentation may include relevant cells from the cytological smear, potentially focusing on the specific cell populations related to the threshold crossing. By implementing this contextually aware routing approach, the system may optimize human reviewer resources by prioritizing cases where the adjusted thresholds suggest findings that warrant expert examination. The threshold crossing determination may incorporate various mathematical comparisons, such as greater than, less than, equality testing, or range evaluation, depending on the specific cell population and clinical significance of different deviation types. Cells associated with the sample, may refer to any cells associated with the sample, may include cells classified according to any classification, and may relate to any image or any cytological smear.

In some implementations, the method further comprises receiving cell classifications from an automatic cell classifier and determining a subset of cells to be presented for human review based on the comparison of the HCQMs with the adjusted decision threshold. The system may implement intelligent subset selection mechanisms that determine which specific cells should be presented for human review based on the comparison of HCQMs with adjusted decision thresholds. By receiving classifications from an automatic cell classifier and applying the contextually adjusted thresholds, the system may identify particular cells or cell groups that warrant expert attention. This subset selection process may optimize reviewer efficiency by directing focus to the most relevant cellular elements rather than requiring examination of the entire cytological smear. The selection criteria may incorporate multiple factors including the magnitude of deviation from adjusted thresholds, the clinical significance of the specific cell types showing deviations, and pattern recognition across multiple related cell populations. This focused presentation approach may create more efficient review workflows while maintaining diagnostic thoroughness by ensuring that cells most likely to have clinical significance in the patient's specific context receive appropriate expert examination.

In some implementations, the method further comprises generating a presentation of the subset of cells, wherein the presentation incorporates information about the adjusted decision threshold to provide context for the human review. The presentation of the selected subset of cells may incorporate information about the adjusted decision thresholds, providing important context for human reviewers. This contextual presentation may include visualization of threshold values, indications of how thresholds were adjusted from standard values, and representations of how specific cells relate to these patient-specific thresholds. By providing this threshold information alongside the cellular images, the system may create a more informed review environment that helps reviewers understand why particular cells were selected for examination and how their characteristics relate to expected values for the specific patient. The presentation may utilize various visualization techniques such as reference lines indicating threshold values, color gradients reflecting proximity to thresholds, or numerical indicators showing quantitative relationships between measured values and adjusted thresholds. This contextually enhanced presentation may improve reviewer understanding of findings within the patient's specific profile, potentially leading to more accurate interpretations.

In some implementations of the method, the generating of the presentation comprises including information about the contextual information that influenced the adjusted decision threshold, providing reviewers with insight into why particular cells are presented for review. The presentation may include information about the specific contextual factors that influenced the threshold adjustments, providing reviewers with insight into why particular cells are presented for review. This contextual information may include relevant patient factors such as age-related reference ranges, demographic-specific expectations, medication effects, or condition-related baseline shifts that contributed to threshold adjustments. By presenting this explanatory information alongside the selected cells, the system may help reviewers interpret findings in the appropriate clinical context. The contextual factor presentation may take various forms, such as explanatory text annotations, graphical representations of factor influence, comparison views showing standard versus adjusted thresholds, or highlighting of specific factors most relevant to the particular case. This approach may provide reviewers with a more comprehensive understanding of how patient-specific factors influenced the analysis, potentially improving the quality and relevance of their expert assessment.

In some implementations of the method, the generating of the presentation comprises visualizing a degree to which cell types deviate from the adjusted decision threshold, wherein the visualization differentially emphasizes cell types with HCQM values relatively near the adjusted decision threshold and cell types with HCQM values relatively far from the adjusted decision threshold, with different types of deviation highlighted using distinct visual indicators. The presentation may implement visual emphasis techniques that differentiate cell types based on their proximity to adjusted decision thresholds. Cell types with HCQM values relatively near the adjusted thresholds may receive distinct visual treatment compared to cell types with values farther from thresholds, creating an intuitive visualization of borderline cases and workflow optimization by drawing attention to the important cell types first. This differential emphasis may utilize various visual indicators such as order selection, color intensity gradations, size variations, border treatments, or opacity adjustments to communicate threshold proximity relationships. The visualization may also employ distinct indicators for different deviation types, such as using different colors or symbols to distinguish between cell types exceeding upper thresholds versus cell types falling below lower thresholds. This threshold-proximity visualization may help reviewers quickly identify cases that represent borderline classifications where expert judgment adds the most value, potentially improving review efficiency while maintaining focus on clinically significant edge cases.

In some implementations of the method, the generating of the presentation comprises creating parallel views showing analysis results using both a standard decision threshold and the adjusted decision threshold, enabling human reviewers to understand an impact of the adjusted decision threshold on classification outcomes. The presentation may incorporate parallel views that simultaneously display analysis results using both standard decision thresholds and the adjusted decision thresholds. This comparative visualization approach may enable reviewers to understand how threshold adjustments impact diagnostic outcomes for the specific patient. The parallel views may include side-by-side cellular presentations, overlaid threshold indicators with distinct visual coding for standard versus adjusted thresholds, or toggle capabilities that allow reviewers to switch between standard and adjusted threshold views. By providing this comparative perspective, the system may help reviewers appreciate the significance of the contextual adjustments and how they affect the interpretation of findings. The parallel presentation may include quantitative indicators showing the magnitude of threshold shifts and the resulting changes in outcomes, potentially highlighting cases where threshold adjustments significantly alter clinical interpretations. This comparative approach may provide valuable insight into how patient-specific factors influence cytological analysis results, potentially improving diagnostic precision.

In some implementations of the method, the contextual information comprises the patient's age, and wherein the adjustment function increases lymphocyte count thresholds for younger patients to account for age-related variations in normal lymphocyte populations. The adjustment function may modify lymphocyte count thresholds based on the patient's age, recognizing that normal lymphocyte populations typically vary significantly across age groups. For babies, the function may increase lymphocyte count thresholds to account for the typically higher baseline lymphocyte levels observed in pediatric populations. These age-based adjustments may incorporate various mathematical transformations including linear scaling, curve-fitted adjustments based on age distribution data, or discrete threshold shifts for defined age brackets. The specific adjustment magnitudes may be derived from population studies, clinical reference ranges, or statistical models that characterize age-related hematological variations. By implementing these age-aware threshold adjustments, the system may reduce false positive flags for younger patients with physiologically appropriate elevated lymphocyte counts while maintaining appropriate sensitivity for detecting significant lymphocytosis in older patients.

In some implementations of the method, the contextual information comprises the patient's medical history, and wherein the adjustment function modifies the standard decision threshold based on at least one of: previous cytological analysis results for the patient, medication prescribed to the patient, radiation therapy received by the patient, or chemotherapy received by the patient. The adjustment function may modify standard decision thresholds based on the patient's medical history, including previous cytological analysis results, medication regimens, radiation therapy history, or chemotherapy treatments. These medical history factors may significantly influence expected cell population baselines and normal ranges for individual patients. For example, patients with documented baseline abnormalities from previous analyses may receive adjusted thresholds that account for their established patterns. Patients receiving medications known to affect specific cell populations may have thresholds modified to account for these expected pharmacological effects. Similarly, patients undergoing or recovering from radiation or chemotherapy may receive threshold adjustments that reflect the expected hematological impacts of these interventions. The medical history-based adjustments may incorporate temporal factors that account for the recency of treatments or interventions, with more significant adjustments applied to recent treatments and gradually decreasing adjustments as time elapses. By accounting for these medical history factors, the system may reduce false positive flags for expected treatment-related changes while maintaining sensitivity for detecting unexpected deviations that may indicate clinically significant developments.

In some implementations of the method, the patient's medical history indicates a chronic lymphocytic leukemia diagnosis, and wherein the adjustment function increases lymphocyte count thresholds to account for an expected elevated baseline lymphocyte level. For patients with documented chronic lymphocytic leukemia (CLL), the adjustment function may implement specialized threshold modifications that account for the expected elevated lymphocyte levels associated with this condition. The function may increase lymphocyte count thresholds to establish a new baseline that reflects the patient's specific disease state rather than applying standard reference ranges that would consistently flag the patient's expected lymphocytosis. The magnitude of this threshold adjustment may vary based on factors such as disease stage, treatment status, or established baseline patterns from the patient's previous cytological analyses. The adjusted thresholds may help distinguish between stable disease-related lymphocytosis and significant changes that may indicate disease progression or treatment response. This condition-specific adjustment approach may be applied to various hematological and non-hematological conditions that manifest with characteristic cellular presentations, with the specific threshold modifications tailored to the expected cellular patterns associated with each condition. By implementing these condition-aware threshold adjustments, the system may reduce unnecessary flagging of stable disease-related findings while maintaining sensitivity for detecting clinically significant changes within the context of the patient's known condition.

In some implementations of the method, the patient's medical history indicates active chemotherapy treatment, and wherein the adjustment function decreases neutrophil count thresholds to account for expected neutropenia associated with the chemotherapy. For patients undergoing active chemotherapy treatment, the adjustment function may implement specialized threshold modifications that account for the expected hematological effects of these interventions. The function may decrease neutrophil count thresholds to reflect the expected neutropenia commonly associated with many chemotherapy regimens, establishing a new evaluation framework that accounts for treatment-induced cellular changes rather than applying standard reference ranges. The magnitude of these threshold adjustments may vary based on factors such as the specific chemotherapy agents being used, the treatment cycle timing, cumulative dose effects, or individual patient response patterns established from previous cycles. Similar adjustments may be applied to other cell populations commonly affected by chemotherapy, such as modified thresholds for lymphocytes, platelets, or red blood cells. These treatment-aware adjustments may help distinguish between expected treatment-related effects and unexpected changes that may indicate complications requiring intervention. By implementing these treatment status-based threshold adjustments, the system may reduce unnecessary flagging of anticipated treatment effects while maintaining sensitivity for detecting clinically significant deviations that fall outside expected treatment response patterns.

In some implementations of the method, the contextual information comprises the patient's demographic information that includes at least one of: ethnicity, sex, or geographical location, and wherein the adjustment function modifies the standard decision threshold based on statistical distributions of cell populations observed within specific demographic groups. The adjustment function may modify standard decision thresholds based on the patient's demographic information, including ethnicity, sex, or geographical location. These demographic factors may influence expected baseline hematological profiles due to genetic, environmental, or lifestyle factors that vary across different demographic groups. The function may apply threshold adjustments derived from statistical distributions of cell populations observed within specific demographic categories, creating more relevant evaluation frameworks for individual patients. For example, ethnicity-specific adjustments may account for known variations in normal neutrophil ranges across different ethnic groups, while sex-specific adjustments may reflect differences in red blood cell parameters between male and female populations. Geographical adjustments may account for regional variations influenced by factors such as altitude, endemic disease exposure, or nutritional patterns. These demographic-based adjustments may incorporate various statistical approaches including group-specific reference ranges, mathematical transformations based on distribution analysis, or multifactor models that account for interactions between demographic variables. By implementing these demographically informed threshold adjustments, the system may reduce false flagging of normal variations associated with demographic factors while maintaining appropriate sensitivity for detecting clinically significant findings within the patient's relevant demographic context.

In some implementations of the method, the adjustment function applies different weighting factors to different components of the contextual information based on their relative clinical significance for the cell type being analyzed. The adjustment function may implement weighting mechanisms that apply different influence factors to various components of the contextual information based on their relative clinical significance for the specific cell type being analyzed. This weighted approach may recognize that certain contextual factors may have greater relevance for particular cell populations, creating a nuanced adjustment framework that prioritizes the most clinically relevant factors for each analysis target. The weighting system may incorporate various mathematical approaches including linear weighted combinations, multiplicative factor applications, conditional weighting based on value ranges, or machine learning-derived weight assignments that reflect relationships between contextual factors and cell parameters. The specific weighting configurations may vary across different cell types, with certain contextual factors receiving higher weights for some cell populations and lower weights for others based on established clinical relationships. For example, age-related factors may receive higher weighting for lymphocyte threshold adjustments, while medication factors may receive higher weighting for neutrophil threshold adjustments. By implementing this weighted approach to factor integration, the system may create more clinically relevant threshold adjustments that reflect the interplay between multiple contextual influences, potentially improving classification precision across diverse patient scenarios.

In some implementations of the method, the contextual information includes timing information related to sample collection relative to a medical treatment or intervention, and wherein the adjustment function modifies the standard decision threshold according to expected temporal variations in cellular profiles following the medical treatment or intervention. The adjustment function may modify standard decision thresholds based on timing information related to sample collection relative to medical treatments or interventions. This temporal context awareness may account for expected chronological variations in cellular profiles following treatments such as surgery, medication administration, transfusions, or other medical procedures. The function may implement time-dependent adjustment models that vary threshold modifications according to established temporal patterns of cellular response to specific interventions. For example, immediately following certain procedures, more significant threshold adjustments may be applied to account for acute responses, while gradually reducing these adjustments as time elapses and cellular patterns typically normalize. For cyclical treatments such as chemotherapy or dialysis, the function may apply cyclical adjustment patterns that align with expected nadir and recovery phases. These temporal adjustments may incorporate various mathematical approaches including decay functions that reduce adjustment magnitude over time, wave functions that model cyclical response patterns, or stepwise functions that apply discrete adjustment levels at different time intervals. By implementing these temporally aware threshold adjustments, the system may reduce false flagging of expected time-dependent changes while maintaining sensitivity for detecting deviations from typical temporal response patterns that may indicate complications or unexpected reactions.

In some implementations, the method further comprises presenting the report to a human reviewer, wherein the report includes information about how the contextual information influenced the adjustment function that modified the standard decision threshold. The system may present the generated report to human reviewers with explanatory information about how the contextual information influenced the adjustment function. This explanatory component may provide transparency into the decision-making process behind threshold modifications, potentially helping reviewers understand why particular adjustments were made for the specific patient. The report may include visualization of the standard decision thresholds alongside the adjusted decision thresholds, with annotations indicating which contextual factors contributed to significant modifications. For example, the report may highlight that age-related factors resulted in a 15% increase in lymphocyte thresholds, or that medication history led to modified neutrophil parameters. This transparency in threshold adjustment rationale may provide reviewers with important context for interpreting the analysis results, potentially improving their ability to make informed clinical judgments based on the patient's specific circumstances. The presentation format may include textual explanations, graphical representations of adjustment factors, or interactive elements that allow reviewers to explore the relationship between specific contextual factors and threshold modifications.

In some implementations, the method further comprises receiving feedback from the human reviewer about the adjusted decision threshold; and using the feedback to improve an algorithm for the adjustment function used to modify standard decision thresholds. The system may implement a feedback mechanism that captures reviewer assessments of threshold adjustment appropriateness and utilizes this input to refine future adjustment algorithms. After presenting the report with threshold adjustment rationale to human reviewers, the system may provide interfaces for reviewers to indicate whether the adjusted decision thresholds were clinically appropriate, too conservative, or too aggressive for the specific patient's context. This feedback may be collected through various methods including structured rating scales, binary approval indicators, or free-text comments about specific adjustment aspects. The system may then incorporate this feedback into algorithm improvement processes, potentially adjusting weighting factors for contextual elements, refining mathematical transformation functions, or modifying decision rules based on expert input. This continuous improvement cycle may help the system progressively align threshold adjustments with clinical expertise, potentially enhancing the system's ability to generate appropriate patient-specific thresholds over time. The feedback incorporation may utilize various approaches including statistical aggregation of reviewer responses, machine learning algorithms that identify patterns in feedback data, or rule-based modifications based on consensus reviewer suggestions.

In some implementations, the method further comprises determining whether to present the report for human review based on the comparison of the HCQMs with the adjusted decision threshold, wherein the determining applies more stringent review criteria for analysis results associated with higher clinical significance in the context of the patient's medical history; and presenting the report for human review when the determining indicates that human review is required. The system may implement context-sensitive review criteria that determine whether a report should be presented for human review based on the clinical significance of findings in the context of the patient's specific medical history. After generating the report with analysis results based on adjusted decision thresholds, the system may apply additional evaluation layers to determine human review requirements. For findings with higher clinical significance relative to the patient's specific medical context, the system may apply more stringent review criteria that increase the likelihood of human expert examination. For example, when the patient's medical history includes conditions that elevate the importance of certain cell parameters, even smaller deviations from adjusted thresholds for these parameters may trigger human review. Conversely, for findings with lower clinical significance in the patient's specific context, the system may apply more permissive review criteria that may not necessitate human review. When the determination indicates that human review is required, the system may present the report to appropriate reviewers through various interfaces or notification mechanisms. This context-sensitive approach may optimize reviewer resource allocation by focusing human expertise on findings with high potential clinical impact for each specific patient. The determination of clinical significance may incorporate multiple factors including the relationship between detected cell patterns and the patient's documented conditions, the potential implications of specific findings given the patient's treatment status, and the relevance of particular parameters to monitoring known disease processes in the patient's history.

In some implementations, the method further comprises determining whether to present the report for human review based on a magnitude of differences between the HCQMs and the adjusted decision threshold; determining a level of human reviewer expertise required based on the magnitude of differences, wherein larger differences require human reviewers with higher expertise levels in a corresponding cell type classification domain; and presenting the report to a human reviewer having the determined level of expertise when the determining indicates that human review is required. The system may implement expertise-matching algorithms that determine whether to present the report for human review and identify appropriate reviewer expertise levels based on the magnitude of deviations between the HCQMs and the adjusted decision thresholds. After generating the report with analysis results based on the adjusted thresholds, the system may evaluate the degree to which findings deviate from expected ranges to determine if human review is required and, if so, the corresponding expertise requirements for such review. Cases with larger deviations may present more complex analytical challenges or indicate potentially serious clinical issues that benefit from higher levels of specialist expertise. The system may implement graduated expertise matching, where progressively larger deviations trigger routing to reviewers with increasingly specialized knowledge in relevant domains. For example, moderate deviations in lymphocyte parameters might route to general laboratory professionals, while substantial deviations might route to hematopathologists with lymphocyte disorder specialization. When the determination indicates that human review is required, the system may present the report to a reviewer with the appropriate expertise level through various notification or workflow integration mechanisms. This magnitude-based expertise matching may help optimize the application of specialized knowledge by directing the most challenging or clinically significant cases to reviewers with the most relevant expertise. The expertise determination may incorporate various approaches including tiered reviewer categorization, expertise domain mapping that connects cell types to specialist knowledge areas, or case complexity algorithms that consider both deviation magnitude and the specific parameters involved.

In some implementations, the method further comprises computing a review necessity score based on both the adjusted decision threshold and classification confidence levels associated with the HCQMs; determining whether to present the report for human review based on the review necessity score; and presenting the report for human review when the review necessity score exceeds a predetermined review threshold, wherein the review necessity score further determines when and to whom the report is presented. The system may compute a review necessity score that integrates multiple factors to determine whether the report should be presented for human review. This multifactorial score may combine information about how the HCQMs compare to the adjusted decision thresholds along with classification confidence levels that indicate the reliability of the underlying cell classifications. The review necessity score may be calculated through various mathematical approaches such as weighted combinations of deviation magnitudes and confidence metrics, probabilistic models that estimate error likelihood based on threshold proximity and confidence values, or machine learning algorithms trained to predict when human review adds significant value based on historical patterns. When the review necessity score exceeds a predetermined review threshold, the system may present the report for human review through appropriate channels or interfaces. Higher review necessity scores may trigger immediate human review presentation, while lower scores that still exceed the minimum review threshold may place the report in lower-priority review queues. The score may also influence reviewer selection by mapping score ranges to different expertise requirements and determine the timing of review presentation based on urgency assessments derived from the score. This integrated scoring approach may recognize that review requirements depend not only on whether findings cross adjusted thresholds but also on the confidence with which those findings were determined. For example, a borderline finding with low classification confidence may generate a higher review necessity score than the same borderline finding with high classification confidence. By incorporating both threshold comparisons and classification confidence into routing decisions, the system may optimize human review resource allocation based on where expert judgment adds the most value.

In some implementations, the method further comprises determining a subset of cells for presentation based on the comparison of the HCQMs with the adjusted decision threshold; presenting the subset of cells to a human reviewer; and visually highlighting cells differently based on whether their classification uncertainty is related to patient age factors, demographic information factors, or medical history factors that influenced the adjustment function. The system may implement differential visual highlighting that distinguishes cells based on the specific contextual factors that most influenced their classification uncertainty. After determining a subset of cells for presentation based on the comparison between HCQMs and adjusted decision thresholds, the system may present these cells to human reviewers with visual encoding that communicates the source of uncertainty. Cells whose classification uncertainty primarily relates to patient age factors may receive distinct visual treatment (such as specific border colors or patterns) compared to cells whose uncertainty stems primarily from demographic information factors (which might receive different highlighting schemes) or medical history factors (which might be highlighted using yet another visual encoding). This source-differentiated highlighting may help reviewers more efficiently understand the nature of classification challenges for specific cells and apply their expertise more effectively based on the underlying contextual factors. The visual differentiation may be implemented through various techniques including color coding, border treatments, background shading, symbolic annotations, or interactive elements that reveal factor influence details on demand. This approach may enhance review efficiency by communicating not only which cells warrant attention but also why they warrant attention from a contextual factor perspective, potentially enabling more targeted and informed expert assessment.

In some implementations, the method further comprises determining a subset of cells for presentation based on the comparison of the HCQMs with the adjusted decision threshold; presenting the subset of cells to a human reviewer; and annotating the presented cells with context-specific information that explains how the adjusted decision threshold affected their classification confidence level or clinical significance assessment. The system may implement context-specific annotation mechanisms that provide explanatory information about how adjusted decision thresholds influenced classification outcomes for specific cells. After determining a subset of cells for presentation based on the comparison between HCQMs and adjusted decision thresholds, the system may annotate these cells with explanatory text or visual indicators that communicate the relationship between threshold adjustments and classification parameters. These annotations may include explanations of how classification confidence levels were affected by threshold adjustments, such as indicating that a cell's confidence level was marginal under standard thresholds but became more definitive under adjusted thresholds based on the patient's age. The annotations may also explain changes in clinical significance assessment, such as noting that a particular cell population would be considered abnormal under standard thresholds but falls within expected ranges when accounting for the patient's medication history. These explanatory annotations may provide reviewers with important context for interpreting findings, potentially enhancing their understanding of how patient-specific factors influence classification outcomes. The annotation format may include hover-activated information displays, linked explanation panels, embedded textual notes, or graphical indicators that represent the nature and magnitude of threshold adjustment effects on specific cells or cell populations. In some implementations of the method, a non-transitory computer-readable medium comprising instructions that, when executed by a processor, cause the processor to perform the computer-implemented method.

In various implementations, a system for threshold adjustment according to contextual information, comprising: a computer configured to: receive hematological cell or cell type quantification metrics (HCQMs) for classifications of cells in an image of a cytological smear associated with a sample obtained from a patient; receive contextual information related to at least one of: the patient's age, the patient's demographic information, or the patient's medical history; apply an adjustment function that modifies a standard decision threshold for a cell type based on the contextual information, wherein the adjustment function outputs an adjusted decision threshold that accounts for expected variations in cell populations specific to the patient's context; compare the HCQMs with the adjusted decision threshold; and generate and store a report in an electronic medical information system, wherein the report presents analysis results according to the adjusted decision threshold.

In some implementations of the system, the report indicates cellular abnormalities based on the comparison of the HCQMs with the adjusted decision threshold. In some implementations of the system, the computer is further configured to, responsive to identifying that the HCQMs cross the adjusted decision threshold, cause cells associated with the sample to be presented as part of default human review workflow.

In some implementations of the system, the computer is further configured to receive cell classifications from an automatic cell classifier and determine a subset of cells to be presented for human review based on the comparison of the HCQMs with the adjusted decision threshold. In some implementations of the system, the computer is further configured to generate a presentation of the subset of cells, wherein the presentation incorporates information about the adjusted decision threshold to provide context for the human review. In some implementations of the system, the generating of the presentation comprises including information about the contextual information that influenced the adjusted decision threshold, providing reviewers with insight into why particular cells are presented for review. In some implementations of the system, the generating of the presentation comprises visualizing a degree to which cell types deviate from the adjusted decision threshold, wherein the visualization differentially emphasizes cell types with HCQM values relatively near the adjusted decision threshold and cell types with HCQM values relatively far from the adjusted decision threshold, with different types of deviation highlighted using distinct visual indicators. In some implementations of the system, the generating of the presentation comprises creating parallel views showing analysis results using both a standard decision threshold and the adjusted decision threshold, enabling human reviewers to understand an impact of the adjusted decision threshold on classification outcomes. In some implementations of the system, the contextual information comprises the patient's age, and wherein the adjustment function increases lymphocyte count thresholds for younger patients to account for age-related variations in normal lymphocyte populations.

In some implementations of the system, the contextual information comprises the patient's medical history, and wherein the adjustment function modifies the standard decision threshold based on at least one of: previous cytological analysis results for the patient, medication prescribed to the patient, radiation therapy received by the patient, or chemotherapy received by the patient. In some implementations of the system, the patient's medical history indicates a chronic lymphocytic leukemia diagnosis, and wherein the adjustment function increases lymphocyte count thresholds to account for an expected elevated baseline lymphocyte level. In some implementations of the system, the patient's medical history indicates active chemotherapy treatment, and wherein the adjustment function decreases neutrophil count thresholds to account for expected neutropenia associated with the chemotherapy.

In some implementations of the system, the contextual information comprises the patient's demographic information that includes at least one of: ethnicity, sex, or geographical location, and wherein the adjustment function modifies the standard decision threshold based on statistical distributions of cell populations observed within specific demographic groups. In some implementations of the system, the adjustment function applies different weighting factors to different components of the contextual information based on their relative clinical significance for the cell type being analyzed.

In some implementations of the system, the contextual information includes timing information related to sample collection relative to a medical treatment or intervention, and wherein the adjustment function modifies the standard decision threshold according to expected temporal variations in cellular profiles following the medical treatment or intervention. In some implementations of the system, the computer is further configured to cause the report to be presented to a human reviewer, wherein the report includes information about how the contextual information influenced the adjustment function that modified the standard decision threshold. In some implementations of the system, the computer is further configured to: receive feedback from the human reviewer about the adjusted decision threshold; and use the feedback to improve an algorithm for the adjustment function used to modify standard decision thresholds.

In some implementations of the system, the computer is further configured to: determine whether to present the report for human review based on the comparison of the HCQMs with the adjusted decision threshold, wherein the determining applies more stringent review criteria for analysis results associated with higher clinical significance in the context of the patient's medical history; and cause the report to be presented for human review when the determining indicates that human review is required.

In some implementations of the system, the computer is further configured to: determine whether to present the report for human review based on a magnitude of differences between the HCQMs and the adjusted decision threshold; determine a level of human reviewer expertise required based on the magnitude of differences, wherein larger differences require human reviewers with higher expertise levels in a corresponding cell type classification domain; and cause the report to be presented to a human reviewer having the determined level of expertise when the determining indicates that human review is required.

In some implementations of the system, the computer is further configured to: compute a review necessity score based on both the adjusted decision threshold and classification confidence levels associated with the HCQMs; determine whether to present the report for human review based on the review necessity score; and cause the report to be presented for human review when the review necessity score exceeds a predetermined review threshold, wherein the review necessity score further determines when and to whom the report is presented.

In some implementations of the system, the computer is further configured to: determine a subset of cells for presentation based on the comparison of the HCQMs with the adjusted decision threshold; cause the subset of cells to be presented to a human reviewer; and visually highlight cells differently based on whether their classification uncertainty is related to patient age factors, demographic information factors, or medical history factors that influenced the adjustment function.

In some implementations of the system, the computer is further configured to: determine a subset of cells for presentation based on the comparison of the HCQMs with the adjusted decision threshold; cause the subset of cells to be presented to a human reviewer; and annotate the presented cells with context-specific information that explains how the adjusted decision threshold affected their classification confidence level or clinical significance assessment.

The following implementation addresses the challenge of making classification decisions when multiple parameters individually fall below reporting thresholds but collectively may indicate a significant finding. By analyzing combinations of borderline parameters, the system may identify clinically relevant patterns that may be missed by single-parameter threshold analyses. It may improve detection of subtle but important findings that manifest across multiple measurements, and thus supports more comprehensive analysis by considering the relationships between different classification parameters.

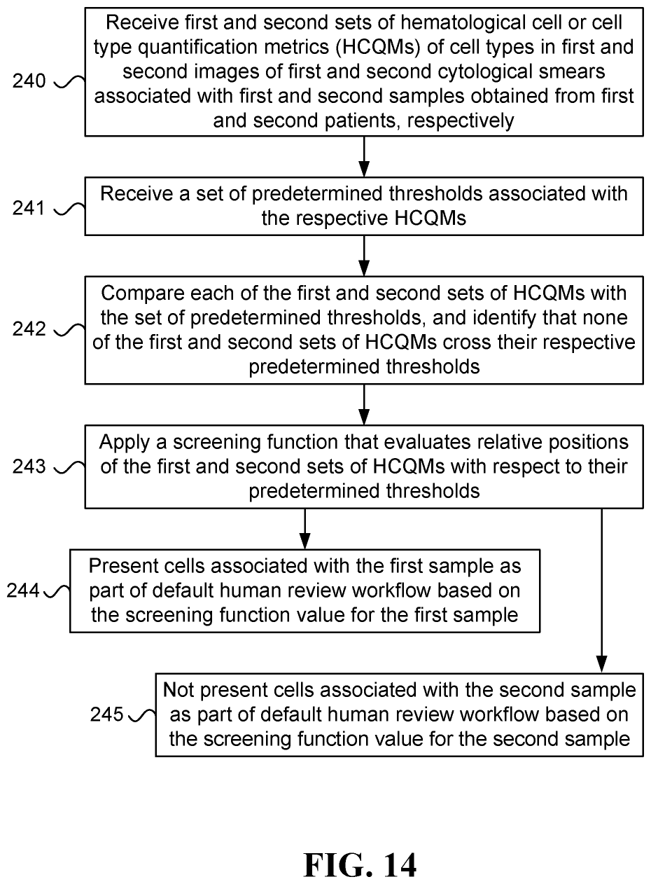

In various implementations, a computer-implemented method for analyzing combinations of parameters in cytological smears, comprising: receiving, first and second sets of hematological cell or cell type quantification metrics (HCQMs) of cell types in first and second images of first and second cytological smears associated with first and second samples obtained from first and second patients, respectively; receiving a set of predetermined thresholds associated with the respective HCQMs; comparing each of the first and second sets of HCQMs with the set of predetermined thresholds, and identifying that none of the first and second sets of HCQMs cross their respective predetermined thresholds; applying a screening function that evaluates relative positions of the first and second sets of HCQMs with respect to their predetermined thresholds; presenting, as part of default human review workflow, cells associated with the first sample based on a value of the screening function for the first sample; and not presenting, as part of default human review workflow, cells associated with the second sample based on a value of the screening function for the second sample. FIG. 14 illustrates an embodiment of a computer-implemented method for analyzing combinations of parameters in cytological smears. The method includes the following steps. In step 240, receive first and second sets of hematological cell or cell type quantification metrics (HCQMs) of cell types in first and second images of first and second cytological smears associated with first and second samples obtained from first and second patients, respectively. In step 241, receive a set of predetermined thresholds associated with the respective HCQMs. In step 242, compare, optionally by a processor, each of the first and second sets of HCQMs with the set of predetermined thresholds, and identify that none of the first and second sets of HCQMs cross their respective predetermined thresholds. In step 243, apply a screening function that evaluates relative positions of the first and second sets of HCQMs with respect to their predetermined thresholds. In step 244, present, as part of default human review workflow, cells associated with the first sample based on the screening function value for the first sample. And in step 245, not present, as part of default human review workflow, cells associated with the second sample based on the screening function value for the second sample.

Optionally, the HCQM is calculated based on classifications from the automatic cell classifier. In various embodiments, the system may implement parameter combination analysis to identify clinically significant patterns even when individual parameters remain within their respective thresholds. While some embodiments may trigger human review when individual parameters cross predetermined thresholds, this embodiment may recognize that combinations of parameters approaching but not crossing their thresholds can possess diagnostic significance. The screening function may evaluate the relative positions of multiple HCQMs with respect to their predetermined thresholds, potentially identifying cases where several parameters simultaneously approach their respective thresholds without individually exceeding them. This combinatorial approach may detect subtle patterns that could be missed by single-parameter threshold analysis. The screening function may calculate various metrics that quantify the collective behavior of multiple parameters, such as aggregate proximity scores, pattern recognition algorithms, or statistical distribution analyses. Based on the screening function value, the system may selectively route cases for human review, presenting cases with potentially significant parameter combinations while avoiding unnecessary reviews for cases where parameters are well within normal ranges. The HCQMs may be derived from various sources, including but not limited to automatic cell classifiers, image analysis algorithms, or computational analysis of cytological features. This approach may enhance the system's ability to identify borderline cases with potential clinical significance while maintaining efficient workflow by limiting human review to cases with the highest likelihood of clinical relevance. Cells associated with the sample, may refer to any cells associated with the sample, may include cells classified according to any classification, and may relate to any image or any cytological smear.

In the context of this embodiment, “not presenting, as part of default human review workflow, cells associated with the second sample” refers to excluding the second sample from the standard review process based on the value of the screening function for the second sample, even though none of the second set of HCQMs cross their respective predetermined thresholds. While the first sample is presented for human review because the screening function value for the first sample indicates that the relative positions of the first set of HCQMs with respect to their predetermined thresholds warrant human verification (such as when multiple HCQMs approach but do not cross their thresholds simultaneously), the second sample is not presented because the screening function value for the second sample indicates that the relative positions of the second set of HCQMs do not warrant human review (such as when the HCQMs remain well within normal ranges despite not crossing thresholds). This screening function-based routing recognizes that combinations of parameters approaching their thresholds may have clinical significance even when individual parameters remain within acceptable ranges, and the system selectively routes samples based on these collective patterns rather than individual threshold crossings. The not presenting may be implemented through various mechanisms including: excluding the second sample from review queues, routing the second sample directly to automated reporting pathways, preventing the second sample from appearing in reviewer worklists, marking the second sample with metadata flags indicating completion without human review requirement while marking the first sample with flags indicating review requirement, applying different marking schemes to the first and second samples that are distinguishable through visual indicators, textual labels, symbolic representations, or color variations, storing the second sample in repositories designated for completed analyses that bypassed human review based on their screening function values, presenting the screening function results and HCQM values for the second sample to a human without an indication that review is required whereas the screening function results and HCQM values for the first sample are presented with an indication that review is required, and/or displaying the second sample in a manner distinguishable from the first sample in the default human review workflow, wherein such distinguishing manners may include distinct borders, different background colors, modified interaction controls, presence or absence of review requirement indicators, different positioning within the interface hierarchy, alternative display formats, or textual annotations that identify the second sample as excluded from default human review workflow based on its screening function value. By not presenting the second sample while presenting the first sample based on screening function values, the system may detect and route for review subtle patterns requiring human expertise while avoiding unnecessary review of samples with HCQM patterns that remain within acceptable ranges. The system may maintain audit records indicating that the second sample was processed through automated pathways based on its screening function value falling below the routing threshold, while preserving the capability for the second sample to be accessed through review mechanisms outside the default workflow, such as when specifically requested by a physician, when flagged by subsequent quality assurance protocols that employ different screening criteria, when laboratory policies require periodic review of samples processed through automated pathways, when a reviewer indicates a desire to examine samples with specific screening function value ranges, when retrospective analysis protocols are initiated, and/or when clinical circumstances warrant examination of samples that initially bypassed human review based on their screening function evaluations not meeting the criteria for default human review workflow inclusion.

In some implementations of the method, the screening function comprises at least one of: (a) calculating distance metrics between each HCQM in the first and second sets and their respective predetermined thresholds; (b) applying different weights to different distance metrics based on clinical significance of the corresponding HCQMs; or (c) applying threshold proximity analysis that identifies samples with multiple HCQMs simultaneously approaching but not crossing their respective predetermined thresholds. The screening function may implement various approaches for quantifying the relationship between HCQMs and their respective thresholds. In one implementation, the system may calculate distance metrics that measure how close each HCQM is to its predetermined threshold, potentially using absolute differences, percentage differences, standard deviation units, or other mathematical distance formulations. These distance metrics may provide quantitative measures of threshold proximity that can be further analyzed for patterns across multiple parameters. In another implementation, the system may apply differential weighting to distance metrics based on the clinical significance of their corresponding HCQMs, potentially assigning higher weights to parameters with greater diagnostic importance or those associated with more severe conditions. This weighted approach may allow the system to prioritize clinically significant parameters while still considering the full range of available metrics. Additionally, the system may implement threshold proximity analysis that specifically identifies samples with multiple HCQMs simultaneously approaching their thresholds, which may indicate a coordinated physiological response or emerging pathological condition even when no individual parameter crosses its threshold. This multi-parameter proximity detection may capture subtle patterns that could be missed by single-parameter threshold analysis.

In some implementations of the method, the screening function comprises at least one of: (a) identifying patterns of simultaneous variations across multiple HCQMs, even when individual HCQMs do not cross their respective predetermined thresholds; (b) applying statistical distribution analysis to determine abnormal combinations of HCQMs; (c) applying statistical distribution analysis to determine clinical significant combinations of HCQMs; or (d) calculating correlation coefficients between pairs of HCQMs and comparing the correlation coefficients with expected correlation patterns. The screening function may implement various pattern recognition and statistical analysis approaches to identify clinically relevant HCQM combinations. In one implementation, the system may identify patterns of simultaneous variations across multiple HCQMs, potentially revealing coordinated changes that may indicate physiological responses or pathological conditions even when individual parameters remain within normal ranges. This pattern detection may utilize various algorithmic approaches including but not limited to time-series analysis, clustering techniques, or change-point detection methods. In another implementation, the system may apply statistical distribution analysis to determine abnormal or clinically significant HCQM combinations, potentially comparing observed parameter distributions against reference distributions from healthy populations or specific disease states. This analysis may identify samples where the constellation of parameters, while individually normal, collectively forms an unusual or clinically significant pattern. Additionally, the system may calculate correlation coefficients between pairs of HCQMs and compare these correlations with expected patterns, potentially identifying cases where the relationships between parameters deviate from typical patterns. These correlation analyses may reveal altered physiological relationships between cell populations or parameters that may signal underlying pathological processes even when the absolute values remain within normal ranges.

In some implementations of the method, the screening function comprises at least one of: (a) calculating a composite score by combining multiple HCQMs according to a predetermined formula; (b) employing a machine learning model trained to identify clinically significant combinations of HCQMs; or (c) incorporating confidence intervals associated with the HCQMs. The screening function may implement various computational approaches for evaluating combinations of HCQMs. In one implementation, the system may calculate composite scores by combining multiple HCQMs according to predetermined formulas, potentially including weighted sums, ratios, mathematical transformations, or other algorithmic combinations designed to capture relevant parameter relationships. These composite scores may distill complex multi-parameter information into more manageable numerical indicators of potential clinical significance. In another implementation, the system may employ machine learning models specifically trained to identify clinically significant HCQM combinations, potentially utilizing neural networks, random forests, support vector machines, or other machine learning architectures capable of discovering complex relationships in high-dimensional data. These models may be trained on datasets containing known outcomes, allowing them to learn subtle patterns associated with various clinical conditions. Additionally, the system may incorporate confidence intervals associated with the HCQMs, potentially accounting for measurement uncertainty, biological variability, or statistical reliability in the evaluation process. This approach may provide a more nuanced assessment by considering not just the parameter values themselves but also the degree of certainty associated with those measurements.

In some implementations of the method, the screening function comprises at least one of: (a) comparing the first and second sets of HCQMs with historical HCQMs associated with the first and second patients, respectively; (b) analyzing time-based trends in the HCQMs for each patient; or (c) calculating rate-of-change metrics for the HCQMs compared to previous measurements from the same patient. The screening function may implement various temporal analysis approaches that leverage historical patient data. In one implementation, the system may compare current HCQM sets with historical measurements from the same patients, potentially identifying deviations from established baselines that may indicate changing physiological conditions. This patient-specific comparative approach may account for individual variability in baseline parameters that might otherwise be misinterpreted within population-based reference ranges. In another implementation, the system may analyze time-based trends in HCQMs for each patient, potentially identifying gradual shifts or oscillatory patterns that may have clinical significance even when individual measurements remain within threshold limits. These trend analyses may utilize various time-series analytical techniques including but not limited to regression analysis, exponential smoothing, or frequency analysis. Additionally, the system may calculate rate-of-change metrics that quantify how rapidly parameters are changing compared to previous measurements, potentially identifying accelerating or decelerating trends that may indicate disease progression or response to treatment. By incorporating temporal dimensions into the analysis, the system may detect clinically significant patterns that might be missed in isolated evaluations of current measurements.

In some implementations of the method, the screening function applies different algorithms based on patient demographic information and/or medical history. The screening function may implement personalization features that tailor analysis based on patient-specific context. The system may apply different algorithms or parameter weightings based on patient demographic information such as age, sex, ethnicity, or geographic location, potentially accounting for known variations in hematological parameters across different demographic groups. For example, reference ranges and significance thresholds may differ for pediatric versus geriatric populations, or for patients from regions with endemic conditions affecting blood parameters. Similarly, the system may adapt its analytical approach based on medical history, potentially applying condition-specific algorithms for patients with known disorders that affect blood cell parameters. For instance, patients with documented hematological conditions, immune disorders, or ongoing treatments affecting blood composition may receive specialized analysis tailored to their specific clinical context. This personalization may include modified threshold relationships, specialized pattern detection algorithms, or adjusted weighting schemes that reflect the unique considerations relevant to specific patient subgroups. By contextualizing the analysis within the patient's demographic and medical profile, the system may provide more relevant assessments that account for individual variations in normal ranges and parameter relationships.

In some implementations of the method, the set of predetermined thresholds are derived from clinical guidelines; and wherein the screening function is adjusted based on cell population, such that the screening function generally permits values further from the set of predetermined thresholds when the cell population of a cell type is lower. The screening function may implement adaptive threshold behavior that accounts for statistical considerations related to cell population sizes. The predetermined thresholds themselves may be derived from established clinical guidelines, potentially incorporating standards from professional medical organizations, published literature, regulatory frameworks, or institutional protocols. When evaluating parameters associated with lower cell populations, the screening function may apply modified criteria that account for the increased statistical variability inherent in smaller sample sizes. This adjustment may allow for wider acceptable ranges when cell populations are smaller, recognizing that measurements based on fewer cells naturally exhibit greater statistical uncertainty. For example, parameters derived from rare cell types that typically appear in smaller numbers may be evaluated with more permissive proximity criteria than those derived from abundant cell types. This population-sensitive approach may help balance statistical reliability with clinical vigilance, potentially reducing false positives from statistically unstable measurements while maintaining appropriate sensitivity for clinically significant findings. The relationship between cell population size and threshold permissiveness may be implemented through various mathematical formulations, potentially including scaling factors, confidence interval adjustments, or statistical significance calculations that explicitly incorporate sample size considerations.

In some implementations, the method further comprises presenting, as part of the default human review workflow related to the first sample, explanatory information that identifies the reason for the value of screening function resulting in presenting the cells associated with the first sample. The system may enhance the human review experience by providing contextual explanatory information alongside the presented cells. This explanatory information may identify the specific reasons why the screening function determined that human review was warranted, potentially including detailed breakdowns of the parameter combinations that triggered the review. Such explanations may include visualizations showing the relationship between multiple parameters and their respective thresholds, highlighting patterns of simultaneous threshold approaches, or identifying unusual parameter combinations detected by the screening function. The explanatory information may also include relevant statistical context, such as how the observed parameter combination compares to reference distributions or historical patient data. By providing this contextual information, the system may help human reviewers focus their attention on the most relevant aspects of the case, potentially improving review efficiency and diagnostic accuracy. The explanatory information may be presented through various interfaces including but not limited to textual summaries, graphical visualizations, highlighted image regions, or interactive displays that allow reviewers to explore the underlying data that triggered the review determination. This approach may create a more transparent review process where human experts can understand and evaluate the computational reasoning behind the system's decision to present specific cases for review.

In some implementations, the method further comprises receiving classification confidence level (CCL) information related to the first and second sets of HCQMs in the first and second cytological smears, respectively; and wherein the screening function incorporates the CCL information in calculating the values of the screening function for the first and second samples. The screening function may incorporate classification confidence level (CCL) information to enhance its evaluation capabilities. The system may receive CCL information that quantifies the degree of certainty associated with cell classifications that contribute to the HCQMs, potentially expressed as percentages, probability scores, statistical confidence measures, or other numerical indicators of classification reliability. When calculating the value of the screening function, the system may incorporate this CCL information through various mechanisms, potentially including confidence-weighted parameter evaluations, uncertainty-adjusted threshold relationships, or probabilistic modeling approaches that explicitly account for classification uncertainty. This integration of CCL information may allow the system to make more nuanced assessments that consider not just the parameter values themselves but also the reliability of the underlying classifications. For example, the system may apply more conservative threshold proximity criteria for parameters derived from classifications with lower confidence levels, recognizing the increased potential for measurement error in these cases. Conversely, parameters based on high-confidence classifications may be evaluated with more standard criteria. This confidence-aware approach may help balance sensitivity and specificity in the review routing process, potentially reducing unnecessary reviews triggered by uncertain classifications while maintaining appropriate vigilance for parameters with clinical significance despite moderate classification uncertainty.

In some implementations of the method, the screening function assigns different weights to different cell types in the first and second sets of HCQMs based on their clinical relevance, statistical reliability, or proximity to their respective predetermined thresholds. The screening function may implement differential weighting schemes that reflect the varying importance of different cell types and their associated parameters. The system may assign weights to different cell types and their HCQMs based on multiple factors that influence their diagnostic significance. Clinical relevance weightings may prioritize cell types known to have greater diagnostic or prognostic value for specific conditions or broader health assessment, potentially giving higher weight to parameters associated with conditions of greater severity or clinical concern. Statistical reliability weightings may adjust the influence of parameters based on their measurement stability or confidence, potentially reducing the impact of metrics derived from smaller cell populations or those with greater inherent variability. Proximity-based weightings may dynamically adjust parameter influence based on how close they are to their thresholds, potentially increasing the weight of parameters as they approach their threshold values to create a progressive alerting system. These weighting schemes may be implemented through various mathematical formulations, potentially including linear weighting factors, non-linear transformations, conditional weighting rules, or more complex algorithmic approaches. By applying differential weights, the screening function may create a more nuanced evaluation that aligns with clinical priorities and statistical considerations, potentially improving the clinical relevance of its determinations.

In some implementations of the method, the screening function further comprises comparing the first and second sets of HCQMs to historical patterns associated with confirmed diagnoses to assess likelihood of clinical significance. The screening function may implement reference pattern comparison capabilities that leverage historical diagnostic knowledge. The system may compare current HCQM patterns against a repository of historical patterns associated with confirmed diagnoses, potentially identifying similarities to known disease signatures even when individual parameters remain within normal ranges. This pattern matching approach may utilize various computational techniques including but not limited to similarity metrics, distance calculations in multi-dimensional parameter space, classification algorithms, or case-based reasoning methods. The historical patterns may be derived from various sources, potentially including anonymized patient records with confirmed diagnoses, published case studies, research databases, or institutional knowledge repositories. When current parameter combinations show similarities to patterns associated with specific conditions, the system may factor this similarity into its screening function calculation, potentially increasing the likelihood of human review for cases that resemble known pathological patterns. This approach may enhance the screening function's ability to identify subtle clinical patterns that might not be captured by simple threshold-based rules, potentially improving detection of emerging conditions or rare presentations that have been previously documented in similar parameter combinations.

In some implementations, the method further comprises providing a human reviewer with information indicative of the individual HCQMs and their combined significance, with visual representations showing the relationship between the HCQMs. The system may enhance the human review experience through comprehensive parameter reporting and visualization capabilities. When presenting cases for human review, the system may provide information that includes both individual parameter values and analyses of their combined significance, potentially helping reviewers understand both the specific measurements and their collective implications. The individual HCQM information may include the raw values, their relationship to reference ranges, historical trends for the specific patient, and confidence metrics associated with each measurement. The combined significance information may include analysis of parameter patterns, correlation structures, similarity to known disease profiles, or other insights derived from multi-parameter evaluation. The system may present this information through various visual representations that illustrate the relationships between parameters, potentially including scatter plots showing parameter clustering, radar charts displaying multi-parameter profiles, heat maps indicating correlation structures, or other graphical formats that effectively communicate parameter relationships. These visualizations may include reference overlays showing typical patterns for various conditions, highlighting deviations from expected relationships, or indicating specific parameter combinations that triggered the review. By providing this comprehensive context, the system may facilitate more informed human interpretation that considers the full complexity of the parameter relationships rather than focusing solely on individual measurements.

In some implementations of the method, the screening function comprises employing a machine learning model trained to identify combinations of HCQMs which are statistically rare or that fall near decision boundaries in the multi-dimensional feature space. The screening function may implement advanced modeling approaches specifically designed to identify noteworthy parameter combinations. The system may incorporate models trained to recognize combinations of HCQMs that warrant attention, even when individual parameters remain within normal ranges. These models may be trained to identify statistically rare parameter combinations that deviate from typical multi-dimensional distribution patterns observed in reference populations, potentially highlighting unusual constellations of measurements that may have clinical significance despite their components appearing normal in isolation. Additionally, the models may be designed to detect parameter combinations that fall near decision boundaries in multi-dimensional feature space, identifying cases that reside in transitional regions between different classification outcomes where small parameter shifts could alter clinical interpretations. The models may be implemented through various computational approaches including but not limited to density estimation techniques, anomaly detection algorithms, boundary-focused classification methods, or deep learning architectures capable of learning complex patterns in high-dimensional data. Training data for these models may be derived from various sources, potentially including labeled clinical datasets, synthetic data generated from physiological models, or hybrid approaches combining real and simulated data. By focusing specifically on combination properties rather than individual parameter thresholds, these models may capture complex physiological relationships that manifest through parameter constellations rather than extreme individual values.

In some implementations of the method, the screening function comprises employing a machine learning model trained to detect unusual combinations of features in the HCQMs that deviate from expected patterns for known cell types. The screening function may implement specialized anomaly detection capabilities focusing on feature-level deviations. The system may incorporate models specifically trained to identify unusual combinations of features within the HCQMs that depart from expected patterns for known cell types, potentially revealing subtle abnormalities or emerging conditions that manifest through atypical feature relationships rather than extreme parameter values. These models may analyze various feature dimensions including but not limited to morphological characteristics, staining properties, size distributions, granularity patterns, nuclear configurations, or other measurable cellular attributes that contribute to the HCQMs. The unusual combination detection may occur at multiple levels, potentially identifying anomalies within individual cell populations, between different cell populations, or across the entire parameter profile. The models may be trained through various approaches, potentially including supervised learning with labeled examples of normal and abnormal feature combinations, unsupervised learning techniques that model typical feature distributions, or semi-supervised methods that learn primarily from normal examples to detect deviations. By focusing specifically on feature combination anomalies, these models may detect subtle but clinically significant deviations that might be missed by threshold-based approaches or even by models examining only high-level parameter combinations, potentially improving sensitivity for early-stage conditions or rare presentations that manifest through distinctive feature patterns. In some implementations of the method, a non-transitory computer-readable medium comprising instructions that, when executed by a processor, cause the processor to perform the computer-implemented method.

In various implementations, a system for analyzing combinations of parameters in cytological smears, comprising: a computer configured to: receive first and second sets of hematological cell or cell type quantification metrics (HCQMs) of cell types in first and second images of first and second cytological smears associated with first and second samples obtained from first and second patients, respectively; receive a set of predetermined thresholds associated with the respective HCQMs; compare each of the first and second sets of HCQMs with the set of predetermined thresholds, and identify that none of the first and second sets of HCQMs cross their respective predetermined thresholds; apply a screening function that evaluates relative positions of the first and second sets of HCQMs with respect to their predetermined thresholds; cause cells associated with the first sample to be presented as part of default human review workflow based on a value of the screening function for the first sample; and cause cells associated with the second sample to be excluded from presentation as part of default human review workflow based on a value of the screening function for the second sample.

In some implementations of the system, the screening function comprises at least one of: (a) calculating distance metrics between each HCQM in the first and second sets and their respective predetermined thresholds; (b) applying different weights to different distance metrics based on clinical significance of the corresponding HCQMs; or (c) applying threshold proximity analysis that identifies samples with multiple HCQMs simultaneously approaching but not crossing their respective predetermined thresholds. In some implementations of the system, the screening function comprises at least one of: (a) identifying patterns of simultaneous variations across multiple HCQMs, even when individual HCQMs do not cross their respective predetermined thresholds; (b) applying statistical distribution analysis to determine abnormal combinations of HCQMs; (c) applying statistical distribution analysis to determine clinical significant combinations of HCQMs; or (d) calculating correlation coefficients between pairs of HCQMs and comparing the correlation coefficients with expected correlation patterns.

In some implementations of the system, the screening function comprises at least one of: (a) calculating a composite score by combining multiple HCQMs according to a predetermined formula; (b) employing a machine learning model trained to identify clinically significant combinations of HCQMs; or (c) incorporating confidence intervals associated with the HCQMs. In some implementations of the system, the screening function comprises at least one of: (a) comparing the first and second sets of HCQMs with historical HCQMs associated with the first and second patients, respectively; (b) analyzing time-based trends in the HCQMs for each patient; or (c) calculating rate-of-change metrics for the HCQMs compared to previous measurements from the same patient. In some implementations of the system, the screening function applies different algorithms based on patient demographic information and/or medical history.

In some implementations of the system, the set of predetermined thresholds are derived from clinical guidelines; and wherein the screening function is adjusted based on cell population, such that the screening function generally permits values further from the set of predetermined thresholds when the cell population of a cell type is lower. In some implementations of the system, the computer is further configured to cause presentation, as part of the default human review workflow related to the first sample, of explanatory information that identifies the reason for the value of the screening function resulting in presenting the cells associated with the first sample. In some implementations of the system, the computer is further configured to: receive classification confidence level (CCL) information related to the first and second sets of HCQMs in the first and second cytological smears, respectively; and wherein the screening function incorporates the CCL information in calculating the values of the screening function for the first and second samples.

In some implementations of the system, the screening function assigns different weights to different cell types in the first and second sets of HCQMs based on their clinical relevance, statistical reliability, or proximity to their respective predetermined thresholds. In some implementations of the system, the screening function further comprises comparing the first and second sets of HCQMs to historical patterns associated with confirmed diagnoses to assess likelihood of clinical significance. In some implementations of the system, the computer is further configured to provide a human reviewer with information indicative of the individual HCQMs and their combined significance, with visual representations showing the relationship between the HCQMs. In some implementations of the system, the screening function comprises a model trained to identify combinations of HCQMs which are statistically rare or that fall near decision boundaries in the multi-dimensional feature space. In some implementations of the system, the screening function comprises a model trained to detect unusual combinations of features in the HCQMs that deviate from expected patterns for known cell types.

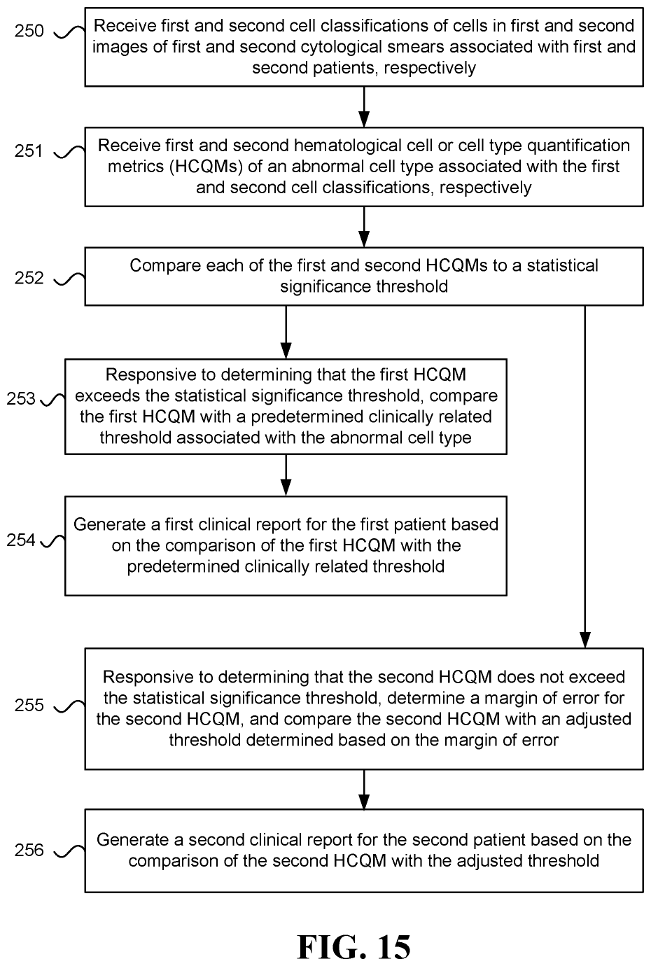

In various implementations, a computer-implemented method for adjusting thresholds based on statistical significance of cell populations, comprising: receiving first and second cell classifications of cells in first and second images of first and second cytological smears associated with first and second patients, respectively; receiving first and second hematological cell or cell type quantification metrics (HCQMs) of an abnormal cell type associated with the first and second cell classifications, respectively; comparing each of the first and second HCQMs to a statistical significance threshold; responsive to determining that the first HCQM exceeds the statistical significance threshold, comparing the first HCQM with a predetermined clinically related threshold associated with the abnormal cell type; responsive to determining that the second HCQM does not exceed the statistical significance threshold, determining a margin of error for the second HCQM, and comparing the second HCQM with an adjusted threshold determined based on the margin of error; generating a first clinical report for the first patient based on the comparison of the first HCQM with the predetermined clinically related threshold; and generating a second clinical report for the second patient based on the comparison of the second HCQM with the adjusted threshold. FIG. 15 illustrates an embodiment of a computer-implemented method for adjusting thresholds based on statistical significance of cell populations. The method includes the following steps. In step 250, receive first and second cell classifications of cells in first and second images of first and second cytological smears associated with first and second patients, respectively. In step 251, receive first and second hematological cell or cell type quantification metrics (HCQMs) of an abnormal cell type associated with the first and second cell classifications, respectively. In step 252, compare each of the first and second HCQMs to a statistical significance threshold. In step 253, responsive to determining that the first HCQM exceeds the statistical significance threshold, compare, optionally by a processor, the first HCQM with a predetermined clinically related threshold associated with the abnormal cell type. In step 254, generate a first clinical report for the first patient based on the comparison of the first HCQM with the predetermined clinically related threshold. In step 255, responsive to determining that the second HCQM does not exceed the statistical significance threshold, determine a margin of error for the second HCQM, and compare, optionally by the processor, the second HCQM with an adjusted threshold determined based on the margin of error. And in step 256, generate a second clinical report for the second patient based on the comparison of the second HCQM with the adjusted threshold.

In some implementations, the method further comprises presenting the first and second clinical reports in an electronic medical information system, wherein the second clinical report includes a visual indication denoting use of the adjusted threshold. In some implementations of the method, determining the margin of error for the second HCQM is based on at least one of: a quantity of the abnormal cell type in the second cell classification, a quantity of cells of a lineage to which the abnormal cell type belongs in the second cell classification, or a ratio between the quantity of the abnormal cell type and the quantity of cells of the lineage in the second cell classification. In some implementations of the method, the statistical significance threshold is based on predetermined acceptable error margins to reflect a level of certainty. The system may implement dynamic threshold adjustments that account for the statistical implications of varying cell population sizes in cytological analysis. When analyzing cytological smears, the statistical reliability of hematological cell or cell type quantification metrics (HCQMs) may be influenced by the total number of cells from which they are derived, with larger cell populations typically providing more statistically robust measurements compared to smaller populations, and further, by the number of cells in that lineage that have been measured. For example, if 1000 white blood cells were measured, out of which 10 classified as blasts, the confidence that 1% of white blood cells are blasts is higher than if 100 white blood cells were measured out of which just one classified as a blast. The determination of the margin of error may be implemented through various statistical approaches, including confidence interval calculations, standard error assessments, or probability distribution models that quantify the expected variability based on sample size principles. For example, the system may apply statistical principles such as binomial distribution models for presence/absence metrics or normal approximation methods for continuous metrics, with the specific approach selected based on the properties of the particular HCQM being analyzed. The adjustment of thresholds may be implemented through various mathematical transformations, including proportional scaling based on confidence intervals, adaptive boundary adjustments linked to standard error calculations, or variable thresholds derived from probability distributions. These adjustments may recognize that smaller cell populations inherently carry greater statistical uncertainty, which may be addressed through more conservative threshold interpretations that accommodate the higher potential for sampling variability. The visual indication included in the report may help clinicians understand that an adjusted threshold has been applied due to population size considerations, potentially enhancing clinical interpretation by providing context about the statistical confidence of the reported comparisons. The system may implement various statistical methodologies to calculate appropriate margins of error that reflect the uncertainty associated with cell population measurements. These calculations may incorporate multiple relevant factors that influence statistical confidence. The total number of cells analyzed in the lineage may serve as a primary input to confidence calculations as well as the number of cells of the cell type, potentially utilizing statistical principles such as the law of large numbers or the central limit theorem to quantify how sampling size affects measurement reliability. For example, confidence interval width may decrease proportionally to the square root of the sample size, reflecting the established statistical relationship between population size and estimate precision. The prevalence of the abnormal cell type may influence the statistical approach, with rare cell types potentially requiring specialized statistical handling such as Poisson distribution models for low-prevalence events or binomial confidence intervals for presence/absence determinations. Established statistical models for specific abnormal cell types may incorporate domain-specific knowledge about measurement characteristics, known biological variation, or classification challenges associated with particular cell populations. These models may include weighted error calculations that account for misclassification probabilities, Bayesian confidence estimates that incorporate prior distribution knowledge, or specialized algorithms developed for specific diagnostic contexts. The system may select the appropriate statistical methodology based on cell type characteristics, measurement properties, or clinical context, potentially applying different approaches to different cell populations within the same sample to optimize statistical validity across diverse measurement scenarios.

The statistical significance of cell populations may relate to the reliability and confidence level of measurements derived from analyzing specific quantities of cells within cytological samples. When cell populations contain larger numbers of cells, the resulting HCQMs may exhibit greater statistical reliability due to reduced sampling variability, whereas smaller cell populations may produce measurements with inherently higher uncertainty due to sampling statistics. The statistical significance threshold may represent a predetermined boundary value that distinguishes between cell populations of sufficient size to produce statistically reliable measurements using standard clinical thresholds and those requiring adjusted thresholds to account for increased statistical uncertainty. This threshold may be established through various approaches, including statistical power calculations, empirical validation studies, or clinical laboratory standards, and may reflect the minimum population size at which the probability of sampling error falls within acceptable bounds for clinical interpretation. When HCQMs derived from cell populations exceed the statistical significance threshold, the measurements may be considered sufficiently reliable for direct comparison with predetermined clinically related thresholds, whereas HCQMs falling below this threshold may require statistical adjustments to account for the increased uncertainty associated with smaller sample sizes.

In some implementations of the method, the visual indication comprises one or more of: a textual mention of the adjusted threshold, the determined margin of error, or an allowable range for the second HCQM based on the adjusted threshold. The system may implement various textual approaches to communicate threshold adjustments and their statistical context within clinical reports. These textual indications may include explicit mentions of the adjusted threshold value alongside the standard threshold, potentially presented as paired values (e.g., “Standard threshold: 5%, Adjusted threshold: 6.2%”) or as explanatory notes detailing the adjustment rationale and magnitude. The determined margin of error may be presented through various textual formats, including percentage representations, confidence interval notations, or explicit uncertainty ranges that quantify the statistical variability associated with the particular cell population size. Allowable ranges for the HCQM may be communicated through interval notations, boundary specifications, or tolerance statements that define the acceptable range within which the measured value may still be considered consistent with expectations despite the smaller sample size. These textual elements may be incorporated at various locations within the report, including within result sections adjacent to the relevant measurements, in dedicated statistical notation sections, within interpretive comments, or as footnotes with appropriate reference markers. The specific textual approach may be selected based on reporting conventions, electronic health record formatting constraints, or laboratory information system capabilities, with the overarching goal of providing clear communication about the statistical context without obscuring the primary clinical information.

In some implementations of the method, the visual indication comprises a graphic representation, comprising at least one of: reference lines indicating the predetermined clinically related threshold and the adjusted threshold, color gradients reflecting proximity to the adjusted threshold, numerical indicators showing the determined margin of error, or visual elements indicating statistical confidence levels associated with the second HCQM. The system may implement various graphical approaches to visually communicate threshold adjustments and statistical confidence information within clinical reports. These graphical representations may enhance the interpretation of results by providing intuitive visual cues about statistical context. Reference lines may be implemented as horizontal or vertical demarcations on charts or graphs, potentially using different line styles, weights, or colors to distinguish between standard and adjusted thresholds. These lines may appear on bar charts, distribution plots, or trend visualizations where the measured values are displayed. Color gradients may provide continuous visual mapping of statistical confidence or proximity to thresholds, potentially implemented through background shading that transitions between colors (such as green to yellow to red) as measurements approach or cross adjusted threshold boundaries. These gradients may be applied to tabular data cells, chart elements, or dedicated confidence indicators. Numerical indicators may include error bars, confidence interval brackets, or explicit ±notation adjacent to measurements, visually representing the calculated statistical uncertainty associated with the specific cell population size. Additional visual elements may include confidence meters resembling speedometer-style gauges, certainty stars where the number or opacity indicates confidence level, or iconography such as information symbols that provide visual cues about statistical considerations. These graphical approaches may be implemented through various technical means including vector graphics, CSS styling in electronic reports, or specialized medical visualization libraries, with the specific implementation potentially varying based on the reporting platform capabilities and clinical context.

In some implementations of the method, the predetermined clinically related threshold is between 1%-5% of total cells for the cell type lineage to which the abnormal cell type belongs. The predetermined clinically related threshold may reflect established clinical guidelines, reference ranges, or diagnostic criteria associated with various abnormal cell types. The range of 1%-5% may represent clinically significant boundaries for various abnormal cell populations relative to their respective lineages. For abnormal lymphocytes, a threshold within this range may align with criteria for conditions such as chronic lymphocytic disorders or reactive lymphocytosis. For immature granulocytes, thresholds in this range may correspond to boundaries between reactive left shifts and more significant myeloproliferative processes. For blast cells, values in the lower portion of this range may align with criteria for minimal residual disease detection or early relapse identification in hematological malignancies. The specific value selected within this range may depend on multiple factors including the particular abnormal cell type, the clinical context, the patient population characteristics, and/or established laboratory protocols. The threshold may be implemented as an absolute percentage value, as a relative increase compared to baseline values, or as a statistical deviation from reference population distributions. The system may maintain different thresholds within this range for different cell types based on their clinical significance, with lower thresholds typically applied to more diagnostically significant cell populations such as blast cells and higher thresholds applied to less critical abnormalities.

In some implementations of the method, the adjusted threshold is between 0.75-1.25 times the predetermined clinically related threshold, with the specific adjustment factor determined based on the size of the cell population from which the second HCQM is derived. The system may implement adjustment factors that modify the predetermined clinically related threshold within a defined range to account for statistical considerations related to cell population size. The range of 0.75-1.25 times the original threshold may provide boundaries for adjustment that balance statistical considerations with clinical interpretability. The specific adjustment factor within this range may be determined through various approaches, including continuous scaling functions that provide gradually increasing adjustments as population size decreases, stepped adjustments that apply different factors at defined population size breakpoints, or algorithm-derived factors based on calculated confidence intervals specific to each measurement scenario. For example, when cell population sizes approach the minimum acceptable statistical sample size, adjustment factors near the upper boundary (1.25×) may be applied to increase the threshold, requiring stronger evidence before crossing into abnormal territory. Conversely, for population sizes closer to optimal statistical sampling, minimal adjustments may be applied. The direction of adjustment may vary depending on whether the threshold represents an upper or lower boundary of normal, with upper boundary thresholds potentially increased (making them more conservative) and lower boundary thresholds potentially decreased (also making them more conservative) when working with smaller populations. The specific mathematical relationship between population size and adjustment factor may be implemented through various functions, including inverse proportional relationships, logarithmic scaling, or statistically-derived confidence boundary calculations, selected based on the particular statistical properties of the cell type being measured.

In some implementations, the method further comprises determining a cell population size threshold for the cell type or cell lineage that represents a minimum sample size for reliable statistical assessment of the abnormal cell type; comparing the cell population size from which the second HCQM is derived with the cell population size threshold; and wherein generating the adjusted threshold is performed when the cell population size falls below the cell population size threshold. The system may implement conditional threshold adjustment mechanisms that activate when cell population sizes fall below statistically significant thresholds. These cell population size thresholds may be established through various approaches, including statistical power calculations that determine minimum sample sizes required for specific confidence levels and error margins; empirical analyses of measurement stability across different population sizes; established laboratory or clinical guidelines for minimum acceptable counts; the number of cells from the lineage to which the abnormal cell belongs that were measured in the sample, or cell type-specific requirements based on prevalence expectations and classification challenges. Different abnormal cell types may be associated with different population size thresholds based on their clinical significance, classification complexity, or typical prevalence, with rare and diagnostically critical cell types potentially requiring larger minimum populations to achieve acceptable statistical confidence. The comparison between actual population size and the established threshold may trigger different system behaviors, potentially implementing a binary decision model where adjustments are applied below the threshold, or a graduated response where adjustment magnitudes increase progressively as population size decreases further below the threshold. This conditional approach may optimize processing efficiency by applying adjustments when statistically warranted while maintaining standard threshold interpretations when population sizes provide adequate statistical power. The system may document the population size threshold used and the outcome of the comparison in analysis metadata, potentially for quality assurance purposes or to inform future threshold calibration efforts across the patient population.

In some implementations of the method, the magnitude of adjustment to the predetermined clinically related threshold is inversely proportional to the square root of the number of cells of the cell type or cell lineage, analyzed for deriving the second HCQM. The system may implement mathematically rigorous adjustment formulas derived from established statistical principles to determine appropriate threshold modifications. The inverse square root relationship may reflect the well-established statistical principle that standard error is inversely proportional to the square root of sample size, a relationship that derives from fundamental statistical theory including the central limit theorem. This mathematical relationship may be implemented through various formulas, including direct scaling where the adjustment factor equals a constant multiplied by the inverse square root of the cell count; more complex formulations that incorporate additional factors such as observed prevalence or expected variance; or modified versions that implement upper and lower boundaries to prevent extreme adjustments for very small or very large populations. The specific formula implementation may vary based on the statistical properties of different cell types and clinical contexts, potentially with different coefficient values or boundary conditions applied to different measurement scenarios. This mathematically grounded approach may provide a statistically valid scaling of thresholds that directly reflects the quantitative relationship between population size and measurement confidence. The system may apply this formula through various technical implementations, including real-time calculations during result analysis, lookup tables that approximate the formula for common population sizes, or precomputed adjustment factors for standard sampling scenarios, with the specific implementation potentially selected based on computational efficiency considerations and precision requirements.

In some implementations of the method, different adjustment factors are applied to the predetermined clinically related threshold based on the clinical significance of the abnormal cell type, with larger adjustments applied for rare cell types with higher diagnostic significance. The system may implement cell type-specific adjustment methods that recognize the varying clinical implications and statistical characteristics of different abnormal cell populations. These differentiated adjustment approaches may account for multiple factors that influence the relationship between population size and measurement reliability for particular cell types. Cells with higher diagnostic significance, such as blast cells or diagnostically critical abnormal forms, may receive adjustments that maintain higher sensitivity even with smaller populations, reflecting their clinical importance in disease detection and monitoring. Rare cell types may be subject to specialized statistical handling that accounts for the particular challenges of low-prevalence measurements, potentially incorporating Poisson distribution principles, rare event statistics, or specialized confidence calculations appropriate for low-count scenarios. The adjustment factors may be calibrated based on empirical analysis of detection reliability across different population sizes for specific cell types, clinical validation studies, or published literature regarding statistical confidence requirements for particular diagnostic applications. The system may maintain a comprehensive mapping between cell types and their corresponding adjustment parameters, potentially organized according to cell lineages, diagnostic categories, or statistical behavior classes. This cell type-specific approach may optimize the balance between statistical validity and clinical sensitivity for each abnormal cell population, rather than applying uniform adjustment methods that might be inappropriate for certain cell types.

In some implementations, the method further comprises determining a statistical confidence level associated with the second HCQM; comparing the statistical confidence level with a predetermined confidence threshold; and including in the report associated with the second patient a recommendation for additional testing when the statistical confidence level falls below the predetermined confidence threshold. The system may implement recommendation mechanisms that suggest appropriate follow-up actions when statistical confidence falls below acceptable levels for clinical decision-making. The statistical confidence level may be calculated through various approaches, including: formal confidence interval determinations expressed as percentage confidence (e.g., 95% confidence); statistical power calculations that assess the probability of detecting true abnormalities at the observed population size; precision metrics such as coefficient of variation or standard error relative to the measured value; or composite confidence scores that integrate multiple statistical indicators. This calculated confidence value may be compared against predetermined thresholds that represent minimum acceptable statistical reliability for clinical interpretation, potentially with different thresholds applied to different clinical scenarios or cell types based on their diagnostic implications. When confidence falls below these thresholds, the system may generate recommendations for additional testing, potentially including suggestions for repeat analysis with larger sample volumes, alternative testing methodologies with higher sensitivity for the particular cell type, confirmatory testing using different analytical approaches, or monitoring with shortened time intervals to verify findings. These recommendations may be presented in various report locations including result interpretation sections, notes fields, recommendation blocks, or follow-up care sections, and may include explanatory context about the statistical limitations that triggered the recommendation. This confidence-based recommendation approach may help direct additional diagnostic resources toward cases where limited statistical confidence may affect clinical reliability, potentially improving overall diagnostic quality while optimizing resource utilization.

In various implementations, a system for adjusting thresholds based on statistical significance of cell populations, comprising: a computer configured to: receive first and second cell classifications of cells in first and second images of first and second cytological smears associated with first and second patients, respectively; receive first and second hematological cell or cell type quantification metrics (HCQMs) of an abnormal cell type associated with the first and second cell classifications, respectively; compare each of the first and second HCQMs to a statistical significance threshold; responsive to determining that the first HCQM exceeds the statistical significance threshold, compare the first HCQM with a predetermined clinically related threshold associated with the abnormal cell type; responsive to determining that the second HCQM does not exceed the statistical significance threshold, determine a margin of error for the second HCQM, and compare the second HCQM with an adjusted threshold determined based on the margin of error; generate a first clinical report for the first patient based on the comparison of the first HCQM with the predetermined clinically related threshold; and generate a second clinical report for the second patient based on the comparison of the second HCQM with the adjusted threshold.

In some implementations of the system, the computer is further configured to: cause the first and second clinical reports to be presented in an electronic medical information system, wherein the second clinical report includes a visual indication denoting use of the adjusted threshold. In some implementations of the system, the visual indication comprises one or more of: a textual mention of the adjusted threshold, the determined margin of error, or an allowable range for the second HCQM based on the adjusted threshold. In some implementations of the system, the visual indication comprises a graphic representation, comprising at least one of: reference lines indicating the predetermined clinically related threshold and the adjusted threshold, color gradients reflecting proximity to the adjusted threshold, numerical indicators showing the determined margin of error, or visual elements indicating statistical confidence levels associated with the second HCQM.

In some implementations of the system, determining the margin of error for the second HCQM is based on at least one of: a quantity of the abnormal cell type in the second cell classification, a quantity of cells of a lineage to which the abnormal cell type belongs in the second cell classification, or a ratio between the quantity of the abnormal cell type and the quantity of cells of the lineage in the second cell classification. In some implementations of the system, the statistical significance threshold is based on predetermined acceptable error margins to reflect a level of certainty. In some implementations of the system, the predetermined clinically related threshold is between 1%-5% of total cells for the cell type lineage to which the abnormal cell type belongs.

In some implementations of the system, the adjusted threshold is between 0.75-1.25 times the predetermined clinically related threshold, with the specific adjustment factor determined based on the size of the cell population from which the second HCQM is derived. In some implementations of the system, the computer is further configured to: determine a cell population size threshold for the cell type or cell lineage that represents a minimum sample size for reliable statistical assessment of the abnormal cell type; compare the cell population size from which the second HCQM is derived with the cell population size threshold; and wherein generating the adjusted threshold is performed when the cell population size falls below the cell population size threshold.

In some implementations of the system, the magnitude of adjustment to the predetermined clinically related threshold is inversely proportional to the square root of the number of cells of the cell type or cell lineage, analyzed for deriving the second HCQM. In some implementations of the system, different adjustment factors are applied to the predetermined clinically related threshold based on the clinical significance of the abnormal cell type, with larger adjustments applied for rare cell types with higher diagnostic significance. In some implementations of the system, the computer is further configured to: determine a statistical confidence level associated with the second HCQM; compare the statistical confidence level with a predetermined confidence threshold; and include in the report associated with the second patient a recommendation for additional testing when the statistical confidence level falls below the predetermined confidence threshold.

Some embodiments may address the optimization of human review resource allocation in cytological smear analysis through multi-dimensional feature analysis. In some cases, the decision of whether human expertise adds value depends on the relationships between multiple features rather than single values. The following embodiment extracts multiple morphological, colorimetric, textural, and spatial features from cells and applies decision models in this multi-dimensional feature space to determine which samples warrant human review. By evaluating feature relationships rather than individual metrics, the system may more precisely identify cases where human expertise adds diagnostic value, potentially reducing unnecessary reviews while enhancing the likelihood of clinically significant cases to receive appropriate expert attention. The multi-dimensional analysis may support more nuanced routing decisions that consider the specific characteristics of the various analysis scenarios, creating a more efficient and effective workflow that optimizes the allocation of valuable human expertise.

In various embodiments, the computer-implemented method for determining human review requirements in cytological smear analysis using multi-dimensional feature analysis may provide an approach to identifying cases requiring expert evaluation. The method may involve receiving an image of a cytological smear associated with a patient and extracting multiple features from cells in the image. These features may include, but are not limited to: morphological features (such as cell size, shape, nuclear-to-cytoplasmic ratio, membrane irregularities, and/or nuclear characteristics), colorimetric features (such as staining intensity, color distribution, chromatin patterns, and/or cytoplasmic color variations), textural features (such as granularity, homogeneity, entropy measures, and/or fractal dimensions of cellular components), and/or spatial relationship features (such as cell clustering patterns, neighborhood characteristics, relative positioning of cells, and/or tissue architecture indicators). The feature extraction process may utilize various image processing techniques including but not limited to edge detection, color space transformations, texture analysis algorithms, and/or spatial correlation methods to characterize the cells comprehensively across multiple dimensions of analysis.

The multi-dimensional decision model applied to these features may evaluate relationships between multiple features rather than relying on individual numerical thresholds for single features. This approach may recognize that the diagnostic significance of certain features may depend on their co-occurrence with other features rather than their absolute values. For example, a particular nuclear shape may have different implications depending on accompanying chromatin patterns, or cell size variations may be interpreted differently based on associated textural characteristics. The multi-dimensional model may implement various computational approaches including but not limited to multivariate statistical methods, machine learning algorithms, pattern recognition techniques, and/or knowledge-based systems that capture feature interdependencies. This relational analysis may potentially identify subtle patterns that might be missed by threshold-based approaches that evaluate features independently. The classification of cells based on this model may reflect not just cell type identification but may also incorporate confidence assessments and uncertainty quantification based on the patterns of feature relationships observed.

When determining whether to route at least a subset of the image for human review, the system may identify cells that fall within uncertainty regions of the multi-dimensional decision model. These uncertainty regions may be defined by feature combinations that are statistically rare or that fall near decision boundaries in the multi-dimensional feature space. Statistical rarity may be established through reference population analysis, where feature combination frequencies are calculated across large datasets to identify combinations that occur below certain probability thresholds. Decision boundary proximity may be quantified through distance metrics in the multi-dimensional feature space, identifying cells whose feature combinations place them in regions where classification changes with small feature variations. This approach may direct human expertise toward cases where automated analysis faces classification challenges, potentially optimizing the use of specialist time while maintaining diagnostic reliability.

The determination of whether to route images for human review may also involve identifying subsets of cells with feature combinations associated with higher clinical significance. The clinical significance may be determined based on correlations between feature combinations and potential pathological conditions, recognizing that certain feature patterns may have greater implications for patient care than others, even when classification confidence is similar. This approach may incorporate correlation data from clinical outcomes, pathology reports, and/or expert-annotated datasets that link specific feature patterns to diagnostic or prognostic significance. The system may maintain and update a database of feature combination patterns sorted by clinical importance, potentially allowing for dynamic adjustment of review thresholds based on the specific clinical context or the patient's medical history. By prioritizing review based on clinical significance rather than purely on classification uncertainty, the system may help ensure that human expertise is directed toward cases with the greatest potential impact on patient management.

Another criterion that may trigger human review involves detecting unusual combinations of features that deviate from expected patterns for known cell types, even when classification confidence level for individual cells is high. This “anomaly detection” approach may recognize that automated systems may sometimes classify cells with high confidence despite unusual characteristics that warrant expert evaluation. The deviation assessment may involve comparing observed feature combinations against established multi-dimensional models of expected feature relationships for each cell type, identifying outliers through various statistical or machine learning-based anomaly detection methods. This capability may be particularly valuable for identifying atypical presentations of pathological conditions or rare variants that may not be well-represented in the system's training data. By flagging unusual patterns regardless of classification confidence, the system may help identify novel or rare cellular presentations that might otherwise be overlooked.

When routing cases for human review, the system may further select a specific type of human reviewer expertise based on the nature of the feature combinations identified by the multi-dimensional decision model. Different feature patterns may benefit from different types of specialist knowledge, and the system may match cases to the most appropriate expertise. For example, feature combinations suggesting potential hematological malignancies may be routed to hematopathologists, while those indicating possible inflammatory processes may be directed to specialists in immunopathology. This expertise matching may be implemented through a mapping system that associates feature pattern categories with specialist knowledge domains, potentially incorporating feedback mechanisms that refine these associations based on review outcomes. The expertise selection may also consider workload balancing, urgency factors, and/or reviewer availability to optimize the review workflow. This targeted routing may help ensure that cases are evaluated by reviewers with the most relevant expertise, potentially improving diagnostic accuracy while optimizing specialist resource allocation.

In various implementations, the subset of the image routed for human review may be less than the entire image, with the specific subset selected based on the multi-dimensional decision model. Rather than requiring review of complete cytological smears, which may contain thousands of cells, the system may identify and extract regions of interest or specific cells that warrant expert attention. The selection criteria may consider not only individual cells that trigger review thresholds but also their surrounding context, neighboring cells, and/or spatial distribution patterns that may provide important diagnostic context. The system may generate focused view packages that present these selected regions to reviewers in ways that facilitate efficient evaluation, potentially including multiple magnification levels, annotation overlays, and/or comparative reference images. This selective routing approach may significantly reduce the review burden compared to whole-slide review while still providing sufficient context for accurate assessment. The specific implementation may balance the benefits of focused review against the potential value of broader contextual information, with the selection algorithm potentially adjustable based on case characteristics or reviewer preferences.

The system may assign priority levels for human review based on the complexity of feature relationships identified by the multi-dimensional decision model, wherein more complex feature relationships may be associated with higher review priorities. Complexity may be quantified through various metrics such as the number of interacting features, the rarity of the specific combination pattern, the degree of deviation from expected relationships, and/or the difficulty in resolving classification uncertainty. Cases with highly complex feature relationships may present greater analytical challenges and may benefit more from expert interpretation than those with simpler patterns. This prioritization approach may help optimize workflow by ensuring that the most challenging cases receive prompt attention, potentially improving turnaround times for difficult cases while allowing more straightforward cases to be processed more efficiently. The priority assignment may be implemented through a scoring system that combines multiple complexity indicators and may also incorporate factors such as clinical urgency, patient history, and/or institutional priorities to generate a holistic priority score for each case requiring review.

For cases routed to human review, the system may generate explanatory information that identifies the specific feature relationships that triggered the routing decision. This explanatory information may guide a human reviewer to focus on relevant aspects of the cells, potentially reducing review time and improving assessment consistency. The explanations may highlight which particular feature combinations led to uncertainty, clinical significance flags, or anomaly detection, providing reviewers with a starting point for their evaluation. Implementation approaches may include visual highlighting of relevant features, comparative displays showing similar reference cases, quantitative information about how specific features relate to classification boundaries, and/or natural language explanations of the reasoning behind the review recommendation. By providing this targeted guidance, the system may potentially facilitate more efficient and accurate human review while also creating an educational feedback loop that helps reviewers develop deeper understanding of complex feature relationships. The explanatory component may be customizable based on reviewer preferences or institutional protocols, allowing the level of detail and presentation format to be adjusted according to specific needs or user experience levels.

The following embodiment addresses the determination of when additional cytological smear preparation is warranted based on multi-dimensional feature analysis. In some cases, the adequacy of a smear for diagnostic purposes may be determined by relationships between multiple factors rather than by single measurements. It extracts multiple morphological, colorimetric, textural, and spatial features from cells and applies decision models in this multi-dimensional feature space to determine whether another smear would provide valuable additional diagnostic information. By evaluating feature relationships rather than individual metrics, the system may more precisely identify cases where additional sampling would improve diagnostic confidence, potentially reducing unnecessary repeat procedures while enhancing the likelihood of having comprehensive analysis of clinically significant cases. The multi-dimensional analysis may support more informed decisions about additional smear preparation that consider the specific characteristics of different analysis scenarios, creating a more efficient and effective diagnostic process.

In various embodiments, the computer-implemented method for determining additional cytological smear requirements using multi-dimensional feature analysis may provide an approach to identifying cases requiring additional samples. The method may involve receiving an image of a cytological smear and extracting multiple features from cells, including morphological features (such as cell size, shape, nuclear characteristics), colorimetric features (such as staining intensity, color distribution), textural features (such as granularity, homogeneity), and/or spatial relationship features (such as cell clustering patterns, relative positioning). The multi-dimensional decision model applied to these features may evaluate relationships between multiple features rather than individual thresholds, recognizing that diagnostic significance often depends on feature co-occurrence patterns. This approach may potentially identify subtle patterns that might be missed by threshold-based approaches that evaluate features independently.

When determining whether to command preparation of another cytological smear, the system may evaluate the quality of the cytological smear based on feature combinations that indicate preparation artifacts or inadequacies. Different types of preparation issues may be identified through specific patterns of feature relationships rather than individual quality metrics. For example, staining inadequacies may be recognized through particular combinations of color intensity variations, texture abnormalities, and morphological distortions, while smearing problems may manifest in characteristic spatial distribution anomalies combined with cell deformation patterns. This multi-dimensional quality assessment may potentially detect subtle preparation issues that might not trigger threshold violations in any single metric but collectively indicate preparation inadequacies that could compromise diagnostic reliability.

The determination to command preparation of another cytological smear may involve assessing whether the quantity of cells with particular feature combinations provides adequate statistical significance for diagnostic purposes. This assessment may consider the clinical importance of the feature combinations, recognizing that different cell presentations require different levels of statistical support depending on their diagnostic implications. For rare but clinically significant feature combinations, higher statistical confidence may be required, potentially necessitating additional sampling to achieve sufficient cell counts. The system may implement statistical adequacy calculations that consider both the absolute number of cells displaying particular feature combinations and their relative proportion within the broader cell population, potentially incorporating confidence interval calculations that reflect the diagnostic weight of specific findings.

Based on the multi-dimensional decision model, the system may determine specific preparation parameters to modify when commanding the automated slide maker-stainer to prepare another cytological smear. These modifications may address specific inadequacies identified through feature relationship analysis. Rather than applying generic protocol adjustments, the system may implement targeted modifications that correspond to the particular preparation issues detected. For example, if feature relationships suggest inadequate nuclear staining with otherwise appropriate cytoplasmic characteristics, staining duration or concentration parameters might be specifically adjusted while maintaining other protocol elements. This targeted approach may potentially improve the likelihood that subsequent preparations resolve the specific inadequacies identified in the initial sample.

Another criterion that may trigger preparation of another cytological smear involves identifying cells with feature combinations that approach but do not clearly exceed decision boundaries in the multi-dimensional feature space. These borderline presentations may indicate potential diagnostic significance requiring additional sampling for confirmation. Unlike cases where features clearly indicate specific cell types or conditions, borderline cases exist in transition zones between classification categories, creating ambiguity that additional sampling may help resolve. The system may identify these cases through proximity metrics in feature space, potentially flagging cells whose feature combinations place them within certain distance thresholds of classification boundaries. By obtaining additional samples in these borderline cases, the system may help distinguish between genuine transitional presentations and preparation-induced ambiguities.

The system may evaluate whether rare cell types with particular feature combinations are represented in sufficient quantities for reliable analysis when determining whether to command preparation of another cytological smear. This evaluation may be based on the clinical significance of the particular feature combinations, recognizing that different rare cell presentations carry different diagnostic weights. For highly significant rare cells, even small quantity inadequacies may trigger additional sampling, while for less critical rare populations, greater quantity deficiencies might be tolerated before requiring another smear. The system may maintain references for minimum quantity requirements based on cell rarity and clinical importance, potentially incorporating statistical models that estimate the probability of missing significant rare cell populations given the observed quantities.

The determination to command preparation of another cytological smear may be based on a combined assessment of both preparation quality and cell content adequacy derived from the multi-dimensional decision model. This integrated approach recognizes that preparation issues and content limitations may interact in ways that compound their individual impacts on diagnostic reliability. Preparation issues that would not independently warrant another smear may do so when combined with borderline content adequacy, as the cumulative effect may cross a threshold of diagnostic uncertainty that requires additional sampling. The system may implement various integration mechanisms, potentially including weighted scoring systems that combine quality and content factors, decision matrices that map combined states to sampling recommendations, or more complex algorithmic approaches that model the interactive effects of multiple factors on diagnostic confidence.

When commanding preparation of another cytological smear, the system may generate a report that identifies the specific feature relationships that triggered the additional smear decision and explains how the additional smear is expected to improve diagnostic confidence. This explanatory component may support quality improvement processes by providing clear documentation of the reasoning behind additional sampling decisions. The report may include identification of specific inadequacies detected, quantification of statistical limitations, visualization of borderline feature presentations, and/or projections of how modified preparation parameters are expected to address identified issues. By providing this contextual information, the system may potentially facilitate more effective preparation adjustments while also creating a knowledge base that could inform future protocol refinements and quality improvement initiatives.

The following implementation addresses the integration of patient medical history data to enhance cytological smear analysis. By incorporating information from an electronic medical information system, the system may adjust analysis parameters to account for known conditions and expected cellular presentations, which may improve diagnostic accuracy by considering the patient's medical context when interpreting cell morphology and distributions, and may support more personalized analysis that takes into account the unique medical profile of the patient. The integration of medical history data enables more intelligent routing decisions for human review, potentially reducing unnecessary expert consultations while enhancing the likelihood of clinically significant findings to receive appropriate human attention. Additionally, the system may implement differential reviews that focus human expertise on specific cell populations most relevant to the patient's medical context, enhancing review efficiency and diagnostic precision.

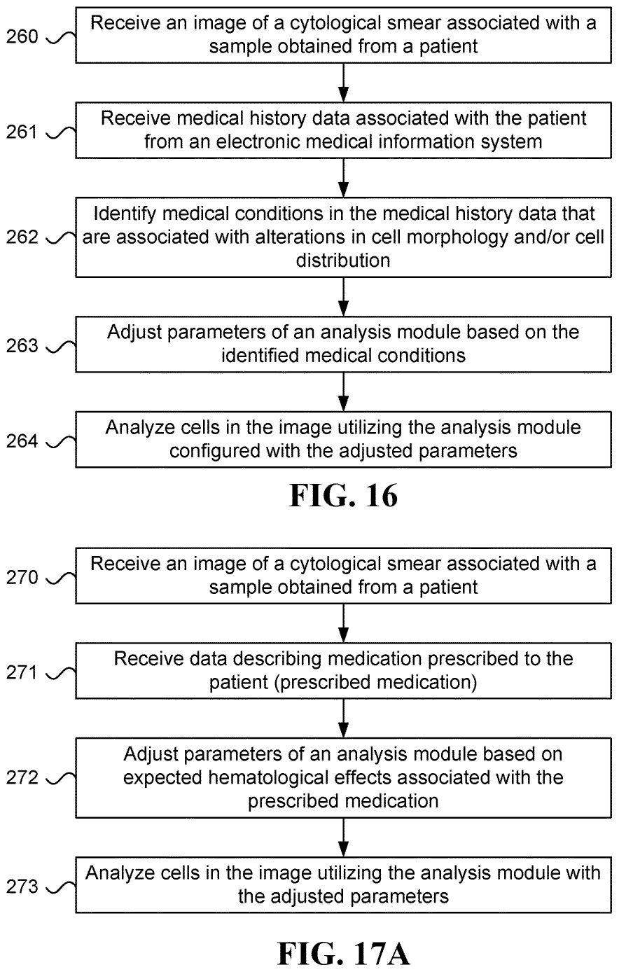

In various implementations, a computer-implemented method for integrating medical history in cytological smear analysis, comprising: receiving an image of a cytological smear associated with a sample obtained from a patient; receiving medical history data associated with the patient from an electronic medical information system; identifying medical conditions in the medical history data that are associated with alterations in cell morphology and/or cell distribution; adjusting parameters of an analysis module, based on the identified medical conditions; and analyzing cells in the image utilizing the analysis module configured with the adjusted parameters. FIG. 16 illustrates an embodiment of a computer-implemented method for integrating medical history in cytological smear analysis. The method includes the following steps. In step 260, receive an image of a cytological smear associated with a sample obtained from a patient. In step 261, receive medical history data associated with the patient from an electronic medical information system. In step 262, identify medical conditions in the medical history data that are associated with alterations in cell morphology and/or cell distribution. In step 263, adjust parameters of an analysis module, based on the identified medical conditions. And in step 264, analyze cells in the image utilizing the analysis module configured with the adjusted parameters.

In the context of this embodiment, cell morphology refers to the physical characteristics and/or structural features of individual cells observable in the cytological smear, including at least one of cell size, shape, membrane integrity, nuclear-to-cytoplasmic ratio, nuclear configuration, chromatin pattern, nucleolar characteristics, cytoplasmic features, inclusion bodies, granulation patterns, vacuolation, or staining characteristics. Changes in cell morphology may be indicative of various physiological and pathological processes, including cellular maturation, degeneration, activation, or transformation due to disease states. Furthermore, in the context of this embodiment, cell distribution refers to the spatial arrangement, relative proportions, and/or population densities of different cell types within a cytological smear. Cell distribution may include at least one of numerical relationships between different cell types, spatial patterns of cell arrangement (including clustering, isolation, or specific localization within the smear), gradient patterns that may form during smear preparation, population density variations across different regions of the smear, or relative frequencies of specific cell types compared to expected baseline proportions. Both cell morphology and cell distribution provide complementary diagnostic information, as certain medical conditions may manifest through altered morphological features of individual cells, changes in the quantities or relative proportions of different cell populations, or combinations thereof. The adjustment of the parameters of the analysis module based on identified medical conditions can account for these potential alterations in both morphological characteristics and distributional patterns, thereby improving classification accuracy in the context of the patient's specific medical history. Optionally, the analysis module includes at least one of an automatic cell classifier, or thresholds applied to results of the automatic cell classifier.

The parameters of the analysis module may encompass multiple categories of adjustable settings that influence the classification process, which may include: (1) model parameters, including weighting factors applied to specific cellular features, decision boundaries between cell type classifications, confidence thresholds for cell type assignment, and/or computational coefficients within classification algorithms; (2) output interpretation parameters, such as threshold levels applied to classification confidence scores, statistical significance requirements for rare cell population identification, and/or permitted ranges for morphological measurements; (3) image preprocessing parameters, including contrast enhancement settings, color normalization factors to account for staining variations, noise reduction parameters, and/or background correction coefficients that affect feature extraction; and (4) expected value parameters, comprising reference ranges for cell characteristics that vary based on medical conditions, demographic factors, and/or treatment status. The adjustment of these parameters based on identified medical conditions enables the classification system to account for expected variations in cellular presentation associated with specific medical contexts. For example, when a patient's medical history indicates chemotherapy treatment, the system may adjust threshold parameters for neutrophil identification to account for expected morphological changes, or when a patient has a known hematological disorder, the system may modify feature weighting to emphasize characteristics most relevant to monitoring that specific condition. These parameter adjustments enhance classification accuracy by incorporating patient-specific medical context into the analysis process.

In some implementations of the method, the analysis module comprises at least one of an automatic cell classifier, or thresholds applied to results of the automatic cell classifier, and further comprising presenting to a human reviewer at least some of the analyzed cells. In some implementations of the method, the adjusting of parameters comprises modifying a threshold applied to classification confidence level (CCL) outputs, wherein the adjusting accounts for known classification challenges associated with the identified medical conditions. In some implementations of the method, the adjusting of parameters comprises modifying image preprocessing parameters that affect feature extraction from the image, wherein the modifications compensate for expected staining variations or cellular presentation characteristics associated with the identified medical conditions. In some implementations of the method, the adjusting of parameters comprises updating a threshold applied to a hematological cell or cell type quantification metric (HCQM) for a cell type based on the identified medical conditions.

In some implementations of the method, the adjusting of parameters comprises modifying at least one of: feature weighting factors, decision boundaries, or confidence thresholds within the automatic cell classifier based on expected cellular characteristics associated with the identified medical conditions. In some implementations, the method further comprises selecting the adjusted parameters from a set of predetermined values.

In some implementations, the method further comprises determining whether to present, as part of default human review workflow, cells associated with the sample for specialist review or laboratory technician review based on severity and complexity of cell classifications, wherein the determining comprises comparing quantities of abnormal cell types against predetermined thresholds, presenting, as part of default human review workflow, cells associated with the sample directly to a specialist when the quantities exceed a first predetermined threshold, and presenting, as part of default human review workflow, cells associated with the sample to a laboratory technician for initial assessment when the quantities fall between the first predetermined threshold and a second predetermined threshold. This tiered review routing mechanism based on severity and complexity of cell classifications utilizes predetermined thresholds to determine the appropriate level of human expertise required for verification. When quantities of abnormal cell types exceed a first predetermined threshold (such as when multiple abnormal cell populations are detected or when the concentration of specific abnormal cells exceeds clinically significant levels), the system may route at least a subset of the image directly to a specialist, recognizing that cases with high complexity or severity benefit most from immediate specialist attention, bypassing preliminary review stages. For cases where the quantities of abnormal cell types fall between the first predetermined threshold and a second predetermined threshold, the system may route at least a subset of the image to a laboratory technician for initial assessment, recognizing that these cases exhibit abnormalities warranting human verification, but at levels that may be appropriately handled by a technician with the option for subsequent specialist escalation if necessary. When quantities of abnormal cells fall below the second predetermined threshold, the system may refrain from routing the image for human review, as the automated analysis results are considered sufficiently reliable for these cases with minimal detected abnormalities. This multi-tiered approach optimizes the allocation of human expertise while maintaining appropriate quality controls for cases of varying complexity. Cells associated with the sample, may refer to any cells associated with the sample, may include cells classified according to any classification, and may relate to any image or any cytological smear.

In some implementations, the method further comprises presenting, as part of default human review workflow, cells associated with the sample for human review based on their relevance to the identified medical conditions, wherein cells exhibiting characteristics associated with the patient's medical history are assigned a higher priority and cells with normal presentations are assigned relatively lower priority in the presentation to the human reviewer. The assignment of relatively lower priority to cells with normal presentations within the routing process may encompass multiple implementation approaches. It may involve exclusion of normal cells from the routed subset, thereby focusing reviewer attention on cells with potentially abnormal characteristics. Alternatively, lower priority may be implemented through visual de-emphasis within the review interface, where normal cells remain accessible but are displayed with reduced prominence or accessible through secondary navigation options. In other implementations, lower priority may manifest as sequenced presentation, where normal cells are presented after the reviewer has examined higher-priority cells, or as filtered presentation that allows reviewers to optionally view normal cells through explicit interface selections. The specific implementation of priority differentiation may be selected based on laboratory workflow requirements, reviewer preferences, and the clinical context of the analysis.

In some implementations, the method further comprises evaluating cell classifications against the identified medical conditions to determine review requirements; and selecting, when review is required, a human reviewer from available reviewers based on correspondence between the identified medical conditions and reviewer expertise profiles. The expertise-matched reviewer selection mechanism can enhance diagnostic quality by aligning reviewer specialization with patient-specific medical conditions. The system evaluates cell classifications against the identified medical conditions to determine whether human review is warranted, considering factors such as classification confidence, deviation from expected patterns, and/or clinical significance in the patient's specific medical context. When review is required, the system may select a human reviewer from available reviewers based on correspondence between the identified medical conditions and reviewer expertise profiles. This selection process may incorporate multiple expertise dimensions including subspecialty training, years of experience with specific conditions, certification status, and historical performance metrics for similar cases. The expertise profiles may be maintained in a structured repository that enables matching algorithms to identify the appropriate available reviewer for each case. This targeted routing to personnel with the relevant specialized knowledge for the patient's specific medical context potentially improves diagnostic accuracy while creating opportunities for expertise development through appropriate case assignments.

In some implementations of the method, the adjusting comprises modifying hematological cell or cell type quantification metric (HCQM) thresholds for certain cell types based on prevalence expectations associated with the identified medical conditions. The system may adjust classification thresholds for specific cell types based on their expected prevalence in patients with particular medical conditions. For example, patients with known myeloproliferative disorders may have higher baseline levels of immature granulocytes, which may necessitate adjusted classification thresholds to avoid false positive flags. Conversely, for patients with conditions where early detection of specific cell types is critical, such as monitoring for blast cells in leukemia remission, the thresholds may be lowered to increase sensitivity. These condition-specific threshold adjustments improve classification accuracy by accounting for the patient's specific medical context.

In some implementations of the method, the medical history data includes previous hematological test results, and the adjusting accounts for established baseline values and trends specific to the patient. The system may utilize the patient's previous hematological test results to establish personalized baseline values and identify individual variation patterns. This patient-specific context may allow the classifier to distinguish between longstanding stable abnormalities and newly emerging changes that may have greater clinical significance. For patients with chronic conditions that produce consistent cellular abnormalities, such as hereditary spherocytosis or thalassemia traits, the system adjusts classification parameters to recognize these as constitutive features of the patient's blood profile rather than acute pathological changes, thereby reducing unnecessary flags for stable conditions while remaining sensitive to new developments.

In some implementations, the method further comprises generating a report that contextualizes the cytological smear analysis results within the framework of the patient's medical history, and highlighting findings that represent changes from the patient's baseline or expected presentation. The system may generate reports that integrate cytological analysis results with relevant medical history to enhance clinical interpretation. These reports may highlight cellular changes that represent deviations from the patient's established baseline or expected patterns based on their medical conditions. For example, a moderate lymphocytosis may be flagged as significant in most patients but contextualized as an expected finding in a patient with known chronic lymphocytic leukemia. The report may include comparative visualizations showing current findings against historical trends, annotations explaining the clinical significance of specific findings in the patient's particular medical context, and prioritized highlighting of unexpected findings that warrant further investigation.

In some implementations, the method further comprises comparing current analysis results with previous analysis results for the patient; determining whether cell population changes are consistent with expected progression of identified medical conditions; and presenting, as part of default human review workflow, cells associated with the sample for human review when the changes deviate from expected patterns based on the medical history data. The system may implement temporal analysis by comparing current results against the patient's previous analyses to detect clinically significant changes. This comparison evaluates whether observed cellular changes align with expected disease progression patterns for the identified medical conditions. When changes deviate from expected patterns-such as when a patient in remission shows unexpected emergence of immature cell forms, or when a patient on therapy shows cellular changes inconsistent with the expected treatment response—the system routes at least the relevant subset of the image for human expert review. This temporal analysis provides context to detect subtle but significant deviations that may indicate disease progression, treatment failure, or secondary complications.

In some implementations of the method, the presenting, as part of default human review workflow, cells associated with the sample, comprises presenting identified subsets of cells grouped according to their relevance to specific medical conditions identified in the medical history data, with contextual medical information provided alongside at least some of the groups. When routing cells for human review, the system may organize the presentation by grouping cells according to their relevance to specific medical conditions in the patient's history. This organizational approach allows reviewers to efficiently evaluate cells within their appropriate clinical context. For example, cells relevant to monitoring leukemia remission may be grouped together with contextual information about the patient's treatment history and previous blast percentages. This contextualized presentation may reduce cognitive load for reviewers by structuring the review process around clinically meaningful categories, potentially improving review accuracy and efficiency.

In some implementations of the method, the adjusting comprises modifying statistical significance thresholds for cell population analysis based on the identified medical conditions, wherein rarer medical conditions are associated with reduced statistical significance thresholds to trigger human review. The system may adjust statistical significance requirements based on the rarity and clinical importance of the identified medical conditions. For common conditions with well-established cellular presentation patterns, standard statistical thresholds may be appropriate. However, for rare conditions where limited reference data exists, or where early detection is required, the system may implement reduced statistical significance thresholds. This adjustment recognizes that for rare conditions, waiting for highly statistically significant changes may delay critical interventions. By calibrating statistical requirements to the specific medical context, the system can balance false positive risk against the potential consequences of delayed detection for various condition types.

In some implementations of the method, the medical history data associated with the patient comprises: receiving treatment status information associated with the patient; and adjusting the parameters based on the treatment status information, wherein at least two different treatment statuses result in different parameter adjustments. The system may implement treatment status-based parameter adjustments that recognize the expected cellular abnormalities associated with different types of medical treatments. By receiving treatment status information from the electronic medical information system, the system can apply appropriate parameters that account for treatment-related cellular changes. For example, for patients undergoing chemotherapy, the system may lower neutrophil CCL thresholds by a first percentage and relax cell count thresholds, since neutropenia is an expected consequence of many chemotherapy regimens. For patients in remission monitoring phases, the system may implement stricter thresholds for blast cells, increasing them by a second percentage to improve detection sensitivity for early relapse indicators. For patients on maintenance therapy, the system may adjust thresholds based on medication cycle timing, applying more lenient thresholds during expected nadir periods when cellular abnormalities are anticipated as part of the treatment process. This treatment-aware process may reduce unnecessary additional testing or specialist referrals for patients with treatment-induced cellular changes while maintaining appropriate vigilance for unexpected deviations that might indicate disease progression or treatment complications.

In some implementations of the method, the treatment status information includes treatment cycle timing data, and wherein the adjusting comprises modifying the parameters according to at least some of the expected cellular profile variations at different points in the treatment cycle. Many treatment regimens, particularly those for hematological disorders and/or conditions that manifest in cells, follow cyclical patterns with predictable effects on blood cell populations at different points in the cycle. The system may leverage treatment cycle timing data to implement dynamic parameter adjustments that align with expected cellular profile variations throughout the cycle. For example, immediately following chemotherapy administration, the system may apply relaxed parameters for neutrophil and platelet counts, as these are expected to decrease. As the cycle progresses toward the typical recovery phase, the system may gradually return parameters toward standard thresholds. For maintenance chemotherapy regimens with predictable nadirs (points of lowest cell counts), the system may implement a temporal adjustment model that varies thresholds according to the number of days since treatment administration. Similarly, for treatments with cumulative effects over multiple cycles, the system may progressively adjust parameters to account for the expected increased severity of cellular changes as treatment accumulates. This cycle-aware process can help distinguish between expected treatment-related cellular changes and unexpected findings that may indicate treatment complications or disease progression.

This embodiment may incorporate interactive validation workflows that enhance the automated medical history integration process without disrupting the core parameter adjustment functionality. Laboratory personnel may engage with the system through collaborative interfaces that facilitate verification of medical condition identification, where clinical staff can review and validate automated parsing of medical history data before parameter adjustments are finalized. The system may implement dynamic feedback mechanisms that allow medical technologists to confirm treatment status information during the receiving of medical history data from the electronic medical information system, ensuring accuracy of temporal treatment cycle data that influences parameter modifications. Interactive workflows may enable specialist reviewers to participate in the adjusting of parameters by providing clinical context for medical conditions, validating that parameter modifications appropriately reflect expected cellular alterations, and establishing patient-specific parameter profiles for recurring analysis requirements. The system may provide collaborative tools for medical staff to interactively verify that identified medical conditions correctly correspond to expected alterations in cell morphology and/or cell distribution, with interface elements that support annotation of unusual medical history patterns and confirmation of treatment cycle timing data. Additionally, the system may facilitate expert consultation workflows where pathologists can validate parameter adjustment decisions for medical histories, confirm that analysis module configurations appropriately account for multiple concurrent medical conditions, and establish institutional protocols for parameter modification in specialized clinical scenarios. These optional interactive validation processes may operate alongside the automated identifying of medical conditions and adjusting of parameters, creating collaborative workflows that leverage both automated efficiency and human medical expertise while maintaining the automated integration of medical history data into cytological smear analysis. In some implementations of the method, a non-transitory computer-readable medium comprising instructions that, when executed by a processor, cause the processor to perform the computer-implemented method.

In various implementations, a system for integrating medical history in cytological smear analysis, comprising: a computer configured to: receive an image of a cytological smear associated with a sample obtained from a patient; receive medical history data associated with the patient from an electronic medical information system; identify medical conditions in the medical history data that are associated with alterations in cell morphology and/or cell distribution; adjust parameters of an analysis module, based on the identified medical conditions; and analyze cells in the image utilizing the analysis module configured with the adjusted parameters.

In some implementations of the system, the analysis module comprises at least one of an automatic cell classifier, or thresholds applied to results of the automatic cell classifier, and wherein the computer is further configured to cause at least some of the analyzed cells to be presented to a human reviewer. In some implementations of the system, the computer is further configured to adjust the parameters by modifying a threshold applied to classification confidence level (CCL) outputs, wherein the adjustment accounts for known classification challenges associated with the identified medical conditions.

In some implementations of the system, the computer is further configured to adjust the parameters by modifying image preprocessing parameters that affect feature extraction from the image, wherein the modifications compensate for expected staining variations or cellular presentation characteristics associated with the identified medical conditions. In some implementations of the system, the computer is further configured to adjust the parameters by updating a threshold applied to a hematological cell or cell type quantification metric (HCQM) for a cell type based on the identified medical conditions.

In some implementations of the system, the computer is further configured to adjust the parameters by modifying at least one of: feature weighting factors, decision boundaries, or confidence thresholds within the automatic cell classifier based on expected cellular characteristics associated with the identified medical conditions. In some implementations of the system, the computer is further configured to select the adjusted parameters from a set of predetermined values. In some implementations of the system, the computer is further configured to: determine whether to present, as part of default human review workflow, cells associated with the sample for specialist review or laboratory technician review based on severity and complexity of cell classifications, wherein the determining comprises comparing quantities of abnormal cell types against predetermined thresholds; cause cells associated with the sample to be presented directly to a specialist as part of default human review workflow when the quantities exceed a first predetermined threshold; and cause cells associated with the sample to be presented to a laboratory technician for initial assessment as part of default human review workflow when the quantities fall between the first predetermined threshold and a second predetermined threshold.

In some implementations of the system, the computer is further configured to: cause cells associated with the sample to be presented for human review as part of default human review workflow based on their relevance to the identified medical conditions, wherein cells exhibiting characteristics associated with the patient's medical history are assigned a higher priority and cells with normal presentations are assigned relatively lower priority in the presentation to the human reviewer. In some implementations of the system, the computer is further configured to: evaluate cell classifications against the identified medical conditions to determine review requirements; and select, when review is required, a human reviewer from available reviewers based on correspondence between the identified medical conditions and reviewer expertise profiles.

In some implementations of the system, the computer is further configured to adjust the parameters by modifying hematological cell or cell type quantification metric (HCQM) thresholds for certain cell types based on prevalence expectations associated with the identified medical conditions. In some implementations of the system, the medical history data includes previous hematological test results, and wherein the computer is further configured to adjust the parameters to account for established baseline values and trends specific to the patient. In some implementations of the system, the computer is further configured to generate a report that contextualizes the cytological smear analysis results within the framework of the patient's medical history, and highlight findings that represent changes from the patient's baseline or expected presentation.

In some implementations of the system, the computer is further configured to: compare current analysis results with previous analysis results for the patient; determine whether cell population changes are consistent with expected progression of identified medical conditions; and cause cells associated with the sample to be presented for human review as part of default human review workflow when the changes deviate from expected patterns based on the medical history data. In some implementations of the system, the computer is further configured to cause identified subsets of cells to be presented grouped according to their relevance to specific medical conditions identified in the medical history data, with contextual medical information provided alongside at least some of the groups.

In some implementations of the system, the computer is further configured to adjust the parameters by modifying statistical significance thresholds for cell population analysis based on the identified medical conditions, wherein rarer medical conditions are associated with reduced statistical significance thresholds to trigger human review. In some implementations of the system, the computer is further configured to: receive treatment status information associated with the patient; and adjust the parameters based on the treatment status information, wherein at least two different treatment statuses result in different parameter adjustments. In some implementations of the system, the treatment status information includes treatment cycle timing data, and wherein the computer is further configured to adjust the parameters by modifying the parameters according to at least some of the expected cellular profile variations at different points in the treatment cycle.

The following implementation focuses specifically on adjusting cytological smear analysis based on the patient's medication profile. By incorporating knowledge of how medications affect blood cell parameters, the system may reduce false positives and negatives in cell classification. It may improve diagnostic accuracy by distinguishing between medication-induced changes and pathological findings. The embodiment supports more informed interpretation of cytological smears associated with patients on medications known to affect hematological parameters. Additionally, the system may implement routing for human review that account for medication-induced cellular changes, potentially reducing some of the unnecessary expert consultations for expected medication effects while improving review of unexpected findings. Differential review may be employed to focus human expertise on specific cell populations most likely to demonstrate clinically significant deviations beyond expected medication effects.

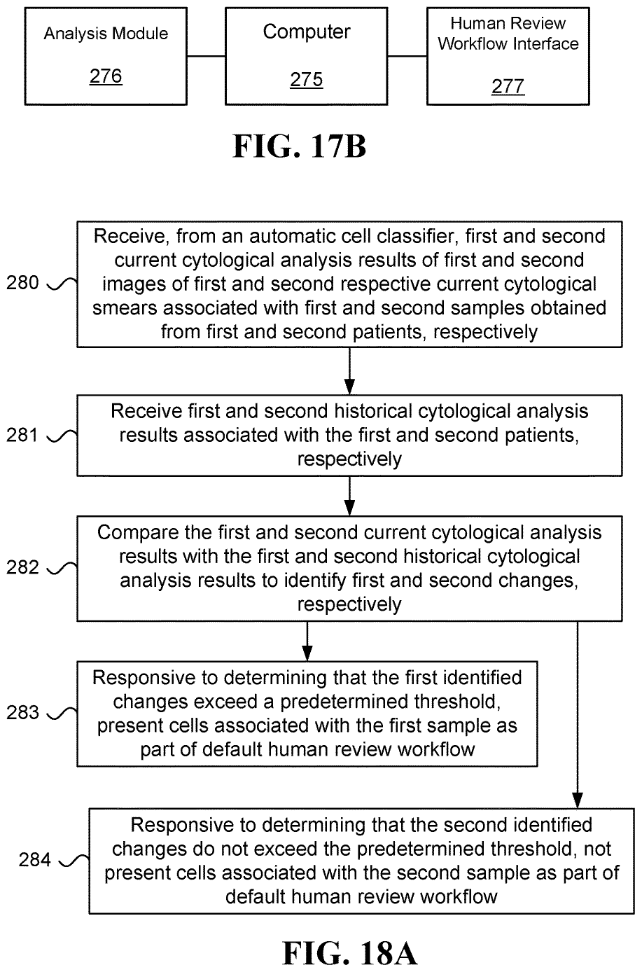

In various implementations, a computer-implemented method for medication-aware automatic cytological smear analysis, comprising: receiving an image of a cytological smear associated with a sample obtained from a patient; receiving data describing medication prescribed to the patient (prescribed medication); adjusting parameters of an analysis module based on expected hematological effects associated with the prescribed medication; and analyzing cells in the image utilizing the analysis module with the adjusted parameters. FIG. 17A illustrates an embodiment of a computer-implemented method for medication-aware automatic cytological smear analysis. The method includes the following steps. In step 270, receive an image of a cytological smear associated with a sample obtained from a patient. In step 271, receive data describing medication prescribed to the patient (prescribed medication). In step 272, adjust parameters of an analysis module based on expected hematological effects associated with the prescribed medication. And in step 273, analyze cells in the image utilizing the analysis module with the adjusted parameters.

The adjusting of parameters may be implemented through multiple approaches depending on the specific requirements and capabilities of the analysis module. In a cell-specific adjustment approach, parameters are modified for individual cells based on their morphological characteristics in relation to expected medication effects. In a cell type-based adjustment approach, parameters are modified categorically for specific cell populations (such as neutrophils, lymphocytes, or monocytes) known to be affected by the prescribed medication. Additional adjustment approaches may include: medication category-based adjustments that apply parameter modifications based on the therapeutic class or mechanism of action of the medication; protocol-specific adjustments that modify parameters based on established analysis protocols for patients on specific medication regimens; temporal pattern adjustments that account for expected chronological changes in cell presentation throughout medication treatment cycles; and/or global classifier adjustments that modify core classification algorithms to better accommodate medication-induced variations across multiple cell types simultaneously. The system may implement one or multiple adjustment approaches in combination depending on the specific medication profile and clinical context.

In some implementations of the method, the analysis module comprises at least one of an automatic cell classifier, or thresholds applied to results of the automatic cell classifier. In some implementations, the method further comprises selecting the adjusted parameters from a set of predetermined values, and presenting to a human reviewer at least some of the analyzed cells.

In some implementations of the method, for prescribed medication known to cause neutropenia, the adjusting comprises adjusting classification confidence level (CCL) thresholds for neutrophil identification to allow for expected morphology changes, or modifying thresholds applied to neutrophil hematological cell or cell type quantification metric (HCQM) parameters to account for expected medication-induced reductions. For medications known to cause neutropenia, such as certain chemotherapeutic agents, immunosuppressants, psychiatric drugs, and antibiotics, the system implements specialized parameter adjustments focused on neutrophil analysis. These adjustments include adjusting CCL thresholds to improve neutrophil detection sensitivity in samples with reduced neutrophil populations, wherein higher CCL is associated with higher confidence this may include lowering the CCL threshold, modifying morphological feature weights to accommodate medication-induced changes in neutrophil appearance, and/or adjusting statistical significance thresholds for neutrophil HCQM evaluations. These medication-aware parameter modifications can help prevent misclassification of expected medication-induced neutropenia as pathological neutropenia requiring clinical intervention, while maintaining sensitivity to detect neutrophil reductions that exceed expected medication effects. This approach may reduce unnecessary clinical alerts for anticipated medication effects while preserving the ability to identify neutrophil abnormalities that may indicate adverse medication reactions or concurrent pathological processes.

In some implementations of the method, the adjusting comprises modifying morphological classification parameters to account for medication-induced changes in cell appearance that may otherwise be interpreted as pathological. Many medications induce predictable alterations in cellular morphology that, without proper context, might be misinterpreted as pathological changes. The system modifies morphological classification parameters to accommodate these expected medication-induced appearances. For example, certain antibiotics may cause specific granulation patterns in neutrophils, antimalarials can induce vacuolation in white blood cells, and antipsychotics may affect erythrocyte membrane characteristics. Parameter adjustments may include: modifying feature recognition thresholds for specific cellular structures; adjusting weighting factors applied to certain morphological characteristics; expanding acceptable ranges for size, shape, or granularity metrics; and implementing medication-specific decision boundaries in the classification algorithm. These adjustments enable the classifier to distinguish between medication-induced morphological changes and similar-appearing pathological changes, reducing false positive classifications while maintaining sensitivity to detect truly abnormal morphological features that may coexist with medication effects.

In some implementations of the method, the adjusting is performed based on prescribed medication dosage information, with parameter adjustments proportionally related to dosage levels of the prescribed medication. The system may implement dose-dependent parameter adjustments that reflect the relationship between medication dosage and expected hematological effects. As medication effects on blood cells often demonstrate dose-dependent relationships, the parameters are modified proportionally to the prescribed dosage levels. For medications with established dose-response curves for hematological effects, such as many chemotherapeutic agents or immunosuppressants, the system may apply mathematical transformations that correlate classifier parameters with dosage levels. For example, neutrophil identification thresholds may be adjusted by a percentage proportional to the chemotherapy dose relative to standard protocol levels, or platelet detection parameters may be modified according to the specific relationship between anticoagulation medication dosage and expected inhibition effects. This proportional adjustment approach may create a more nuanced classification system that accounts for the variable intensity of medication effects based on dosage, rather than applying binary adjustments that treat all doses of a medication as having equivalent effects.

In some implementations of the method, the adjusting accounts for the temporal relationship between the prescribed medication administration and sample collection time, with different parameter adjustments applied based on medication half-life and known time-dependent effects. The system may incorporate temporal relationship analysis between medication administration and sample collection to optimize parameter adjustments. Many medications demonstrate predictable time-course effects on blood cells, with varying effects during initial administration, peak action, steady-state, and washout periods. The system may apply different parameter adjustments based on where the sample collection falls within this temporal profile, utilizing medication half-life data and established pharmacokinetic models. For example, immediately following administration of medications known to cause transient leukocytosis (such as corticosteroids), WBC identification and counting parameters may be adjusted to account for the expected temporary increase, while different adjustments would be applied during steady-state treatment phases. For cyclical treatment regimens like chemotherapy protocols, the system may implement phase-specific parameter adjustments that align with expected cellular recovery and nadir patterns, which may improve classification accuracy by accounting for the dynamic nature of medication effects over time.

In some implementations, the method further comprises determining, based on the analysis of the image, whether findings deviate from expected medication-induced changes; and presenting, as part of default human review workflow, cells associated with the sample for human review when the findings exceed expected effects of the prescribed medication or when the findings include unexpected cell populations not typically affected by the prescribed medication. The system may implement intelligent human review routing based on deviation analysis between observed cellular changes and expected medication effects. After analyzing the image using the adjusted parameters, the system may compare the findings against medication-specific effect profiles to identify unexpected deviations. This comparison evaluates both the magnitude of effects on expected target cell populations (such as when neutrophil reductions exceed typical ranges for a given chemotherapy regimen) and the presence of effects in cell populations not typically impacted by the medication (such as when a medication known to affect primarily neutrophils is associated with unexpected lymphocyte abnormalities). When such deviations are detected, the system routes a relevant image subset for human expert review, prioritizing findings that may indicate adverse medication reactions, concurrent pathological processes, or unusual patient-specific medication responses. This targeted routing approach focuses human expertise on cases where findings fall outside the expected medication effect profile, potentially improving detection of clinically significant abnormalities while reducing unnecessary reviews of expected medication-induced changes. Cells associated with the sample, may refer to any cells associated with the sample, may include cells classified according to any classification, and may relate to any image or any cytological smear.

In some implementations of the method, responsive to determining to present, as part of default human review workflow, cells associated with the sample for human review, further comprising: identifying specific subsets of cells that exhibit characteristics deviating from expected medication-induced changes; and presenting the identified subsets of cells for human review. When routing for human review, the system may implement targeted cell selection that focuses reviewer attention on specific cells exhibiting characteristics that deviate from expected medication-induced changes. Rather than routing the entire image, which may contain numerous cells displaying anticipated medication effects, the system may identify and extract specific cells or cell clusters that warrant expert evaluation. This targeted approach may include cells displaying morphological features that exceed expected medication-induced alterations, cells from populations showing quantitative deviations beyond expected ranges, or cells exhibiting combinations of characteristics that are inconsistent with known medication effect profiles. By routing these specific cell subsets with precise deviation characteristics, the system creates a more efficient review process that directs human expertise to the clinically relevant findings while reducing the time spent examining cells displaying expected medication effects.

In some implementations, the method further comprises presenting the identified specific subsets of cells alongside relevant medication information comprising at least one of: expected prescribed medication effects, dosage, administration timing, or visualization of the boundary between expected and unexpected cellular presentations. The system may enhance the review experience by presenting identified cell subsets within a comprehensive medication context framework. This contextual presentation may include relevant medication information displayed alongside the cellular images, creating an integrated review environment that facilitates more informed interpretation. The medication information may include visualization of expected effect ranges for specific cell parameters with the patient's actual values overlaid, graphical representations of typical temporal effect patterns with indicators showing the sample collection timing, dose-dependent effect curves with the patient's dosage highlighted, or comparative reference images showing typical medication-induced cellular changes versus pathological changes with similar appearances. By providing this medication context simultaneously with the cell images, the system enables reviewers to more effectively distinguish between expected pharmacological effects and potential pathological findings, potentially improving review accuracy and efficiency for patients on medications with known hematological effects.

In some implementations of the method, responsive to determining to present, as part of default human review workflow, cells associated with the sample for human review, further comprising selecting a specific human reviewer based on expertise in diagnosis of cases in field of treatment, expertise in pharmacological effects of the prescribed medication, or expertise in the specific cell populations affected by the prescribed medication. The system may implement intelligent reviewer selection that matches cases with human experts possessing relevant pharmacological or hematological expertise. When routing for human review, the system may consider the specific medication involved and the affected cell populations to identify reviewers with appropriate specialized knowledge. For complex medication regimens such as multi-agent chemotherapy protocols, the system may select reviewers with oncological pharmacy expertise; for immunosuppressive therapies, reviewers with transplant medicine or immunology backgrounds; and for novel therapeutics with less established hematological effect profiles, reviewers with advanced training in medication effects or adverse reaction assessment.

In some implementations, the method further comprises determining a priority level for human review based on the degree of deviation from expected effects of the prescribed medication and clinical significance of the affected cell populations; and adjusting the quantity of cells presented for review based on current reviewer workload, wherein higher priority deviations maintain comprehensive presentation essentially regardless of workload while lower priority deviations lead to presenting less cells during high workload periods. The system may implement adaptive routing methods that balance clinical significance with resource constraints. For each case requiring human review, the system may assign a priority level based on the deviation magnitude from expected medication effects and the clinical significance of the affected cells. This prioritization enables workload-aware routing decisions that optimize reviewer resource allocation while maintaining appropriate clinical vigilance. During high workload periods, the system may implement selective routing for lower-priority cases, directing the most significant subset of deviant cells for review, while maintaining comprehensive routing for high-priority cases essentially regardless of workload conditions. This adaptive approach may include dynamic threshold adjustments that become more selective as workload increases, automated batching of similar deviation types to improve review efficiency, or temporary routing to secondary review teams with appropriate expertise when primary reviewers reach capacity thresholds. The workload-adaptive routing mechanisms can help laboratories maintain throughput during high-volume periods while preserving review quality for clinically significant findings.

In some implementations, the method further comprises identifying multiple parameters that individually fall within expected ranges for the prescribed medication but collectively represent an unusual pattern; and presenting, as part of default human review workflow, cells associated with the sample for human review based on the unusual collective pattern even when individual parameters alone would not trigger human review. The system may implement pattern recognition that identifies clinically significant collective abnormalities that may be missed through individual parameter assessment. While single hematological parameters often remain within expected ranges for medication effects, certain combinations of individually normal parameters may represent significant patterns requiring expert evaluation. The system may analyze relationships between multiple parameters, such as ratios between different cell populations, correlations between morphological features, or temporal patterns across sequential measurements, to detect these collective abnormalities. When such patterns are identified—such as when lymphocyte morphology, neutrophil count, and red cell distribution width individually fall within medication-expected ranges but collectively suggest an emerging pattern inconsistent with medication effects alone—the system may route relevant cells for human review despite the lack of individual parameter deviations. This pattern-based routing can help identify subtle but clinically significant findings that may be overlooked by systems that evaluate parameters only in isolation.

In some implementations, the method further comprises distinguishing between sample preparation artifacts and medication-induced cellular changes using medication-specific artifact reference patterns; and prioritizing for human review the cellular changes that cannot be attributed to expected effects of the prescribed medication or known artifacts. The system may implement specialized artifact differentiation capabilities that distinguish between preparation-induced artifacts and medication effects. Certain sample preparation artifacts can mimic medication-induced cellular changes, creating potential confusion during analysis. The system utilizes medication-specific artifact reference patterns to differentiate between these phenomena through feature analysis or trained models comparing observed characteristics against known artifact presentation patterns. For example, certain staining artifacts may produce vacuole-like appearances similar to those induced by specific medications, but typically display different distribution patterns or optical characteristics that the system can detect. When cellular changes are observed, the system may classify them into three categories: expected medication effects, known preparation artifacts, or unexplained changes that cannot be attributed to either medication effects or artifacts. The third category receives priority for human review, as these changes may represent clinically significant findings that warrant expert evaluation. This differentiation process can help reduce false positives from artifact misclassification while maintaining appropriate sensitivity for detecting abnormalities. The system may access expected hematological effects from a medication effects repository that aggregates knowledge about medication impacts on blood cells, which may be implemented through various technical approaches, including structured databases containing quantitative effect profiles, neural networks with encoded medication effect knowledge distributed across hidden layers, deep learning model parameters trained on medication-specific datasets, or inference model weights calibrated to detect specific medication-induced patterns. The repository may incorporate multiple knowledge sources including published literature, regulatory documentation, pharmacovigilance data, and anonymized patient outcome data that contribute to a comprehensive representation of medication effects on hematological parameters. In some implementations of the method, a non-transitory computer-readable medium comprising instructions that, when executed by a processor, cause the processor to perform the computer-implemented method.

In various implementations, a system for medication-aware automatic cytological smear analysis, comprising: a computer configured to: receive an image of a cytological smear associated with a sample obtained from a patient; receive data describing medication prescribed to the patient (prescribed medication); adjust parameters of an analysis module based on expected hematological effects associated with the prescribed medication; and analyze cells in the image utilizing the analysis module with the adjusted parameters. FIG. 17B illustrates an embodiment of a block diagram of a system for medication-aware automatic cytological smear analysis. The system includes a computer 275 that adjusts parameters of an analysis module 276, and is further coupled to a human review workflow interface 277.

In some implementations of the system, the analysis module comprises at least one of an automatic cell classifier, or thresholds applied to results of the automatic cell classifier. In some implementations of the system, the computer is further configured to: select the adjusted parameters from a set of predetermined values, and cause at least some of the analyzed cells to be presented to a human reviewer. In some implementations of the system, for prescribed medication known to cause neutropenia, the adjusting comprises adjusting classification confidence level (CCL) thresholds for neutrophil identification to allow for expected morphology changes, or modifying thresholds applied to neutrophil hematological cell or cell type quantification metric (HCQM) parameters to account for expected medication-induced reductions.

In some implementations of the system, the adjusting comprises modifying morphological classification parameters to account for medication-induced changes in cell appearance that may otherwise be interpreted as pathological. In some implementations of the system, the adjusting is performed based on prescribed medication dosage information, with parameter adjustments proportionally related to dosage levels of the prescribed medication. In some implementations of the system, the adjusting accounts for the temporal relationship between the prescribed medication administration and sample collection time, with different parameter adjustments applied based on medication half-life and known time-dependent effects.

In some implementations of the system, the computer is further configured to: determine, based on the analysis of the image, whether findings deviate from expected medication-induced changes; and cause cells associated with the sample to be presented as part of default human review workflow for human review when the findings exceed expected effects of the prescribed medication or when the findings include unexpected cell populations not typically affected by the prescribed medication. In some implementations of the system, responsive to determining to cause cells associated with the sample to be presented as part of default human review workflow for human review, the computer is further configured to: identify specific subsets of cells that exhibit characteristics deviating from expected medication-induced changes; and cause the identified subsets of cells to be presented for human review. In some implementations of the system, the computer is further configured to cause the identified specific subsets of cells to be presented alongside relevant medication information comprising at least one of: expected prescribed medication effects, dosage, administration timing, or visualization of the boundary between expected and unexpected cellular presentations.

In some implementations of the system, responsive to determining to cause cells associated with the sample to be presented as part of default human review workflow for human review, the computer is further configured to select a specific human reviewer based on expertise in diagnosis of cases in field of treatment, expertise in pharmacological effects of the prescribed medication, or expertise in the specific cell populations affected by the prescribed medication. In some implementations of the system, the computer is further configured to: determine a priority level for human review based on the degree of deviation from expected effects of the prescribed medication and clinical significance of the affected cell populations; and adjust the quantity of cells presented for review based on current reviewer workload, wherein higher priority deviations maintain comprehensive presentation essentially regardless of workload while lower priority deviations lead to presenting less cells during high workload periods.

In some implementations of the system, the computer is further configured to: identify multiple parameters that individually fall within expected ranges for the prescribed medication but collectively represent an unusual pattern; and cause cells associated with the sample to be presented as part of default human review workflow for human review based on the unusual collective pattern even when individual parameters alone would not trigger human review.

In some implementations of the system, the computer is further configured to: distinguish between sample preparation artifacts and medication-induced cellular changes using medication-specific artifact reference patterns; and prioritize for human review the cellular changes that cannot be attributed to expected effects of the prescribed medication or known artifacts.

The following implementation addresses the analysis of current cytological smear results in the context of the patient's previous test results. By incorporating historical data, the system may identify changes that warrant human attention while reducing unnecessary reviews for stable conditions, which may improve efficiency by focusing human review on cases showing clinically significant changes, and may support longitudinal monitoring of patients' conditions by highlighting meaningful variations over time.

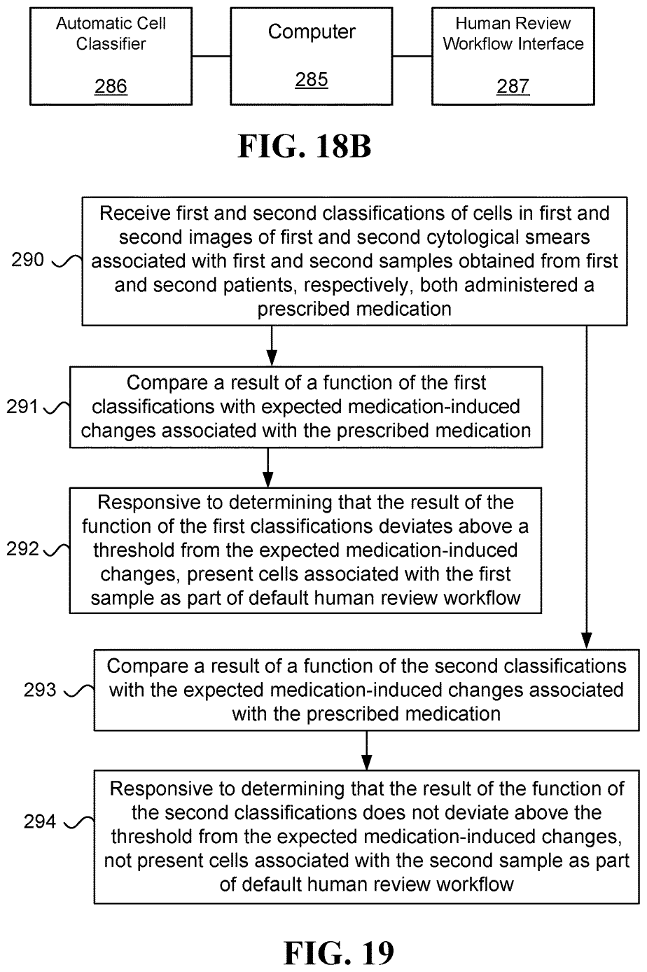

In various implementations, a computer-implemented method for comparative analysis of cytological smears, comprising: receiving, from an automatic cell classifier, first and second current cytological analysis results of first and second images of first and second respective current cytological smears associated with first and second samples obtained from first and second patients, respectively; receiving first and second historical cytological analysis results associated with the first and second patients, respectively; comparing the first and second current cytological analysis results with the first and second historical cytological analysis results to identify first and second changes, respectively; responsive to determining that the first identified changes exceed a predetermined threshold, presenting, as part of default human review workflow, cells associated with the first sample; and responsive to determining that the second identified changes do not exceed the predetermined threshold, not presenting, as part of default human review workflow, cells associated with the second sample. FIG. 18A illustrates an embodiment of a computer-implemented method for comparative analysis of cytological smears. The method includes the following steps. In step 280, receive, from an automatic cell classifier, first and second current cytological analysis results of first and second images of first and second respective current cytological smears associated with first and second samples obtained from first and second patients, respectively. In step 281, receive first and second historical cytological analysis results associated with the first and second patients, respectively. In step 282, compare the first and second current cytological analysis results with the first and second historical cytological analysis results to identify first and second changes, respectively. In step 283, responsive to determining that the first identified changes exceed a predetermined threshold, present, as part of default human review workflow, cells associated with the first sample. And in step 284, responsive to determining that the second identified changes do not exceed the predetermined threshold, not present, as part of default human review workflow, cells associated with the second sample.

In the context of this embodiment, “not presenting, as part of default human review workflow, cells associated with the second sample” refers to excluding the second sample from the default review process when the changes identified through comparison with the second historical cytological analysis results do not exceed the predetermined threshold. While the first sample is presented for human review because the first identified changes exceed the predetermined threshold (potentially indicating clinically significant evolution from the first patient's baseline), the second sample is not presented because the second identified changes remain below the predetermined threshold (indicating for example stability or insignificant variation from the second patient's historical patterns). The not presenting may be implemented through various mechanisms including: excluding the second sample from review queues, routing the second sample directly to automated reporting pathways that bypass human verification, preventing the second sample from appearing in reviewer worklists or task assignments, marking the second sample with metadata flags indicating minimal change from historical baseline while marking the first sample with flags indicating significant change requiring review, applying different marking schemes to the first and second samples that are distinguishable through visual indicators, textual annotations, numerical indicators representing degree of change, storing the second sample in repositories designated for stable or unchanged cases rather than in pending review repositories, presenting the comparative analysis results for the second sample to a human without an indication that review is required whereas the comparative analysis results for the first sample are presented with an indication that review is required due to significant changes, displaying indicators of the degree of change that visually differentiate samples requiring review from those not requiring review based on comparison with respective historical baselines, and/or displaying the second sample in a manner distinguishable from the first sample in the default human review workflow, wherein such distinguishing manners may include distinct borders, different background shading, modified interaction controls, presence or absence of change alert indicators, different positioning within the interface based on the extent of change from historical results, alternative display formats for stable versus changed results, graphical representations showing the second sample within historical variance bands versus the first sample exceeding such bands, or textual labels that identify the second sample as excluded from default human review workflow based on minimal change from historical results. The system may additionally implement temporal context displays showing that the second sample falls within expected variation patterns while the first sample deviates significantly from established baselines, wherein the degree of change may be quantified through various metrics such as percentage differences in cell counts, morphological parameter variations, classification distribution shifts, or composite scores derived from multiple cytological features compared between current and historical results. By not presenting the second sample while presenting the first sample based on historical comparisons, the system may focus human expertise on cases with potentially significant changes from baseline while avoiding unnecessary review of stable cases. The system may maintain comprehensive audit records documenting the historical comparison results and the basis for routing decisions, while preserving the capability for the second sample to be accessed through review mechanisms outside the default workflow, such as when specifically requested by a physician monitoring longitudinal trends, when flagged by quality assurance protocols that evaluate historical comparison accuracy, when laboratory policies require periodic review of samples classified as stable, when a reviewer indicates interest in examining samples with specific patterns of minimal change, when retrospective analysis of stable cases is initiated for research or validation purposes, when subsequent test results suggest re-evaluation of previously stable findings, and/or when clinical circumstances warrant examination of samples that initially bypassed human review based on their minimal deviation from historical baselines falling below the predetermined threshold for triggering default human review workflow.

It is noted that the method operations in the various embodiments may be performed with flexible timing relationships between steps. For example, there is no requirement for temporal proximity between receiving the first and second images, between receiving the first and second current cytological analysis results, or between receiving the first and second historical cytological analysis results. Additionally, the comparing steps may be performed at different times without temporal dependency on one another or on the receiving steps. In some implementations, operations related to the first patient may be completed before operations related to the second patient begin, while in other implementations, the operations may be performed in parallel or in an interleaved manner. Similarly, the routing decisions may occur at any time after the respective comparisons are complete. More generally, unless explicitly specified otherwise, method operations described throughout this disclosure may be performed sequentially, in parallel, simultaneously, or with arbitrary temporal separation, and the temporal order of operations may vary depending on computational resources, system architecture, network conditions, load balancing considerations, and/or other factors. For instance, batches of multiple patients' data may be processed at scheduled intervals, or individual patients' data may be processed on demand as they become available. This temporal flexibility applies to operations across various embodiments described herein, including but not limited to receiving, analyzing, comparing, determining, routing, and any other processing steps, thereby accommodating diverse implementation environments, workflow patterns, and processing requirements. In some embodiments, historical cytological analysis results refer to past test results or results calculated based on past test results, which may include past reports, images, hematological cell quantification metrics, thresholds, and/or classifications of past tests. Historical cytological analysis results may further include results calculated from multiple historical tests, such as a baseline or trend. Cells associated with the first sample, may refer to any cells associated with the first sample, they may be cells classified in any classification, any image or any cytological smear.

In some implementations of the method, the magnitude of the first and second identified changes comprises numerical differences in hematological cell or cell type quantification metric (HCQM) values between the current cytological analysis results and the historical cytological analysis results for each respective patient. The numerical differences in HCQM values may provide a quantitative basis for determining the clinical significance of changes between current and historical results. These differences may be calculated through various methodologies, including but not limited to: absolute numerical differences, percentage changes, normalized differences accounting for baseline variability, or statistically derived measures such as z-scores or standard deviations from established patient baselines. The system may implement different comparison approaches for different cell types and/or parameters, recognizing that changes of identical numerical magnitude may carry different clinical significance depending on the specific HCQM being evaluated. For example, a 5% increase in a rare cell population may represent a more significant change than the same percentage increase in an abundant cell type. The numerical differences may be evaluated not only for their magnitude but also for their direction (increase versus decrease), pattern (sustained versus fluctuating), and relationship to established clinical reference ranges, providing a multidimensional assessment of change significance. This approach may enable precise identification of clinically relevant changes while accommodating the inherent variability and context-dependency of hematological parameters.

In some implementations of the method, the magnitude of the first and second identified changes comprises a degree of alteration in cell morphological characteristics between the current cytological analysis results and the historical cytological analysis results for each respective patient. The degree of alteration in cell morphological characteristics may be assessed through various quantitative and/or qualitative approaches that capture changes in cellular appearance beyond simple numerical counts. These approaches may include, but are not limited to: changes in cell size distribution metrics such as mean cell volume or diameter; alterations in nuclear-to-cytoplasmic ratio distributions; shifts in cellular granularity or vacuolation patterns; changes in chromatin structure characteristics; modifications in membrane irregularity indicators; variations in staining intensity or pattern distributions; or composite indices that combine multiple morphological parameters. The system may implement specialized image analysis algorithms designed to quantify these morphological characteristics with high precision and reproducibility, potentially enabling detection of subtle changes that may not be apparent in standard numerical metrics. The degree of alteration may be calculated using various mathematical frameworks including Euclidean distance in multidimensional morphological feature space, statistical distribution comparisons, or machine learning-derived similarity scores. This morphological change assessment may be particularly valuable for detecting early disease progression, treatment response, or the emergence of atypical cell populations.

In some implementations of the method, the magnitude of the first and second identified changes comprises a composite score derived from changes across multiple cytological parameters between the current cytological analysis results and the historical cytological analysis results for each respective patient. The composite score may integrate changes across diverse cytological parameters to provide a more comprehensive assessment of overall hematological status evolution. This approach may recognize that clinically significant changes often manifest across multiple parameters simultaneously, and that the correlation structure between these changes may carry diagnostic information beyond what individual parameters convey. The composite score may be constructed through various methodological frameworks, including but not limited to: weighted averaging of standardized change metrics across parameters; multivariate statistical techniques such as principal component analysis or factor analysis to identify key patterns of coordinated change; machine learning algorithms trained to recognize clinically significant change patterns; rule-based scoring systems derived from clinical guidelines; or Bayesian probability models that incorporate prior knowledge about parameter relationships. The weighting or importance assigned to different parameters within the composite score may be determined through various approaches such as expert-defined clinical significance, statistical measures of variance contribution, or data-driven optimization based on outcome prediction performance. This integrated assessment approach may enhance the system's ability to distinguish between clinically meaningful changes requiring review and normal physiological fluctuations or technical variations, potentially improving both the sensitivity and specificity of the review workflow routing process.

In some implementations, the method further comprises receiving a third image of a third current cytological smear associated with a third sample obtained from a third patient; receiving third historical cytological analysis results associated with the third patient; analyzing the third image to generate third current cytological analysis results; comparing the third current cytological analysis results with the third historical cytological analysis results to identify third changes; determining that the third changes indicate stability or improvement in previously identified abnormal findings; and not presenting, as part of default human review workflow, cells associated with the third sample for human review despite the presence of findings in the third current cytological analysis results that would otherwise trigger review if evaluated independently of the third historical cytological analysis results. The determination that changes indicate stability or improvement may be implemented through various analytical approaches that assess the directionality, magnitude, and clinical significance of identified changes. These approaches may include, but are not limited to: quantitative comparison of abnormal cell population sizes or percentages to evaluate numerical stability or reduction; morphological assessment algorithms that detect transitions toward more normal cellular characteristics; statistical trend analysis that identifies plateauing or improving trajectories in previously abnormal parameters; pattern recognition techniques that identify characteristic evolution patterns associated with resolving pathological conditions; or multiparameter assessment frameworks that integrate changes across related cell populations and features to evaluate overall hematological status direction. The system may implement different stability criteria for different clinical contexts, potentially applying more stringent stability definitions for high-risk conditions and more flexible criteria for benign or chronic conditions with expected fluctuations. This context-aware approach may reduce unnecessary human review of cases where abnormalities are present but stable or improving according to expected clinical progression patterns, while still ensuring that new or worsening abnormalities receive appropriate expert attention. By not presenting cells that would otherwise trigger review when evaluated in isolation, the system may optimize laboratory workflow efficiency without compromising patient safety, potentially redirecting specialist time toward cases with evidence of new or progressing abnormalities that require intervention or clinical decision-making. The stability determination may incorporate temporal factors such as the duration of stability, the rate of improvement, or the relationship to treatment milestones, potentially applying different review thresholds for recent versus long-established abnormalities.

In some implementations of the method, comparing the first current cytological analysis results with the first historical cytological analysis results comprises analyzing trends across multiple historical cytological analysis results of the first patient to determine whether gradual changes over time have reached a magnitude that exceeds the predetermined threshold; and comparing the second current cytological analysis results with the second historical cytological analysis results comprises analyzing trends across multiple historical cytological analysis results of the second patient to determine whether gradual changes over time have reached a magnitude that exceeds the predetermined threshold. The analyzing of trends across multiple historical cytological analysis results may be implemented through various temporal analytical methodologies designed to detect meaningful patterns of change over time. These methodologies may include, but are not limited to: linear or non-linear regression analyses that characterize rate-of-change parameters; time-series decomposition techniques that separate cyclical, seasonal, and progressive components of variation; statistical process control approaches that establish control limits for expected variation and identify significant deviations; cumulative sum analyses that track progressive accumulation of small deviations; slope analysis of key parameters to determine acceleration or deceleration of change rates; area-under-the-curve calculations for time-integrated assessment of parameter trajectories; or machine learning algorithms trained to recognize clinically significant temporal patterns in longitudinal data. The trend analysis may incorporate various temporal weighting schemes, potentially giving greater emphasis to more recent historical results while still accounting for longer-term patterns. The determination of whether gradual changes have reached a magnitude exceeding the predetermined threshold may involve comparing derived trend metrics such as slope, acceleration, or cumulative change against clinical significance criteria rather than simply evaluating the current value in isolation. This approach may enable detection of subtle but persistent directional changes that individually fall below acute change thresholds but collectively represent clinically meaningful shifts requiring attention. For example, a series of small but consistent increases in blast cells over multiple analyses may trigger review even when no single increment exceeds standard thresholds. This longitudinal perspective may enhance the system's ability to identify slowly evolving conditions while reducing false positives from random fluctuations or measurement variability, potentially improving both the sensitivity and specificity of the review workflow routing process.

In some implementations of the method, the first historical cytological analysis results comprise a baseline or trend of changes over time, calculated from multiple historical cytological analysis results of the first patient and wherein comparing the first current cytological analysis result comprises calculating a difference between a current first hematological cell or cell type quantification metric (HCQM) and value expected from the trend or baseline to determine whether the difference exceeds the predetermined threshold; and wherein the second historical cytological analysis results comprise a baseline or trend of changes over time, calculated from multiple historical cytological analysis results of the second patient and wherein comparing the second current cytological analysis result comprises calculating a difference between a current second HCQM and value expected from the trend or baseline to determine whether the difference exceeds the predetermined threshold. The baseline or trend of changes over time may be calculated through various mathematical and statistical approaches that characterize a patient's typical values or established patterns of change. These approaches may include, but are not limited to: calculation of personalized reference ranges derived from statistical analysis of the patient's historical values; establishment of stable-state baselines during periods of clinical stability; fitting of mathematical functions to historical progression data to generate predictive models; time-series forecasting techniques that project expected future values based on established patterns; development of personalized trajectory models that incorporate both patient-specific factors and population-derived parameters; or machine learning algorithms trained to predict expected values based on historical patterns and contextual variables. The value expected from the trend or baseline may incorporate various modifying factors including treatment effects, seasonal variations, age-related progression factors, or known cyclical patterns in the patient's condition. The calculation of the difference between current and expected values may be implemented through various methods including absolute numerical difference, percentage deviation, standardized difference (such as z-scores based on personalized variation metrics), or more complex distance measures in multiparameter space. These personalized expectation models may enable more precise detection of meaningful deviations by accounting for each patient's unique patterns and trajectories rather than applying uniform thresholds, potentially improving the specificity of review workflows for patients with stable but abnormal baselines or known progressive conditions with expected change patterns, including predicting a future of crossing of the threshold. For example, a patient with a known gradually increasing monocyte count may trigger review when the current count exceeds the expected trend line by a significant margin, rather than triggering review at every analysis due to values that consistently exceed population reference ranges but remain consistent with the patient's established pattern.

In some implementations of the method, the predetermined threshold comprises a set of cell-specific delta thresholds defined separately for at least two different cell types and cytological parameters, wherein presenting, as part of default human review workflow, cells associated with the first sample occurs when at least one of the first identified changes exceeds its corresponding cell type specific delta threshold, and wherein not presenting, as part of default human review workflow, cells associated with the second sample occurs when none of the second identified changes exceeds its corresponding cell type specific delta threshold. The set of cell-specific delta thresholds may recognize that different cell types and cytological parameters may exhibit varying degrees of natural biological fluctuation and may have different levels of clinical significance when changes occur. This differentiated threshold approach may enable more precise triggering of human review by accounting for the unique characteristics of each cell type or parameter. For instance, rare cell populations that typically demonstrate low natural variability may be assigned more sensitive delta thresholds that trigger review when relatively small numerical or morphological changes occur, while more abundant cell types with greater inherent biological variation may be assigned less sensitive thresholds to avoid excessive flagging of clinically insignificant fluctuations. The delta thresholds may be established through various methodologies, including but not limited to: statistical analysis of intra-individual variation in stable patient populations; evidence-based clinical guidelines regarding significant change magnitudes; machine learning approaches that optimize thresholds based on historical review outcomes; or expert-defined thresholds based on clinical experience. The thresholds may be implemented as absolute values, percentage changes, statistical deviation measures, or more complex algorithmic constructs depending on the nature of the parameter being evaluated. This parameter-specific approach may enhance workflow efficiency by focusing human expertise on the most clinically relevant changes while reducing time spent reviewing expected variations, potentially improving both laboratory throughput and diagnostic quality. Cells associated with the sample, may refer to any cells associated with the sample, may include cells classified according to any classification, and may relate to any image or any cytological smear.

In some implementations, the method further comprises adjusting reporting parameters for the first current cytological analysis results based on comparing the first current cytological analysis results with the first historical cytological analysis results to highlight clinically relevant trends when presenting cells associated with the first sample for human review. The adjusting of reporting parameters may involve modifying various aspects of how cytological analysis results are presented to human reviewers to optimize their attention allocation and analytical efficiency. These adjustments may include, but are not limited to: dynamically emphasizing specific cell types or parameters showing significant changes through visual highlighting, alternative sorting orders, or prominent placement within the interface; modifying visualization characteristics such as color coding, scaling, or graphical representations to accentuate the magnitude and direction of identified changes; contextualizing current results with appropriate historical reference points, trend lines, or statistical context to facilitate rapid comprehension of change significance; adjusting the level of detail presented for different parameters based on the degree of change detected; customizing default views to prioritize parameters with the most significant deviations; or applying specialized visualization techniques for specific change patterns such as progressive trends, oscillatory patterns, or sudden deviations. The reporting parameter adjustments may be implemented through rule-based systems, machine learning algorithms trained to predict reviewer information needs, or hybrid approaches that combine predefined rules with adaptive elements. These presentation optimizations may enhance reviewer efficiency by directing attention to the most clinically relevant aspects of complex cytological data, potentially reducing interpretation time while maintaining or improving diagnostic accuracy, especially for cases with subtle but significant changes that might be overlooked in standard presentation formats.

In some implementations, the method further comprises when presenting cells associated with the first sample for human review, providing contextual information about the first historical cytological analysis results and the first identified changes to guide the reviewer's attention to highly relevant aspects of the analysis. The contextual information provided may encompass a range of historical data, analytical insights, and change metrics designed to enhance the reviewer's understanding of the clinical significance of the current findings. This information may include, but is not limited to: temporal visualization of relevant parameters across multiple historical analyses to illustrate trends, patterns, or inflection points; statistical summaries of the patient's baseline values, variability ranges, or established personal reference ranges derived from historical data; explicit quantification of changes between current and historical results, potentially including absolute differences, percentage changes, or statistical significance metrics; automatic identification and highlighting of parameters showing the most significant deviations from historical patterns; relevant clinical context from the patient record that may influence interpretation, such as known conditions, treatments, or previous diagnostic conclusions; comparative assessments against population norms or condition-specific reference profiles; visualization of change trajectories with predictive extensions based on current trends; or annotation of specific cells or features that exemplify the most significant changes detected. The contextual information may be presented through various methods including integrated dashboards, hover-over tooltips, side-by-side comparisons, annotated images, or interactive visualizations that allow the reviewer to explore relationships between current and historical data. This rich contextual presentation may support more informed and efficient review decisions by providing the reviewer with a comprehensive understanding of how the current results relate to the patient's established patterns, potentially improving both the accuracy and speed of cytological interpretation, particularly for complex cases with subtle or multidimensional changes.

In some implementations, the method further comprises receiving first and second treatment status information associated with the first and second patients, respectively; and adjusting the predetermined threshold based on the respective treatment status information, wherein at least two different treatment statuses result in different threshold adjustments. The treatment status information may encompass various aspects of therapeutic interventions that may influence cellular profiles and the clinical significance of changes in cytological parameters. This information may include, but is not limited to: the type of treatment being administered (such as chemotherapy, immunotherapy, radiation therapy, or surgical intervention); the specific agents or protocols within treatment categories; the stage or phase of treatment (such as induction, consolidation, or maintenance); the timing relative to treatment cycles or interventions; the patient's response category based on previous assessments; or combination therapy details. The threshold adjustments may be implemented through various mechanisms including multiplication by treatment-specific factors, addition or subtraction of treatment-specific offsets, application of treatment-specific replacement thresholds, or implementation of treatment-specific algorithmic modifications. These adjustments may account for treatment-induced changes in cellular profiles that would be expected or considered clinically non-significant, potentially increasing thresholds for certain parameters during treatments known to cause predictable cellular alterations while potentially decreasing thresholds for parameters serving as early indicators of adverse reactions or treatment failure. For example, during active chemotherapy, thresholds for neutrophil count changes may be increased to prevent flagging of expected treatment-induced neutropenia, while thresholds for detection of abnormal lymphocyte morphology might be decreased to enable early detection of secondary malignancies. This treatment-contextual approach may improve review workflow efficiency by reducing unnecessary reviews of expected treatment effects while maintaining or enhancing sensitivity for detecting unexpected or clinically significant changes that warrant medical attention.

In some implementations of the method, the first and second treatment status information includes treatment cycle timing data, and wherein the adjusting comprises modifying the predetermined threshold according to expected cellular profile variations at different points in the treatment cycle for each respective patient. The treatment cycle timing data may provide temporal context that enables more precise threshold adjustments based on expected cellular dynamics throughout treatment cycles. This timing information may include, but is not limited to: days since last treatment administration; position within standardized treatment cycles (such as day number within a defined cycle length); cumulative treatment exposure measures; time since treatment initiation; or temporal relationship to specific interventions such as stem cell transplantation or growth factor administration. The threshold modifications may be implemented through various approaches including continuous time-dependent functions that model expected parameter evolution throughout the cycle; discrete phase-based threshold sets that apply different standards to early, mid, and late cycle timepoints; dynamically calculated adjustments based on pharmacokinetic or pharmacodynamic models of the specific therapeutic agents; or hybrid approaches that combine standard cycle templates with patient-specific response characteristics from previous cycles. These modifications may account for well-characterized temporal patterns in cellular responses to treatments, such as predictable nadirs in specific cell lineages following cytotoxic therapy, expected recovery patterns, or transient phenomena such as growth factor-induced cellular shifts. For example, neutrophil count change thresholds might be automatically increased during expected nadir phases of myelosuppressive chemotherapy cycles but returned to more sensitive levels during expected recovery phases when failure to demonstrate improvement might indicate complications requiring intervention. This cycle-aware approach may enhance both the specificity and sensitivity of the review workflow routing by dynamically adjusting significance thresholds to match the expected biological context at the specific point in the treatment timeline, potentially reducing false positive review triggers while maintaining vigilance for deviations from expected treatment response patterns. In some implementations of the method, a non-transitory computer-readable medium comprising instructions that, when executed by a processor, cause the processor to perform the computer-implemented method.

In various implementations, a system for comparative analysis of cytological smears, comprising: a computer configured to: receive, from an automatic cell classifier, first and second current cytological analysis results of first and second images of first and second respective current cytological smears associated with first and second samples obtained from first and second patients, respectively; receive first and second historical cytological analysis results associated with the first and second patients, respectively; compare the first and second current cytological analysis results with the first and second historical cytological analysis results to identify first and second changes, respectively; responsive to determining that the first identified changes exceed a predetermined threshold, cause cells associated with the first sample to be presented as part of default human review workflow; and responsive to determining that the second identified changes do not exceed the predetermined threshold, cause cells associated with the second sample to be excluded from presentation as part of default human review workflow. FIG. 18B illustrates an embodiment of a block diagram of a system for comparative analysis of cytological smears. The system includes a computer 285 that receives current cytological analysis results from an automatic cell classifier 286, and is further coupled to a human review workflow interface 287.

In some implementations of the system, the magnitude of the first and second identified changes comprises numerical differences in hematological cell or cell type quantification metric (HCQM) values between the current cytological analysis results and the historical cytological analysis results for each respective patient. In some implementations of the system, the magnitude of the first and second identified changes comprises a degree of alteration in cell morphological characteristics between the current cytological analysis results and the historical cytological analysis results for each respective patient.

In some implementations of the system, the magnitude of the first and second identified changes comprises a composite score derived from changes across multiple cytological parameters between the current cytological analysis results and the historical cytological analysis results for each respective patient. In some implementations of the system, the computer is further configured to: receive a third image of a third current cytological smear associated with a third sample obtained from a third patient; receive third historical cytological analysis results associated with the third patient; analyze the third image to generate third current cytological analysis results; compare the third current cytological analysis results with the third historical cytological analysis results to identify third changes; determine that the third changes indicate stability or improvement in previously identified abnormal findings; and cause cells associated with the third sample to be excluded from presentation as part of default human review workflow despite the presence of findings in the third current cytological analysis results that would otherwise trigger review if evaluated independently of the third historical cytological analysis results.

In some implementations of the system, comparing the first current cytological analysis results with the first historical cytological analysis results comprises analyzing trends across multiple historical cytological analysis results of the first patient to determine whether gradual changes over time have reached a magnitude that exceeds the predetermined threshold; and comparing the second current cytological analysis results with the second historical cytological analysis results comprises analyzing trends across multiple historical cytological analysis results of the second patient to determine whether gradual changes over time have reached a magnitude that exceeds the predetermined threshold.

In some implementations of the system, the first historical cytological analysis results comprise a baseline or trend of changes over time, calculated from multiple historical cytological analysis results of the first patient and wherein comparing the first current cytological analysis result comprises calculating a difference between a current first hematological cell or cell type quantification metric (HCQM) and value expected from the trend or baseline to determine whether the difference exceeds the predetermined threshold; and wherein the second historical cytological analysis results comprise a baseline or trend of changes over time, calculated from multiple historical cytological analysis results of the second patient and wherein comparing the second current cytological analysis result comprises calculating a difference between a current second HCQM and value expected from the trend or baseline to determine whether the difference exceeds the predetermined threshold.

In some implementations of the system, the predetermined threshold comprises a set of cell-specific delta thresholds defined separately for at least two different cell types and cytological parameters, wherein causing cells associated with the first sample to be presented as part of default human review workflow occurs when at least one of the first identified changes exceeds its corresponding cell type specific delta threshold, and wherein causing cells associated with the second sample to be excluded from presentation as part of default human review workflow occurs when none of the second identified changes exceeds its corresponding cell type specific delta threshold.

In some implementations of the system, the computer is further configured to adjust reporting parameters for the first current cytological analysis results based on comparing the first current cytological analysis results with the first historical cytological analysis results to highlight clinically relevant trends when presenting cells associated with the first sample for human review. In some implementations of the system, the computer is further configured to, when causing cells associated with the first sample to be presented for human review, provide contextual information about the first historical cytological analysis results and the first identified changes to guide the reviewer's attention to highly relevant aspects of the analysis.

In some implementations of the system, the computer is further configured to: receive first and second treatment status information associated with the first and second patients, respectively; and adjust the predetermined threshold based on the respective treatment status information, wherein at least two different treatment statuses result in different threshold adjustments. In some implementations of the system, the first and second treatment status information includes treatment cycle timing data, and wherein the adjusting comprises modifying the predetermined threshold according to expected cellular profile variations at different points in the treatment cycle for each respective patient.

In various implementations, a computer-implemented method for medication-aware presentation of cytological analysis for human review, comprising: receiving, from an automatic cell classifier, first and second classifications of cells in first and second images of first and second cytological smears associated with first and second samples obtained from first and second patients, respectively; wherein the first and second patients are administered a prescribed medication; comparing a result of a function of the first classifications with expected medication-induced changes associated with the prescribed medication; responsive to determining that the result of the function of the first classifications deviates above a threshold from the expected medication-induced changes associated with the prescribed medication, presenting, as part of default human review workflow, cells associated with the first sample; comparing a result of a function of the second classifications with the expected medication-induced changes associated with the prescribed medication; and responsive to determining that the result of the function of the second classifications does not deviate above the threshold from the expected medication-induced changes associated with the prescribed medication, not presenting, as part of default human review workflow, cells associated with the second sample. FIG. 19 illustrates an embodiment of a computer-implemented method for medication-aware presentation of cytological analysis for human review. The method includes the following steps. In step 290, receive, from an automatic cell classifier, first and second classifications of cells in first and second images of first and second cytological smears associated with first and second samples obtained from first and second patients, respectively, wherein the first and second patients are administered a prescribed medication. In step 291, compare, optionally by a processor, a result of a function of the first classifications with expected medication-induced changes associated with the prescribed medication. In step 292, responsive to determining that the result of the function of the first classifications deviates above a threshold from the expected medication-induced changes associated with the prescribed medication, present, as part of default human review workflow, cells associated with the first sample. In step 293, compare, optionally by the processor, a result of a function of the second classifications with the expected medication-induced changes associated with the prescribed medication. And in step 294, responsive to determining that the result of the function of the second classifications does not deviate above the threshold from the expected medication-induced changes associated with the prescribed medication, not present, as part of default human review workflow, cells associated with the second sample.

The medication-aware presentation system may selectively filter cytological samples for human review based on their deviation from expected medication-induced changes, potentially reducing unnecessary review of samples showing typical medication effects while maintaining appropriate scrutiny of samples with unexpected cellular presentations. The automatic cell classifier may implement various classification technologies including, but not limited to, machine learning algorithms, computer vision techniques, morphological analysis methods, and/or pattern recognition systems to identify and categorize cells within the cytological smears. The function of the classifications may encompass various computational transformations including statistical aggregations, distribution analyses, relationship mappings between cell types, and/or comparative metrics against reference models. The expected medication-induced changes may be derived from multiple sources including pharmaceutical reference databases, clinical literature, population studies, and/or adaptive learning from verified cases. The comparison process may implement various analytical approaches including direct value comparison, statistical hypothesis testing, similarity scoring, and/or multidimensional pattern matching to evaluate how closely the observed cellular presentations align with medication-expected patterns. The threshold determination may incorporate factors such as medication class, dosage levels, treatment duration, clinical context, cell type significance, and/or statistical confidence requirements to establish appropriate sensitivity levels. The default human review workflow may represent the standard operational process for cytological evaluation, potentially including predetermined review sequences, standard cell presentation protocols, and/or typical reviewer assignments. This selective presentation approach may optimize reviewer resources by focusing human expertise on samples most likely to contain clinically significant findings while appropriately filtering samples showing expected medication effects. Cells associated with the sample, may refer to any cells associated with the sample, may include cells classified according to any classification, and may relate to any image or any cytological smear.

In the context of this embodiment, “not presenting, as part of default human review workflow, cells associated with the second sample” refers to excluding the second sample from the default review process when the result of the function of the second classifications does not deviate above the threshold from the expected medication-induced changes. While the first sample is presented for human review because the first classifications deviate above the threshold from expected medication-induced changes (potentially indicating unexpected cellular responses, adverse medication effects, or concurrent pathological processes), the second sample is not presented because the second classifications remain within the expected range of medication-induced changes. The not presenting may be implemented through various mechanisms including: excluding the second sample from review queues designated for samples requiring verification, routing the second sample directly to automated reporting pathways as an expected medication response, preventing the second sample from appearing in reviewer worklists or task assignments, marking the second sample with metadata flags indicating conformity with expected medication effects while marking the first sample with flags indicating unexpected deviations requiring review, applying different marking schemes to the first and second samples that are distinguishable through visual indicators, medication response codes, deviation magnitude indicators, or pharmacological classification labels, storing the second sample in repositories designated for typical medication responses rather than in repositories for samples requiring human verification, presenting the medication deviation analysis results for the second sample to a human without an indication that review is required whereas the deviation analysis results for the first sample are presented with an indication that review is required due to unexpected medication responses, displaying medication response indicators that visually differentiate samples with expected responses from those with unexpected responses, providing graphical representations showing the second sample within expected medication effect ranges versus the first sample exceeding such ranges, and/or displaying the second sample in a manner distinguishable from the first sample in the default human review workflow, wherein such distinguishing manners may include distinct borders, different background colors or patterns, modified interaction controls, presence or absence of deviation alert indicators, different positioning within the interface based on deviation magnitude, alternative display formats for expected versus unexpected medication responses, pharmacological context annotations, or textual labels that identify the second sample as excluded from default human review workflow based on conformity with expected medication-induced changes. The system may additionally implement medication-specific displays showing that the second sample exhibits cellular patterns consistent with known pharmacological effects of the prescribed medication while the first sample shows patterns requiring expert evaluation. By not presenting the second sample while presenting the first sample based on medication deviation analysis, the system may focus human expertise on cases with potentially significant unexpected medication responses or adverse effects while avoiding unnecessary review of samples showing expected pharmacological cellular changes. The system may maintain comprehensive audit records documenting the medication comparison results, the specific medication involved, dosage information, and the basis for routing decisions, while preserving the capability for the second sample to be accessed through review mechanisms outside the default workflow, such as when specifically requested by a physician monitoring medication effects, when flagged by pharmacovigilance protocols, when laboratory policies require periodic review of samples classified as showing expected medication responses, when a reviewer indicates interest in examining samples from patients on specific medications, when retrospective analysis of medication effects is initiated for research purposes, when subsequent test results suggest re-evaluation of medication response patterns, when changes in medication regimen warrant comparison with previous responses, and/or when clinical circumstances warrant examination of samples that initially bypassed human review based on their conformity with expected medication-induced changes falling within the threshold for typical pharmacological cellular responses.

In some implementations of the method, the expected medication-induced changes include expected medication-induced cellular morphological changes associated with the prescribed medication, wherein the threshold is determined based on typical cellular responses to the prescribed medication across a population of patients. The expected medication-induced cellular morphological changes may encompass a wide variety of alterations in cell appearance, structure, size, shape, staining properties, granularity, nuclear characteristics, and/or cytoplasmic features that typically occur when patients are administered specific medications. The threshold for determining deviation may be established based on statistical analysis of cellular responses observed across diverse patient populations who have received the prescribed medication, which may include data collected from multiple sources such as clinical trials, ongoing patient monitoring, and/or medical literature. The threshold determination process may implement various statistical methodologies including, but not limited to, standard deviation calculations, percentile rankings, and/or machine learning algorithms that account for natural variability while identifying clinically significant outliers. The threshold may be dynamically adjusted based on factors such as medication class, dosage patterns, administration schedules, and/or patient subpopulation characteristics. In various embodiments, multiple thresholds may be established for different cell types, medication classes, and/or morphological parameters to provide more nuanced analysis capabilities. The system may periodically update these thresholds as new information about medication-induced cellular responses becomes available, potentially incorporating feedback from human reviewers to refine the threshold calibration over time.

In some implementations of the method, determining that the result of the function of the first classifications deviates above the threshold comprises identifying a degree of presence of cell populations in the first cytological smear that exceeds the threshold for cell populations not typically affected by the prescribed medication, and wherein determining that the result of the function of the second classifications does not deviate above the threshold comprises identifying a degree of presence of cell populations in the second cytological smear that remains below the threshold for cell populations not typically affected by the prescribed medication. The identification of cell populations not typically affected by the prescribed medication may involve a multi-faceted analysis approach that distinguishes between expected and unexpected cellular responses. The system may maintain a medication effects database that catalogs which cell populations are typically affected and which are typically unaffected by various medications, potentially categorized by medication class, dosage range, and/or administration duration. The degree of presence may be quantified through various metrics including, but not limited to, absolute cell counts, percentage relative to total cells, distribution patterns within the smear, and/or ratios between different cell populations. The threshold application may implement different sensitivity levels for different cell types based on their clinical significance, with potentially serious indicators receiving more stringent threshold settings than those with limited clinical impact. The analysis process may account for natural biological variability by incorporating statistical confidence intervals appropriate to the cell population being evaluated. In some embodiments, the system may examine not only the presence of unexpected cell populations but also the absence of expected cell populations, which may provide additional indicators of unusual medication responses. The comparative analysis between affected and unaffected cell populations may enhance the system's capability to distinguish between medication-induced changes and changes that may signal other underlying conditions requiring human review.

In some implementations, the method further comprises, responsive to determining that the result of the function of the first classifications deviates above the threshold from the expected medication-induced changes: identifying specific subsets of cells in the first image that exhibit characteristics deviating above the threshold from the expected medication-induced changes; and presenting the identified specific subsets of cells for human review rather than the entire first image. The identification of specific subsets of cells that exhibit characteristics deviating above the threshold may implement various computational approaches to isolate and highlight cells of interest. The system may utilize image processing algorithms to extract individual cells from the first image based on their morphological characteristics, staining patterns, and/or spatial relationships. These cells may then be analyzed against medication-specific reference models to determine which specific characteristics deviate from expected medication-induced changes. The presentation process may organize the identified cells into contextually relevant groupings based on the nature and degree of their deviation, potentially including classification into categories such as “mild deviation,” “moderate deviation,” and/or “severe deviation” to facilitate efficient human review. In various embodiments, the system may present these cell subsets alongside comparable reference cells showing both normal morphology and typical medication-induced changes to provide visual context for the reviewer. The selective presentation approach may significantly reduce reviewer workload by focusing attention specifically on cells that warrant human assessment while filtering out cells exhibiting expected medication responses. This targeted presentation may additionally include metadata about each cell such as classification confidence scores, specific deviation metrics, and/or potential alternative classifications, which may enhance the reviewer's ability to make accurate assessments efficiently.

In some implementations of the method, the identified specific subsets of cells are presented alongside relevant medication information comprising at least one of: expected prescribed medication effects, dosage, administration timing, or visualization of the boundary between expected and unexpected cellular presentations relative to the threshold. The presentation of relevant medication information alongside the identified specific subsets of cells may create a comprehensive contextual framework that enhances reviewer understanding and decision-making efficiency. The expected prescribed medication effects information may include detailed descriptions, visual examples, and/or scientific literature references regarding typical cellular changes induced by the specific medication or medication class. Dosage information may encompass not only the current dosage but also dosage history, including any recent adjustments that may influence cellular presentation. Administration timing details may include information about when the medication was last administered relative to sample collection, duration of therapy, and/or compliance patterns, which may significantly impact cellular morphology. The visualization of boundaries between expected and unexpected cellular presentations may be implemented through various graphical representations, potentially including overlay highlighting, side-by-side comparisons, gradient color coding indicating deviation magnitude, and/or statistical distribution charts showing where the presented cells fall relative to population norms. This contextual information may be customizable based on reviewer preferences or institutional protocols, potentially allowing different visualization modes or information density levels. In some embodiments, the system may provide interactive elements that allow reviewers to dynamically adjust threshold visualization parameters or toggle between different contextual information views to support more nuanced analysis.

In some implementations, the method further comprises, responsive to determining that the result of the function of the first classifications deviates above the threshold from the expected medication-induced changes: selecting a specific human reviewer based on expertise in the pharmacological effects of the prescribed medication or based on expertise in the specific cell populations in the first cytological smear that deviate above the threshold. The selection of a specific human reviewer based on expertise may implement a matching system that aligns case complexity with reviewer specialization. The system may maintain expertise profiles for available reviewers that catalog their knowledge areas, experience levels, and/or certification status regarding specific medications, medication classes, cellular pathologies, and/or affected organ systems. The matching process may incorporate multiple factors including, but not limited to, the specific medication involved, the nature and severity of the observed deviations, the clinical context of the patient, and/or the potential urgency of the findings. In various embodiments, the system may employ a tiered routing approach where cases with modest deviations may be routed to reviewers with general expertise, while cases with significant or unusual deviations may be directed to reviewers with specialized knowledge in the relevant medication effects or cell populations. The expertise matching algorithm may additionally consider workload balance, availability, historical performance with similar cases, and/or institutional priorities when selecting appropriate reviewers. This targeted routing approach may potentially improve diagnostic accuracy while optimizing the utilization of specialized expertise within the reviewing team. The system may adaptively learn from review outcomes to refine its matching algorithms over time, potentially identifying patterns in which specific types of cases benefit most from particular expertise profiles.

In some implementations, the method further comprises determining a priority level for human review based on the degree of deviation of the result of the function of the first classifications above the threshold from the expected medication-induced changes and clinical significance of the affected cell populations; and adjusting the quantity of cells presented for review based on current reviewer workload, wherein higher priority deviations maintain comprehensive routing essentially regardless of workload while lower priority deviations lead to presenting less cells during high workload periods. The priority determination process may implement a multi-dimensional analysis that evaluates both the magnitude of deviation and the clinical significance of the affected cells. The priority levels may be established through various classification schemes, potentially including numeric scales (e.g., 1-5), categorical designations (e.g., “routine,” “elevated,” “urgent”), and/or time-based targets for completion. Clinical significance assessments may incorporate factors such as the cell type's association with serious conditions, the patient's clinical history and risk factors, and/or the potential therapeutic implications of the findings. The workload adjustment mechanism may dynamically modify the presentation based on real-time monitoring of system capacity, reviewer availability, and/or case backlog metrics. For higher priority cases, the system may maintain comprehensive cell presentation essentially regardless of workload conditions, potentially implementing methods such as redistributing lower priority cases, activating additional review resources, and/or providing specialized interface options to facilitate efficient review of critical findings. For lower priority cases during high workload periods, the system may implement various presentation reduction methods, potentially including statistical sampling of representative cells, focusing on cells with the highest deviation magnitudes, and/or grouping similar cells to reduce redundant review. The workload adaptation mechanisms may incorporate predictive modeling that anticipates peak periods and adjusts routing patterns proactively rather than reactively. These load-balancing methods may help maintain consistent quality standards while acknowledging the practical constraints of review resources.

In some implementations of the method, determining that the result of the function of the first classifications deviates above the threshold comprises: identifying multiple first classifications that individually fall within expected ranges for the prescribed medication but collectively represent an unusual pattern that exceeds the threshold; and wherein determining that the result of the function of the second classifications does not deviate above the threshold comprises identifying that multiple second classifications do not collectively represent an unusual pattern that exceeds the threshold. The identification of unusual patterns formed by multiple classifications that individually appear normal may implement pattern recognition techniques that analyze relationships, ratios, and/or distributions across different cell classifications. The system may evaluate various combinatorial aspects including, but not limited to, unexpected co-occurrences of certain cell types, atypical proportional relationships between normally related cell populations, unusual spatial distribution patterns within the sample, and/or temporal evolution of cell population profiles compared to baseline expectations. The pattern analysis may incorporate domain-specific knowledge about how different cell populations typically respond to the prescribed medication, potentially identifying situations where individual responses fall within normal ranges but their collective presentation is inconsistent with typical medication effects. In various embodiments, the system may utilize machine learning approaches such as anomaly detection algorithms, clustering techniques, and/or rule-based expert systems to identify these subtle combined deviations that might not be apparent when examining individual classifications in isolation. The threshold application for combined patterns may implement different sensitivity levels based on the clinical significance of the particular combination, with potentially concerning combinations receiving more stringent evaluation than those with limited clinical implications. This combinatorial analysis approach may enhance the system's ability to detect subtle but clinically significant deviations that might otherwise escape notice if only individual cell classifications were evaluated independently.

In some implementations, the method further comprises distinguishing, for both the first and second images, between sample preparation artifacts and medication-induced cellular changes using medication-specific artifact reference patterns; and wherein presenting cells associated with the first sample for human review prioritizes the cellular changes in the first image that cannot be attributed to expected effects of the prescribed medication or known artifacts and that deviate above the threshold from the expected medication-induced changes. The differentiation between sample preparation artifacts and medication-induced changes may implement image analysis techniques that examine visual characteristics, distribution patterns, and/or contextual indicators. The medication-specific artifact reference patterns may be maintained in a comprehensive database that catalogs known visual signatures of artifacts commonly associated with specific medications, potentially including effects such as precipitation patterns, staining interactions, and/or crystallization phenomena. The analysis process may evaluate multiple features including, but not limited to, spatial distribution (artifacts often showing non-biological distribution patterns), morphological consistency (artifacts typically lacking the natural variation of biological structures), and/or relationship to cellular structures (artifacts often appearing disconnected from expected biological boundaries). In various embodiments, the system may apply different analytical approaches based on the specific medication involved, recognizing that different pharmaceutical agents may produce distinct artifact profiles requiring specialized detection methods. The prioritization mechanism for human review may implement a multi-level filtering process that first excludes expected medication effects, then removes identified artifacts, and finally ranks the remaining cellular changes based on their deviation magnitude and clinical significance. This differentiation capability may enhance review efficiency by reducing false positives due to preparation artifacts that might otherwise be misinterpreted as clinically significant findings, allowing reviewers to focus attention on cellular changes most likely to represent meaningful biological responses requiring professional assessment.

In some implementations of the method, determining whether the result of the function of the first and second classifications deviate above the threshold is performed based on prescribed medication dosage information for the first and second patients, with expected medication-induced changes proportionally related to dosage levels of the prescribed medication administered to the first and second patients. The incorporation of dosage information into the threshold determination process may implement various mathematical models that relate medication concentrations to cellular response magnitudes. The system may utilize pharmacokinetic and pharmacodynamic principles to establish expected dose-response relationships for different cell types and morphological characteristics, potentially accounting for factors such as bioavailability, distribution volume, and/or elimination half-life of the prescribed medication. The proportional relationship between dosage levels and expected changes may be modeled through various mathematical functions including, but not limited to, linear correlations, logarithmic relationships, sigmoid curves, and/or more complex multivariate models that incorporate additional patient factors. In various embodiments, the system may maintain medication-specific dose-response curves derived from population studies, clinical trial data, and/or ongoing analysis of patient outcomes, which may be periodically updated as new information becomes available. The threshold adjustment process may implement dosage normalization techniques that allow for appropriate comparisons between patients receiving different medication amounts, potentially including relative scaling based on body weight, body surface area, organ function parameters, and/or genetic factors known to influence medication metabolism. This dosage-aware approach may enhance the system's ability to distinguish between expected variation based on prescribed dosage and unexpected deviations that warrant human review, potentially reducing unnecessary reviews while maintaining appropriate sensitivity for clinically significant findings across different dosage regimens.

In some implementations, the method further comprises retrieving historical cytological data for the first and second patients; and modifying the expected medication-induced changes and the threshold based on the first and second patients' prior documented responses to the prescribed medication as indicated in the historical cytological data. The integration of historical cytological data may implement a personalized analysis approach that recognizes individual variations in medication response patterns. The retrieval process may access various data sources including, but not limited to, electronic health records, laboratory information systems, digital slide archives, and/or dedicated cytological databases to compile comprehensive historical profiles. The modification of expected medication-induced changes may involve adaptive algorithms that detect consistent patterns in how a specific patient's cells respond to the prescribed medication over time, potentially identifying stable idiosyncratic responses that differ from population norms but represent normal findings for that particular patient. The threshold adjustments may implement various computational approaches including, but not limited to, statistical modeling of the patient's personal baseline and variance, trend analysis to identify progressive changes over time, and/or comparison of current results with historical response patterns. In various embodiments, the system may apply different weighting factors to historical data based on recency, sample quality, clinical context at the time of collection, and/or similarity to current medication regimens. The personalization process may be particularly valuable for patients with chronic conditions requiring ongoing medication or those with known atypical responses to pharmaceutical agents, potentially reducing unnecessary reviews of consistent idiosyncratic patterns while maintaining appropriate sensitivity to new or changing deviations. This historically-informed approach may enhance the system's ability to distinguish between stable, clinically insignificant individual variations and new deviations that may signal clinically important changes requiring human assessment.

In some implementations, the method further comprises categorizing cells from the first sample into distinct abnormality types; and prioritizing presentation of specific abnormality types for human review based on their correlation with known adverse medication effects associated with the prescribed medication. The categorization of cells into distinct abnormality types may implement a multi-stage classification process that examines morphological characteristics, functional indicators, and/or staining properties. The system may establish various categorization schemes based on different classification frameworks including, but not limited to, cell lineage alterations, maturation abnormalities, structural deviations, and/or functional marker expressions. The correlation analysis with known adverse medication effects may utilize comprehensive medication safety databases that catalog documented associations between specific medications and cellular abnormalities, potentially including data from clinical trials, post-marketing surveillance, published case reports, and/or regulatory safety communications. The prioritization mechanism may implement a weighted ranking system that considers multiple factors including the strength of the correlation between the abnormality and the medication, the clinical significance of the potential adverse effect, the rarity of the abnormality type, and/or the magnitude of deviation from expected patterns. In various embodiments, the system may apply different prioritization methods based on medication class, patient risk factors, treatment duration, and/or concomitant medications that might influence adverse effect profiles. This correlation-based prioritization approach may enhance reviewer efficiency by directing attention first to abnormalities most likely to represent medication-related issues requiring intervention, potentially improving patient safety through earlier identification of adverse medication effects while optimizing review resource allocation. The system may adapt its correlation knowledge over time through the incorporation of new safety information and feedback from confirmed review findings.

In some implementations, the method further comprises determining, for the first sample, an urgency classification based on the degree of deviation above the threshold and clinical significance of the affected cell populations; and routing the cells from the first sample to different reviewer queues based on the urgency classification. The urgency classification determination may implement a multi-factor analysis framework that evaluates both quantitative deviation metrics and qualitative clinical significance assessments. The system may establish various urgency categories such as “routine,” “priority,” “urgent,” and/or “stat,” with specific routing and handling protocols associated with each classification level. The deviation degree assessment may incorporate various mathematical approaches including, but not limited to, standard deviation multiples, percentile rankings, and/or weighted composite scores that integrate multiple deviation parameters. The clinical significance evaluation may consider factors such as the cell population's association with serious or progressive conditions, the potential therapeutic implications of the findings, the patient's clinical context and risk factors, and/or the time-sensitivity of potential interventions. The routing to different reviewer queues may implement workflow management techniques that match case urgency with appropriate review pathways, potentially including specialized high-priority queues with dedicated reviewers, accelerated processing protocols, and/or enhanced notification systems. In various embodiments, the system may incorporate dynamic queue management that adjusts routing based on current workload conditions, available expertise, institutional priorities, and/or time-since-submission metrics. The urgency-based routing approach may enhance overall system effectiveness by ensuring that time-critical findings receive appropriately expedited review while maintaining efficient processing of routine cases. The urgency classification system may incorporate feedback mechanisms that allow for adjustment of classification algorithms based on outcomes and reviewer input, potentially refining the system's ability to appropriately prioritize cases over time.

In some implementations, the method further comprises analyzing patient demographic data associated with the first patient; and adjusting the threshold based on demographic-specific expected medication-induced changes, wherein cells from the first sample are presented for human review when the result of the function of the first classifications deviates above the demographic-adjusted threshold. The analysis of patient demographic data may implement a comprehensive evaluation of factors known to influence medication responses including, but not limited to, age, sex, ethnicity, genetic markers, comorbidities, and/or physiological parameters. The system may maintain demographic-specific reference databases that catalog known variations in medication responses across different population segments, potentially derived from clinical studies, pharmacogenomic research, and/or ongoing analysis of patient outcomes. The threshold adjustment process may apply various computational approaches including scaling factors, customized reference ranges, and/or multivariate models that account for interactions between demographic factors. For example, age-related adjustments may account for known differences in medication metabolism, cellular resilience, and/or baseline morphological characteristics across different life stages. Similarly, ethnicity-based adjustments may incorporate known pharmacogenetic variations that influence medication processing and cellular responses. In various embodiments, the system may implement different adjustment methods based on the specificity and strength of evidence for demographic influences on the particular medication and cell types involved. The demographic-adjusted thresholds may be particularly valuable for medications with well-documented population-specific response patterns, potentially reducing unnecessary reviews due to expected demographic variations while maintaining appropriate sensitivity for truly abnormal findings within the patient's demographic context. This demographic-aware approach may enhance the system's ability to distinguish between normal population variation and clinically significant deviations requiring human assessment, potentially improving both review efficiency and diagnostic accuracy across diverse patient populations.

In some implementations, the method further comprises determining a medication class for the prescribed medication; selecting a medication-class-specific analysis protocol specialized for detecting abnormalities associated with the determined medication class; and applying the medication-class-specific analysis protocol when comparing the result of the function of the first classifications with the expected medication-induced changes. The determination of medication class may implement pharmaceutical classification systems such as the Anatomical Therapeutic Chemical (ATC) classification, pharmacological mechanism groupings, structural similarity classifications, and/or therapeutic application categories. The system may maintain a comprehensive medication taxonomy database that maps individual medications to their appropriate classes and subclasses, potentially including multiple classification schemes to allow for different analytical perspectives. The selection of medication-class-specific analysis protocols may draw from a library of specialized analytical frameworks developed to address the particular cellular effects and potential abnormalities associated with different medication categories. These specialized protocols may incorporate various elements including, but not limited to, targeted cell population assessments, specific morphological feature evaluations, specialized staining interpretation parameters, and/or class-specific artifact recognition patterns. For example, protocols for cytotoxic medications may focus on apoptotic indicators and cellular fragmentation patterns, while protocols for immunomodulatory agents may emphasize specific white blood cell population changes and activation markers. In various embodiments, the system may implement different analytical approaches based on medication subclasses, combination therapies, dosage ranges, and/or administration routes, recognizing that these factors may significantly influence cellular presentation patterns. The class-specific analytical approach may enhance the system's ability to detect subtle but clinically significant abnormalities that might not be apparent using general analysis frameworks, potentially improving both the sensitivity and specificity of the review routing process. The protocol library may be periodically updated to incorporate new understanding of medication class effects and emerging analytical techniques relevant to specific pharmaceutical categories. In some implementations of the method, a non-transitory computer-readable medium comprising instructions that, when executed by a processor, cause the processor to perform the computer-implemented method.

In various implementations, a system for medication-aware presentation of cytological analysis for human review, comprising: a computer configured to: receive, from an automatic cell classifier, first and second classifications of cells in first and second images of first and second cytological smears associated with first and second samples obtained from first and second patients, respectively, wherein the first and second patients are administered a prescribed medication; compare a result of a function of the first classifications with expected medication-induced changes associated with the prescribed medication; responsive to determining that the result of the function of the first classifications deviates above a threshold from the expected medication-induced changes associated with the prescribed medication, cause cells associated with the first sample to be presented as part of default human review workflow; compare a result of a function of the second classifications with the expected medication-induced changes associated with the prescribed medication; and responsive to determining that the result of the function of the second classifications does not deviate above the threshold from the expected medication-induced changes associated with the prescribed medication, cause cells associated with the second sample to be excluded from presentation as part of default human review workflow.

In some implementations of the system, the expected medication-induced changes include expected medication-induced cellular morphological changes associated with the prescribed medication, wherein the threshold is determined based on typical cellular responses to the prescribed medication across a population of patients. In some implementations of the system, determining that the result of the function of the first classifications deviates above the threshold comprises identifying a degree of presence of cell populations in the first cytological smear that exceeds the threshold for cell populations not typically affected by the prescribed medication, and wherein determining that the result of the function of the second classifications does not deviate above the threshold comprises identifying a degree of presence of cell populations in the second cytological smear that remains below the threshold for cell populations not typically affected by the prescribed medication.

In some implementations of the system, the computer is further configured to, responsive to determining that the result of the function of the first classifications deviates above the threshold from the expected medication-induced changes: identify specific subsets of cells in the first image that exhibit characteristics deviating above the threshold from the expected medication-induced changes; and cause the identified specific subsets of cells to be presented for human review rather than the entire first image. In some implementations of the system, the computer is further configured to cause the identified specific subsets of cells to be presented alongside relevant medication information comprising at least one of: expected prescribed medication effects, dosage, administration timing, or visualization of the boundary between expected and unexpected cellular presentations relative to the threshold.

In some implementations of the system, the computer is further configured to, responsive to determining that the result of the function of the first classifications deviates above the threshold from the expected medication-induced changes: select a specific human reviewer based on expertise in the pharmacological effects of the prescribed medication or based on expertise in the specific cell populations in the first cytological smear that deviate above the threshold. In some implementations of the system, the computer is further configured to: determine a priority level for human review based on the degree of deviation of the result of the function of the first classifications above the threshold from the expected medication-induced changes and clinical significance of the affected cell populations; and adjust the quantity of cells presented for review based on current reviewer workload, wherein higher priority deviations maintain comprehensive routing essentially regardless of workload while lower priority deviations lead to presenting less cells during high workload periods.

In some implementations of the system, determining that the result of the function of the first classifications deviates above the threshold comprises: identifying multiple first classifications that individually fall within expected ranges for the prescribed medication but collectively represent an unusual pattern that exceeds the threshold; and wherein determining that the result of the function of the second classifications does not deviate above the threshold comprises identifying that multiple second classifications do not collectively represent an unusual pattern that exceeds the threshold. In some implementations of the system, the computer is further configured to: distinguish, for both the first and second images, between sample preparation artifacts and medication-induced cellular changes using medication-specific artifact reference patterns; and wherein causing cells associated with the first sample to be presented for human review prioritizes the cellular changes in the first image that cannot be attributed to expected effects of the prescribed medication or known artifacts and that deviate above the threshold from the expected medication-induced changes.

In some implementations of the system, determining whether the result of the function of the first and second classifications deviate above the threshold is performed based on prescribed medication dosage information for the first and second patients, with expected medication-induced changes proportionally related to dosage levels of the prescribed medication administered to the first and second patients. In some implementations of the system, the computer is further configured to: retrieve historical cytological data for the first and second patients; and modify the expected medication-induced changes and the threshold based on the first and second patients' prior documented responses to the prescribed medication as indicated in the historical cytological data.

In some implementations of the system, the computer is further configured to: categorize cells from the first sample into distinct abnormality types; and prioritize presentation of specific abnormality types for human review based on their correlation with known adverse medication effects associated with the prescribed medication. In some implementations of the system, the computer is further configured to: determine, for the first sample, an urgency classification based on the degree of deviation above the threshold and clinical significance of the affected cell populations; and route the cells from the first sample to different reviewer queues based on the urgency classification. In some implementations of the system, the computer is further configured to: analyze patient demographic data associated with the first patient; and adjust the threshold based on demographic-specific expected medication-induced changes, wherein cells from the first sample are caused to be presented for human review when the result of the function of the first classifications deviates above the demographic-adjusted threshold. In some implementations of the system, the computer is further configured to: determine a medication class for the prescribed medication; select a medication-class-specific analysis protocol specialized for detecting abnormalities associated with the determined medication class; and apply the medication-class-specific analysis protocol when comparing the result of the function of the first classifications with the expected medication-induced changes.

The following implementation addresses the optimization of human expertise utilization in cytological analysis through a structured review hierarchy that matches automatically classified cells with appropriate reviewer expertise, which may improve efficiency while maintaining diagnostic quality. It may reduce costs by allocating specialized expertise where necessary, while leveraging less specialized resources for appropriate verification tasks. The embodiment supports a workflow that can adapt to varying case complexities, resource availability, and geographic constraints, potentially enabling more cost-effective cytological analysis without compromising diagnostic standards. By creating escalation pathways between review tiers, the system provides relatively complex or uncertain cases with the appropriate level of expertise while optimizing the overall use of human resources across the organization. The system starts with an initial review tier for verification of classification results, and escalates to the second review tier (certified physician) when the initial review tier results indicate a clinically related threshold has been crossed, saving money and time, and focusing clinical experts on cases requiring their expertise, which is expected to increase their work satisfaction and utilization while reducing their overall workload.

In various implementations, a computer-implemented method for implementing expertise-based review hierarchies in cytological smear analysis, comprising: receiving, from an automatic cell classifier, first and second classifications of cells in first and second images of first and second cytological smears associated with first and second samples obtained from first and second patients, respectively; presenting, as part of default human review workflow of an initial review tier, wherein the initial review tier is trained in relevant cell type recognition, cells associated with the first and second images for verification; receiving initial review tier results for the first and second classifications, comprising first and second hematology cell or cell type quantification metrics (HCQMs), respectively; receiving a set of clinically related thresholds for cell types appearing in the first and second classifications; comparing the first and second HCQMs associated with the first and second initial review tier results against the set of clinically related thresholds, respectively; determining that at least one of the first HCQMs crosses a clinically related threshold; determining that none of the second HCQMs crosses a clinically related threshold; presenting, as part of default human review workflow of a second review tier, wherein the second review tier is a certified physician, cells associated with the first image and the first initial review tier results; and not presenting, as part of default human review workflow of a second review tier, cells associated with the second image. The expertise-based review hierarchy may optimize resource allocation by directing samples to appropriate review tiers based on clinical significance indicators. The initial review tier may include laboratory technicians, technologists, or other personnel trained in cell type recognition who perform primary verification of automatic classifications. The second review tier may include certified physicians with specialized expertise for interpreting clinically significant findings. In another embodiment, the initial review tier may include cytotechnologists, medical laboratory scientists, hematology technicians, or other personnel who have received training in the recognition of cells including but not limited to training in morphological identification of normal and abnormal cell types, differentiation of cell lineages, recognition of cellular maturation stages, identification of cellular artifacts, and/or assessment of staining quality and cellular preservation; and wherein the certified physician belonging to the second review tier may possess higher expertise in the recognition of cells than members of the initial review tier, such higher expertise potentially encompassing advanced diagnostic interpretation of subtle morphological variations, correlation of cellular findings with clinical conditions, recognition of rare cell types and pathological conditions, interpretation of complex cellular patterns indicative of specific disease processes, differentiation of mimicking conditions that may present similar cellular morphologies, integration of cellular findings with patient history and other laboratory results, and/or specialized knowledge in subspecialty areas such as hematopathology, cytopathology, or specific disease categories. The tiered approach may reduce physician workload by filtering samples that require advanced clinical interpretation from those adequately verified at the initial tier level. The clinically related thresholds may be established based on medical guidelines, institutional protocols, and/or diagnostic significance levels that determine when physician expertise becomes necessary for proper clinical interpretation.

In the context of this embodiment, “not presenting, as part of default human review workflow of a second review tier, cells associated with the second image” refers to not escalating the second sample to the certified physician tier when none of the second HCQMs crosses a clinically related threshold following initial review tier verification. While cells associated with the first image are presented to the second review tier because at least one of the first HCQMs crosses a clinically related threshold (requiring physician expertise for clinical interpretation), cells associated with the second image are not presented to the second review tier because the second HCQMs remain within clinically acceptable ranges after initial tier verification. The not presenting to the second review tier may be implemented through various mechanisms including: completing the review process at the initial tier level, routing the second sample directly to final reporting without physician review, preventing the second sample from appearing in physician review queues, marking the second sample as completed with initial tier verification while marking the first sample as requiring physician review, applying different tier completion indicators to distinguish samples requiring escalation from those completed at initial tier, storing the second sample in repositories for completed initial-tier-only reviews, presenting the initial tier results for the second sample to physicians without review requirement indicators whereas the first sample results are presented with escalation indicators, and/or displaying the second sample in a manner distinguishable from the first sample in physician interfaces. Such distinguishing manners may include different visual formatting, presence or absence of review action buttons, tier completion badges, positional separation in the interface, or annotations indicating initial-tier-only completion. The system may maintain records of tier assignments while preserving capability for accessing the second sample through non-default mechanisms such as physician-initiated retrieval, quality assurance reviews, when institutional policies require periodic physician sampling of initial-tier-only cases, or when clinical circumstances warrant retrospective physician evaluation of samples that initially completed review at the first tier without escalation to the second tier.

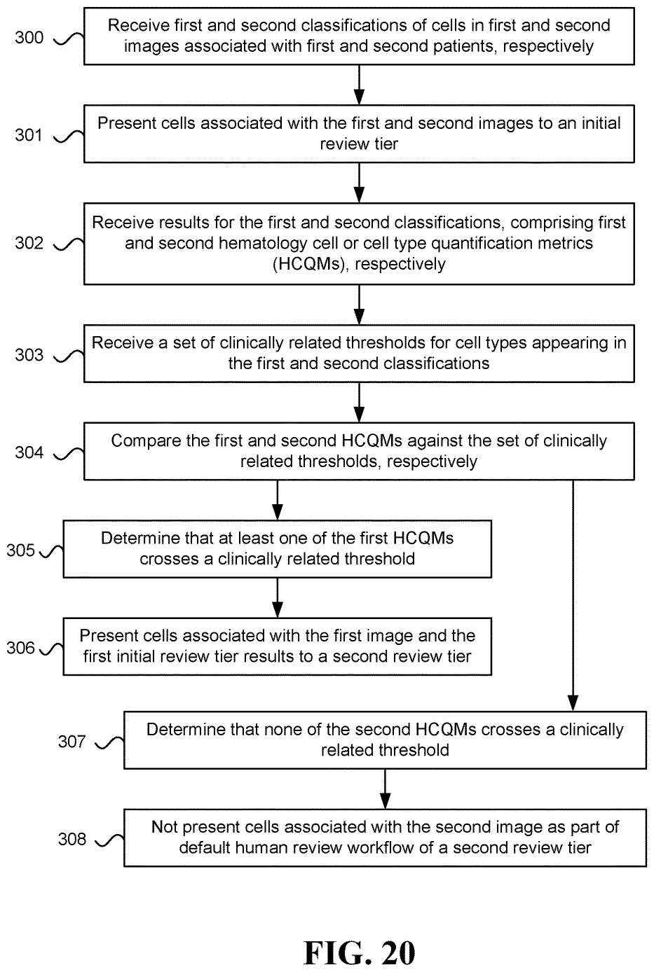

FIG. 20 illustrates an embodiment of a computer-implemented method for implementing expertise-based review hierarchies in cytological smear analysis. The method includes the following steps. In step 300, receive, from an automatic cell classifier, first and second classifications of cells in first and second images of first and second cytological smears associated with first and second samples obtained from first and second patients, respectively. In step 301, present, as part of default human review workflow of an initial review tier, wherein the initial review tier is trained in relevant cell type recognition, cells associated with the first and second images for verification. In step 302, receive initial review tier results for the first and second classifications, comprising first and second hematology cell or cell type quantification metrics (HCQMs), respectively. In step 303, receive a set of clinically related thresholds for cell types appearing in the first and second classifications. In step 304, compare the first and second HCQMs associated with the first and second initial review tier results against the set of clinically related thresholds, respectively. In step 305, determine that at least one of the first HCQMs crosses a clinically related threshold. In step 306, present, as part of default human review workflow of a second review tier, wherein the second review tier is a certified physician, cells associated with the first image and the first initial review tier results. In step 307, determine that none of the second HCQMs crosses a clinically related threshold. And in step 308, not present, as part of default human review workflow of a second review tier, cells associated with the second image.

In various embodiments, the method for implementing expertise-based review hierarchies in cytological smear analysis may facilitate efficient allocation of human expertise across different verification tiers. The system may route cytological smear analyses through a structured review pathway that matches reviewer expertise to analytical complexity. The initial review tier may include laboratory technicians, cytotechnologists, medical laboratory scientists, or other personnel with specialized training in cell morphology recognition and classification. These initial reviewers may possess relevant certification in laboratory sciences, and/or may have completed specialized training modules focused on cytological pattern recognition. The second review tier may include hematologists, pathologists, hematopathologists, or other physicians with advanced training and certification in relevant medical subspecialties. The clinically related thresholds that trigger escalation to the second review tier may be established through various mechanisms, such as published clinical guidelines, laboratory-specific protocols, regulatory requirements, and/or literature-based reference ranges. These thresholds may be dynamically updated based on emerging research, changing clinical standards, and/or accumulated performance data from the review system. The hematology cell or cell type quantification metrics (HCQMs) may represent various quantitative aspects of the cytological analysis, including absolute cell counts, relative proportions, morphological feature measurements, and/or deviation statistics from established reference ranges. The system may implement different escalation pathways for different cell types and/or abnormalities, recognizing that certain findings may warrant specialized expertise regardless of their quantitative measurements. The presentation of cells associated with the first and second images may include various display formats, annotation tools, measurement capabilities, comparison views, and/or interactive navigation features designed to optimize review efficiency and accuracy across different expertise tiers.

In some implementations, the method further comprises implementing a verification tier structure wherein reviewers at the initial review tier verify the automatically classified cells and escalate uncertain or relatively complex cells to specialist tiers. The verification tier structure may be organized according to multiple implementation approaches that reflect varying levels of expertise, specialization, and authority within the review hierarchy. Lower tiers may perform initial verification of automated classifications, applying their expertise in cell morphology and pattern recognition to confirm or correct the automatic cell classifier results. When encountering cells or patterns that exceed their expertise boundaries or established escalation criteria, these reviewers may flag or escalate these findings to higher tiers with more specialized knowledge. The escalation process may follow predefined pathways based on cell types, suspected conditions, classification confidence levels, and/or clinical significance of the findings. Specialist tiers may include hematology experts, oncologists, pathologists with subspecialty training, and/or other medical professionals with advanced expertise relevant to specific cell types or pathological conditions. These higher tiers may provide definitive assessments for complex or unusual cell presentations that require deeper subspecialty knowledge. The verification structure may incorporate branch points where certain findings trigger specialized review pathways tailored to particular medical contexts, such as oncology, infectious disease, or autoimmune conditions. Throughout this multi-tiered process, the system may maintain comprehensive documentation of decisions made at each tier, creating an audit trail that captures the evolution of the analysis across different expertise levels. This structured approach may optimize resource utilization by reserving higher-cost specialist expertise for cases where their advanced knowledge provides meaningful diagnostic value, while leveraging the capabilities of initial tiers for routine verification tasks.

In some implementations of the method, verification tasks performed by the initial review tier are routed to reviewers with lower labor cost compared to the second tier reviewers, and second tier reviewers are certified in their locations. The cross-border expertise utilization approach may leverage global workforce distribution to optimize both cost efficiency and diagnostic quality. Reviewers with lower labor costs may be located in regions where qualified personnel can be engaged at compensation rates that improve overall system economics while maintaining quality standards. These regions may include developing countries with strong medical education systems, areas with lower costs of living but high educational standards, or locations with specialized medical training but different economic environments. The certification of second tier reviewers in their respective locations may involve various credentialing mechanisms, including local medical licensure, international certification programs, specialized cytology training credentials, and/or organization-specific qualification processes that verify appropriate expertise levels. The routing of verification tasks to these initial reviewers may occur through secure digital workflows that maintain data privacy and security while enabling distributed review capabilities. This approach may help organizations balance competing priorities of cost management, expertise access, turnaround time requirements, and quality assurance. The global distribution of review responsibilities may also enable operations across multiple time zones, potentially providing extended coverage hours and faster turnaround times for urgent cases. The system may incorporate location-specific training modules, quality assurance protocols, and/or performance monitoring to maintain consistent standards across geographically dispersed review teams. This approach also supports regulatory requirements, as clinical evaluation is often required to be performed by a person licensed in the same country, whereas classification verification does not have that limitation.

In some implementations of the method, reviewers of the initial review tier are physically based in locations with lower labor costs compared to the second review tier, and the data is sent via secure cross-border data transmission protocols; implementing standardized classification protocols across geographic locations to maintain consistency. The physical location of initial review tier personnel in regions with lower labor costs may create opportunities for cost-efficient resource allocation without compromising analytical quality. Secure cross-border data transmission protocols may incorporate multiple security layers to protect patient data during international transfer, such as end-to-end encryption, anonymization processes, secure authentication mechanisms, and/or compliance with international data protection regulations like GDPR, HIPAA, or equivalent regional frameworks. The transmission infrastructure may include dedicated secure connections, virtual private networks, blockchain-based verification systems, and/or other technologies that maintain data integrity and privacy across international boundaries. Standardized classification protocols implemented across geographic locations may include consistent training methodologies, reference materials, quality assessment procedures, terminology definitions, and/or analytical frameworks that ensure reviewer decisions follow comparable standards regardless of location. These protocols may be reinforced through regular cross-location calibration exercises, shared quality controls, and/or periodic review of classification concordance among reviewers from different regions. The system may implement automated monitoring of classification patterns across locations to identify and address any emerging regional variations. Language standardization tools, translation services, and/or multilingual interfaces may support consistent classification terminology and reporting across locations with different primary languages. The global distribution model may also incorporate contingency protocols for addressing regional disruptions, such as internet outages, local emergencies, or regulatory changes that affect specific locations.

In some implementations of the method, the presenting, as part of default human review workflow of a second review tier, comprises matching specific medical subspecialties in accordance with the initial review tier results. The expertise matching process may dynamically align specialized medical knowledge with specific cytological findings requiring advanced interpretation or clinical decision making. When presenting cases to the second review tier, the system may analyze initial review tier results to identify particular medical subspecialties most relevant to the identified abnormalities or patterns. This matching process may consider multiple dimensions of expertise relevance, including hematological specialties, oncological focus areas, particular disease experience, research backgrounds, or specialized training in rare conditions. The matching algorithms may incorporate classification of cytological findings into medical specialty domains, creating connections between observed cellular abnormalities and the subspecialty knowledge most appropriate for their interpretation. For instance, findings suggesting possible lymphoproliferative disorders may be matched with reviewers having specific expertise in lymphoid malignancies; suspected myelodysplastic changes may be routed to experts in myeloid disorders; and pediatric abnormalities may be directed to specialists with particular experience in childhood hematological conditions. The expertise matching system may utilize machine learning approaches that continuously refine the matching rules based on feedback about review quality, diagnostic accuracy, and/or appropriateness of specialist assignments. This dynamic matching capability may help ensure that cytological findings receive expert assessment from reviewers whose specialized knowledge aligns most closely with the specific medical questions raised by the initial review tier results, potentially improving diagnostic precision and clinical value.

In some implementations, the method further comprises maintaining a repository of reviewer expertise profiles including at least one of specialization areas, certification levels, or performance metrics; matching cells to reviewers based on an algorithm that considers at least one of expertise relevance, reviewer availability, or workload distribution. The repository of reviewer expertise profiles may include a structured knowledge base that captures multiple dimensions of reviewer capabilities, qualifications, and performance histories. The specialization areas documented may include medical subspecialties, research focus areas, rare disease expertise, specific cell type experience, and/or particular analytical techniques. Certification levels recorded may span formal medical board certifications, laboratory credentialing, specialized training program completions, continuing education achievements, and/or internal qualification assessments. Performance metrics maintained in the repository may encompass accuracy rates, consistency measures, speed of review, concordance with final diagnoses, peer assessment results, quality assurance scores, and/or other quantitative and qualitative measures of review quality. The repository may be continuously updated through automated tracking of review outcomes, manual entry of new credentials, periodic reassessment of expertise areas, and/or integration with external certification databases. The algorithm for matching cells to reviewers may employ various computational approaches, including rule-based systems, machine learning models, statistical matching techniques, and/or hybrid approaches that combine multiple methods. This matching process may consider not only the relevance of reviewer expertise to specific cell presentations, but also practical factors that affect workflow optimization. Reviewer availability factors may include scheduled working hours, current queue depth, time since last assignment, anticipated response time, and/or leave status. Workload distribution considerations may incorporate fairness measures, expertise development needs, reviewer fatigue prevention, priority-based assignment protocols, and/or load balancing across the available reviewer pool. The matching system may implement feedback loops that continuously refine the assignment algorithms based on outcome data, potentially improving assignment appropriateness over time.

In some implementations of the method, the presenting, as part of default human review workflow of a second review tier, comprises accessing a repository of condition-specific escalation rules that define quantitative and/or qualitative criteria for specialist review; evaluating initial tier review findings against these criteria to identify samples requiring specialized expertise. The repository of condition-specific escalation rules may constitute a comprehensive knowledge base that codifies the decision criteria for directing cases to specialist reviewers. These escalation rules may be structured according to various medical conditions, cell abnormalities, or diagnostic categories, with each rule set specifying the particular circumstances that warrant higher-tier review. Quantitative criteria within these rules may include numerical thresholds for specific cell populations, morphological measurement boundaries, statistical deviations from reference ranges, confidence intervals for classification certainty, and/or mathematical combinations of multiple parameters. Qualitative criteria may encompass morphological patterns requiring specialized interpretation, unusual cell presentations, rare abnormalities, contextual factors from patient history, and/or combinations of findings that collectively suggest complex conditions. The process of accessing this repository during review workflows may involve automated rule evaluation, context-aware filtering of applicable criteria, real-time application of decision algorithms, and/or integration with clinical decision support frameworks. The evaluation of initial tier review findings against these criteria may employ various analytical approaches, including direct threshold comparisons, pattern matching algorithms, probabilistic assessments, decision tree methodologies, and/or machine learning models trained to recognize complex escalation patterns. This structured approach to escalation may help standardize when specialized expertise is deployed, potentially reducing subjectivity in referral decisions while ensuring appropriate utilization of specialist resources. The escalation criteria may be periodically updated based on emerging medical knowledge, feedback from specialist reviewers, outcome analysis, and/or evolving clinical practice guidelines to maintain alignment with current diagnostic standards.

In some implementations, the method further comprises generating tier-specific contextual information packages that provide second tier reviewers with at least one of: rationales for previous review decisions; highlight specific areas of uncertainty identified by previous reviewers; include relevant patient history data filtered for the specific expertise level; and present comparison data between automated and human classification results. The generation of tier-specific contextual information packages may enhance review efficiency and accuracy through targeted knowledge delivery tailored to each tier's particular needs and expertise levels. These contextual packages may be dynamically assembled from various information sources, including patient records, previous analysis results, automatic classifier outputs, review history, and/or reference materials relevant to the specific case context. The rationales for previous review decisions that are provided to second tier reviewers may include documented reasoning, annotated images, confidence assessments, reference comparisons, and/or explanatory notes that clarify the thought process behind initial classifications or verification decisions. Highlighted areas of uncertainty may be visually mapped within images, quantitatively expressed through confidence metrics, contextually explained through comparative references, and/or flagged with specific questions or concerns that prompted escalation. The patient history data included in these packages may be filtered and prioritized according to the specific expertise level of the receiving tier, with different information emphasized for different specialties or review contexts. This filtering process may consider the medical relevance of various history elements to the specific findings being reviewed, the expertise profile of the receiving reviewer, the suspected conditions indicated by cellular findings, and/or the specific questions raised during prior review stages. The comparison data between automated and human classification results may include concordance statistics, highlighted discrepancies, visualization of classification confidence levels, alternative classifications considered, and/or explanations for classification changes made during human review. This contextual information may be presented through interactive interfaces that allow reviewers to explore different information dimensions, toggle between visualization modes, access supporting reference materials, and/or dynamically adjust the presentation to emphasize different contextual elements according to their specific review focus.

In some implementations of the method, the determining that at least one of the first HCQMs crosses a clinically related threshold comprises evaluating combinations of factors that individually fall below clinically related thresholds or have no clinically related thresholds, but collectively indicate potential complex conditions; identifying patterns of minor deviations across multiple cell parameters that correlate with specific pathological conditions; and triggering escalation when such combinatorial patterns are detected even when single-parameter classifications would not warrant escalation. The evaluation of combinations of factors for escalation triggers may implement pattern recognition that identifies clinically significant constellations of findings that might be missed through single-parameter assessment alone. The system may analyze interrelationships between multiple cell parameters, morphological features, population distributions, and/or classification confidence levels to detect complex patterns indicative of particular pathological conditions. Even when individual parameters fall below established clinical thresholds or lack defined thresholds altogether, their specific combinations may carry diagnostic significance that warrants specialist review. The identification of patterns of minor deviations across multiple cell parameters may employ various analytical methodologies, including multivariate statistical analysis, machine learning pattern recognition, correlation detection algorithms, clustering techniques, and/or rule-based combinations of parameter assessments. These patterns may be correlated with specific pathological conditions through knowledge bases derived from medical literature, expert-defined rule sets, learning algorithms trained on outcome data, and/or statistical correlations established through previous case analyses. The triggering of escalation based on combinatorial patterns may occur through various mechanisms, including automated pattern detection algorithms, calculated compound risk scores, probability assessments of potential pathological conditions, and/or specialized rules for particular parameter constellations. This multi-factor approach may expand the system's capability to identify subtle but clinically significant presentations that might escape detection through traditional single-parameter threshold evaluations, potentially enhancing the system's sensitivity for complex conditions and edge cases that benefit most from specialist expertise.

In some implementations, the method further comprises capturing explicit feedback from second tier reviewers about initial tier review results; automatically detecting patterns in review modifications and escalations; and refining choice of initial tier reviewers. The capturing of explicit feedback from second tier reviewers about initial tier review results may create a structured knowledge loop that continuously improves routing decisions and reviewer selection. This feedback may be collected through various mechanisms, including structured assessment forms, scoring systems for review quality, categorized feedback options, annotation tools for specific comments, and/or integrated feedback interfaces within the review workflow. The feedback content may address multiple quality dimensions, such as classification accuracy, appropriate application of established criteria, completeness of analysis, proper identification of abnormalities, and/or effectiveness of uncertainty flagging. The automatic detection of patterns in review modifications and escalations may employ various analytical approaches, including statistical trend analysis, machine learning pattern recognition, aggregate performance metrics, discrepancy categorization, and/or correlation analyses between reviewer characteristics and modification patterns. These analyses may identify systematic variation in review quality across different initial reviewers, particular strengths and weaknesses of specific reviewers, pattern-specific performance differences, and/or contextual factors that influence review accuracy. The refinement of initial tier reviewer choice based on this feedback analysis may involve multiple adjustment methods, including personalized routing rules that direct specific finding types to reviewers with demonstrated strength in those areas, dynamic adjustment of reviewer assignment algorithms, targeted training interventions for identified weaknesses, and/or optimization of reviewer-case matching based on empirical performance data. This feedback-driven refinement process may create a continuously learning system that progressively improves the alignment between case characteristics and reviewer capabilities, potentially enhancing overall system accuracy and efficiency through increasingly optimized routing decisions.

In some implementations, the method further comprises capturing explicit feedback from initial tier reviewers about automatic cell classifier results; automatically detecting patterns in review modifications and escalations; and updating the cell classifier to reduce human review correction rates. The capturing of explicit feedback from initial tier reviewers about automatic cell classifier results may establish a structured learning loop that progressively refines the classifier's performance. This feedback may be collected through various interfaces, including integrated correction tools, classification change tracking, structured assessment forms, annotation capabilities for specific cells, and/or categorized feedback selection options that document the nature of needed corrections. The feedback content may address multiple dimensions of classifier performance, such as cell identification accuracy, morphological feature assessment, appropriate classification confidence levels, detection of rare or abnormal cells, and/or specific error patterns observed across different cell types or conditions. The automatic detection of patterns in review modifications and escalations may employ various analytical methodologies, including statistical pattern analysis, machine learning approaches to error categorization, correlation detection between cell characteristics and misclassification rates, identification of context-specific performance variations, and/or trend analysis of classifier accuracy across different sample types or patient populations. The updating of the cell classifier to reduce human review correction rates may involve multiple improvement methods, including retraining with corrected examples, algorithm refinement focused on identified error patterns, adjustment of classification parameters for problematic cell types, enhancement of feature detection capabilities for commonly missed characteristics, threshold adjustment and/or implementation of specialized classification rules for challenging edge cases. This continuous feedback and improvement cycle may create a progressively more accurate classification system that learns from human expertise, potentially reducing the correction burden over time while maintaining appropriate human verification for critical or complex classifications.

In some implementations, the method further comprises tracking review progression through the review hierarchy, including documenting reviewer decisions, time spent at different tiers, and modifications made at successive review levels. The tracking of review progression through the review hierarchy may create a comprehensive audit trail that documents the evolution of analysis across different expertise tiers. This tracking may capture multiple dimensions of the review process, including sequential decision points, modifications to classifications or interpretations, escalation rationales, verification confirmations, and/or uncertainty resolutions. The documentation of reviewer decisions may encompass various elements, such as verification status for each classification, specific modifications made to automatic or previous human classifications, confidence assessments for verified results, rationales for classification changes, and/or annotations explaining particular interpretations. The tracking of time spent at different tiers may include timestamps for review initiation and completion, duration metrics for different review components, comparison of actual versus expected review times, pattern analysis of time requirements for different finding types, and/or identification of workflow bottlenecks or efficiency opportunities. The documentation of modifications made at successive review levels may capture the progression of analytical refinement, including specific classification changes, diagnostic interpretations added, confidence assessments adjusted, quantitative measurements corrected, and/or contextual interpretations enhanced. This comprehensive tracking may serve multiple purposes, including quality assurance monitoring, workflow optimization, educational feedback for reviewers, performance assessment, regulatory compliance documentation, and/or continuous improvement analysis for the overall system. The progression tracking data may be structured to support various analytical approaches, including pattern detection in escalation decisions, quality metrics across different review tiers, efficiency analysis of the review hierarchy, and/or identification of recurring challenges that may benefit from process improvements or additional training.

In some implementations, the method further comprises monitoring real-time workload and availability across review tiers; dynamically adjusting routing decisions to optimize resource utilization; implementing surge capacity protocols that temporarily modify escalation thresholds during high-volume periods; and prioritizing most clinically significant cells over workload conditions. The monitoring of real-time workload and availability across review tiers may enable dynamic optimization of resource allocation in response to changing conditions. The system may track multiple workload indicators, including queue depths, average review times, backlog metrics, incoming case volumes, reviewer availability status, and/or priority distribution of pending cases. This monitoring may employ various analytical approaches, including real-time dashboard views, predictive models of workload evolution, statistical trend analysis, capacity modeling, and/or alert mechanisms for approaching capacity limits. The dynamic adjustment of routing decisions may implement various optimization methods, including load-balancing algorithms that distribute cases evenly among available reviewers, expertise-weighted assignment that accounts for both workload and reviewer capabilities, priority-based queue management that ensures timely handling of urgent cases, and/or predictive routing that anticipates forthcoming capacity constraints. Surge capacity protocols may provide structured responses to high-volume periods through multiple mechanisms, including temporary threshold modifications that adjust escalation criteria to focus specialist resources on the most important cases, cross-training utilization that enables reviewers to work across broader case types during peak periods, expanded availability scheduling that increases reviewer capacity during surge events, and/or prioritization frameworks that ensure the most significant cases receive timely review even during capacity constraints. Throughout these adaptive processes, the system may maintain differentiated handling of cases based on clinical significance, ensuring that important findings receive appropriate attention regardless of overall workload conditions. This significance-based prioritization may consider multiple factors, including potential diagnostic importance, time sensitivity of findings, patient risk factors, and/or criticality of suspected conditions, creating differentiated processing pathways that align review resources with clinical priorities during capacity-constrained periods.

In some implementations, the method further comprises automatically generating and storing a report in an electronic medical information system for the second patient, wherein the report presents analysis results according to the verified initial review tier results. The automatic generation and storage of a report in an electronic medical information system may streamline the documentation process while ensuring appropriate integration of verified results into the patient's medical record. The report may be dynamically created through various approaches, including templated document generation, structured data formatting, narrative text synthesis, and/or hybrid methods that combine standardized elements with case-specific details. The presentation of analysis results according to the verified initial review tier results may incorporate multiple information elements, including quantitative cell counts, morphological descriptions, classification results, reference range comparisons, trend analysis relative to previous results, visualization of key findings, and/or interpretative comments added during verification. The formatting and content of these reports may be tailored to different clinical contexts, specialties, or usage scenarios through rules-based content selection, recipient-specific formatting, customizable presentation options, and/or integration with specialty-specific terminology or reference standards. The automatic storage process may implement various integration mechanisms with the electronic medical information system, including direct database connections, HL7 messaging, FHIR-based integration, API communications, and/or other health information exchange standards that ensure secure and reliable data transfer. The system may include verification mechanisms that confirm successful storage and availability of the report within the patient's record, potentially including acknowledgment receipts, integration confirmation checks, accessibility verification, and/or audit trail documentation of the storage process. This automated reporting may reduce manual documentation burden while maintaining comprehensive recording of analysis results, potentially improving both efficiency and consistency in the documentation process.

In some implementations, the method further comprises automatically generating and storing a report in an electronic medical information system for the first patient, wherein the report presents analysis results according to the verified second review tier results. The automatic generation and storage of a report in an electronic medical information system for the first patient may integrate the enhanced diagnostic insights provided by second review tier verification. The report may be created through various approaches, including conditional template systems that adapt based on verification level, structured data models with tiered verification flags, narrative generation frameworks that incorporate specialist-level terminology, and/or integrated formats that present both initial and second tier assessment results. The presentation of analysis results according to the verified second review tier results may include multiple information dimensions, such as specialist interpretations of findings, refined classification assessments, advanced morphological descriptions, clinical correlation suggestions, diagnostic possibilities raised by the findings, recommended follow-up testing, and/or contextual interpretations that place the cellular findings within the broader clinical picture. These reports may implement different formatting approaches based on the verification level, potentially including distinctive sections for specialist assessments, highlighted modifications from initial reviews, confidence indicators for different findings, and/or specialized visualization of findings that required expert interpretation. The integration with the electronic medical information system may employ various mechanisms that maintain the distinction between different verification levels, potentially including tiered verification flags, digital signature indicators for specialist review, differentiated access controls based on verification status, and/or version tracking that maintains the progression of interpretations across review tiers. The system may implement notification protocols that alert relevant clinical personnel when specialist verification has substantially modified initial findings, potentially including automated communication triggers, significance-based alerting rules, and/or integrated messaging with the electronic medical information system. This specialized reporting for cases receiving second tier review may help ensure that the additional expertise applied during verification translates effectively into the clinical documentation available to treating physicians. In some implementations of the method, a non-transitory computer-readable medium comprising instructions that, when executed by a processor, cause the processor to perform the computer-implemented method.

In various implementations, a system for implementing expertise-based review hierarchies in cytological smear analysis, comprising: a computer configured to: receive, from an automatic cell classifier, first and second classifications of cells in first and second images of first and second cytological smears associated with first and second samples obtained from first and second patients, respectively; cause cells associated with the first and second images to be presented as part of default human review workflow of an initial review tier, wherein the initial review tier is trained in relevant cell type recognition, for verification; receive initial review tier results for the first and second classifications, comprising first and second hematology cell or cell type quantification metrics (HCQMs), respectively; receive a set of clinically related thresholds for cell types appearing in the first and second classifications; compare the first and second HCQMs associated with the first and second initial review tier results against the set of clinically related thresholds, respectively; determine that at least one of the first HCQMs crosses a clinically related threshold; determine that none of the second HCQMs crosses a clinically related threshold; cause cells associated with the first image and the first initial review tier results to be presented as part of default human review workflow of a second review tier, wherein the second review tier is a certified physician; and cause cells associated with the second image to be excluded from presentation as part of default human review workflow of a second review tier.

In some implementations of the system, the computer is further configured to implement a verification tier structure wherein reviewers at the initial review tier verify the automatically classified cells and escalate uncertain or relatively complex cells to specialist tiers. In some implementations of the system, the computer is further configured to route verification tasks performed by the initial review tier to reviewers with lower labor cost compared to the second tier reviewers, and second tier reviewers are certified in their locations. In some implementations of the system, reviewers of the initial review tier are physically based in locations with lower labor costs compared to the second review tier, wherein the computer is further configured to: send data via secure cross-border data transmission protocols; and implement standardized classification protocols across geographic locations to maintain consistency.

In some implementations of the system, the computer, when causing cells associated with the first image and the first initial review tier results to be presented as part of default human review workflow of a second review tier, is further configured to match specific medical subspecialties in accordance with the initial review tier results. In some implementations of the system, the computer is further configured to: maintain a repository of reviewer expertise profiles including at least one of specialization areas, certification levels, or performance metrics; and match cells to reviewers based on an algorithm that considers at least one of expertise relevance, reviewer availability, or workload distribution.

In some implementations of the system, the computer, when causing cells associated with the first image and the first initial review tier results to be presented as part of default human review workflow of a second review tier, is further configured to: access a repository of condition-specific escalation rules that define quantitative and/or qualitative criteria for specialist review; and evaluate initial tier review findings against these criteria to identify samples requiring specialized expertise. In some implementations of the system, the computer is further configured to generate tier-specific contextual information packages that provide second tier reviewers with at least one of: rationales for previous review decisions; highlight specific areas of uncertainty identified by previous reviewers; include relevant patient history data filtered for the specific expertise level; and present comparison data between automated and human classification results.

In some implementations of the system, the computer, when determining that at least one of the first HCQMs crosses a clinically related threshold, is further configured to: evaluate combinations of factors that individually fall below clinically related thresholds or have no clinically related thresholds, but collectively indicate potential complex conditions; identify patterns of minor deviations across multiple cell parameters that correlate with specific pathological conditions; and trigger escalation when such combinatorial patterns are detected even when single-parameter classifications would not warrant escalation. In some implementations of the system, the computer is further configured to: capture explicit feedback from second tier reviewers about initial tier review results; automatically detect patterns in review modifications and escalations; and refine choice of initial tier reviewers.

In some implementations of the system, the computer is further configured to: capture explicit feedback from initial tier reviewers about automatic cell classifier results; automatically detect patterns in review modifications and escalations; and update the cell classifier to reduce human review correction rates. In some implementations of the system, the computer is further configured to track review progression through the review hierarchy, including documenting reviewer decisions, time spent at different tiers, and modifications made at successive review levels. In some implementations of the system, the computer is further configured to: monitor real-time workload and availability across review tiers; dynamically adjust routing decisions to optimize resource utilization; implement surge capacity protocols that temporarily modify escalation thresholds during high-volume periods; and prioritize most clinically significant cells over workload conditions.

In some implementations of the system, the computer is further configured to automatically generate and store a report in an electronic medical information system for the second patient, wherein the report presents analysis results according to the verified initial review tier results. In some implementations of the system, the computer is further configured to automatically generate and store a report in an electronic medical information system for the first patient, wherein the report presents analysis results according to the verified second review tier results.

A computer-implemented method for prioritizing cytological smear reviews may address the efficient allocation of review resources by ordering cases according to their clinical urgency and significance rather than simple queue position. The method may involve receiving automatically classified cells in images of cytological smears designated for human review and calculating priority scores based on multiple factors. These priority calculations may incorporate both clinical significance of preliminary findings and review urgency considerations, potentially allowing the most critical cases to receive prompt attention regardless of when they entered the review pipeline. By implementing this prioritization approach, the system may help ensure that limited review resources are allocated to cases where timely review may have the greatest potential impact on patient outcomes.

When calculating priority scores, the system may assign higher weights to sets of automatically classified cells with preliminary findings suggestive of conditions requiring rapid intervention. This weighting approach may recognize that certain cellular presentations have greater time-sensitivity than others due to their potential clinical implications. For example, findings potentially indicative of acute leukemia, severe infections, or other critical conditions may receive higher priority weights than those suggesting stable or chronic conditions. The specific weighting factors may be derived from clinical guidelines, institutional protocols, and/or expert consensus regarding intervention timeframes for different pathological presentations.

The priority calculation may further incorporate adjustments based on patient context factors including previous diagnoses, treatment status, and specialist referral requirements. This context-aware prioritization may recognize that the same cellular findings may carry different urgency implications depending on the patient's clinical situation. For example, certain findings may warrant higher priority in patients undergoing active treatment monitoring, those with previous related diagnoses, or cases requiring specialist input for treatment decisions. The contextual adjustment may be implemented through modifier coefficients applied to base priority scores or through separate scoring components that are integrated into the overall priority calculation.

As laboratory workflow progresses, the system may update priority scores using a temporal escalation model that increases priority for sets of automatically classified cells that have remained in the queue beyond predetermined time thresholds. This approach may help prevent lower-priority cases from experiencing excessive delays by progressively elevating their priority as wait time increases. The temporal model may implement various escalation patterns, such as stepwise increases at specific time points or continuous functions that accelerate priority growth over time. This mechanism may create a balanced system that both prioritizes critical cases and ensures reasonable turnaround times for routine reviews.

The system may implement an emergency override mechanism that elevates certain sets of automatically classified cells to the top of the queue based on predefined critical finding triggers or explicit urgency flags. This mechanism may provide immediate attention to cases where automated analysis detects patterns strongly associated with life-threatening conditions or where external information indicates exceptional urgency. The override criteria may include specific cellular patterns, combinations of findings, or external flags such as STAT orders or critical patient status indicators. This capability may ensure appropriate response to genuine emergencies while maintaining the overall priority-based workflow for routine cases.

The updating of priority scores and queue positions may occur at various times, including at predetermined time intervals, when new automatically classified cells are received, when changes occur to the review queue, when reviews are completed, or when laboratory conditions change. This flexible update timing may allow the prioritization system to adapt to changing conditions while balancing computational efficiency considerations. The specific implementation may select optimal update triggers based on laboratory volume, priority volatility, and available computing resources, potentially employing different update strategies during peak versus off-peak periods.

For efficiency purposes, the updating process may selectively recalculate priority scores for a subset of the sets of automatically classified cells rather than recalculating all queue items. This selective approach may focus computational resources on cases most likely to experience significant priority changes, such as those affected by temporal escalation thresholds, newly added contextual information, or changing laboratory conditions. The selection of which cases to update may be based on various factors including time since last update, proximity to temporal thresholds, or sensitivity to specific types of laboratory condition changes.

The priority calculation parameters may be adjusted based on current reviewer workload, potentially modifying priority thresholds for escalation in response to available review capacity. During periods of high capacity, the system may implement more granular prioritization to optimize reviewer efficiency, while during low-capacity periods, it may elevate more cases to higher priority levels to ensure critical reviews aren't delayed. The workload-based adjustments may consider factors such as reviewer availability, backlog size, anticipated incoming volume, and current throughput rates to dynamically tune the prioritization system to laboratory conditions.

As part of the updating process, the system may redistribute review resources across different priority levels based on laboratory throughput metrics and current queue composition. This resource allocation approach may ensure appropriate staffing for different priority tiers based on current needs rather than fixed allocations. For example, when the high-priority queue contains more cases than can be promptly addressed, additional review resources may be temporarily shifted from lower-priority reviews. The redistribution decisions may incorporate factors such as priority distribution patterns, estimated review time requirements, reviewer expertise, and throughput targets for different priority levels.

The following implementation addresses the generation of preliminary automated reports that are subsequently updated after human verification. By implementing progressive reporting, the system may provide timely preliminary results while maintaining quality through human verification, and expedite result delivery while preserving the value of expert review. The embodiment supports efficient laboratory workflows by separating initial automated reporting from subsequent expert verification and updates, potentially reducing turnaround time for urgent information while supporting appropriate expert validation of the complete results.

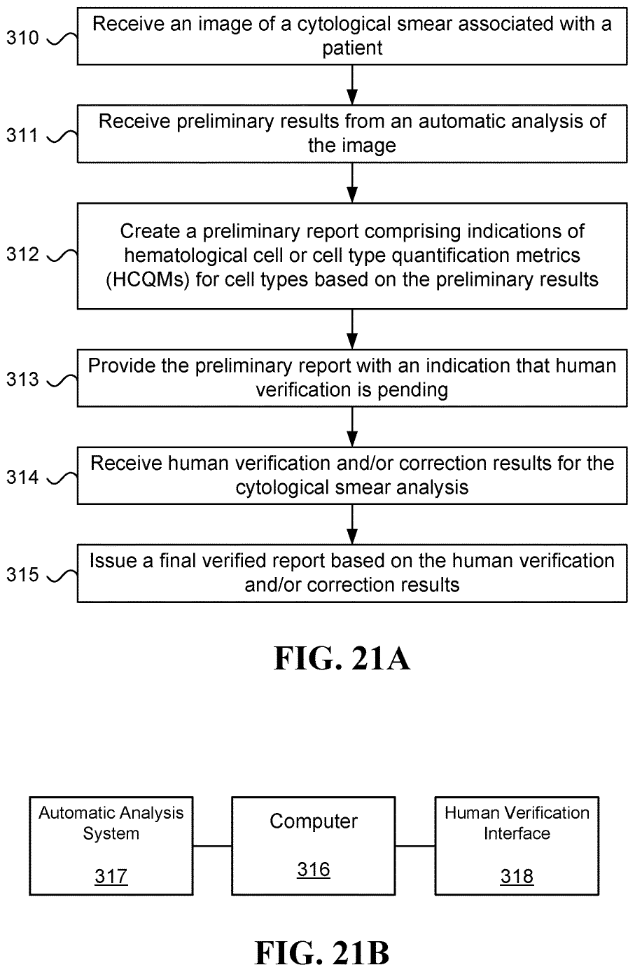

In various implementations, a computer-implemented method for progressive cytological reporting with human verification, comprising: receiving an image of a cytological smear associated with a patient; receiving preliminary results from an automatic analysis of the image; creating a preliminary report comprising indications of hematological cell or cell type quantification metrics (HCQMs) for cell types, based on the preliminary results; providing the preliminary report with an indication that human verification is pending; receiving human verification and/or correction results for the cytological smear analysis; and issuing a final verified report based on the human verification and/or correction results. FIG. 21A illustrates an embodiment of a computer-implemented method for progressive cytological reporting with human verification. The method includes the following steps. In step 310, receive an image of a cytological smear associated with a patient. In step 311, receive preliminary results from an automatic analysis of the image. In step 312, create a preliminary report comprising indications of hematological cell or cell type quantification metrics (HCQMs) for cell types, based on the preliminary results. In step 313, provide the preliminary report with an indication that human verification is pending. In step 314, receive human verification and/or correction results for the cytological smear analysis. And in step 315, issue a final verified report based on the human verification and/or correction results.

The progressive reporting approach may implement a multi-stage workflow that balances timeliness with accuracy in cytological analysis reporting. The preliminary report may be generated promptly after automatic analysis without waiting for human verification, which may reduce turnaround time for initial results that could guide immediate clinical decisions. This preliminary report may include quantitative metrics such as cell counts, percentages, ratios, and/or other derived values that provide initial insights into the patient's hematological status. The indication that human verification is pending may be implemented through various mechanisms including but not limited to visual markers, electronic flags, specific report sections, header notations, watermarks, or automated notifications that clearly communicate the preliminary nature of the report. The human verification and/or correction process may involve expert review of the automatic analysis results with capabilities for confirming accurate findings and/or correcting any identified discrepancies. The final verified report may incorporate all human verification inputs while maintaining traceability between preliminary and final findings. The system may be configured to operate within various laboratory information systems, electronic health record platforms, or dedicated cytological analysis environments, with appropriate data exchange protocols to maintain integrity throughout the progressive reporting workflow.

In some implementations, the method further comprises detecting when human verification results alter the clinical significance of the preliminary results; generating an alert for such alterations; and expediting the issuing of the final verified report for cases where the clinical significance has changed. The system may implement mechanisms to identify when human verification substantially alters the clinical interpretation or significance of findings initially presented in the preliminary report. Such alterations may include changes that affect normal vs. abnormal results for cell types or cell type combinations, diagnostic categorization, treatment recommendations, monitoring intervals, prognostic indicators, and/or other clinically meaningful parameters. The detection process may involve comparing key metrics before and after human verification against predetermined thresholds defined for clinical significance, or may employ algorithmic assessment of whether changes cross clinically important boundaries. Alert generation may include various notification mechanisms such as automated messaging, visual indicators within laboratory systems, priority flags, or escalation pathways appropriate to the magnitude and urgency of the clinical significance alteration. The expedition of final verified report issuance may involve prioritization within reporting queues, accelerated approval workflows, or dedicated fast-track protocols that reduce administrative delays. This enhanced handling of significance-altering verifications may help maintain appropriate clinical response timelines when human expert input substantially modifies the preliminary assessment. The specific implementation of significance thresholds may vary based on the type of cytological analysis, patient demographics, clinical context, and/or laboratory protocols to appropriately balance sensitivity to clinically meaningful changes against unnecessary alert generation.

In some implementations, the method further comprises implementing a retraction protocol when human verification contradicts the preliminary results with potential clinical impact above a predetermined threshold; wherein the retraction protocol comprises notifying recipients of the preliminary report about the contradiction, and issuing a final verified report with indication of changes related to the clinical impact. The retraction protocol may be triggered when human verification identifies contradictions to preliminary results that exceed established clinical impact thresholds. These thresholds may incorporate factors such as the magnitude of quantitative discrepancies, qualitative reclassifications of cell types, identification of critical findings missed in preliminary analysis, and/or other parameters that could substantially influence patient management decisions. The notification process may utilize multiple communication channels to ensure that all recipients of the preliminary report are promptly informed about the contradiction, potentially including automated alerts, direct communications, electronic health record notifications, and/or documented phone contacts depending on urgency and institutional protocols. The indications of changes in the final verified report may include explicit highlighting of contradictions, side-by-side comparisons of preliminary and verified findings, detailed explanations of discrepancies, and/or standardized change indicators that facilitate rapid identification of modified results. Optionally, the retraction protocol may also include documenting the basis for the contradiction, which may encompass technical factors such as staining artifacts, algorithmic limitations, sample quality considerations, rare cell type identification, and/or other explanatory details that provide context for the discrepancy. These documentation elements may serve quality improvement purposes by identifying patterns in preliminary reporting limitations while also providing transparency in the analytical process. Additionally, the retraction protocol may include mechanisms for tracking resolution of contradicted results, confirmation of receipt of retraction notifications, and/or systematic review of similar cases to identify potential systemic issues requiring adjustment. Optionally, the retraction protocol also includes documenting the basis for the contradiction.

In some implementations, the method further comprises identifying when human verification changes a finding from one with routine findings to one requiring immediate intervention or vice versa, and modifying report delivery and clinical alert mechanisms accordingly to facilitate appropriate clinical response. The continuous monitoring update protocol may implement adaptive reporting pathways that respond to the clinical urgency revealed through human verification. The identification of clinically significant shifts may employ rule-based assessment, comparison against significant value thresholds, evaluation of trend significance, pattern recognition algorithms, and/or other analytical approaches that detect meaningful changes in patient status implications. When human verification elevates clinical urgency by transforming routine findings into those requiring immediate intervention, the system may automatically activate accelerated communication protocols including but not limited to direct clinician notification, integration with significant results management systems, escalation to supervisory personnel, or activation of urgent care pathways appropriate to the specific finding. Conversely, when human verification reduces urgency by reclassifying preliminary findings that appeared to require immediate intervention into routine findings, the system may appropriately adjust alert levels to prevent unnecessary clinical responses while still maintaining standard reporting workflows. The modification of delivery mechanisms may include changes to report formatting, delivery pathways, notification recipients, accompanying documentation requirements, and/or follow-up verification protocols. This adaptive approach may help allocate clinical response resources proportionally to verification-confirmed urgency, potentially reducing unnecessary emergency responses while ensuring appropriate attention to genuinely urgent situations identified through human verification. The specific thresholds and response protocols may be customizable based on institutional policies, patient population characteristics, resource availability, and/or other relevant operational factors.

In some implementations of the method, the preliminary report includes comparisons with previous cytological results from the patient's medical history, and further comprising validating, during the human verification, significant changes from the patient's baseline. The temporal comparison functionality may incorporate historical patient data to provide context for current findings in both preliminary and verified reporting stages. In the preliminary report, automated comparisons may highlight quantitative trends, directional shifts, rate of change calculations, stability assessments, and/or other metrics that place current findings within the patient's longitudinal context. These comparisons may draw from various historical sources including previous cytological analyses, related laboratory studies, documented baseline values, and/or other relevant clinical data points available in the patient's medical history. The specific comparison parameters may adapt based on the clinical context, potentially emphasizing different metrics for monitoring known conditions versus screening contexts. The human verification process may specifically address the validity of identified temporal changes, with reviewers evaluating whether apparent trends represent genuine physiological shifts versus artifacts introduced by technical variations, specimen quality differences, or analytical inconsistencies between time points. Reviewers may assess the clinical significance of confirmed trends, potentially adding interpretive context not captured in automated comparisons. The validation of significant changes may include assessment of coherence with the patient's overall clinical trajectory, consistency with expected disease progression patterns, correlation with therapeutic interventions, and/or congruence with other diagnostic findings. This human-verified temporal context may enhance clinical interpretation by distinguishing between expected variations, significant but explained changes, and unexpected deviations requiring further investigation or intervention. The system may maintain traceability of temporal assessments through both preliminary and verified stages to document how expert verification modifies the interpretation of longitudinal patterns.

In some implementations, the method further comprises specially marking instances where human verification reverses previous trend assessments in temporal comparisons, and providing detailed explanations for such reversals to facilitate proper clinical interpretation of the patient's disease progression. The temporal significance tracking functionality may implement enhanced documentation when human verification substantially modifies trend interpretations established in previous analyses or preliminary assessments. The special marking may utilize distinctive visual indicators, dedicated report sections, standardized nomenclature, explicit flags, or other attention-directing mechanisms that clearly signal reversed trend assessments to report recipients. These markers may vary in prominence based on the clinical significance of the reversal, with more consequential reversals receiving more prominent indicators. The detailed explanations accompanying these reversals may address multiple dimensions including methodological factors (such as changes in analytical techniques, staining protocols, or classification algorithms), specimen-related considerations (such as quality variations, sampling differences, or preparation artifacts), clinical context elements (such as therapeutic effects, physiological fluctuations, or intercurrent conditions), and interpretive reassessments (such as recognition of rare variants, reclassification of cell types, or identification of previously unrecognized patterns). These explanations may include both technical components that document the basis for the reversal and clinical interpretation elements that translate the significance of the reversal for patient management. The system may maintain a longitudinal record of trend assessment modifications to establish a comprehensive history of how interpretations have evolved, potentially revealing patterns in assessment challenges for specific conditions or cell types. This detailed tracking of interpretive reversals may help prevent clinical misunderstandings that could arise from seemingly contradictory assessments across time, potentially improving continuity of care when multiple clinicians are involved in patient management. The system may implement varying levels of explanation detail based on the recipient type, potentially providing more technical information in laboratory-facing documentation and more clinical implication-focused content in physician-facing reports.

In some implementations, the method further comprises automatically recommending appropriate ancillary testing when human verification identifies conditions requiring confirmatory or additional testing that were not indicated by the preliminary results, and including these recommendations in the final verified report. The verification-triggered ancillary testing functionality may implement automated recommendation mechanisms that activate when human verification identifies findings suggesting the need for supplementary diagnostic evaluation. These recommendations may be generated based on rule-based systems, clinical guidelines, institutional protocols, expert knowledge bases, and/or machine learning models that correlate specific cytological findings with appropriate follow-up testing pathways. The conditions triggering such recommendations may include identification of abnormal cells requiring further characterization, ambiguous findings necessitating confirmatory testing, unexpected results suggesting alternative diagnostic considerations, incidental findings warranting specialized evaluation, or combinations of results that collectively indicate the need for comprehensive workup protocols. The recommendation process may incorporate patient-specific factors such as medical history, demographic information, medication usage, and/or prior test results to optimize the relevance and appropriateness of suggested ancillary testing. The system may provide varying levels of recommendation specificity ranging from general test categories to detailed testing protocols with specific methodologies, timing considerations, sample requirements, and/or preferred testing sequences. The inclusion of these recommendations in the final verified report may involve standardized formatting, clear differentiation from diagnostic conclusions, appropriate clinical rationales, and/or indications of recommendation urgency or priority. Additionally, the system may facilitate direct ordering of recommended tests through integration with laboratory information systems, electronic health records, or computerized provider order entry systems, potentially including pre-populated order details to streamline the implementation of verification-triggered recommendations. The system may track recommendation patterns to identify opportunities for refinement of preliminary analysis capabilities or to inform updates to recommendation algorithms based on observed patterns of human verification-triggered testing needs.

In some implementations of the method, the creating the preliminary report comprises including in the preliminary report findings that fall on the sufficient confidence side of a predetermined classification confidence level (CCL) threshold, while designating other findings as awaiting verification. The confidence-based preliminary reporting approach may implement selective inclusion criteria that balance timely reporting of high-confidence findings with appropriate caution regarding less certain results. The predetermined CCL thresholds may be established through various methodologies including statistical modeling, validation studies, historical performance analysis, expert consensus processes, and/or alignment with clinical significance considerations for different finding types. These thresholds may vary across different cell types, quantification metrics, or analytical parameters based on their respective clinical implications, technical reliability characteristics, and verification complexity requirements. The inclusion of findings exceeding CCL thresholds in preliminary reports may involve full reporting with quantitative details, interpretive comments, and comparative assessments similar to verified findings, while clearly indicating their preliminary status. For findings falling below CCL thresholds, the designation as “awaiting verification” may be implemented through various approaches including complete omission from preliminary reports with notation of pending parameters, inclusion with explicit verification-pending indicators, presentation in separate report sections with distinct formatting, or display with visual differentiation that clearly communicates their provisional status. The system may implement different presentation approaches for different recipient types, potentially providing laboratory personnel with comprehensive data including below-threshold findings while presenting clinicians with a more focused view emphasizing above-threshold results with clear indication of pending verifications. The CCL threshold determination may incorporate factors such as algorithm performance characteristics, clinical significance of potential misclassifications, resource constraints affecting verification timelines, and/or institutional risk tolerance profiles. Additionally, the system may adapt thresholds based on operational factors such as staffing availability, urgency of particular case types, or historical verification modification patterns observed for specific parameters or conditions.

In some implementations, the method further comprises maintaining a verification status tracking system that records a current verification state of the preliminary report and updates one or more stakeholders about progress toward the final verified report. The verification status tracking system may implement comprehensive monitoring capabilities that provide transparency and accountability throughout the verification workflow. The recorded verification states may include multiple status categories such as “pending initial review,” “partially verified,” “verification in progress,” “pending specialized review,” “verification complete/awaiting approval,” “approved for final reporting,” and/or other workflow-specific designations that communicate the precise stage of progression. The tracking system may maintain detailed metadata including timestamps for status transitions, identification of personnel involved at each stage, documentation of verification priorities assigned, records of any escalations or special handling requirements, and/or notations of verification challenges encountered. Stakeholder updates may be delivered through various mechanisms including dashboard visualizations, automated notifications, status change alerts, progress summaries, estimated completion projections, and/or integration with broader laboratory information or hospital management systems including lab information systems. The frequency and detail level of these updates may be customizable based on stakeholder roles, case priorities, institutional protocols, and/or individual preferences. The system may implement role-based visibility controls that provide appropriate levels of status detail to different stakeholders, potentially offering comprehensive workflow visibility to laboratory management while providing referring clinicians with more focused updates regarding expected availability of final results. Advanced implementations may include predictive elements that estimate verification completion timelines based on current workloads, case complexity factors, staffing availability, and/or historical performance metrics. The tracking system may also facilitate workflow optimization by identifying bottlenecks, monitoring turnaround time compliance, analyzing verification resource utilization patterns, and/or measuring performance against service level targets. This comprehensive tracking approach may enhance laboratory operations by providing clear visibility into verification workflows while managing stakeholder expectations through transparent communication of progress toward final verified reporting.

In some implementations, the method further comprises selectively applying the human verification to a subset of the preliminary results based on confidence levels and clinical significance, such that the higher the confidence is and the lower the finding significance is, the higher the chance of bypassing the human verification. The selective verification routing approach may implement prioritization algorithms that optimize human expert resource allocation based on both technical and clinical factors. The confidence level assessment may incorporate multiple dimensions including algorithm performance metrics, statistical reliability indicators, consistency with expected patterns, alignment with patient history, sample quality parameters, and/or other factors affecting result reliability. Clinical significance evaluation may consider diagnostic implications, treatment decision impacts, monitoring value, prognostic information, and/or other aspects of clinical utility. The selective application may utilize various methodologies including threshold-based filtering, multi-factor scoring systems, decision trees, risk stratification models, and/or machine learning approaches that balance verification resource constraints against quality assurance requirements. The routing process may implement a spectrum of verification intensities rather than binary verification decisions, potentially including options such as comprehensive review, targeted verification of specific findings, statistical sampling verification, automated rule-based validation with human confirmation for exceptions, and/or other approaches that match verification depth to specific case requirements. The relationship between confidence, significance, and verification probability may be implemented through mathematical functions, lookup tables, or algorithmic processes that quantify the inverse relationship between confidence and required verification intensity, as well as the direct relationship between clinical significance and verification priority. The system may maintain appropriate documentation of the specific verification pathway applied to each case, including recording which elements received human verification and which were processed through alternative validation mechanisms. This approach may support efficient resource utilization while maintaining appropriate quality assurance by focusing human expertise on results where verification adds the most value. The implementation may include override mechanisms that allow manual designation of verification requirements regardless of calculated priorities, ensuring flexibility for special cases, research protocols, quality assurance initiatives, or specific clinician requests.

In some implementations, the method further comprises providing incremental report updates as verification progresses through different aspects of the analysis, rather than waiting for complete verification to issue the final verified report. The incremental update approach may implement a phased reporting mechanism that balances comprehensive verification with timely information delivery. The progression of verification through different aspects of the analysis may follow structured pathways including sequential cell type verification, priority-based parameter verification, verification of quantitative metrics followed by morphological assessments, or other organized approaches that enable logical segmentation of the verification process. The incremental report updates may be delivered through various mechanisms including partial reports with clear completion status indicators, supplemental findings notifications, progressive result releases, staged reporting with version control, and/or dashboard updates that communicate verification progress. These updates may include explicit documentation of which report components have been verified and which remain pending, potentially using visual indicators, sectional completion markers, color coding, or other methods that clearly communicate verification status at the component level. The system may implement rules governing which verified components can be released incrementally based on their interdependencies, clinical significance, or potential impact on interpretation of other pending results. The delivery of incremental updates may be tailored to recipient needs, potentially providing detailed progressive results to treating specialists while offering more consolidated updates to primary care providers. This phased approach may help address scenarios where some critical parameters require timely communication while others necessitate more extensive verification processes, potentially reducing overall time-to-information for high-priority findings while maintaining thorough verification standards. The system may maintain comprehensive audit trails of incremental updates to document the evolution of findings throughout the verification process, facilitating transparency in how verified results developed and potentially supporting quality improvement analysis of verification patterns.

In some implementations, the method further comprises prioritizing human verification of findings based on their clinical significance, wherein findings with higher potential impact on patient care are verified before findings with lower clinical significance. The clinical significance prioritization approach may implement a stratified verification workflow that optimizes the sequence of human expert involvement. The assessment of clinical significance may incorporate multiple factors including diagnostic implications, therapeutic decision points, prognostic indicators, monitoring parameters, screening outcomes, and/or other aspects of medical relevance. The prioritization process may utilize various methodologies including categorical classification systems (such as critical/urgent/routine designations), numerical scoring frameworks that quantify potential impact, condition-specific protocols that reflect established clinical priorities, risk stratification models, and/or customized prioritization algorithms that incorporate institutional preferences or population-specific considerations. The verification sequence optimization may implement queue management systems, case assignment protocols, workload distribution algorithms, deadline management tools, and/or other workflow mechanisms that translate prioritization into operational execution. The system may incorporate temporal factors that adjust priorities based on contextual elements such as time since sample collection, proximity to treatment decision points, coordination with scheduled clinical encounters, and/or alignment with other diagnostic processes. Advanced implementations may include dynamic reprioritization capabilities that respond to emerging information such as new clinical data, related test results, or updated patient status indicators that may alter the relative significance of specific findings. The prioritization approach may maintain appropriate documentation of both the assigned priorities and their clinical rationales to support quality assurance review and continuous improvement of the prioritization methodology. This temporally optimized verification sequence may enhance clinical utility by delivering the most impactful verified findings when they have the greatest potential to positively influence patient care decisions, potentially improving outcome opportunities while maintaining comprehensive verification standards for all results regardless of prioritization sequence.

In some implementations, the method further comprises determining an appropriate verification tier for different preliminary reports, wherein routine findings are presented to standard reviewers while relatively complex or unusual findings are presented to specialist reviewers. The verification tier selection approach may implement skill-matching algorithms that align verification requirements with appropriate expertise levels. The determination of verification tiers may consider multiple factors including finding complexity, pattern rarity, diagnostic ambiguity, technical difficulty, clinical significance, specialized knowledge requirements, and/or other dimensions affecting verification challenges. The categorization of findings into different complexity levels may utilize rule-based classification systems, pattern recognition algorithms, statistical rarity assessments, confidence level indicators, and/or other methodologies that quantify verification difficulty. The reviewer categorization system may implement structured expertise classifications such as generalist/specialist designations, subspecialty qualifications, experience-based tiers, demonstrated proficiency levels, certification categories, and/or other frameworks that formalize reviewer capabilities. The matching process may employ various mechanisms including workflow routing algorithms, case assignment systems, tiered review queues, escalation pathways, collaborative verification protocols, and/or other approaches that direct findings to appropriate expertise levels. The system may incorporate workload balancing considerations that distribute verification tasks across available reviewers while maintaining appropriate expertise matching, potentially implementing algorithms that optimize both queue management efficiency and verification quality. The system may maintain documentation of the verification tier applied to each finding, creating transparency regarding the level of expertise involved in verification while potentially supporting quality assessment of tier selection appropriateness. This expertise-aligned approach may enhance verification quality by ensuring that relatively complex or unusual findings receive assessment from reviewers with appropriate specialized knowledge, while optimizing resource utilization by reserving specialist involvement for cases where their expertise adds substantial value.

In some implementations, the method further comprises including in the preliminary report statistical confidence metrics for findings based on a quantity of supporting cells identified; and using these metrics to inform decisions about a necessity of human verification for specific findings. The statistical confidence indicators approach may implement quantitative reliability measures that communicate the statistical foundation for preliminary findings while informing verification requirement decisions. The statistical metrics may include various measures such as confidence intervals, standard errors, sample size indicators, statistical power calculations, comparison with minimum reliability thresholds, coefficients of variation, and/or other quantitative measures that characterize result reliability. The relationship between supporting cell quantity and statistical confidence may be established through statistical modeling, analytical validation studies, literature-based standards, professional guideline requirements, and/or institutional policy determinations that define quantity-dependent reliability thresholds for different parameters. The presentation of these metrics in preliminary reports may utilize various formats including numerical expressions, visual confidence indicators, categorical reliability classifications, contextual comparison with established minimums, and/or other approaches that effectively communicate statistical foundation strength. The influence of these metrics on verification necessity decisions may be implemented through rule-based systems, algorithmic assessment frameworks, threshold comparison protocols, risk-based verification models, and/or other methodologies that translate statistical confidence into appropriate verification requirements. The system may implement different confidence thresholds for different finding types based on their clinical significance, technical difficulty, or historical verification modification rates. These statistical indicators may help optimize verification resource allocation by directing human expertise toward findings where statistical limitations create genuine uncertainty while reducing unnecessary verification for findings with robust statistical foundations. The specific statistical metrics and thresholds may be customizable based on institutional preferences, patient population characteristics, clinical application contexts, or regulatory requirements applicable to particular testing scenarios. Advanced implementations may incorporate multiple statistical dimensions simultaneously, potentially considering not only cell quantity but also distribution characteristics, morphological consistency, staining quality metrics, and/or other factors affecting statistical reliability.

In some implementations, the method further comprises performing the human verification by focusing review on specific cell subsets identified in the preliminary results rather than comprehensive review of the entire image. The differential verification approach may implement targeted review protocols that optimize human expert engagement by focusing attention on the most verification-critical elements. The identification of specific cell subsets for focused review may utilize various selection methodologies including confidence-based filtering, clinical significance prioritization, anomaly detection algorithms, statistical sampling approaches, representative subset selection, and/or other techniques that isolate verification-critical cellular elements. The presentation of these cell subsets to human reviewers may employ various interface approaches including region-of-interest highlighting, automated navigation to selected cells, side-by-side comparative views, gallery-style presentations of filtered cells, contextual visualization with surrounding cells, and/or other display methods that facilitate efficient focused verification. The system may implement different subset selection methods based on verification objectives, potentially employing comprehensive review of rare or clinically significant cell types while using statistical sampling for abundant cell populations with lower individual clinical significance. The focused review process may include mechanisms for reviewers to expand verification scope when initial subset examination raises concerns that warrant broader assessment, ensuring appropriate flexibility while maintaining efficiency benefits. The system may maintain documentation of which specific cellular elements received human verification versus which were assessed through other validation approaches, creating transparency regarding verification coverage. This targeted approach may enhance verification efficiency by reducing time spent on cellular elements with limited verification value while maintaining or potentially improving verification quality by enabling more thorough assessment of the most critical elements. The specific implementation of subset selection algorithms may be customizable based on verification objectives, reviewer preferences, institutional protocols, or particular clinical scenarios, allowing appropriate adaptation to different verification contexts while maintaining the core efficiency benefits of differential verification.

In some implementations, the method further comprises implementing an escalated verification and reporting pathway when human verification identifies critical results not detected in the preliminary results, wherein the escalated verification and reporting pathway comprises at least one of: prompt physician notification, documentation of a time delay between the preliminary report and the final verified report, or analysis of why the preliminary analysis failed to identify the critical finding. The critical result escalation pathway may implement specialized handling protocols that address significant discrepancies between preliminary and human-verified findings. The identification of critical results may utilize various defining criteria including established critical value thresholds, life-threatening condition indicators, urgent intervention triggers, significant diagnostic reclassifications, unexpected malignancy findings, and/or other parameters representing findings with substantial and time-sensitive clinical implications. The escalated verification component may include expedited secondary review, supervisory confirmation, consultation with subject matter experts, rapid consensus determination, or other approaches that ensure appropriate validation of critical findings before communication. The prompt physician notification processes may employ multiple communication mechanisms including direct telephone contact, secure messaging, automated alerting systems, electronic health record notifications, and/or other channels appropriate to urgency level and institutional protocols. The documentation of time delays may include comprehensive timestamp recording capturing preliminary report issuance, critical finding identification, verification completion, physician notification, and/or other relevant chronological milestones that establish the temporal progression from preliminary to final reporting. The analytical component focusing on preliminary detection failure may utilize systematic root cause analysis, classification algorithm assessment, pattern recognition evaluation, specimen quality review, and/or other investigative approaches that identify specific factors contributing to the preliminary oversight. This analytical information may feed into continuous improvement processes including algorithm refinement, threshold adjustment, training dataset enhancement, or other modifications addressing identified limitations. The escalation pathway may incorporate documentation mechanisms that create detailed records of the escalation process, potentially including verification steps taken, personnel involved, communication timestamps, recipient acknowledgments, and/or other elements establishing comprehensive accountability. This systematic approach to critical finding discrepancies may enhance patient safety by ensuring appropriate communication of verification-identified critical results while simultaneously supporting quality improvement through structured analysis of preliminary detection limitations.

In some implementations, the method further comprises applying special handling procedures when human verification causes a diagnostic classification to shift across clinically significant thresholds or categories, wherein the special handling procedures comprise at least one of: documentation of a reclassification rationale, secondary expert verification of the change, or prioritized communication to treating physicians. The diagnostic category shift handling procedures may implement enhanced protocols when human verification substantially alters the clinical interpretation framework established in preliminary reporting. The identification of clinically significant threshold or category crossings may utilize various defining criteria including established diagnostic boundaries, treatment decision thresholds, risk stratification category shifts, prognostic grouping changes, monitoring intensity determinants, and/or other classification transitions with meaningful clinical management implications. The documentation of reclassification rationale may include detailed explanations addressing morphological considerations, quantitative reassessments, pattern recognition elements, reference standard comparisons, literature-based justifications, guideline alignment considerations, and/or other factors supporting the verification-based reclassification. The secondary expert verification component may employ various approaches including independent review by a different qualified expert, collaborative assessment involving multiple specialists, consensus determination processes, departmental consultation procedures, and/or other verification intensification mechanisms appropriate to the significance of the diagnostic shift. The prioritized communication processes may utilize multiple approaches including expedited reporting pathways, direct clinician contacts, specially formatted notifications highlighting the classification change, comparative presentations of preliminary versus verified categorizations, and/or other communication enhancements that ensure appropriate awareness of the diagnostic framework modification. The system may implement different handling intensities proportional to the clinical significance of the specific category shift, potentially applying more comprehensive protocols to shifts with greater treatment implication differences. This specialized approach to category-shifting verifications may enhance clinical decision quality by ensuring appropriate attention to verification-based diagnostic framework modifications while maintaining efficient handling of verifications that confirm preliminary categorizations. The specific implementation of shift significance thresholds may reflect institutional protocols, specialty-specific standards, patient population characteristics, or particular clinical program requirements, allowing appropriate customization while maintaining the core enhanced handling for clinically meaningful category transitions.

In some implementations of the method, providing the preliminary report comprises making it available for presentation to a physician. The preliminary report may be made available through various delivery channels including electronic health record integration, laboratory information system access, secure messaging platforms, clinical portal availability, mobile application delivery, printed report distribution, and/or other mechanisms aligned with institutional workflows and physician preferences. The presentation format may incorporate distinct visual elements, specialized terminology, explicit status indicators, estimated verification timeline information, confidence level annotations, and/or other design features that clearly communicate the preliminary nature of the report while presenting available information in a clinically useful manner. The system may implement contextual notifications that accompany preliminary report availability, potentially including explanations of which components await verification, indications of expected verification completion timeframes, guidance regarding appropriate clinical use of preliminary information, and/or other elements that support informed utilization. Access controls may include acknowledgment requirements, disclaimer acceptance steps, specialized training prerequisites, role-based restrictions, and/or other mechanisms that ensure appropriate understanding of preliminary report limitations. The system may maintain comprehensive records of preliminary report access including user identification, timestamp information, access context details, and/or other tracking elements that document information availability throughout the progressive reporting process. Different implementation approaches may include making preliminary reports available to all treating physicians, limiting availability to ordering providers, implementing specialty-based access controls, or other access management strategies aligned with institutional policies and regulatory requirements. This physician availability approach may support informed clinical decision-making by providing timely access to preliminary information with appropriate contextual guidance while verification processes continue, potentially enhancing care efficiency while maintaining appropriate utilization boundaries through clear communication of verification status. In some implementations of the method, a non-transitory computer-readable medium comprising instructions that, when executed by a processor, cause the processor to perform the computer-implemented method.

In various implementations, a system for progressive cytological reporting with human verification, comprising: a computer configured to: receive an image of a cytological smear associated with a patient; receive preliminary results from an automatic analysis of the image; create a preliminary report comprising indications of hematological cell or cell type quantification metrics (HCQMs) for cell types, based on the preliminary results; provide the preliminary report with an indication that human verification is pending; receive human verification and/or correction results for the cytological smear analysis; and issue a final verified report based on the human verification and/or correction results. FIG. 21B illustrates an embodiment of a block diagram of a system for progressive cytological reporting with human verification. The system includes a computer 316 that receives images of cytological smears associated with patients from an automatic analysis system 317, and is further coupled to a human verification interface 318.

In some implementations of the system, the computer is further configured to: detect when human verification results alter the clinical significance of the preliminary results; generate an alert for such alterations; and expedite the issuing of the final verified report for cases where the clinical significance has changed. In some implementations of the system, the computer is further configured to: implement a retraction protocol when human verification contradicts the preliminary results with potential clinical impact above a predetermined threshold; wherein the retraction protocol comprises notifying recipients of the preliminary report about the contradiction, and issuing a final verified report with indication of changes related to the clinical impact.

In some implementations of the system, the computer is further configured to: identify when human verification changes a finding from one with routine findings to one requiring immediate intervention or vice versa, and modify report delivery and clinical alert mechanisms accordingly to facilitate appropriate clinical response. In some implementations of the system, the preliminary report includes comparisons with previous cytological results from the patient's medical history, and wherein the computer is further configured to validate, during the human verification, significant changes from the patient's baseline. In some implementations of the system, the computer is further configured to: specially mark instances where human verification reverses previous trend assessments in temporal comparisons, and provide detailed explanations for such reversals to facilitate proper clinical interpretation of the patient's disease progression.

In some implementations of the system, the computer is further configured to: automatically recommend appropriate ancillary testing when human verification identifies conditions requiring confirmatory or additional testing that were not indicated by the preliminary results, and include these recommendations in the final verified report. In some implementations of the system, the computer, when creating the preliminary report, is configured to include in the preliminary report findings that fall on the sufficient confidence side of a predetermined classification confidence level (CCL) threshold, while designating other findings as awaiting verification.

In some implementations of the system, the computer is further configured to maintain a verification status tracking system that records a current verification state of the preliminary report and updates one or more stakeholders about progress toward the final verified report. In some implementations of the system, the computer is further configured to selectively apply the human verification to a subset of the preliminary results based on confidence levels and clinical significance, such that the higher the confidence is and the lower the finding significance is, the higher the chance of bypassing the human verification.

In some implementations of the system, the computer is further configured to provide incremental report updates as verification progresses through different aspects of the analysis, rather than waiting for complete verification to issue the final verified report. In some implementations of the system, the computer is further configured to prioritize human verification of findings based on their clinical significance, wherein findings with higher potential impact on patient care are verified before findings with lower clinical significance. In some implementations of the system, the computer is further configured to determine an appropriate verification tier for different preliminary reports, wherein routine findings are presented to standard reviewers while relatively complex or unusual findings are presented to specialist reviewers.

In some implementations of the system, the computer is further configured to: include in the preliminary report statistical confidence metrics for findings based on a quantity of supporting cells identified; and use these metrics to inform decisions about a necessity of human verification for specific findings. In some implementations of the system, the computer is further configured to perform the human verification by focusing review on specific cell subsets identified in the preliminary results rather than comprehensive review of the entire image. In some implementations of the system, the computer is further configured to implement an escalated verification and reporting pathway when human verification identifies critical results not detected in the preliminary results, wherein the escalated verification and reporting pathway comprises at least one of: prompt physician notification, documentation of a time delay between the preliminary report and the final verified report, or analysis of why the preliminary analysis failed to identify the critical finding.

In some implementations of the system, the computer is further configured to apply special handling procedures when human verification causes a diagnostic classification to shift across clinically significant thresholds or categories, wherein the special handling procedures comprise at least one of: documentation of a reclassification rationale, secondary expert verification of the change, or prioritized communication to treating physicians. In some implementations of the system, the computer is further configured to make the preliminary report available for presentation to a physician.

The following implementation addresses the challenge of handling multiple cytological smears from the same patient that may exhibit varying degrees of consistency. By calculating discrepancy levels between smears and comparing them against predetermined thresholds, the system may intelligently determine whether to base reports on a single smear or combined results. When significant discrepancies exist, selecting the most reliable smear may prevent potentially misleading averaged results. Conversely, when smears show consistency, combining results may provide enhanced statistical confidence through increased sample size. This approach may improve reporting reliability while optimizing the use of available sample data, potentially enhancing diagnostic value for physicians. The embodiment supports both scenarios where multiple smears yield consistent information and where discrepancies suggest that one smear may be more representative than others, which may help laboratories provide more reliable and clinically relevant information to healthcare providers without requiring manual intervention for each case.

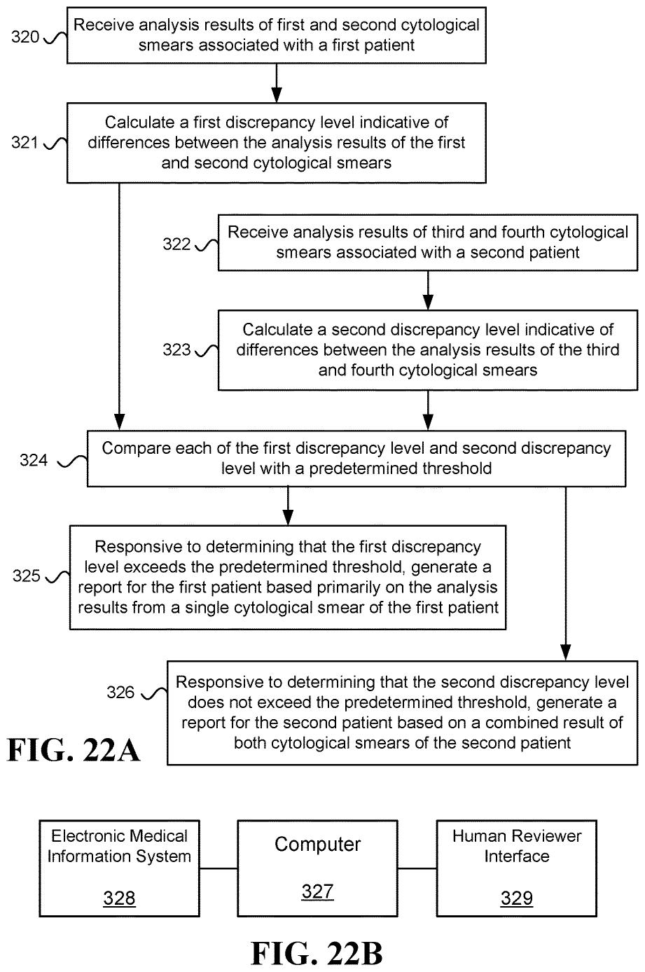

In various implementations, a computer-implemented method for combining results from cytological smears, comprising: receiving analysis results of first and second cytological smears associated with a first patient; receiving analysis results of third and fourth cytological smears associated with a second patient; calculating a first discrepancy level indicative of differences between the analysis results of the first and second cytological smears; calculating a second discrepancy level indicative of differences between the analysis results of the third and fourth cytological smears; comparing each of the first discrepancy level and second discrepancy level with a predetermined threshold; responsive to determining that the first discrepancy level exceeds the predetermined threshold, generating a report for the first patient based primarily on the analysis results from a single cytological smear of the first patient; responsive to determining that the second discrepancy level does not exceed the predetermined threshold, generating a report for the second patient based on a combined result of both cytological smears of the second patient. FIG. 22A illustrates an embodiment of a computer-implemented method for combining results from cytological smears. The method includes the following steps. In step 320, receive analysis results of first and second cytological smears associated with a first patient. In step 321, calculate a first discrepancy level indicative of differences between the analysis results of the first and second cytological smears. In step 322, receive analysis results of third and fourth cytological smears associated with a second patient. In step 323, calculate a second discrepancy level indicative of differences between the analysis results of the third and fourth cytological smears. In step 324, compare each of the first discrepancy level and second discrepancy level with a predetermined threshold. In step 325, responsive to determining that the first discrepancy level exceeds the predetermined threshold, generate a report for the first patient based primarily on the analysis results from a single cytological smear of the first patient. And in step 326, responsive to determining that the second discrepancy level does not exceed the predetermined threshold, generate a report for the second patient based on a combined result of both cytological smears of the second patient.

The method may employ various approaches for comparing cytological smears from the same patient and making intelligent reporting decisions based on the degree of consistency between smears. The discrepancy levels may be calculated through multiple potential mechanisms, including but not limited to: statistical measures of difference, variance calculations, weighted comparison algorithms, or feature-based differential analysis. These discrepancy calculations may consider aspects such as cell population distributions, morphological characteristics, or quantitative metrics derived from the cytological analysis. The predetermined threshold may serve as a decision boundary that differentiates between cases where smears exhibit sufficient consistency to warrant combined reporting versus cases where discrepancies suggest potential preparation variations, sampling differences, or other factors that may make one smear more representative than the other. When the discrepancy level exceeds the threshold, generating a report based primarily on a single cytological smear may prevent potentially misleading averaged results that could obscure clinically significant findings. Conversely, when discrepancies fall below the threshold, combining results from multiple smears may leverage the additional statistical power of a larger effective sample size. This adaptive approach to report generation may accommodate the biological and technical realities of cytological analysis while optimizing the clinical value of the reported information.

In the context of generating a report based primarily on the analysis results from a single cytological smear, the term “primarily” indicates that while the report is based predominantly on one cytological smear selected from the first and second cytological smears, the report may still incorporate limited information from the other cytological smear. For example, when generating a report based primarily on the first cytological smear, at least 80%, at least 90%, or at least 95% of the quantitative values, diagnostic conclusions, or cell population metrics in the report may be derived from the first cytological smear, while the remaining portion may incorporate data from the second cytological smear. The non-primary cytological smear may contribute to the report through supplementary information such as confirmation of rare cell findings, validation of extreme values, or notation of discrepancies that may have clinical significance. This approach of basing the report primarily on one cytological smear when discrepancies exceed the threshold allows the system to avoid potentially misleading averaged results while still preserving relevant information from both cytological smears when clinically appropriate.

In some implementations of the method, the analysis results comprise hematological cell or cell type quantification metrics (HCQMs), and wherein generating the report based primarily on the analysis results from a single cytological smear comprises deriving at least 80% of the HCQMs in the report from the single cytological smear while deriving no more than 20% of the HCQMs from the other cytological smear of the first patient. The analysis results may include various forms of HCQMs that represent the presence, abundance, or characteristics of different cell populations identified in the cytological smears. These metrics may include absolute counts representing the total number of cells of specific types identified; normalized counts representing cells per unit area or volume; relative counts or percentage representations indicating proportions of specific cell types within broader cell populations; statistical distributions of morphological features within cell populations; and/or derived metrics that incorporate multiple measurement factors. The specific HCQMs may vary based on the cell types being analyzed and the diagnostic context, potentially including metrics for standard hematological parameters such as different white blood cell populations, red blood cell characteristics, or platelet assessments, as well as metrics for abnormal or rare cell populations that may have specific diagnostic significance. These quantitative representations of cell populations and their characteristics may provide the foundational data upon which discrepancy assessments and subsequent reporting decisions are based. When generating a report based primarily on a single cytological smear, the majority of these HCQMs are derived from that single cytological smear, with at least 80% of the metrics originating from the selected cytological smear, thereby ensuring that the report predominantly reflects the characteristics of the more representative or reliable cytological smear while still allowing incorporation of supplementary information from the other cytological smear when appropriate.

In some implementations of the method, calculating the first and second discrepancy levels comprises calculating differences in respective HCQMs from the different cytological smears of a patient. The calculation of discrepancy levels may involve various mathematical approaches to quantify differences between corresponding HCQMs from multiple cytological smears. These approaches may include calculating absolute numerical differences between corresponding metrics; determining percentage or proportional differences relative to the magnitude of the measurements; applying statistical variance calculations such as standard deviations or coefficients of variation; implementing weighted difference algorithms that account for the clinical significance of specific cell types; and/or utilizing multivariate comparison methods that integrate differences across multiple cell populations. The specific calculation method may be selected based on factors such as the type of HCQM being compared, the clinical context, or established laboratory protocols. For certain cell types, absolute differences may be most appropriate, while for others, proportional differences may better reflect clinical significance. These discrepancy calculations may be performed at various levels of granularity, potentially comparing overall cell distributions, specific cell type populations, or even subcategories within major cell types to generate a comprehensive assessment of inter-smear consistency.

In some implementations, the method further comprises selecting the single cytological smear from the first and second cytological smears, wherein the selection is based on a confidence level for the first and second cytological smears, and further comprising selecting the cytological smear with the higher confidence level. The selection of a single cytological smear when discrepancy levels exceed the predetermined threshold may be implemented through various confidence assessment mechanisms. These mechanisms may evaluate factors indicative of sample quality, analytical reliability, or clinical relevance to identify which smear may provide the most representative results. The confidence level may be derived through multiple approaches including algorithmic assessment of technical quality parameters; statistical measures of classification reliability; historical performance metrics for similar sample types; and/or integration of human assessment inputs when available. This selection process may prioritize the cytological smear that demonstrates higher confidence indicators, recognizing that when significant discrepancies exist between smears, the one exhibiting stronger quality and reliability characteristics may more accurately represent the patient's actual cellular profile. The selection may occur through various implementation methods, including binary comparison of overall confidence scores, weighted multi-factor evaluation of quality indicators, or rule-based selection frameworks that incorporate contextual considerations. This confidence-based selection approach may help ensure that when reports must be based primarily on a single smear due to inter-smear discrepancies, the selected smear represents the most reliable data source available.

In some implementations of the method, the confidence level is based on at least one of: classification confidence levels (CCLs), human review results, whether or not a cytological smear has been reviewed by a human, or a probability level associated with the hematological cell or cell type quantification metrics (HCQMs). The confidence level used for cytological smear selection may incorporate multiple factors that collectively indicate reliability and analytical quality. Classification confidence levels (CCLs) may reflect the algorithmic certainty in cell identification and categorization, potentially derived from classifier output scores, statistical confidence intervals, or probability distributions associated with cell type assignments. Human review results may provide expert assessment of sample quality, cell identification accuracy, or preparation adequacy, potentially captured through review notes, corrections made to classifications, verification flags, or quality scores assigned during manual examination. The review status itself—whether a smear has undergone human review—may serve as a confidence indicator, as verified samples may carry additional reliability weight in selection decisions. Probability levels associated with HCQMs may reflect statistical confidence based on sample size, distribution characteristics, or comparative analysis against reference ranges. These diverse confidence indicators may be integrated through various mechanisms including weighted scoring algorithms, hierarchical evaluation frameworks, or composite indices that synthesize multiple quality factors. By incorporating this range of confidence measures, the selection process may identify the cytological smear most likely to provide accurate and clinically meaningful results when reporting must be based primarily on a single sample.

In some implementations, the method further comprises calculating the combined result of the third and fourth cytological smears by taking a sum, an average, or a weighted average of the analysis results of the third and fourth cytological smears. The calculation of combined results from multiple cytological smears may be implemented through various mathematical approaches depending on the specific characteristics of the data being combined. A sum approach may be appropriate for absolute cell counts, potentially providing aggregate totals across the combined samples. Average calculations may normalize results across multiple smears, potentially compensating for volume or distribution variations between samples. Weighted averaging may incorporate quality or confidence factors to give greater influence to more reliable measurements, potentially optimizing the statistical validity of the combined result. The specific calculation method may be selected based on factors such as the type of analysis results being combined, the relative quality characteristics of the different smears, laboratory protocols, or clinical objectives. The combined result may be calculated at various levels of granularity, potentially combining overall population metrics, specific cell type measurements, or even individual morphological parameters to create a comprehensive integrated assessment that leverages the increased statistical power of multiple samples while maintaining appropriate analytical validity.

In some implementations, the method further comprises selecting between the sum, the average, and the weighted average based on at least one of: the discrepancy level, the hematological cell or cell type quantification metrics (HCQMs), or a confidence level, related to the analysis results of the third and fourth cytological smears. The selection between different calculation methods for combining results may be implemented through various decision frameworks that optimize the combination approach based on specific case characteristics. These selection mechanisms may evaluate factors such as the discrepancy level between smears, which may influence whether simple averaging is appropriate or whether weighted approaches might better account for partial inconsistencies. The specific HCQMs being combined may also influence the selection, as different cell populations or metrics may benefit from different combination approaches based on their statistical properties, clinical significance, or typical variance patterns. Confidence levels associated with the different cytological smears may factor into the selection, potentially favoring weighted approaches when smears exhibit meaningful differences in quality indicators or reliability metrics. The selection mechanism may utilize rules-based frameworks, algorithmic decision trees, statistical optimization methods, or adaptive approaches that learn from historical performance data. This tailored selection of calculation methods may enhance the clinical value of combined results by applying the most appropriate mathematical approach to each specific analytical context, rather than implementing a one-size-fits-all combination strategy. In some cases, different cell types may be combined in different ways; for example, rare cells may be combined by summing their counts, while more abundant cells may be combined by averaging or using a weighted average of their percentages.

In some implementations, the method further comprises generating the report for the first patient to prominently display the results based on the single cytological smear; and including in the report, if there is reference to a non-selected cytological smear of the first patient, one or more of the following: a mention of another cytological smear without the results, results based on the non-selected cytological smear shown with an indication that they are not the results used as the official report results, a reason for the selection, combined results from the first and second cytological smears with an indication they are not the results used for the official report result. The report generation for cases with significant inter-smear discrepancies may implement various presentation approaches that maintain clarity while providing appropriate context. Prominently displaying the results based on the selected single cytological smear may help ensure that primary clinical decision-making is based on the most reliable data source, potentially through visual emphasis, ordering priority, or explicit labeling as the official results. References to non-selected smears may be included to maintain transparency about available data while preventing confusion about which results should inform clinical decisions. Such references may take multiple forms, including simple acknowledgment of additional testing without detailed results; presentation of alternate results with clear indicators distinguishing them from the official results; explanatory notes regarding the selection rationale to provide context for the reporting decision; or inclusion of hypothetical combined results with appropriate caveats about their limited applicability given the identified discrepancies. These presentation approaches may balance the need for clear, actionable information with appropriate transparency about available data and analytical decision-making, potentially enhancing the clinical utility of the report while maintaining scientific integrity in cases where multiple smears yield inconsistent results.

In some implementations, the method further comprises generating the report for the second patient to prominently display the combined result; and including in the report, if there is reference to analysis results beyond the combined result, one or more of the following: analysis results from one or both of the third and fourth cytological smears shown with an indication that they are not the results used for the official report result, results of the discrepancy level, a reason for the decision to combine the results. The report generation for cases with consistent inter-smear results may implement various presentation approaches that emphasize the statistical advantages of combined data while maintaining appropriate context about the underlying samples. Prominently displaying the combined result may leverage the enhanced statistical confidence derived from multiple consistent samples, potentially through visual emphasis, ordering priority, or explicit labeling as the official results. References to individual smear results may provide transparency about the underlying data sources while maintaining clarity about the primary results intended for clinical use. Such references may take multiple forms, including detailed presentation of individual smear results with clear indicators distinguishing them from the combined official results; inclusion of discrepancy metrics to demonstrate the consistency between samples that justified combination; or explanatory notes regarding the combination rationale to provide context for the reporting decision. These presentation approaches may communicate the statistical advantages of the combined result while providing sufficient detail for clinicians who may wish to review the component data, potentially enhancing both the clinical utility and scientific transparency of the report in cases where multiple consistent smears allow for statistically robust combined reporting.

In some implementations, the method further comprises setting different predetermined thresholds for different cell types based on at least one of: clinical significance of the cell type, rarity of the cell type, or expected statistical variance of the cell type. The system may implement cell type-specific threshold configurations that recognize the varying clinical implications and statistical characteristics of different cell populations. These tailored thresholds may account for intrinsic differences between cell types, such as higher clinical significance of rare abnormal cells compared to common cell types, or the naturally greater statistical variance observed in certain cell populations. For example, the threshold for lymphocyte count discrepancies may be set higher than for blast cells, as blast cells may have greater diagnostic significance even with smaller numerical differences. The specific threshold values may be determined through various approaches, including: statistical analysis of historical laboratory data showing typical variance patterns; clinical guideline recommendations regarding significant changes for specific cell types; and/or correlation studies between discrepancy magnitudes and clinical outcomes. The thresholds may be implemented as absolute numerical differences, percentage variations, standard deviation multiples, and/or clinical significance weights. This cell type-specific approach may accommodate the biological and clinical realities of different cell populations while maintaining appropriate sensitivity to clinically meaningful discrepancies.

In some implementations of the method, the combined result comprises a cell type-specific selective combination where some hematological cell or cell type quantification metrics (HCQMs) from both the third and fourth cytological smears are combined while other HCQMs are selected from a single cytological smear based on cell type-specific discrepancy levels. The system may implement a granular, cell type-specific combination approach that recognizes discrepancies may affect some cell populations while others remain consistent across multiple smears. Rather than applying an all-or-nothing approach to smear selection or combination, this selective method may apply different handling methods to different cell types based on their specific discrepancy characteristics. For example, if neutrophil counts show minimal discrepancy between smears while lymphocyte counts show significant variance, the system may combine the neutrophil data from both smears while selecting lymphocyte data from the smear with higher confidence metrics. This selective combination may be implemented through various mechanisms, including: cell type-specific discrepancy thresholds that determine combination eligibility; weighted averaging algorithms that adjust influence factors based on discrepancy magnitudes; and/or confidence-based selection criteria that vary by cell type. The granularity of this approach may extend to subcategories within major cell types, potentially addressing variations in subtypes such as segmented versus band neutrophils, or normal versus atypical lymphocytes. By tailoring the combination strategy to the specific discrepancy patterns observed in each cell population, the system may optimize the statistical validity of the combined result while preventing outlier measurements from inappropriately influencing the final metrics.

In some implementations, the method further comprises, responsive to determining that at least one of the first discrepancy level or the second discrepancy level exceeds a presentation threshold: presenting to a human reviewer a review interface comprising a side-by-side comparison of corresponding cytological smears with visual indicators highlighting the differences in analysis results; and receiving human input regarding the discrepancies. The system may implement human review interfaces when significant discrepancies are detected between cytological smears from the same patient. These interfaces may be designed to highlight discrepancies in ways that facilitate efficient assessment and decision-making by human reviewers. The side-by-side comparison may present corresponding regions, cell types, or analytical results from both smears simultaneously, with visual indicators such as color-coding, contrast adjustments, outlining, or graphical overlays that draw attention to areas of discrepancy. Additionally, the interface may incorporate quantitative representations of discrepancies, such as differential charts, variance heat maps, or statistical significance indicators that help contextualize the magnitude and potential clinical relevance of the observed differences. The presentation threshold for triggering this interface may be the same as or different from the threshold used for report generation decisions, potentially with lower thresholds for presentation than for report decisions to provide human reviewers visibility into borderline cases. The system may also provide contextual information alongside the comparison, such as patient history, medication status, or quality indicators for each smear, which may help inform the reviewer's assessment of the discrepancies. The human input received through this interface may influence not only the immediate reporting decision but may also feed into quality improvement processes and/or refinement of discrepancy thresholds over time.

In some implementations, the method further comprises collecting and storing discrepancy data from multiple patients; analyzing patterns in the discrepancy data to identify systematic issues in cytological smear preparation or analysis; and generating quality improvement recommendations based on the identified patterns. The system may implement a quality improvement framework that leverages discrepancy data across multiple patients to identify and address systematic issues in laboratory processes. By collecting, storing, and analyzing discrepancy patterns over time, the system may detect recurring issues that may indicate opportunities for process improvement. These patterns may reveal various systematic challenges, such as: preparation inconsistencies associated with specific equipment, reagents, or techniques; analytical variations linked to particular classification algorithms or threshold settings; timing-related factors that affect sample quality or processing; and/or operator-specific patterns that may suggest training opportunities. The analysis may employ various methodologies including statistical trend analysis, pattern recognition algorithms, correlation studies between discrepancy characteristics and process variables, and/or comparative benchmarking against established quality standards. Based on identified patterns, the system may generate quality improvement recommendations that may include: protocol modifications to address preparation variability; algorithm refinements to improve analytical consistency; training programs targeting specific discrepancy types; and/or equipment calibration or maintenance recommendations. These recommendations may be presented through various mechanisms including automated alerts to laboratory management, periodic quality reports, graphical dashboards showing discrepancy trends, and/or suggested protocol revisions with supporting data.

In some implementations, the method further comprises retrieving medical history data of the first patient from an electronic medical information system; and adjusting at least one of the predetermined threshold or the selection of the single cytological smear based on identified medical conditions in the medical history data. The system may incorporate patient medical history data to contextualize and optimize discrepancy assessments and reporting decisions. By retrieving relevant medical information from electronic health records or laboratory information systems, the system may adapt its analytical parameters to account for the patient's specific medical context. The medical history data may influence threshold adjustments based on conditions known to affect cellular variability, such as certain hematological disorders, autoimmune conditions, or therapeutic interventions that may cause expected fluctuations in cell populations. For example, patients undergoing chemotherapy may experience greater variability in certain cell populations, potentially warranting higher discrepancy thresholds, while monitoring for residual disease in leukemia patients may benefit from lower thresholds to increase sensitivity to small but clinically significant population changes. Additionally, the medical history may inform the selection criteria for single cytological smear reporting when discrepancies exceed thresholds, potentially prioritizing smears with characteristics most relevant to the patient's specific condition or treatment monitoring needs. This integration may be implemented through various mechanisms, including condition-specific parameter adjustment rules, treatment-aware threshold modifications, or contextual weighting factors that influence selection algorithms. By incorporating the patient's medical context, the system may produce more clinically relevant reports that account for individual patient factors that may affect sample characteristics and analytical interpretation.

In some implementations, the method further comprises calculating the combined result of the third and fourth cytological smears utilizing a weighted average comprising applying weights determined by a multi-factor confidence assessment that includes at least two of: classification confidence levels (CCLs), smear preparation quality indicators, statistical power considerations, or confidence levels from human review. The system may implement weighting mechanisms that incorporate multiple confidence factors when combining results from different cytological smears. Rather than applying uniform or simplistic weighting, this multi-factor approach may synthesize various quality and confidence indicators to determine the relative influence each smear should have on the combined result. The weighting factors may include classification confidence levels (CCLs) that reflect the algorithm's certainty in cell identification; smear preparation quality indicators such as staining consistency, cellular distribution, or artifact presence; statistical power considerations related to the number of cells identified in each category; and/or confidence assessments provided during human review processes. These factors may be integrated through various mathematical frameworks, including weighted averaging algorithms that assign influence proportional to quality indicators; Bayesian approaches that model reliability based on multiple evidence sources; or adaptive weighting schemes that emphasize different factors depending on the specific cell types being analyzed. The weights may be applied at various granularity levels, potentially varying between cell types or even individual cells based on their specific confidence characteristics. This nuanced approach to result combination may leverage all available quality indicators to produce a combined result that improves statistical validity by giving greater influence to measurements with higher confidence indicators while minimizing the impact of potentially less reliable data points. In some implementations of the method, a non-transitory computer-readable medium comprising instructions that, when executed by a processor, cause the processor to perform the computer-implemented method.

In various implementations, a system for combining results from cytological smears, comprising: a computer configured to: receive analysis results of first and second cytological smears associated with a first patient; receive analysis results of third and fourth cytological smears associated with a second patient; calculate a first discrepancy level indicative of differences between the analysis results of the first and second cytological smears; calculate a second discrepancy level indicative of differences between the analysis results of the third and fourth cytological smears; compare each of the first discrepancy level and second discrepancy level with a predetermined threshold; responsive to a determination that the first discrepancy level exceeds the predetermined threshold, generate a report for the first patient based primarily on the analysis results from a single cytological smear of the first patient; and responsive to a determination that the second discrepancy level does not exceed the predetermined threshold, generate a report for the second patient based on a combined result of both cytological smears of the second patient. FIG. 22B illustrates an embodiment of a block diagram of a system for combining results from cytological smears. The system includes a computer 327 that can optionally retrieve medical history data from an electronic medical information system 328, and is further coupled to a human reviewer interface 287.

In some implementations of the system, the analysis results comprise hematological cell or cell type quantification metrics (HCQMs), and wherein the generate of the report based primarily on the analysis results from a single cytological smear comprises deriving at least 80% of the HCQMs in the report from the single cytological smear while deriving no more than 20% of the HCQMs from the other cytological smear of the first patient. In some implementations of the system, the computer is configured to calculate the first and second discrepancy levels by calculating differences in respective HCQMs from the different cytological smears of a patient.

In some implementations of the system, the computer is further configured to select the single cytological smear from the first and second cytological smears, wherein the selection is based on a confidence level for the first and second cytological smears, and wherein the computer is configured to select the cytological smear with the higher confidence level. In some implementations of the system, the confidence level is based on at least one of: classification confidence levels (CCLs), human review results, whether or not a cytological smear has been reviewed by a human, or a probability level associated with the hematological cell or cell type quantification metrics (HCQMs).

In some implementations of the system, the computer is further configured to calculate the combined result of the third and fourth cytological smears by taking a sum, an average, or a weighted average of the analysis results of the third and fourth cytological smears. In some implementations of the system, the computer is further configured to select between the sum, the average, and the weighted average based on at least one of: the discrepancy level, the hematological cell or cell type quantification metrics (HCQMs), or a confidence level, related to the analysis results of the third and fourth cytological smears.

In some implementations of the system, the computer is further configured to: generate the report for the first patient to prominently display the results based on the single cytological smear; and include in the report, if there is reference to a non-selected cytological smear of the first patient, one or more of the following: a mention of another cytological smear without the results, results based on the non-selected cytological smear shown with an indication that they are not the results used as the official report results, a reason for the selection, combined results from the first and second cytological smears with an indication they are not the results used for the official report result.

In some implementations of the system, the computer is further configured to: generate the report for the second patient to prominently display the combined result; and include in the report, if there is reference to analysis results beyond the combined result, one or more of the following: analysis results from one or both of the third and fourth cytological smears shown with an indication that they are not the results used for the official report result, results of the discrepancy level, a reason for the decision to combine the results. In some implementations of the system, the computer is further configured to set different predetermined thresholds for different cell types based on at least one of: clinical significance of the cell type, rarity of the cell type, or expected statistical variance of the cell type.

In some implementations of the system, the combined result comprises a cell type-specific selective combination where some hematological cell or cell type quantification metrics (HCQMs) from both the third and fourth cytological smears are combined while other HCQMs are selected from a single cytological smear based on cell type-specific discrepancy levels. In some implementations of the system, the computer is further configured to, responsive to a determination that at least one of the first discrepancy level or the second discrepancy level exceeds a presentation threshold: cause a review interface to be presented to a human reviewer, the review interface comprising a side-by-side comparison of corresponding cytological smears with visual indicators highlighting the differences in analysis results; and receive human input regarding the discrepancies.

In some implementations of the system, the computer is further configured to: collect and store discrepancy data from multiple patients; analyze patterns in the discrepancy data to identify systematic issues in cytological smear preparation or analysis; and generate quality improvement recommendations based on the identified patterns. In some implementations of the system, the computer is further configured to: retrieve medical history data of the first patient from an electronic medical information system; and adjust at least one of the predetermined threshold or the selection of the single cytological smear based on identified medical conditions in the medical history data. In some implementations of the system, the computer is further configured to calculate the combined result of the third and fourth cytological smears utilizing a weighted average comprising applying weights determined by a multi-factor confidence assessment that includes at least two of: classification confidence levels (CCLs), smear preparation quality indicators, statistical power considerations, or confidence levels from human review.

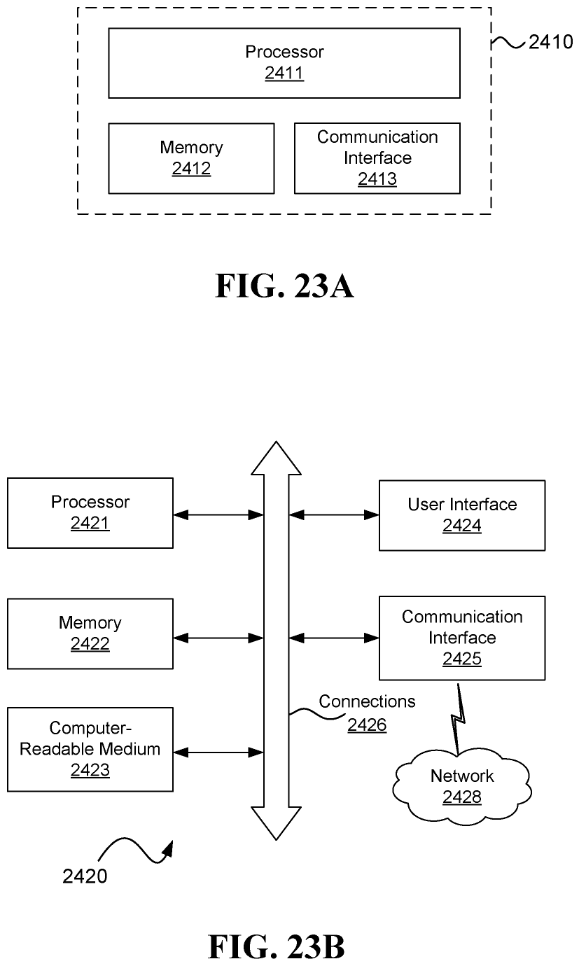

Various embodiments described herein involve interconnected computers. The term “computer” as used herein refers to any device or system that includes some kind of a processor, memory, and a communication interface. The computer may be implemented in various ways, such as, but not limited to, a processor with its required peripherals, a controller (of any type that comprises a processor, memory, and a communication interface), a microcontroller, a computer on a chip, a system-on-chip (SoC), a system-on-module (SOM), a network node, a host computer, a personal computer, a laptops, a tablet, a smartphone, a wearable device, an embedded system, and/or any other computing element capable of executing a set of computer instructions and interfacing for data exchange. Furthermore, references to a computer or a processor include any collection of one or more computers and/or processors (which may be located on different printed circuit boards and/or at different locations) that individually or jointly execute one or more sets of computer instructions. This means that the singular term “computer” is intended to imply one or more computers, which jointly perform the functions attributed to “the computer”. FIG. 23A and FIG. 23B are schematic illustrations of possible embodiments of computers (2410, 2420) that are able to realize one or more of the embodiments discussed herein that include a “computer”. Computer 2410 illustrates a first example of a computer comprising at least the following elements: processor 2411, memory 2412, and a communication interface 2413. The processor may be of any type, including but not limited to, CPU, GPU, embedded controller, accelerator, single-core or multi-core microprocessors, digital signal processors (DSPs), application-specific integrated circuits (ASICs), field-programmable gate arrays (FPGAs), or any combination thereof. The memory component may include any form of volatile or non-volatile, removable or non-removable storage media, such as RAM, ROM, DDR, embedded memory, flash memory, hard drives, or solid-state drives. The communication interface refers to any mechanism that allows the computer to send or receive data, signals, or instructions, and is not limited to external network interfaces but also encompasses internal communication buses and/or interfaces (including internal proprietary interface) that facilitate component-level data exchange. Computer 2420 illustrates a second example of a computer comprising at least the following elements: processor 2421, memory 2422, computer readable medium 2423, user interface 2424, communication interface 2425 optionally coupled to an optional network 2428, and interconnect 2426 such as a bus or any other appropriate structure that connects circuit elements together electrically.

Functionality of various embodiments may be implemented in hardware, software, firmware, or any combination thereof. If implemented at least in part in software, implementing the functionality may involve a computer program that includes one or more instructions or code stored or transmitted on a computer-readable medium and executed by one or more processors. Computer-readable media may include computer-readable storage media, which corresponds to a tangible medium such as data storage media (e.g., SRAM, DRAM, Flash memory), and/or communication media including any medium that facilitates transfer of a computer program from one place to another. Computer-readable medium may be any media that can be accessed by one or more computers to retrieve instructions, code, data, and/or data structures for implementation of the described embodiments. A computer program product may include a computer-readable medium. In one example, the computer-readable medium may include one or more of the following: RAM, ROM, EEPROM, optical storage, magnetic storage, biologic storage, flash memory, or any other medium that can store computer readable data.

In the context of a computer, the term “execute” refers to carrying out operations or implementing functionality performed via software, firmware, hardware, or any combination thereof, including running a program or processing instructions such as executing programming code, performing calculations, implementing algorithms, generating outputs based on inputs, and controlling hardware components. Execution may be implemented through software instructions stored in memory and processed by a processor (for example, when executing an automatic cell classifier), firmware embedded in hardware, dedicated circuitry, FPGAs, ASICs, distributed computing, cloud-based services, or any combination thereof. Execute may refer to scenarios where a computing device initiates, controls, or interfaces with functionality implemented in separate components or services. For instance, when a computer executes an automatic cell classifier, this execution may utilize any implementation method to perform the classification function.

A computer program (also referred to as software, application, script, program code, etc.) may encompass any form of programming language, including but not limited to compiled, interpreted, declarative, and/or procedural languages. It may be deployed in various formats, such as a standalone application or as a module, component, subroutine, object, or other units suitable for a computing environment. It may be represented as a file or part of a file within a file system, possibly coexisting with other programs or data, and/or distributed across multiple files allocated for the program. Furthermore, a computer program may be stored locally on a device, in a network, and/or in the cloud, and may be designed to be executed on one or more computers located at single or multiple locations, optionally connected via one or more communication networks.

Computer-readable medium may include a single medium and/or multiple media (e.g., a centralized or distributed database, and/or associated caches and servers) that store one or more sets of instructions. In various embodiments, a computer program, and/or portions of a computer program, may be stored on a non-transitory computer-readable medium. The non-transitory computer-readable medium may be implemented, for example, via one or more of a volatile computer memory, a non-volatile memory, a hard drive, a flash drive, a magnetic data storage, an optical data storage, and/or any other type of tangible computer memory to be invented that is not transitory signals per se. The computer program may be updated on the non-transitory computer-readable medium and/or downloaded to the non-transitory computer-readable medium via a communication network, such as the Internet. Optionally, the computer program may be downloaded from a central repository, such as an application store. Optionally, the computer program may be downloaded from a repository, such as an open source and/or community run repository (e.g., GitHub).

At least some of the methods described herein are “computer-implemented methods,” which are executed on one or more computing devices, involving performing specific operations by running program instructions on one or more processors within these devices. A computer-implemented method typically requires the interaction of software and hardware components, where the software component is composed of a series of instructions designed to perform a set of tasks or functions. These instructions, when executed by the processor(s), enable the computing device to carry out the method's operations. The execution environment for these methods can vary, encompassing standalone computers, networked systems, cloud-based platforms, or a combination thereof. Additionally, at least some of these instructions may be stored on a non-transitory computer-readable medium.

The methods, algorithms, logics, processes, operations, and system functions described throughout this disclosure are not limited by the particular order, timing, sequence, grouping, or specific implementation described or illustrated unless expressly stated otherwise. Steps, operations, and functions may be performed in any reasonable order, simultaneously or sequentially, in parallel or series, and may be combined, separated, modified, rearranged, omitted, or supplemented based on particular implementation requirements. Any process descriptions, steps, or blocks in flowcharts or other illustrations should be understood as potentially representing modules, segments, portions of code, or operations that may be executed in any reasonable order, combination, or concurrently, and are not necessarily limited to the particular sequence depicted. Alternative implementations and variations are contemplated wherein functional operations may be executed in different orders, segmented differently, combined, distributed across multiple systems or components, or modified according to implementation-specific requirements. The disclosed embodiments can be implemented using various hardware configurations, software architectures, programming languages, data structures, communication protocols, and storage mechanisms, and substitutions, modifications, and alternatives will be apparent to those skilled in the art.

References to “embodiment,” along with its variations like “aspect”, “implementation”, or “case”, indicate that the described features may be present in one or more embodiments of the invention. Additionally, distinct references to embodiments, including terms like “an embodiment,” “another embodiment,” or “some embodiments,” do not necessarily denote separate embodiments. They may describe the same embodiment from different perspectives, highlight various aspects of an embodiment, or pertain to distinct embodiments.

Sentences in the form of “X is indicative of Y” mean that X includes information correlated with Y and/or describing Y, up to the case where X equals Y. Sentences in the form of “provide/receive an indication (of whether X happened)” may refer to any indication method. The word “most” of something is defined as above 51% of the something (including 100% of the something). Words such as portion/subset/region/area of something refer to a value between a fraction of the something and 100% of the something; they indicate an open-ended claim language, thus, for example, a camera said to capture a specific region may capture just a small part of the specific region, the entire specific region, and/or a portion of the specific region together with additional region(s). “Coupled” indicates direct or indirect cooperation and/or interaction, such as direct or indirect physical contact, electrical connection, and/or software and/or hardware interface. The use of “a” or “an” refers to one or more things. The phrase “based on” indicates an open-ended claim language, and is to be interpreted as “based, at least in part, on”. Additionally, stating that a value is calculated “based on X” and following that, in a certain embodiment, that the value is calculated “also based on Y”, means that in the certain embodiment, the value is calculated based on X and Y. Variations of the terms “utilize” and “use” indicate an open-ended claim language, such that sentences in the form of “detecting X utilizing Y” are intended to mean “detecting X utilizing at least Y”, and sentences in the form of “use X to calculate Y” are intended to mean “calculate Y based on X”.

The terms “first”, “second” and so forth are to be interpreted merely as ordinal designations, and shall not be limited in themselves. A predetermined/predefined/preselected value is a fixed value and/or a value determined any time before performing a calculation that utilizes the predetermined value. When appropriate, the word “value” may indicate a “predetermined value”. The word “threshold” indicates a “predetermined threshold”, which means that the value of the threshold, and/or the logic used to determine whether the threshold is reached, is known before start performing computations to determine whether the threshold is reached.

Expressions in the form of “result of a function of a Variable”, such as “result of a function of the CCL”, refer herein to any mathematically or algorithmically derived quantity obtained, at least in part, from the Variable, including a function that returns the Variable itself. Taking CCL as an example, such a function may be calculated by applying one or more arithmetic, logical, statistical, probabilistic or computational operations to the CCL, optionally together with one or more additional parameters. For example, such operations may include scaling, weighting or normalizing the CCL—such as multiplying the CCL by a coefficient, dividing by a reference value or converting the CCL to a percentage; adding, subtracting or otherwise combining the CCL with an offset or bias term; transforming the CCL through linear, non-linear, monotonic or non-monotonic mappings—such as logarithmic, exponential, sigmoid, logit, square-root or power-law transformations; computing a statistical measure or confidence index that incorporates the CCL—such as a moving average, a rolling median, a variance-adjusted score, a Bayesian posterior probability or a z-score; generating an aggregate or composite metric that merges the CCL with one or more other features, such as image-quality indicators, cell-population statistics or patient-specific parameters; or evaluating the CCL within a machine-learning, rule-based, look-up table, or fuzzy-logic model to produce a derived value indicative of overall classification reliability. Unless expressly stated otherwise, no limitation is placed on the nature, complexity or number of operations used by the function of the Variable, nor on the hardware, firmware or software in which those operations may execute. Accordingly, any comparison that employs the Variable itself or any value derived therefrom by one or more of the foregoing operations falls within the scope of “result of a function of the Variable”. “result of a function of” may further refer to group operations, for example “result of a function of classifications” may refer to the calculation of HCQMs for different cell types using the classifications.

The embodiments of an invention may include any variety of combinations and/or integrations of the features of the embodiments described herein. Although some embodiments may describe serial operations, the embodiments may perform certain operations in parallel and/or in different orders from those described. Moreover, the use of repeated reference numerals and/or letters in the text and/or drawings is for the purpose of simplicity and clarity and does not in itself dictate a relationship between the various embodiments and/or configurations discussed. Components and/or modules referred to by different reference numerals may or may not perform the same (or similar) functionality, and the fact they are referred to by different reference numerals and/or letters does not mean that they may not have same or similar functionalities.

Certain features of the embodiments, which may have been, for clarity, described in the context of separate embodiments, may also be provided in various combinations in a single embodiment. Conversely, various features of the embodiments, which may have been, for brevity, described in the context of a single embodiment, may also be provided separately or in any suitable sub-combination. Embodiments described in conjunction with specific examples are presented by way of example, and not limitation. Moreover, it is evident that many alternatives, modifications, and variations will be apparent to those skilled in the art. It is to be understood that other embodiments may be utilized and structural changes may be made without departing from the scope of the embodiments.

The drawings depict some, but not necessarily all, the connections between elements. The depiction of elements as separate entities may be done to emphasize different functionalities of elements that may be implemented by the same software and/or hardware. Programs and/or elements illustrated and/or described as being single may be implemented via multiple programs and/or involve multiple hardware elements possibly in different locations. The embodiments are not limited in their applications to the details of order, or sequence of method steps, or to details of implementation of the devices, set in the description, drawings, or examples. Individual blocks illustrated in the drawings may be functional in nature and therefore may not necessarily correspond to discrete hardware elements. Moreover, the organization of this disclosure into sections, potentially titled, aims to enhance readability without constraining the scope, allowing for the integration of elements, features, components, and steps across various sections.

Accordingly, this disclosure is intended to embrace all such alternatives, modifications, and variations that fall within the spirit and scope of the appended claims and their equivalents.

We claim:

1. A computer-implemented method for cytological smear human review workload management, comprising:
receiving a first image of a first cytological smear associated with a first sample obtained from a first patient;
receiving a second image of a second cytological smear associated with a second sample obtained from a second patient;
determining, by an automatic cell classifier, from the first image, first and second hematological cell or cell type quantification metrics (HCQMs) for the first patient, wherein the first and second HCQMs are for first and second abnormal cell types, respectively, and wherein the second abnormal cell type is different from the first abnormal cell type;
determining, by the automatic cell classifier, from the second image, the first and second HCQMs for the second patient;
receiving first and second predetermined thresholds associated with the first and second HCQMs, respectively;
comparing the first and second HCQMs for the first patient with the first and second predetermined thresholds, respectively, and identifying that the first HCQM is above the first predetermined threshold and the second HCQM is below the second predetermined threshold;
comparing the first and second HCQMs for the second patient with the first and second predetermined thresholds, respectively, and identifying that the first HCQM is below the first predetermined threshold and the second HCQM is above the second predetermined threshold;
presenting, as part of a default human review workflow, cells associated with the first sample; and
not presenting, as part of the default human review workflow, cells associated with the second sample.

2. The method of claim 1, wherein the first abnormal cell type is selected from: blast cell, aberrant lymphocyte, promyelocyte, atypical lymphocyte, Sézary cell, hairy cell, large granular lymphocyte, Pelger-Huet cell.

3. The method of claim 2, wherein the second abnormal cell type is selected from: blast cell, aberrant lymphocyte, promyelocyte, atypical lymphocyte, Sézary cell, hairy cell, large granular lymphocyte, Pelger-Huet cell, plasma cell, schistocyte, smudge cell, helmet cell, bite cell, blister cell or cell containing a microorganism.

4. The method of claim 2, wherein the second abnormal cell type is selected from: myelocyte, metamyelocyte, nucleated red blood cell, sickle cell or cell with Howell-Jolly body.

5. The method of claim 1, wherein the first abnormal cell type is selected from: myelocyte, metamyelocyte, plasma cell, schistocyte, smudge cell, helmet cell, bite cell, nucleated red blood cell, blister cell or cell containing a microorganism.

6. The method of claim 1, further comprising: generating a first report based at least on the first and second HCQMs for the first patient; generating a second report based at least on the first and second HCQMs for the second patient; and automatically transmitting the second report to an electronic medical information system.

7. The method of claim 1, further comprising: receiving a third image of a third cytological smear associated with a third sample obtained from a third patient; receiving a fourth image of a fourth cytological smear associated with a fourth sample obtained from a fourth patient; determining, by the automatic cell classifier, from the third image, a quantity of unclassified cells for the third patient; determining, by the automatic cell classifier, from the fourth image, a quantity of unclassified cells for the fourth patient; receiving a predetermined unclassified cell threshold; comparing the quantity of unclassified cells for the third patient with the predetermined unclassified cell threshold and identifying that the quantity of unclassified cells for the third patient is above the predetermined unclassified cell threshold; comparing the quantity of unclassified cells for the fourth patient with the predetermined unclassified cell threshold and identifying that the quantity of unclassified cells for the fourth patient is below the predetermined unclassified cell threshold; presenting, as part of the default human review workflow, cells associated with the third sample; and not presenting, as part of the default human review workflow, cells associated with the fourth sample.

8. The method of claim 1, further comprising: monitoring human review results over time for multiple cytological smears; and adjusting one or more of the first and second predetermined thresholds based on the monitoring, wherein the adjusting comprises increasing the first predetermined threshold to reduce a number of smears presented for human review.

9. The method of claim 8, wherein the monitoring comprises tracking a rate of agreement between automated analysis results and human review results; and wherein the adjusting comprises automatically increasing the first predetermined threshold when the rate of agreement exceeds a predetermined agreement threshold.

10. The method of claim 8, wherein the adjusting comprises: determining cell type-specific agreement rates between automated analysis results and human review results for different abnormal cell types; and selectively increasing thresholds for specific abnormal cell types based on their respective agreement rates.

11. A non-transitory computer-readable medium comprising instructions that, when executed by a processor, cause the processor to perform the computer-implemented method of claim 1.

12. A system for cytological smear human review workload management, comprising:
a computer configured to:
receive a first image of a first cytological smear associated with a first sample obtained from a first patient;
receive a second image of a second cytological smear associated with a second sample obtained from a second patient;
determine, by an automatic cell classifier, from the first image, first and second hematological cell or cell type quantification metrics (HCQMs) for the first patient, wherein the first and second HCQMs are for first and second abnormal cell types, respectively, and wherein the second abnormal cell type is different from the first abnormal cell type;
determine, by the automatic cell classifier, from the second image, the first and second HCQMs for the second patient;
receive first and second predetermined thresholds associated with the first and second HCQMs, respectively;
compare the first and second HCQMs for the first patient with the first and second predetermined thresholds, respectively, and identify that the first HCQM is above the first predetermined threshold and the second HCQM is below the second predetermined threshold;
compare the first and second HCQMs for the second patient with the first and second predetermined thresholds, respectively, and identify that the first HCQM is below the first predetermined threshold and the second HCQM is above the second predetermined threshold;
cause cells associated with the first sample to be presented as part of a default human review workflow; and
cause cells associated with the second sample to be excluded from presentation as part of the default human review workflow.

13. The system of claim 12, wherein the first abnormal cell type is selected from: blast cell, aberrant lymphocyte, promyelocyte, atypical lymphocyte, Sézary cell, hairy cell, large granular lymphocyte, Pelger-Huet cell.

14. The system of claim 12, wherein the first abnormal cell type is selected from: myelocyte, metamyelocyte, plasma cell, schistocyte, smudge cell, helmet cell, bite cell, nucleated red blood cell (RBC), blister cell or cell containing a microorganism; and wherein the second abnormal cell type is selected from: myelocyte, metamyelocyte, nucleated RBC, sickle cell or cell with Howell-Jolly body.

15. The system of claim 12, wherein the computer is further configured to: monitor human review results over time for multiple cytological smears; and adjust one or more of the first and second predetermined thresholds based on the monitoring, wherein the adjusting comprises increasing the first predetermined threshold to reduce a number of smears presented for human review.

16. The system of claim 12, wherein the computer is further configured to: receive a third image of a third cytological smear associated with a third sample obtained from a third patient; receive a fourth image of a fourth cytological smear associated with a fourth sample obtained from a fourth patient; determine, by the automatic cell classifier, from the third image, a quantity of unclassified cells for the third patient; determine, by the automatic cell classifier, from the fourth image, a quantity of unclassified cells for the fourth patient; receive a predetermined unclassified cell threshold; compare the quantity of unclassified cells for the third patient with the predetermined unclassified cell threshold and identify that the quantity of unclassified cells for the third patient is above the predetermined unclassified cell threshold; compare the quantity of unclassified cells for the fourth patient with the predetermined unclassified cell threshold and identify that the quantity of unclassified cells for the fourth patient is below the predetermined unclassified cell threshold; cause cells associated with the third sample to be presented as part of the default human review workflow; and cause cells associated with the fourth sample to be excluded from presentation as part of the default human review workflow.

17. A computer-implemented method for adaptive management of cytological smear human review workload, comprising:
receiving results from an automatic cell classifier for cytological smears of multiple patients over time;
monitoring performance of the automatic cell classifier by comparing the results with human review results;
adjusting one or more predetermined thresholds for presentation of cells for human review based on the monitored performance, wherein the one or more predetermined thresholds are associated with hematological cell or cell type quantification metrics (HCQMs) for abnormal cell types; and
increasing at least one of the one or more predetermined thresholds when the performance of the automatic cell classifier improves beyond a predetermined performance level, thereby reducing a number of cytological smears presented for human review while maintaining analysis quality.

18. The method of claim 17, further comprising: tracking a rate of agreement between the results from the automatic cell classifier and the human review results; and automatically increasing, or suggesting increasing, the at least one of the one or more predetermined thresholds when the rate of agreement exceeds a predetermined agreement threshold.

19. The method of claim 17, wherein the adjusting comprises: determining cell type-specific agreement rates between the results from the automatic cell classifier and the human review results for different abnormal cell types; and selectively increasing thresholds for specific abnormal cell types based on their respective agreement rates.

20. The method of claim 17, wherein a number of cytological smears presented for human review is less than 15% of a total number of cytological smears analyzed by the automatic cell classifier.
